(19) United States
(12) Patent Application Publication
(10) Pub. No.: US 2024/0409598 A1
(43) Pub. Date: Dec. 12, 2024

(54) PEPTIDE INHIBITORS OF INTERLEUKIN-23 RECEPTOR

(71) Applicants: Janssen Biotech, Inc., Horsham, PA (US); Protagonist Therapeutics, Inc., Newark, CA (US)

(72) Inventors: Stephanie A. Barros, Chalfont, PA (US); Charles Hendrick, North Wales, PA (US); Santhosh Neelamkavil, Edison, NJ (US); Raymond J. Patch, Yardley, PA (US); Sandeep Somani, Princeton, NJ (US); Chengzao Sun, Warrington, PA (US); Jing Zhang, Wilmington, DE (US); Elisabetta Bianchi, Pomezia (Rome) (IT); Danila Branca, Pomezia (Rome) (IT); Roberto Costante, Pomezia (Rome) (IT); Raffaele Ingenito, Pomezia (Rome) (IT); Ashok Bhandari, Pleasanton, CA (US); Brian Frederick, Ben Lomond, CA (US); James Daniel, Newark, CA (US); Tran Trung Tran, Newark, CA (US)

(21) Appl. No.: 18/579,062

(22) PCT Filed: Jul. 14, 2022
(86) PCT No.: PCT/US2022/037221

§ 371 Date: Jan. 12, 2024

Related U.S. Application Data
(60) Provisional application No. 63/221,806, filed on Jul. 14, 2021.

(51) Int. Cl. C07K 14/54; A61K 38/00

(52) U.S. Cl. CPC C07K 14/54; A61K 38/00

(57) ABSTRACT

The present invention relates to novel cyclic peptide inhibitors of the interleukin-23 receptor (IL-23R) or pharmaceutically acceptable salt thereof, corresponding pharmaceutical compositions, methods and/or uses for treatment of autoimmune inflammation and related diseases and disorders. The inhibitor of an interleukin-23 receptor is cyclized by a disulfide bond between penicillamine, cysteine, homocysteine, or alpha methylcysteine residues at positions X4 and X9.

### CROSS-REFERENCE TO RELATED APPLICATION

This application claims the benefit under 35 U.S.C. § 119 of U.S. Provisional Application No. 63/221,806, filed Jul. 14, 2021 (pending), which is herein incorporated by reference in its entirety, including its respective sequence listing.

### PARTIES TO A JOINT RESEARCH AGREEMENT

The present disclosure was made by, or on behalf of, the below listed parties to a joint research agreement. The joint research agreement was in effect on or before the date the claimed invention was made, and the claimed invention was part of the joint research agreement and made as a result of activities undertaken within the scope of the joint research agreement. The parties to the joint research agreement are JANSSEN BIOTECH, INC. and PROTAGONIST THERAPEUTICS, INC.

### INCORPORATION OF SEQUENCE LISTING

The sequence listing in ST.26 XML format entitled 2948-22_ST26.xml, created on Jul. 13, 2022, comprising 2,769,116 bytes, prepared according to 37 CFR 1.822 to 1.824, submitted concurrently with the filing of this application, is incorporated herein by reference in its entirety.

### FIELD OF THE INVENTION

The present invention relates to novel peptide inhibitors of the interleukin-23 receptor (IL-23R) or pharmaceutically acceptable salts, solvates and/or other forms thereof, invention relates to corresponding pharmaceutical compositions, methods and/or uses of the IL-23R inhibitors for treatment of inflammatory, autoimmune inflammation diseases and/or related disorders.

### BACKGROUND

The interleukin-23 (IL-23) cytokine has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs), for example, ulcerative colitis and Crohn's disease. Studies in acute and chronic mouse models of IBD revealed a primary role of interleukin-23 receptor (IL-23R) and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

Production of IL-23 is enriched in the intestine, where it is believed to play a key role in regulating the balance between tolerance and immunity through T-cell-dependent and T-cell-independent pathways of intestinal inflammation through effects on T-helper 1 (Th1) and Th17-associated cytokines, as well as restraining regulatory T-cell responses in the gut, favoring inflammation. In addition, polymorphisms in the IL-23 receptor (IL-23R) have been associated with susceptibility to inflammatory bowel diseases (IBDs), further establishing the critical role of the IL-23 pathway in intestinal homeostasis.

Psoriasis, a chronic skin disease affecting about 2%-3% of the general population has been shown to be mediated by the body's T cell inflammatory response mechanisms. IL-23 has one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and IL-23R has been shown to be increased in tissues of patients with psoriasis, and antibodies that neutralize IL-23 showed IL-23-dependent inhibition of psoriasis development in animal models of psoriasis.

IL-23 is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant, presumably due to a reduced number of CD4⁺ T cells producing IL-6, IL-17, and TNF in the CNS of IL-23-deficient animals. IL-23 binds to IL-23R, which is a heterodimeric receptor composed of IL-12Rβ1 and IL-23R subunits. Binding of IL-23 to IL-23R activates the Jak-Stat signaling molecules, Jak2, Tyk2, and Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4 (+) T cells, IL-23 preferentially acts on memory CD4 (+) T cells.

Therapeutic moieties that inhibit the IL-23 pathway have been developed for use in treating IL-23-related diseases and disorders. A number of antibodies that bind to IL-23 or IL-23R have been identified, including ustekinumab, which has been approved for the treatment of moderate to severe plaque psoriasis (PSO), active psoriatic arthritis (PSA), moderately to severely active Crohn's disease (CD) and moderately to severely active ulcerative colitis (UC). Examples of such identified antibodies, include: Tildrakizumab, an anti-IL23 antibody approved for treatment of plaque psoriasis, Guselkumab, an anti-IL23 antibody approved for treatment of psoriatic arthritis and Risankizumab, an anti-IL23 antibody approved for the treatment of plaque psoriasis in the US, and generalized pustular psoriasis, erythrodermic psoriasis and psoriatic arthritis in Japan.

Although targeted IL-23 antibody therapeutics are used clinically, there are no small-molecule therapeutics that selectively inhibit IL-23 signaling. There are some identified polypeptide inhibitors that bind to IL-23R and inhibit binding of IL-23 to IL-23R (see, e.g., US Patent Application Publication No. US2013/0029907). Thus, there remains a significant need in the art for effective small-molecule and/or polypeptide therapeutic agents to treat and/or prevent IL-23-associated and/or IL23R-associated diseases and disorders, which include, but are not limited to psoriasis (PsO), psoriatic arthritis (PsA), inflammatory bowel diseases (IBD), ulcerative colitis (UC), and Crohn's disease (CD).

In particular:
compounds and methods for specific targeting of IL-23R from the luminal side of the gut may provide therapeutic benefit to IBD patients suffering from local inflammation of the intestinal tissue; and/or
orally bioavailable small molecule and/or polypeptide inhibitors of IL-23 may provide both a non-steroidal treatment option for patients with mild to moderate psoriasis and treatment for moderate to severe psoriasis that does not require delivery by infusion.

Compounds and methods for specific targeting of the IL-23R from the luminal side of the gut may provide therapeutic benefit to IBD patients suffering from local inflammation of the intestinal tissue. In addition, orally bioavailable small molecule and/or polypeptide inhibitors of IL-23 may provide both a non-steroidal treatment option for patients with mild to moderate psoriasis and treatment for moderate to severe psoriasis that does not require delivery by infusion.

The present invention is directed to addressing these needs by providing peptide inhibitors or pharmaceutically acceptable salts, solvates and/or other forms thereof, that bind IL-23R to inhibit IL-23 binding and signaling, via different suitable routes of administration, which may include but is not limited to oral administration.

### BRIEF SUMMARY

In general, the present invention relates to novel peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of inflammatory, autoimmune inflammation diseases and/or related disorders.

In particular, the present invention relates to a compound of Formulas (I′), (I) to (III)), or pharmaceutically acceptable salts, solvates and/or other forms thereof. corresponding pharmaceutical compositions, methods and/or uses for treatment of inflammatory, autoimmune inflammation diseases and/or related disorders.

The peptide inhibitor(s) of the IL-23R of the present invention is represented by linear form structure of Formula (I′):

R1-X3-X4-X5-X6-X7-X8-X9-X10-X11-X12-X13-X14-X15-X16-X17-R2 (I′)

The linear form structure of Formula (I′) is intended for exemplary and non-limiting purposes, which will be apparent from examples set forth and exemplified throughout the instant specification, i.e., e.g., where each such structure may be longer or shorter than the length of eighteen amino acids and/or other corresponding chemical moieties or functional group substituents as defined herein.

Specifically in Formula (I′) of the present invention:
X3-X17, respectively and individually, represent individual amino acid (aa) residues or other corresponding chemical moieties or functional group substituents as described below and in the instant invention;
R1 represents the N-terminal end, which may be, for example a hydrogen or a chemical moiety or functional group substituted on the amino group;
Similarly, R2 represents the carboxyl end, which may be, for example the OH of the carboxyl or a chemical moiety or functional group attached thereto or substituted for the OH group (e.g., an amino group to give a terminal amide e.g., —C(O)HN₂);
Any of the residues as shown in the linear form structure may be present or absent, i.e., e.g., X3 and/or X16-X18 may be absent;
In certain aspects, the peptide inhibitors may have:
a bond between positions X4 and X9 (e.g., a pair of Pen residues or Abu and Cys residues) forming a disulfide bond or thioether bond resulting in formation of a first ring structure; and/or
however the bond forming the first ring structure may, be located between other amino acids or chemical moieties besides X4 and X9; and In other aspects, the peptide inhibitors may have:
a bond forming a second ring structure resulting in a ring that bridges the first ring structure or a separate ring structure connected by an intervening portion of the molecule.

The present invention relates to compounds of Formulas (I′), (I) to (X) or pharmaceutically acceptable salts, solvates, or forms thereof, corresponding pharmaceutical compositions methods and/or uses for inflammatory, autoimmune inflammation diseases and/or related disorders.

In particular, the present invention relates to peptide inhibitor of the IL-23R or a pharmaceutically acceptable salt(s), solvate(s) or other form(s) thereof, corresponding pharmaceutical compositions, methods and/or uses for treatment of disease including inflammatory, autoimmune inflammation diseases and/or related disorders s. In particular, an inhibitor of the IL-23R of the present invention is identified:
Formulas (I′), (I) to (XX) in the instant specification and disclosure; and/or
in Table 1A, Table 1B, Table 1C, Table 1D, Table 1E, Table 1F, Table 1G, Table 1H, and Table 1I, respectively, of the present specification.

In an aspect, the present invention relates to compounds which are inhibitors of an IL-23 receptor comprising an amino acid sequence of Formula (I)

R1-X3-X4-X5-X6-X7-X8-X9-X10-X11-X12-X13-X14-X15-X16-R2 (I)

where:
R1 is hydrogen, CH₃C(O)—, EtC(O)—, MeSO2, AzCO, BHCO, FPrpTriazoleMeCO, SMSBCO, Biotin, BiotinPEG2PEG2CO or DAGSuc;
X3 is absent or dR, dK, PEG6, gEPEG6;
X4 is Pen, aMeC, hC, or C;
X5 is A, N, Q, N-MeAsn, L, Asn(4C13_2N15), I or K (PEG2PEG2Biotin);
X6 is T, MeThr, V, K, Dbu, Dpr, or A;
X7 is W7Me, W, W(4F7Me), 7MeW, 7PhW, 7EtW, 7FW, 7ClW, 5BrW or, 7(3NAcPh)W′
X8 is KAc, Q, N-MeGln, A, or Cit;
X9 is Pen, aMeC, hC, or C;
X10 is F4OMe, AEF, F, F4Me, F4Ad, Nal, AEF(Boc), 4PipPhe, AEF(Ac), Y, 4OMeF, 4AmF, D(Pip), Tzl(mPEG3), 3FTyr, Y(OTzl), Y(OTzl(mPEG3)), Tzl, or Tzl(PEG3OH);
X11 is Nal, Quin_3, Coumarin(7OMe), 2Nal, or 3Quin;
X12 is aMeK, THP, Spiral_Pip_Ac, Spiral_Pip, MeK, aMeLeu, aMeL, or aMeK(Boc)
X13 is KAc, K;
X14 is A, N, L, N-MeAsn, MeLeu, Asn(4C13_2N15), or I;
X15 is absent, 3Pya, bAla, Thiozolidine, H, dL, N, A, F, aMePhe, Aib, dK, h, 3MeH, 1MeH, tetraFPhe, bMePhe(SR), 5PyrimidAla, v, dR, homoF, Y, y, F(CF3), Y(CHF2), or THP;
X16 is MeGly, dL, MeLeu, N-MeNle, y, paf, maf, D3Pya, bAla, P, N(3AmBenzyl)Gly, N(4AmBenzyl)Gly, 4(R)HydroxyPro, 4(S)AminoPro, 5(R)diMePro, or absent
R2 is —OH, —NH₂, —HN(C₁-C₄ alkyl), —N(C₁-C₄ alkyl)₂, MeNH, or CONHMe; and
   where:
the inhibitor of an interleukin-23 receptor is cyclized by forming: a disulfide bond between penicillamine, cysteine, homocysteine, or alpha methylcysteine residues at positions X4 and X9.

The present invention also relates to compounds of Formula I or pharmaceutically acceptable salts, solvates and/or other forms thereof, corresponding pharmaceutical compositions, methods and/or uses for treatment of autoimmune inflammation diseases and related disorders.

The present invention also relates to compounds of Formula II-XVIII, respectively, or pharmaceutically acceptable salts, solvates and/or other forms thereof, corresponding pharmaceutical compositions, methods and/or uses for treatment of inflammatory, autoimmune inflammation diseases and/or related disorders.

The present invention also relates to compounds set forth in any of Tables 1A-I, respectively or pharmaceutically acceptable salts, solvates and/or other forms thereof, corresponding pharmaceutical compositions, methods and/or uses for treatment of inflammatory, autoimmune inflammation diseases and/or related disorders.

The present invention also relates to pharmaceutical composition(s), which comprises a peptide inhibitor compound of the present invention or a pharmaceutically acceptable salt, solvate, or form thereof as described herein, and a pharmaceutically acceptable carrier, excipient, or diluent.

The present invention further relates to use or inclusion of one or more compounds (i.e., e.g., compounds of formulas (I) to (X), Tables 1A to 1I or as defined herein for preparation of pharmaceutical compositions, which may be used for treatment of inflammatory, autoimmune inflammation diseases and/or related disorders as defined herein.

The pharmaceutical compositions of the present invention also may comprise or may exclude an absorption enhancer depending on the intended route of delivery or use thereof for treatment of specific indications. The absorption enhancer may be a permeation enhancer and/or an intestinal permeation enhancer. In one aspect, the absorption enhancer improves oral bioavailability.

The present invention relates to method(s) and/or uses(s) for treating inflammatory, autoimmune inflammation diseases and/or related disorders which comprises administering:
a therapeutically effective amount of one or more herein-described peptide inhibitor compounds of the IL-23R or pharmaceutically acceptable salts, solvates and/or other forms thereof; or
a corresponding pharmaceutical composition, respectively
to a subject or patient in need thereof.

Such inflammatory, autoimmune inflammation diseases and/or related disorders contemplated for use with or defined in the present invention, may include, but are not limited to, inflammatory bowel disease (IBD), Crohn's disease (CD), ulcerative colitis (UC), psoriasis (PsO), or psoriatic arthritis (PsA) and the like.

The present invention provides for the use of one or more herein-described compounds of formulas (I) to (X) or Tables 1A to 1I in the treatment of inflammatory, autoimmune inflammation diseases and/or related disorders as defined herein.

The present invention provides for kits comprising one or more herein-described compounds of formulas (I) to (X) or Tables 1A to 1I and instructions for use in treating a disease, inflammatory, autoimmune inflammation diseases and/or related disorder sin a patient or subject in need thereof.

### DETAILED DESCRIPTION

### I. General

The present invention relates to novel peptide inhibitors of the IL-23R or pharmaceutically acceptable salt thereof, corresponding pharmaceutical compositions, methods and/or uses for treatment inflammatory, autoimmune inflammation diseases and/or related disorders. The present invention provides or relates to peptide inhibitors of an IL-23R. The peptide inhibitors of the present invention may exhibit enhanced properties, such as longer in vivo half-life, compared to the corresponding cyclic peptide inhibitor of an IL-23R without a cyclic structure.

### II. Definitions

Unless otherwise defined herein, scientific and technical terms used in this application shall have the meanings that are commonly understood by those of ordinary skill in the art.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Patient” or “subject”, which are used interchangeably, refer to a living organism, which includes, but is not limited to a human subject suffering from or prone to a disease or condition that can be treated by administration of a pharmaceutical composition as provided herein. Further non-limiting examples may include, but is not limited to humans, other mammals, bovines, rats, mice, dogs, monkeys, goat, sheep, cows, deer, horse, and other mammalian animals and the like. In some aspects, the patient is human.

Unless indicated otherwise the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14 (2), (1975). To the extent that the names and abbreviations of amino acids and aminoacyl residues employed in this specification and appended claims differ from those suggestions, they will be made clear to the reader. In sequences of amino acids that represent IL-23 inhibitors the individual amino acids are separated by a hyphen “-” or brackets e.g. lysine is shown as [K].

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., (D) Asp or D-Asp; (D) Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, L-arginine can be represented as “Arg” or “R.” while D-arginine can be represented as “arg” or “r.” Similarly, L-lysine can be represented as “Lys” or “K.” while D-lysine can be represented as “lys” or “k.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and Abu (2-amino butyric acid).

Amino acids of the D-isomeric form may be located at any of the positions in the IL-23R inhibitors set forth herein (any of X1-X18 appearing in the molecule). In an aspects, amino acids of the D-isomeric form may be located only at any one or more of X3, X5, X6, X8, X13, and optionally one additional position. In other aspects, amino acids of the D-isomeric form may be located only at any one or more of X3, X8, X13, and optionally one additional position. In other aspects, amino acids of the D-isomeric form may be located only at any one or more of X8, X13 (e.g., X8 is dK(Ac) and X13 is dE), and optionally one additional position. In other aspects, amino acids of the D-isomeric form may be located only at X3, and optionally one additional position. In other aspects, amino acids of the D-isomeric form may be located only at X3, and optionally two or three additional positions. In other aspects, amino acids of the D-isomeric form may be located at only one or two of positions X1 to X18 appearing in the IL-23R inhibitors set forth herein. In other aspects, amino acids of the D-isomeric form may be located at only three or four of positions X1 to X18 appearing in the IL-23R inhibitors set forth herein. For example an IL-23R inhibitors set forth herein having only positions X3 to X15 present may have amino acids of the D-form present in 3 or four of those positions. In other aspects, amino acids of the D-isomeric form may be located at only five or six of positions X1 to X18 appearing in the IL-23R inhibitors set forth herein.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the invention, a C-terminal “—OH” moiety may be substituted for a C-terminal “—NH₂” moiety, and vice-versa.

One of skill in the art will appreciate that certain amino acids and other chemical moieties are modified when bound to another molecule. For example, an amino acid side chain may be modified when it forms an intramolecular bridge with another amino acid side chain, e.g., one or more hydrogen may be removed or replaced by the bond.

A “compound of the invention”, an “inhibitor of the present disclosure”, an “IL-23R inhibitor of the present disclosure”, a “compound described herein”, and a “herein-described compound” may include, but are not limited to novel compounds disclosed herein, for example the compounds of any of the Examples, i.e., e.g., which may include compounds of Formula (I) to (X). i.e., e.g., such as those found in Table 1A, Table 1B, Table 1C, Table 1D, Table 1E, Table 1F, Table 1G, Table 1H, or Table 1I.

“Pharmaceutically effective amount” refers to an amount of a compound of the invention in a composition or combination thereof that provides the desired therapeutic or pharmaceutical result.

By “pharmaceutically acceptable” it is meant the carrier(s), diluent(s), salt(s), solvate(s) or excipient(s) must be compatible with the other components or ingredients of the compositions of the present invention, i.e., that which is useful, safe, non-toxic acceptable for pharmaceutical use. In accordance with the present invention pharmaceutically acceptable means approved or approvable as is listed in the U.S. Pharmacopoeia or other generally recognized pharmacopoeia for use in animals, and more particularly, in humans.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Absorption enhancer” refers to a component that improves or facilitates the mucosal absorption of a drug in the gastrointestinal tract, such as a permeation enhancer or intestinal permeation enhancer. As conventionally understood in the art, permeation enhancers (PEs) are agents aimed to improve oral delivery of therapeutic drugs with poor bioavailability. PEs are capable of increasing the paracellular and/or transcellular passage of drugs.

Pharmaceutical excipients that can increase permeation have been termed “absorption modifying excipients” (AMEs). AMEs may be used in oral compositions, for example, as wetting agents (sodium dodecyl sulfate), antioxidants (e.g. EDTA), and emulsifiers (e.g. macrogol glycerides), and may be specifically included in compositions as PEs to improve bioavailability. PEs can be categorized as to how they alter barrier integrity via paracellular or transcellular routes.

“Intestinal permeation enhancer (IPE)” refers to a component that improves the bioavailability of a component. Suitable representative IPEs for use in the present invention, include, but are not limited to, various surfactants, fatty acids, medium chain glycerides, steroidal detergents, acyl carnitine and alkanoylcholines, N-acetylated alpha-amino acids and N-acetylated non-alpha-amino acids, and chitosans, other mucoadhesive polymers and the like. For example, a suitable IPE for use in the present invention may be sodium caprate.

“Composition” or “Pharmaceutical Composition” as used herein is intended to encompass an invention or product comprising the specified active product ingredient (API), which may include pharmaceutically acceptable excipients, carriers or diluents as described herein, such as in specified amounts defined throughout the disclosure. Compositions or Pharmaceutical Compositions result from combination of specific components, such as specified ingredients in the specified amounts as described herein.

Compositions or pharmaceutical compositions of the present invention may be in different pharmaceutically acceptable forms, which may include, but are not limited to a liquid composition, a tablet or matrix composition, a capsule composition, etc. and the like. When the composition is a tablet composition, the tablet may include, but is not limited to different layers two or more different phases, including an internal phase and an external phase that can comprise a core. The tablet composition can also include, but is not limited to one or more coatings.

“Solvate” as used herein, means a physical association of the compound of the present invention with one or more solvent molecules. This physical association involves varying degrees bonding, including hydrogen bonding. In certain instances, the solvate will be capable of isolation. The term “solvate” is intended to encompass both solution-phase and isolatable solvates. Non-limiting examples of suitable solvates include hydrates.

Provided are also pharmaceutically acceptable salts and tautomeric forms of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The IL-23R inhibitors of the present invention, pharmaceutically acceptable salts, solvates and/or other forms thereof may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms of the IL-23R inhibitors of the present disclosure. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s). As used herein, “scalemic mixture” is a mixture of stereoisomers enantiomers at a ratio other than 1:1.

“Racemates” refers to a mixture of enantiomers. The mixture can include equal or unequal amounts of each enantiomer.

“Stereoisomer” and “stereoisomers” refer to compounds that differ in the chirality of one or more stereo centers. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring-NH— and a ring=N— such as pyrazoles, imidazoles, benzimidazoles, triazoles, and tetrazoles.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

“Treatment” or “treat” or “treating” as used herein refers to an approach for obtaining beneficial or desired results. For purposes of the present invention, beneficial or desired results include, but are not limited to, alleviation of a symptom and/or diminishment of the extent of a symptom and/or preventing a worsening of a symptom associated with a disease or condition. In one aspect, “treatment” or “treating” includes one or more of the following: (a) inhibiting the disease or condition (e.g., decreasing one or more symptoms resulting from the disease or condition, and/or diminishing the extent of the disease or condition); (b) slowing or arresting the development of one or more symptoms associated with the disease or condition (e.g., stabilizing the disease or condition, delaying the worsening or progression of the disease or condition); and (c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of a compound that, when administered to a subject for treating a disease is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some aspects, a unit dose of a compound of the invention is administered first, followed within seconds or minutes by administration of a unit dose of one or more additional therapeutic agents. Alternatively, in other aspects, a unit dose of one or more additional therapeutic agents is administered first, followed by administration of a unit dose of a compound of the invention within seconds or minutes. In some aspects, a unit dose of a compound of the invention is administered first, followed, after a period of hours (e.g., 1-12 hours), by administration of a unit dose of one or more additional therapeutic agents. In other aspects, a unit dose of one or more additional therapeutic agents is administered first, followed, after a period of hours (e.g., 1-12 hours), by administration of a unit dose of a compound of the invention. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

Abbreviation, “(w/w)” refers to the phrase “weight for weight”, i.e., the proportion of a particular substance within a mixture, as measured by weight or mass or a weight amount of a component of the composition disclosed herein relative to the total weight amount of the composition. Accordingly, the quantity is unit less and represents a weight percentage amount of a component relative to the total weight of the composition. For example, a 2% (w/w) solution can indicate 2 grams of solute is dissolved in 100 grams of solution.

Systemic routes of administration as conventionally understood in the medicinal or pharmaceutical arts, refer to or are defined as a route of administration of drug, a pharmaceutical composition or formulation, or other substance into the circulatory system so that various body tissues and organs are exposed to the drug, formulation or other substance. As conventionally understood in the art, administration can take place orally (where drug or oral preparations are taken by mouth, and absorbed via the gastrointestinal tract), via enteral administration (absorption of the drug also occurs through the gastrointestinal tract) or parenteral administration (generally injection, infusion, or implantation, etc.

“Systemically active” peptide drug therapy as it relates to the present invention generally refers to treatment by means of a pharmaceutical composition comprising a peptide active ingredient, wherein said peptide resists immediate metabolism and/or excretion resulting in its exposure in various body tissues and organs, such as the cardiovascular, respiratory, gastrointestinal, nervous or immune systems.

Systemic drug activity in the present invention also refers to treatment using substances that travel through the bloodstream, reaching and affecting cells in various body tissues and organs. Systemic active drugs are transported to their site of action and work throughout the body to attack the physiological processes that cause inflammatory diseases.

“Bioavailability” refers to the extent and rate at which the active moiety (drug or metabolite) enters systemic circulation, thereby accessing the site of action. Bioavailability of a drug is impacted by the properties of the dosage form, which depend partly on its design and manufacture.

“Digestive tract tissue” as used herein refers to all the tissues that comprise the organs of the alimentary canal. For example only, and without limitation, “digestive tract tissue” includes tissues of the mouth, esophagus, stomach, small intestine, large intestine, duodenum, and anus.

### III. Compounds

The present invention relates to novel cyclic peptide inhibitors of the interleukin-23 receptor (IL-23R) or pharmaceutically acceptable salt thereof.

In particular, the present invention relates to a cyclic peptide inhibitors of the interleukin-23 receptor (IL-23R) or a pharmaceutically acceptable salt thereof, including those for which a structure is as identified in Table 1A, Table 1B, Table 1C, Table 1D, Table 1E, Table 1F, Table 1G, Table 1H, or Table 11 of the present specification.

In one aspect, a cyclic peptide inhibitor compound of the interleukin-23 receptor (IL-23R) compound, or a pharmaceutically acceptable salt thereof, has a structure of a compound in Table 1A.

In another aspect, a cyclic peptide inhibitor compound of the interleukin-23 receptor (IL-23R) compound or a pharmaceutically acceptable salt thereof, has a structure of a compound in Table 1B.

In another aspect, a cyclic peptide inhibitor compound of the interleukin-23 receptor (IL-23R) compound or a pharmaceutically acceptable salt thereof, has a structure of a compound in Table 1C.

In another aspect, a cyclic peptide inhibitor compound of the interleukin-23 receptor (IL-23R) compound or a pharmaceutically acceptable salt thereof, has a structure of a compound in Table 1D.

In another aspect, a cyclic peptide inhibitor compound of the interleukin-23 receptor (IL-23R) compound or a pharmaceutically acceptable salt thereof, has a structure of a compound in Table 1E.

In another aspect, a cyclic peptide inhibitor compound of the interleukin-23 receptor (IL-23R) compound or a pharmaceutically acceptable salt thereof, has a structure of a compound in Table 1F.

In another aspect, a cyclic peptide inhibitor compound of the interleukin-23 receptor (IL-23R) compound or a pharmaceutically acceptable salt thereof, has a structure of a compound in Table 1G

In another aspect, a peptide inhibitor compound of the interleukin-23 receptor (IL-23R) compound, or a pharmaceutically acceptable salt thereof, has a structure of a compound in Table 1H.

In another aspect, a peptide inhibitor compound of the interleukin-23 receptor (IL-23R) compound, or a pharmaceutically acceptable salt thereof, has a structure of a compound in Table 1I.

Lengthy table referenced here
US20240409598A1-20241212-T00001
Please refer to the end of the specification for access instructions.

Lengthy table referenced here
US20240409598A1-20241212-T00002
Please refer to the end of the specification for access instructions.

Lengthy table referenced here
US20240409598A1-20241212-T00003
Please refer to the end of the specification for access instructions.

Lengthy table referenced here
US20240409598A1-20241212-T00004
Please refer to the end of the specification for access instructions.

Lengthy table referenced here
US20240409598A1-20241212-T00005
Please refer to the end of the specification for access instructions.

Lengthy table referenced here
US20240409598A1-20241212-T00006
Please refer to the end of the specification for access instructions.

Lengthy table referenced here
US20240409598A1-20241212-T00007
Please refer to the end of the specification for access instructions.

Lengthy table referenced here
US20240409598A1-20241212-T00008
Please refer to the end of the specification for access instructions.

Lengthy table referenced here
US20240409598A1-20241212-T00009
Please refer to the end of the specification for access instructions.

### Synthesis

The compounds described herein may be synthesized by many techniques that are known to those skilled in the art. In certain aspects, monomer subunits are synthesized and purified using the techniques described in the accompanying Examples. In some aspects, the present invention provides a method of producing a compound (or monomer subunit thereof) of the invention, comprising chemically synthesizing a peptide having an amino acid sequence described herein, including but not limited to any of the amino acid sequences set forth in the compounds of Formula (I) to Formula (X), Table 1A, Table 1B, Table 1C, Table 1D, Table 1E, Table 1F, Table 1G, Table 1H, and Table 1I herein. In some aspects, a portion of the peptide is recombinantly synthesized, instead of being chemically synthesized. In some aspects, methods of producing a compound further include cyclizing the compound precursor after the constituent subunits have been attached. In particular aspects, cyclization is accomplished via any of the various methods described herein.

Substituted tryptophans may be prepared by any suitable route. Preparation of certain substituted tryptophans including those substituted at the 7 position, such as 7-ethyl-L-tryptophans, are described in, for example WO 2021/146441 A1.

The present invention further describes synthesis of compounds described herein, such as the compounds of Formulas (I) to (XX) and the compounds of Table 1A, Table 1B, Table 1C, Table 1D, Table 1E, Table 1F, Table 1G, Table 1H, and Table 1I. In some aspects, one or more of the amino acid residues or amino acid monomers are lipidated and then covalently attached to one another to form a compound of the invention. In some aspects, one or more of the amino acid residues or amino acid monomers are covalently attached to one another and lipidated at an intermediate oligomer stage before attaching additional amino acids and cyclization to form a compound of the invention. In some aspects, a cyclic peptide is synthesized and then lipidated to form a compound of the invention. Illustrative synthetic methods are described in the Examples.

The present invention further describes synthesis of compounds described herein, such as the compounds of Formulas (I) to Formula (X), and the compounds of Table 1A, Table 1B, Table 1C, Table 1D, Table 1E, Table 1F, Table G, Table 1H, and Table 1I. Illustrative synthetic methods are described in the Examples.

### IV. Pharmaceutical Compositions

The present invention relates to pharmaceutical composition which comprise an IL-23R inhibitor of the present invention. The present invention includes pharmaceutical compositions comprising one or more inhibitors of the present invention and a pharmaceutically acceptable carrier, diluent or excipient. The pharmaceutically acceptable carrier, diluent or excipient may be a solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The pharmaceutical compositions may be administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered orally. A pharmaceutical composition may be formulated for and administered parenterally.

In a particular aspect, an IL-23R inhibitor of the present invention, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

The IL-23R inhibitors of the present invention may be prepared and/or formulated as pharmaceutically acceptable salts, solvates and/or other forms thereof or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

The present invention relates to pharmaceutical compositions comprising an IL-23R inhibitor of the present invention or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5 (12): 524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶C, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

In certain aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsulated matrices of the peptide inhibitor in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the peptide inhibitor can be controlled. Depot injectable formulations are also prepared by entrapping the peptide inhibitor in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous formulations and can be prepared as a dry powder which may be pressurized or non-pressurized. In non-pressurized powder compositions, the active ingredient may be finely divided form may be used in admixture with a larger-sized pharmaceutically acceptable inert carrier comprising particles having a size, for example, of up to 100 micrometers in diameter. Suitable inert carriers include sugars such as lactose.

Alternatively, a pharmaceutical composition of the present invention may be pressurized and contain a compressed gas, such as nitrogen or a liquefied gas propellant. The liquefied propellant medium and indeed the total composition may be such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

A further form of topical administration is to the eye. A peptide inhibitor of the present invention may be delivered in a pharmaceutically acceptable ophthalmic vehicle, such that the peptide inhibitor is maintained in contact with the ocular surface for a sufficient time period to allow the peptide inhibitor to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the peptide inhibitors of the invention may be injected directly into the vitreous and aqueous humor.

Compositions for rectal or vaginal administration include suppositories which may be prepared by mixing the peptide inhibitors of this invention with suitable non-irritating excipients or carriers such as cocoa butter, polyethylene glycol or a suppository wax, which are solid at room temperature but liquid at body temperature and, therefore, melt in the rectum or vaginal cavity and release the active compound.

Peptide inhibitors of the present invention may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a peptide inhibitor of the present invention, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions suitable for parenteral administration in a method or use described herein may comprise sterile aqueous solutions and/or suspensions of the IL:-23R inhibitors made isotonic with the blood of the recipient, generally using sodium chloride, glycerin, glucose, mannitol, sorbitol, and the like.

The present invention provides a pharmaceutical composition for oral delivery. Compositions and peptide inhibitors of the present invention may be prepared for oral administration according to any of the methods, techniques, and/or delivery vehicles described herein. Further, one having skill in the art will appreciate that the peptide inhibitors of the instant invention may be modified or integrated into a system or delivery vehicle that is not disclosed herein, yet is well known in the art and compatible for use in oral delivery of peptides.

Formulations for oral administration may comprise adjuvants (e.g. resorcinols and/or nonionic surfactants such as polyoxyethylene oleyl ether and n-ether) to artificially increase the permeability of the intestinal walls, and/or enzymatic inhibitors (e.g. pancreatic trypsin inhibitors, diisopropylfluorophosphate (DFF) or trasylol) to inhibit enzymatic degradation. In certain embodiments, the peptide inhibitor of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, cascin, albumin, synthetic or semisynthetic polymer, or glyceride. These formulations for oral administration can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In particular aspects, oral dosage forms or unit doses compatible for use with the peptide inhibitors of the present invention may include a mixture of peptide inhibitor and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of peptide inhibitor, wherein the dosage form comprises at least one of a pill, a tablet, a capsule, a gel, a paste, a drink, a syrup, ointment, and suppository. In some instances, an oral dosage form is provided that is designed and configured to achieve delayed release of the peptide inhibitor in the subject's small intestine and/or colon.

Tablets may contain excipients, glidants, fillers, binders and the like. Aqueous compositions are prepared in sterile form, and when intended for delivery by other than oral administration generally will be isotonic. Compositions may optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the compositions ranges from, for example, about 3 to about 11. The pH of the compositions may, for example, range from about 5 to about 7 or from about 7 to about 10.

An oral pharmaceutical composition of the present invention may comprise an IL-23R inhibitor of the present invention may comprise an enteric coating that is designed to delay release of the IL-23R inhibitor in the small intestine. The invention relates to a pharmaceutical composition that comprises an IL-23R inhibitor of the present invention and a protease inhibitor, such as aprotinin, in a delayed release pharmaceutical formulation. Pharmaceutical compositions (e.g., oral pharmaceutical compositions) may comprise an enteric coat that is soluble in gastric juice at a pH of about 5.0 or higher. Such enteric coatings may comprise a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

An oral pharmaceutical composition comprising an IL-23R inhibitor of the present invention that comprises an IL-23R inhibitor may comprise an enteric coating that is designed to protect and release the pharmaceutical composition in a controlled manner within the subject's lower gastrointestinal system, and to avoid systemic side effects. In addition to enteric coatings, the peptide inhibitors of the instant invention may be encapsulated, coated, engaged or otherwise associated within any compatible oral drug delivery system or component. For example, in some embodiments an IL-23R inhibitor of the present invention is provided in a lipid carrier system comprising at least one of polymeric hydrogels, nanoparticles, microspheres, micelles, and other lipid systems.

To overcome peptide degradation of an IL-23R inhibitor of the present invention in the small intestine, the pharmaceutical compositions may comprise a hydrogel polymer carrier system in which a peptide inhibitor of the present invention is contained, whereby the hydrogel polymer protects the IL-23R inhibitor from proteolysis in the small intestine and/or colon. The an IL-23R inhibitor may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more an IL-23R inhibitors of the present invention to provide a pharmaceutical agent for oral delivery. For example, an IL-23R inhibitor of the present invention may be used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration.

In certain aspects, pharmaceutical composition and formulations may include an IL-23R inhibitor of the present invention and one or more absorption enhancers, enzyme inhibitors, or mucoso adhesive polymers. In an embodiment, the absorption enhancer may be an intestinal permeation enhancer.

IL-23R inhibitors of the present invention may be formulated in a formulation vehicle, such as, e.g., emulsions, liposomes, microsphere or nanoparticles.

The present invention provides for a method for treating a subject with an IL-23R inhibitor of the present invention having an increased half-life. In one aspect, the present invention provides a peptide inhibitor having a half-life of at least several hours to one day in vitro or in vivo (e.g., when administered to a human subject) sufficient for daily (q.d.) or twice daily (b.i.d.) dosing of a therapeutically effective amount. In certain embodiments, the IL-23R inhibitor has a half-life of three days or longer sufficient for weekly (q.w.) dosing of a therapeutically effective amount. In certain embodiments, the IL-23R inhibitor has a half-life of eight days or longer sufficient for bi-weekly (b.i.w.) or monthly dosing of a therapeutically effective amount. In certain embodiments, the IL-23R inhibitor is derivatized or modified such that is has a longer half-life as compared to the underivatized or unmodified peptide inhibitor. In certain embodiments, the IL-23R inhibitor contains one or more chemical modifications to increase serum half-life.

When used in at least one of the treatments or delivery systems described herein, a peptide inhibitor of the present invention may be employed in pure form or, where such forms exist, in pharmaceutically acceptable salt form.

The total daily usage of the IL-23R inhibitor and compositions of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide inhibitor employed; e) the duration of the treatment; f) drugs used in combination or coincidental with the specific peptide inhibitor employed, and like factors well known in the medical arts.

In particular embodiments, the total daily dose of a IL-23R inhibitor of the present invention to be administered to a human or other mammal host in single or divided doses may be in amounts, for example, from 0.0001 to 300 mg/kg body weight daily or 1 to 300 mg/kg body weight daily.

The compositions may conveniently be presented in unit dosage form and can be prepared by any of the methods well known in the art of pharmacy. Techniques and compositions generally are found in Remington's Pharmaceutical Sciences (Mack Publishing Co., Easton, PA). Such methods include the step of bringing into association the active ingredient with the carrier which constitutes one or more accessory ingredients. In general the compositions are prepared by uniformly and intimately bringing into association the active ingredient with liquid carriers or finely divided solid carriers or both, and then, if necessary, shaping the product.

Compositions suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. The active ingredient may also be administered as a buccal or sublingual formulation. Buccal or sublingual formulations may comprise an active ingredient in a matrix that releases the active ingredient for transport across the buccal and/or sublingual membranes. The buccal or sublingual formulation may further include a rate controlling matrix that releases the active compounds at a predetermined rate for transport across the buccal and/or sublingual membranes. The buccal or sublingual formulation may further include one or more compounds selected from the group consisting of (i) taste masking agents, (ii) enhancers, (iii) complexing agents, and mixtures thereof; and (iv) other pharmaceutically acceptable carriers and/or excipients. The enhancer may be a permeation enhancer.

A tablet is made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets can optionally be coated or scored and optionally are formulated so as to provide slow or controlled release of the active ingredient therefrom.

### V. Non-Invasive Detection of Intestinal Inflammation

The IL-23R inhibitors of the present invention may be used for detection, assessment and diagnosis of intestinal inflammation by microPET imaging, wherein the peptide inhibitor is labeled with a chelating group or a detectable label, as part of a non-invasive diagnostic procedure. In certain embodiments, an IL-23R inhibitor of the present invention is conjugated with a bifunctional chelator. In certain embodiments, an IL-23R inhibitor of the present invention is radiolabeled. The labeled an IL-23R inhibitor is then administered to a subject orally or rectally. In certain embodiments, the an IL-23R inhibitor is included in drinking water. Following uptake of the an IL-23R inhibitor, microPET imaging may be used to visualize inflammation throughout the subject's bowels and digestive track.

### VI. Methods of Treatments and/or Uses

The present invention relates to methods for treating a subject afflicted with a condition or indication associated with IL-23 or IL-23R (e.g., activation of the IL-23/IL-23R signaling pathway), wherein the method comprises administering to the subject an IL-23R inhibitor disclosed herein. A In one aspect, the present invention relates to a method for treating a subject afflicted with a condition or indication characterized by inappropriate, deregulated, or increased IL-23 or IL-23R activity or signaling, comprising administering to the individual a peptide inhibitor of the present invention in an amount sufficient to inhibit (partially or fully) binding of IL-23 to an IL-23R in the subject. The inhibition of IL-23 binding to IL-23R may occur in particular organs or tissues of the subject, e.g., the stomach, small intestine, large intestine/colon, intestinal mucosa, lamina propria, Peyer's Patches, mesenteric lymph nodes, or lymphatic ducts.

The present invention relates to methods comprising providing a peptide inhibitor described herein to a subject in need thereof. The subject in need thereof may be a subject that has been diagnosed with or has been determined to be at risk of developing a disease or disorder associated with IL-23/IL-23R. The subject may be a mammal. The subject may be, in particular, a human.

The disease or disorder to be treated by treatment with an IL-23R inhibitor of the present invention may be inflammatory, autoimmune inflammation diseases and/or related disorders, such as multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ilcoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

The present invention relates to a method or use of an IL-23R inhibitor for treating an inflammatory disease in a subject that includes administering to the subject a therapeutically effective amount of an IL-23R inhibitor of the present invention or pharmaceutically acceptable solvate or salt thereof, or a composition disclosed herein comprising an IL-23 inhibitor of the present invention.

In some aspects, the present invention provides a method of treating an inflammatory disease or autoimmune inflammation diseases and/or related disorders in a subject that includes administering to the subject a therapeutically effective amount of an IL-23R inhibitor of the present invention or pharmaceutically acceptable solvate or salt thereof, or a composition of the present invention.

Suitable inflammatory, autoimmune inflammation diseases and/or related disorders for treatment with a compound or pharmaceutically acceptable salt thereof, or a composition of the present invention, may include, but are not limited to inflammatory bowel disease (IBD), Crohn's disease (CD), ulcerative colitis (UC), psoriasis (PsO), or psoriatic arthritis (PsA) and the like. The inflammatory disease to be treated may be inflammatory bowel disease (IBD), Crohn's disease, or ulcerative colitis. The inflammatory disease to be treated may be selected from psoriasis, or psoriatic arthritis. The inflammatory disease to be treated may be psoriasis The inflammatory disease to be treated may be psoriatic arthritis. The inflammatory disease to be treated may be IBD.

The present invention relates to methods for treating an inflammatory, autoimmune inflammation diseases and/or related disorders in a subject in need thereof, comprising administering to the subject an IL-23R inhibitor disclosed herein (e.g., a peptide inhibitor or the IL-23R of Formula (I) to Formula (X) or any of Tables 1A-11. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory, autoimmune inflammation diseases and/or related disorders in a subject in need thereof, comprising administering to the subject an IL-23R inhibitor of Formulas I to X) or any of Tables 1A-1I.

The inflammatory, autoimmune inflammation diseases and/or related disorders may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory, autoimmune inflammation diseases and/or related disorders in a subject in need thereof, comprising administering to the subject an IL-23R inhibitor of Formulas I to X) or any of Tables 1A-11. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods of inhibiting IL-23 binding to an IL-23R on a cell, comprising contacting the IL-23R with a peptide inhibitor of the receptor disclosed herein. The cell may be a mammalian cell. The method may be performed in vitro or in vivo. Inhibition of binding may be determined by a variety of routine experimental methods and assays known in the art.

The present invention relates to a method of selectively inhibiting IL-23 or IL-23R signaling (or the binding of IL-23 to IL-23R) in a subject (e.g., in a subject in need thereof), comprising providing to the subject a peptide inhibitor of the IL-23R described herein. The present invention includes and provides a method of selectively inhibiting IL-23 or IL-23R signaling (or the binding of IL-23 to IL-23R) in the GI tract of a subject (e.g., a subject in need thereof), comprising providing to the subject a peptide inhibitor of the IL-23R of the present invention by oral administration. The exposure of GI tissues (e.g., small intestine or colon) to the administered peptide inhibitor may be at least 10-fold, at least 20-fold, at least 50-fold, or at least 100-fold greater than the exposure (level) in the blood. In particular embodiments, the present invention includes a method of selectively inhibiting IL23 or IL23R signaling (or the binding of IL23 to IL23R) in the GI tract of a subject (e.g., a subject in need thereof), comprising providing to the subject a peptide inhibitor, wherein the peptide inhibitor does not block the interaction between IL-6 and IL-6R or antagonize the IL-12 signaling pathway. In a further related embodiment, the present invention includes a method of inhibiting GI inflammation and/or neutrophil infiltration to the GI, comprising providing to a subject in need thereof a peptide inhibitor of the present invention. In some embodiments, methods of the present invention comprise providing a peptide inhibitor of the present invention (i.e., a first therapeutic agent) to a subject (e.g., a subject in need thereof) in combination with a second therapeutic agent. In certain embodiments, the second therapeutic agent is provided to the subject before and/or simultaneously with and/or after the peptide inhibitor is administered to the subject. In particular embodiments, the second therapeutic agent is an anti-inflammatory agent. In certain embodiments, the second therapeutic agent is a non-steroidal anti-inflammatory drug, steroid, or immune modulating agent. In certain embodiments, the method comprises administering to the subject a third therapeutic agent. In certain embodiments, the second therapeutic agent is an antibody that binds IL-23 or IL-23R.

The present invention relates to methods of inhibiting IL-23 signaling by a cell, comprising contacting the IL-23R with a peptide inhibitor described herein. In certain embodiments, the cell is a mammalian cell. In particular embodiments, the method is performed in vitro or in vivo. In particular embodiments, the inhibition of IL-23 signaling may be determined by measuring changes in phospho-STAT3 levels in the cell.

In any of the foregoing methods, IL-23R inhibitor administration to a subject may be conducted orally, but other routes of administration are not excluded. Other routes of administration include, but are not limited to, parenteral, subcutaneous, intravenous, intramuscular, intraperitoneal, transdermal, topical, buccal or ocular routes. Dosages of a peptide inhibitor or the IL-23R described herein (e.g., a compound of Formulas I to X or any of Tables 1A-1I), or salt or solvate thereof to be administered to a subject may be determined by a person of skill in the art taking into account the the disease or condition being treated including its severity, and factors including the age weight, sex, and the like. Exemplary dose ranges include, but are not limited to, from about 1 mg to about 1000 mg, or from about 1 mg to about 500 mg, from about 1 mg to about 100 mg, from about 10 mg to about 50 mg, from about 20 mg to about 40 mg, or from about 20 mg to about 30 mg. A dose range of a peptide inhibitor or the IL-23R described herein may be from about 600 mg to about 1000 mg. A dose range of a peptide inhibitor or the IL-23R described herein may be from about 300 mg to about 600 mg. A dose range of a peptide inhibitor or the IL-23R described herein may be from about 5 mg to about 300 mg. A dose range of a peptide inhibitor or the IL-23R described herein may be from about 25 mg to about 150 mg. A dose range of a peptide inhibitor or the IL-23R described herein may be from about 25 mg to about 100 mg. A dose range of a peptide inhibitor or the IL-23R described herein may be present in a dose range of from about 1 mg to about 100 mg. A dose range of a peptide inhibitor or the IL-23R described herein may be present in a dose range of from about 20 mg to about 40 mg. A dose range of a peptide inhibitor or the IL-23R described herein may be present in a dose range of from about 20 mg to about 30 mg.

### ASPECTS OF THE PRESENT INVENTION

The following aspects illustrate and are not intended to limit scope of the present invention. Instead, these aspects provide guidance to any skilled artisan on how to prepare and use compounds, compositions and methods taught by the present invention, where such skilled artisans will appreciate that modifications may be made without departing from the spirit and scope of the invention.
1. A peptide inhibitor of an interleukin-23 receptor, which comprises an amino acid sequence of Formula I

R1-X3-X4-X5-X6-X7-X8-X9-X10-X11-X12-X13-X14-X15-X16-R2
wherein:
R1 is hydrogen, CH₃C(O)—, EtC(O)—, MeSO2, AzCO, BHCO, FPrpTriazoleMeCO, SMSBCO, Biotin, BiotinPEG2PEG2CO, DAGSuc;
X3 is dR, dK, PEG6, gEPEG6, R, K, or absent;
X4 is Pen, aMeC, hC, or C;
X5 is A, N, Q, N-MeAsn, L, Asn(4C13_2N15), I, K (PEG2PEG2Biotin);
X6 is T, MeThr, V, K, Dbu, Dpr, or A;
X7 is W7Me, W, W(4F7Me), 7MeW, 7PhW, 7EtW, 7FW, 7ClW, 5BrW, 7(3NAcPh)W′
X8 is KAc, Q, NMeGln, A, Cit, dK(Ac), dQ, dNMeGln, dA, or dCit;
X9 is Pen, aMeC, hC, or C;
X10 is F4OMe, AEF, F, F4Me, F4Ad, Nal, AEF(Boc), 4PipPhe, AEF(Ac), Y, 4OMeF, 4AmF, D(Pip), Tzl(mPEG3), 3FTyr, Y(OTzl), Y(OTzl(mPEG3)), Tzl, Tzl(PEG3OH);
X11 is Nal, Quin_3, Coumarin(7OMe), 2Nal, 3Quin;
X12 is aMeK, THP, Spiral_Pip_Ac, Spiral_Pip, MeK, aMeLeu, aMeL, aMeK(Boc)
X13 is KAc, K, dK(Ac), or dK;
X14 is A, N, L, N-MeAsn, MeLeu, Asn(4C13_2N15), I;
X15 is 3Pya, bAla, Thiozolidine, H, dL, N, A, F, aMePhe, Aib, dK, h, 3MeH, 1MeH, tetraFPhe, bMePhe(SR), 5PyrimidAla, v, dR, homoF, Y, y, F(CF3), Y(CHF2), THP, or absent;
X16 is MeGly, dMeGly, dL, MeLeu, dMeLeu, N-MeNle, dN-MeNle, y, paf, maf, d3Pya, bAla, dbAla, P, dP, N(3AmBenzyl)Gly, N(4AmBenzyl)Gly, 4(R)HydroxyPro, 4(S)AminoPro, 5(R)diMePro, or absent;
R2 is —OH, —NH₂, —HN(C₁-C₄ alkyl), —N(C₁-C₄ alkyl)₂, MeNH, CONHMe; and
wherein the inhibitor of an interleukin-23 receptor is cyclized by a disulfide bond between penicillamine, cysteine, homocysteine, or alpha methylcysteine residues at positions X4 and X9.
2. The inhibitor of an interleukin-23 receptor of aspect 1, wherein X4 and X9 are Pen or hC residues that are selected independently.
3. The inhibitor of an interleukin-23 receptor of any of aspects 1 to 2, wherein X15 is 3Pya.
4. The inhibitor of an interleukin-23 receptor of any of aspects 1 to 3, wherein X11 is 2Nal or 3Quin.
5. The inhibitor of an interleukin-23 receptor of any of aspects 1 to 4, wherein X7 is 7MeW, or W.
6. The inhibitor of an interleukin-23 receptor of any of aspects 1 to 5, wherein:
   R1 is hydrogen or CH₃C(O)—; and
   R2 is —NH₂, MeNH, or CONHMe.
7. A peptide inhibitor of an interleukin-23 receptor, which comprises an amino acid sequence of Formula II

R1-X3-Abu-X5-T-X7-X8-X9-AEF-X11-X12-X13-X14-X15-X16-X17-R2 (II)
wherein:
   R1 is hydrogen, or CH₃C(O)—;
   X3 is dR, R, or absent;
   X4 is Abu;
   X5 is Q, N, or T;
   X6 is T;
   X7 is W or 7MeW;
   X8 is Q, K, KAc, dQ, dK, or dK(Ac);
   X9 is Pen, C, hC, or aMeC;
   X10 is AEF;
   X11 is 2Nal, or Nal;
   X12 is THP, Acvc, or Achx;
   X13 is E, KAc, aMeE, Q, AIB, Achx, aMedE, dE, dK(Ac), or dQ;
   X14 is N or S;
   X15 is H, bAla, N, 3Pya, F, aMeF, aMeW, 1Nal, 4AmPhe, 2Nal, aMeFPhe, aMePhe, 3,4diFPhe, DY02, 5FW, or absent;
   X16 is MeGly, AIB, or absent;
   X17 is aMeK or absent;
   R2 is —OH, —NH₂, —HN(C₁-C₄ alkyl), —N(C₁-C₄ alkyl)₂; and
   wherein the inhibitor of an interleukin-23 receptor is cyclized by a thioether bond between the Abu residue at X4 and a cysteine, homocysteine, or alpha methylcysteine residue at X9.
8. The inhibitor of an interleukin-23 receptor of aspect 7, wherein X9 is aMeC.
9. The inhibitor of an interleukin-23 receptor of any of aspects 7 to 8, wherein X5 is N.
10. The inhibitor of an interleukin-23 receptor of any of aspects 7 to 8, wherein X8 is KAc.
11. The inhibitor of an interleukin-23 receptor of any of aspects 7 to 10, wherein X11 is Nal.
12. The inhibitor of an interleukin-23 receptor of any of aspects 7 to 11, wherein X15 is 3Pya.
13. The inhibitor of an interleukin-23 receptor of any of aspects 1 to 12, wherein:
   R1 is CH₃C(O)—; and
   R2 is —NH₂.
14. A peptide inhibitor of an interleukin-23 receptor, which comprises an amino acid sequence of Formula III

R1-X3-X4-X5-X6-X7-X8-X9-X10-X11-X12-X13-X14-X15-X16-R2 (III)
wherein:
R1 is hydrogen, CH₃C(O)—, FPrpTriazoleMeCO, NH₂, EtCO, AzCO, or BHCO;
X3 is dR, R, K, or dK;
X4 is Pen, Abu, AIB, aMeC, C, hC, Ala, 4RAminoPro, or 4SAminoPro;
X5 is N, D, or E;
X6 is T, Hyp, or 3OHPro;
X7 is 7MeW, W, 3Pya, A, 7PyrW, or 7(3NAcPh)W;
X8 is KAc, or dKAc;
X9 is Pen, C, S5H, AIB, D, E, hC, aMeC;
X10 is AEF, AEF(EtCO), AEF(BH), AEF(Ac), bMeAEF(2S3R*), bMeAEF(2S3S*), Y, or A;
X11 is 2Nal, A, Nal, or W;
X12 is THP;
X13 is E, KAc, S5H, dE, dKAc, or R5H;
X14 is N, S, 3Pya;
X15 is 3Pya, H, bAla, v, dR, hF, PAF, F, THP, 1, 4Pya, oAMPhe, 3MeH, D3Pya, N, 5MePyridinAla, 5AmPyridinAla, 3QuinolAla, 6OH3Pya, A
X16 is MeGly;
R2 is —NH₂—HN(C₁-C₄ alkyl), —N(C₁-C₄ alkyl)₂ or —OH; and
wherein the inhibitor of an interleukin-23 receptor is cyclized by a disulfide bond between penicillamine, cysteine, homocysteine, or alpha methylcysteine residues at positions X4 and X9; or
wherein the inhibitor of an interleukin-23 receptor is cyclized by a thioether bond between the Abu residue at X4 and a cysteine, homocysteine, or alpha methylcysteine residue at X9; or
wherein when X4 is 4RAminoPro or 4SAminoPro and X9 is E or D, the inhibitor of an interleukin-23 receptor is cyclized by an amide bond between X4 and X9;
or
wherein when X5 is D or E, and X10 comprises an AEF residue, the inhibitor of an interleukin-23 receptor is cyclized by an amide bond between X5 and X10;
or
wherein when X9 and X13 comprise S5H residues the inhibitor of an interleukin-23 receptor is cyclized by an aliphatic linkage between X9 and X13.
15. The inhibitor of an interleukin-23 receptor of aspect 14, wherein:
   X4 is and X9 is selected independently from Pen, C, and aMeC and X9 is Abu and the peptide is cyclized by formation of a thioether linkage; or
   X4 and X9 are selected independently from Pen, C, hC, and aMeC, and the inhibitor is cyclized by a disulfide bond between amino acids at positions X4 and X9.
16. The inhibitor of an interleukin-23 receptor of aspect 14, wherein X15 is 5MePyridinAla or 5AmPyridinAla.
17. The inhibitor of an interleukin-23 receptor of any of aspects 14 to 16, wherein X3 is dR and X4 is Pen.
18. The inhibitor of an interleukin-23 receptor of any of aspects 14 to 17, wherein X11 is 2Nal and X12 is THP.
19. The inhibitor of an interleukin-23 receptor of any of aspects 14 to 18, wherein one or both of X5 and X14 are N.
20. The inhibitor of an interleukin-23 receptor of any of aspects 14 to 19, wherein R1 is CH₃C(O)—.
21. The inhibitor of an interleukin-23 receptor of any of aspects 14 to 20, wherein R2 is an —NH₂.
22. A peptide inhibitor of an interleukin-23 receptor, which comprises an amino acid sequence of Formula IV

R1-X3-X4-X5-X6-X7-X8-X9-X10-X11-X12-X13-X14-X15-X16-R2 (IV)
wherein:
R1 is hydrogen, CH₃C(O)—, Ac_Morph, or MorphCO;
X3 is K(AcMorp), Kmorp, dK(AcMorp), or absent;
X4 is Pen, C, hC, or aMeC;
X5 is L, N, or nLeu;
X6 is T or L;
X7 is W or 7MeW;
X8 is KAc, K(AcMorph), K(IsoButyl_Ac), K(Butyl_Ac), K(Benzyl_Ac), KMorph, K, dKAc, dK(AcMorph), dK(IsoButyl_Ac), dK(Butyl_Ac), dK(Benzyl_Ac), dKMorph, or dK;
X9 is Pen, C, hC, or aMeC;
X10 is F4OMe, F, AEF, F4Ad, L, F4CN, or 4OMeF;
X11 is 2Nal or Nal;
X12 is L, THP, Spiral_Pip, aMeK, or aMeL;
X13 is L, dL, or nL (i.e., norleucine);
X14 is N or L;
X15 is 3Pya or absent;
X16 is MeGly or absent;
R2 is NH (2-(pyridin3-1)ethyl), —NH₂, —HN(C₁-C₄ alkyl), —N(C₁-C₄ alkyl)₂, or —OH; and
wherein the inhibitor of an interleukin-23 receptor is cyclized by a disulfide bond between penicillamine, cysteine, homocysteine, or alpha methylcysteine residues at positions X4 and X9.
23. The inhibitor of an interleukin-23 receptor of aspect 22, wherein one or both of X4 and X9 are Pen.
24. The inhibitor of an interleukin-23 receptor of any of aspects 22 to 23, wherein X3 is absent.
25. The inhibitor of an interleukin-23 receptor of any of aspects 23 to 24, wherein X8 is KAc or K.
26. The inhibitor of an interleukin-23 receptor of any of aspects 22 to 25, wherein X11 is 2Nal.
27. The inhibitor of an interleukin-23 receptor of any of aspects 22 to 26, wherein X12 is aMeL or THP.
28. The inhibitor of an interleukin-23 receptor of any of aspects 22 to 27, wherein:
   R1 is CH₃C(O)—; and
   R2 is —OH or —NH₂.
29. A peptide inhibitor of an interleukin-23 receptor, which comprises an amino acid sequence of Formula V

R1-X3-X4-X5-X6-X7-X8-X9-X10-X11-THP-X13-X14-X15-R2 (V)
wherein:
R1 is hydrogen, or CH₃C(O), Propionic_acid, EtCO, PentCO, AzCO, MeSO2, NH₂, BHCO, FPrpTriazoleMeCO, (SulfoCy3), (SulfoCy3dPEG2), (SulfoCy3dPEG3), or SMSBCO;
X3 is dR, R, or absent;
X4 is Abu, Pen, C, hC, aMeC, aG, or Dpr;
X5 is Q or N;
X6 is T;
X7 is W, W7Me, 7MeW, bMeW (2S3R), bMeW (2S3S), 7FW, 7ClW, 5BrW, or 5MeW;
X8 is Q, K, KAc, Q, dK, or dKAc;
X9 is C, Pen, hC, aMeC, aG, E, or D;
X10 is AEF, F4OMe, F4Ad, Phe(4(2(Ac)aminoethoxy)), ac, LY02, AEF(Boc), 4PipPhe, AEF(BH), or AEF(SMSB);
X11 is 2Nal or Nal;
X12 is THP;
X13 is E, KAc, K, Q, aMeE, AIB, dE, dKAc, dK, dQ, aMedE, or Achx;
X14 is N;
X15 is H, bAla, N, F, aMePhe, aMeF, aMeW, 1Nal, 4AmPhe, 2Nal, aMeFPhe, 3,4diFPhe, DY02, 5FW, D(NBzl), D(NPh), D(NoAn), D(NPip), D(NPyr), D(NpAn), D(NmAn), D(N4Pyz), D(N5In), D(NPrAm), dH, D(NEtNH2), 3MeH, 1MeH, tetraFPhe, bMePhe(SR), 5PyrimidAla, 3OHPhe, 4PyridinAla, 3Pya, 4TriazolAla, bMePhe(2S3S), 2AmTyr, bMeH(2S3S*), or 5MeH;
R2 is —NH₂, —OH, —HN(C₁-C₄ alkyl), —N(C₁-C₄ alkyl)₂, or CONHMe; and
wherein the inhibitor of an interleukin-23 receptor is cyclized by a disulfide bond between penicillamine, cysteine, homocysteine, or alpha methylcysteine residues at positions X4 and X9; or
wherein the inhibitor of an interleukin-23 receptor is cyclized by a thioether bond between the Abu residue at X4 and a cysteine, homocysteine, or alpha methylcysteine residue at X9; or
wherein when X4 is Dpr and X9 is E or D, the inhibitor of an interleukin-23 receptor is cyclized by an amide bond between X4 and X9; or
wherein when X4 and X9 are aG, the inhibitor of an interleukin-23 receptor is cyclized by an aliphatic bond (generated from a Ring Closing Metathesis “RCM” reaction) between X4 and X9.
30. The inhibitor of an interleukin-23 receptor of aspect 29, wherein X4 and X9 are aG, and the inhibitor of an interleukin-23 receptor is cyclized by an aliphatic bond between X4 and X9.
31. The inhibitor of an interleukin-23 receptor of aspect 29, wherein X4 is Dpr and X9 is E or D, and the inhibitor is cyclized by an amide bond between X4 and X9.
32. The inhibitor of an interleukin-23 receptor of aspect 29, wherein the inhibitor is cyclized by a thioether bond between the Abu residue at X4 and a cysteine, homocysteine, or alpha methylcysteine residue at X9.
33. The inhibitor of an interleukin-23 receptor of aspect 29, wherein X4 and X9 are selected independently from Pen, C, hC, and aMeC and the inhibitor is cyclized by a disulfide bond between amino acids at positions X4 and X9.
34. The inhibitor of an interleukin-23 receptor of any of aspects 29 to 33, wherein X3 is absent.
35. The inhibitor of an interleukin-23 receptor of any of aspects 29 to 34, wherein X7 is W or W7Me.
36. The inhibitor of an interleukin-23 receptor of any of aspects 29 to 35, wherein one or both of X10 is AEF.
37. The inhibitor of an interleukin-23 receptor of any of aspects 29 to 36, wherein X15 is F, aMePhe, D(NPh), bMePhe(SR), 3OHPhe, tetraFPhe, or bMePhe(2S3S).
38. The inhibitor of an interleukin-23 receptor of any of aspects 29 to 37, wherein x15 is H, dH, 3MeH, 1MeH, 3MeH, bMeH(2S3S*), or 5MeH.
39. The inhibitor of an interleukin-23 receptor of any of aspects 29 to 38, wherein R¹ is CH₃C(O)—.
40. The inhibitor of an interleukin-23 receptor of any of aspects 29 to 39, wherein R2 is NH₂.
41. A peptide inhibitor of an interleukin-23 receptor, which comprises an amino acid sequence of Formula VI

R1-X4-X5-X6-X7-X8-X9-X10-X11-X12-X13-X14-X15-X16-R2 (VI)
wherein:
R1 is hydrogen, or CH3C(O);
X4 is Pen, Abu, C, hC, dPen, dC, or aMeC;
X5 is L, N, Q. T, dN or absent;
X6 is T, L, dT, or absent;
X7 is W7Me, W(4F7Me), 7PhW, 7MeW, 7EtW, W, 7BrW, 7(2ClPh)W, 7(4CF3Ph)W, 7(3CF3TAZP)W, 7(4NAcPh)W, 7(3NAcPh)W, 7(40CF3Ph)W, 7(40MePh)W, 7(4Paz)W, 7(7Imzpy)W, 7(6(1)7dMeNDAZ))W, 7(3UrPh)W, 7(5(Ina7Pyr))W, 7(4(CpCNPh))W, 7(6(2MeNDAZ))W, BT, D7MeW;
X8 is KAc, Q, K(Gly), dKAc, dQ, or dK(Gly);
X9 is Pen, C, hC, aMeC, or dPen;
X10 is AEF, F4Ad, F4OMe, F4Me, Nal, F, Spiral_Pip, L, 4AmF, AEF(G), dY, or Y;
X11 is Nal, 3Quin, 2Nal, 2Quin, d2Nal, or W;
X12 is THP, aMeLeu, Acvc, aMeK, or Acpx, A;
X13 is E or dE;
X14 is N, L, or dN;
X15 is 3Pya, THP, N, H, dK, dL, dPaf, PAF, 3MeH, 3pya, or F;
X16 is MeGly, dK, K, or absent; and
R2 is —NH₂, —OH, —HN(C₁-C₄ alkyl), —N(C₁-C₄ alkyl)₂, or CONHMe; and
wherein the inhibitor of an interleukin-23 receptor is cyclized by a disulfide bond between a Pen, C, hC, dPen, dC, or aMeC at X4 and a Pen, C, hC, aMeC, or dPen, residue at X9; or
wherein the inhibitor of an interleukin-23 receptor is cyclized by a thioether bond between the Abu residue at X4 and a Pen, C, hC, or aMeC residue at X9.
42. The inhibitor of an interleukin-23 receptor of aspect 41, wherein the inhibitor is cyclized by a disulfide bond between a Pen, Abu, C, hC, dPen, dC, or aMeC at X4 and a Pen, C, hC, aMeC, or dPen X9 the inhibitor is cyclized by a thioether bond between the Abu residue at X4 and a Pen, C, hC, or aMeC residue at X9.
43. The inhibitor of an interleukin-23 receptor of aspect 41, wherein the inhibitor is cyclized by a disulfide bond between a Pen, C, hC, dPen, dC, or aMeC residue at X4 and a Pen, C, hC, aMeC, or dPen residue at X9.
44. The inhibitor of an interleukin-23 receptor of aspect 43, wherein X4 is Pen or dPen.
45. The inhibitor of an interleukin-23 receptor of aspect 43 or aspect 44, wherein X9 is Pen or dPen.
46. The inhibitor of an interleukin-23 receptor of any of aspects 43 to 45, wherein X5 is N or dN.
47. The inhibitor of an interleukin-23 receptor of any of aspects 43 to 46, wherein X6 is T or dT.
48. The inhibitor of an interleukin-23 receptor of any of aspects 43 to 47, wherein X7 is W, 7MeW, or d7MeW.
49. The inhibitor of an interleukin-23 receptor of any of aspects 43 to 48, wherein X8 is KAc or dKAc.
50. The inhibitor of an interleukin-23 receptor of any of aspects 43 to 49, wherein X10 is AEF(G) or dY.
51. The inhibitor of an interleukin-23 receptor of any of aspects 43 to 50, wherein X10 is AEF, F4Ad, F4OMe, F4Me, Nal, F, Spiral_Pip, L, 4AmF, or Y.
52. The inhibitor of an interleukin-23 receptor of any of aspects 43 to 51, wherein X11 is 2Nal or d2Nal.
53. The inhibitor of an interleukin-23 receptor of any of aspects 43 to 52, wherein X12 is THP.
54. The inhibitor of an interleukin-23 receptor of any of aspects 43 to 53, wherein X14 is N or dN.
55. The inhibitor of an interleukin-23 receptor of any of aspects 43 to 54, wherein X15 is 3Pya, or 3pya.
56. The inhibitor of an interleukin-23 receptor of any of aspects 43 to 54, wherein X15 is THP, N, H, dK, dL, dPaf, PAF, 3MeH, or F.
57. The inhibitor of an interleukin-23 receptor of any of aspects 43 to 56, wherein X16 is MeGly.
58. The inhibitor of an interleukin-23 receptor of any of aspects 43 to 56, wherein R2 is NH₂.
59. A peptide inhibitor of an interleukin-23 receptor, which comprises an amino acid sequence of Formula VII

R1-X5-X6-X7-X8-X9-X10-X11-X12-X13-X14-X15-X16-R2 (VII)
wherein:
R1 is 7Ahp, 6Ahx, 8Aoc, or 5Ava;
X5 is N or absent;
X6 is T or absent;
X7 is 7MeW or absent;
X8 is KAc or absent;
X9 is Pen, Aib, or absent;
X10 is AEF or absent;
X11 is 2Nal;
X12 is THP;
X13 is E, dE, hE, hdE, D, dD, or Q;
X14 is N, D, or E;
X15 is 3Pya or N;
X16 is MeGly; and
R2 is absent, —NH₂—HN(C₁-C₄ alkyl), —N(C₁-C₄ alkyl) 2 or —OH; and
wherein the inhibitor of an interleukin-23 receptor is cyclized by a bond between a residue at R1 and X13, R1 and X14, or between a residue at R1 and X9.
60. A peptide inhibitor of an interleukin-23 receptor, which comprises an amino acid sequence of Formula VIII

R1-X3-X4-X5-X6-X7-X8-X9-X10-X11-X12-X13-X14 (VIII)
wherein:
R1 is hydrogen, CH₃C(O)—, FPrpTriazoleMeCO, NH₂, EtCO, AzCO, or BHCO;
X3 is dR, R, or absent;
X4 is Pen, Abu, C, or aMeC;
X5 is Q or N;
X6 is T;
X7 is W or 7MeW;
X8 is Q, dQ, KAc, dKAc;
X9 is Pen, Abu, C, or aMeC;
X10 is AEF or absent;
X11 is 2Nal or absent;
X12 is THP or absent;
X13 is E, dE, D, dD, KAc, dKAc, or absent; and
X14 is N, THP, bAla, N, Pyr, or absent;
R2 is absent or —NH₂—HN(C₁-C₄ alkyl), —N(C₁-C₄ alkyl) 2 or —OH; and
wherein the inhibitor of an interleukin-23 receptor is cyclized by a disulfide bond between a residue at X4 and a residue at X9.
61. A peptide inhibitor of an interleukin-23 receptor, which comprises an amino acid sequence of Formula IX

R1-X6-X7-X8-X9-X10-X11-X12-X13-X14-X15-X16-R2 (IX)
wherein:
R1 is hydrogen, CH₃C(O)—, NH₂, or EtCO;
X6 is absent, 3OHPro, AIB, or T;
X7 is W, 7MeW, or absent;
X8 is KAc, dKAc, AIB, or absent;
X9 is S5H, S5Me, aMeS5H, aMeK, aMeK (N3), E, K, or aMePra;
X10 is AEF, 4OMeF, or F;
X11 is 2Nal;
X12 is THP, aMeK, or aMeL;
X13 is S5H, S5Me, aMeS5H, aMeK, aMeK (N3), E, dE, D, dD, K, dK, or aMePra;
X14 is N or L;
X15 is 3Pya or absent;
X16 is MeGly, N(iBu)Gly, N(Cyclohexyl)Gly, N(3AmBenzyl)Gly, or N(3AmBenzyl)Gly; and
R2 is absent, —NH₂—HN(C₁-C₄ alkyl), —N(C₁-C₄ alkyl)₂ or —OH; and
wherein the inhibitor of an interleukin-23 receptor is cyclized by a bond between a residue at X9 and a residue at X13.
62. A peptide inhibitor of an interleukin-23 receptor, which comprises an amino acid sequence of Formula X

R1-X6-X7-X8-X9-X10-X11-X12-X13-X14-X15-X16-R2 (X)
wherein:
R1 is hydrogen, CH₃C(O)—, NH₂, or EtCO;
X6 is AIB, 3OHPro, T, or absent;
X7 is W, 7MeW, or absent;
X8 is S5H, KAc, or absent;
X9 is AIB, S5H, A, or absent;
X10 is AEF, S5H, hLys, or 4OMeF;
X11 is 2Nal;
X12 is S5H, aMeK, S5Me, or THP;
X13 is KAc, S5H, E, Q, Pen, Abu, C, aMeC, dKAc, dE, dQ, dC, or aMedC;
X14 is N, hE, S5H, D, or N;
X15 is 3Pya;
X16 is MeGly;
R2 is absent, —NH₂—HN(C₁-C₄ alkyl), —N(C₁-C₄ alkyl)₂ or —OH; and
wherein the inhibitor of an interleukin-23 receptor is cyclized by a bond between a residue at X9 and a residue at X12, a bond between a residue at X9 and a residue at X13, a bond between a residue at X10 and a residue at X14, or a bond between a residue at X4 and a residue at X9.
63. An inhibitor of an interleukin-23 receptor provided in any of Tables 1A to Table 1G.
64. An inhibitor of an interleukin-23 receptor provided in Table 1H.
65. An inhibitor of an interleukin-23 receptor provided in Table 1I.
66. An inhibitor of an interleukin-23 receptor selected from compound 345, 469, 477, and 478.

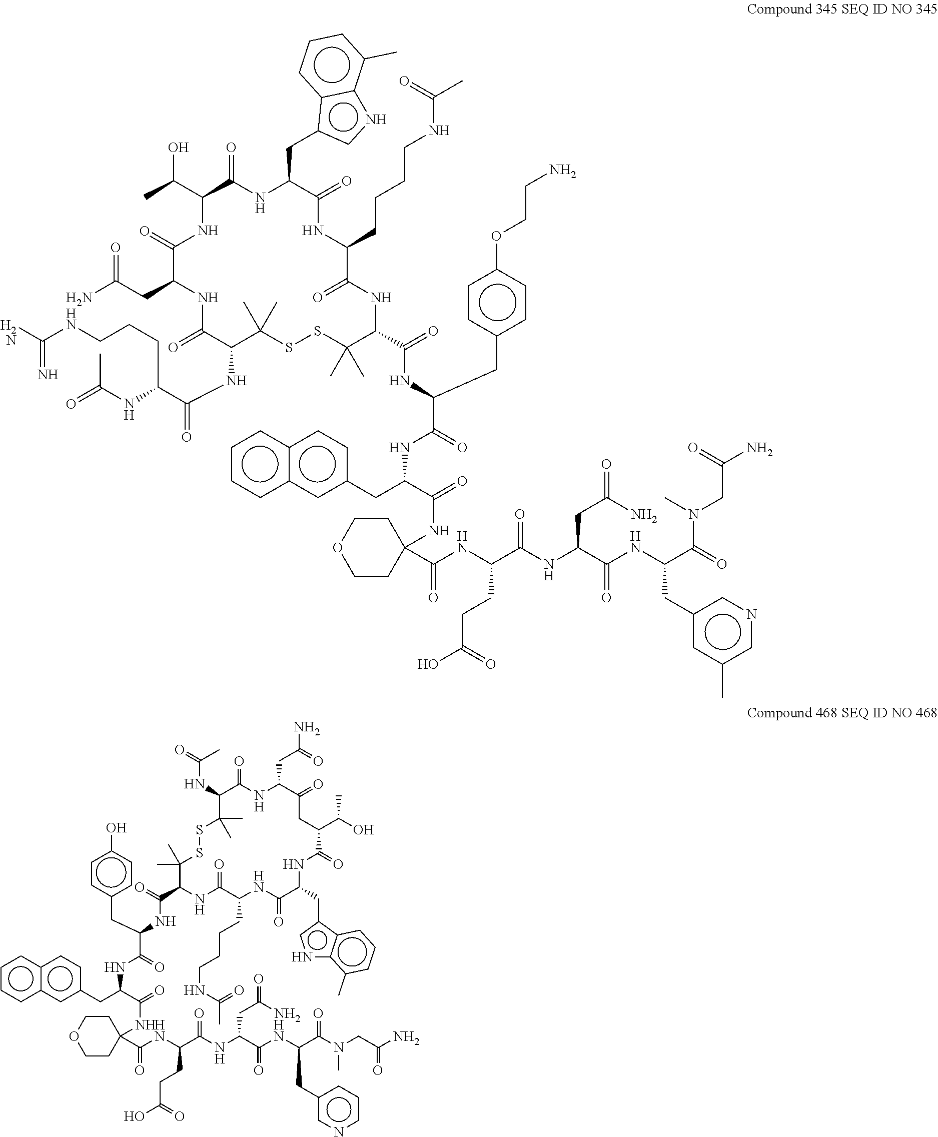

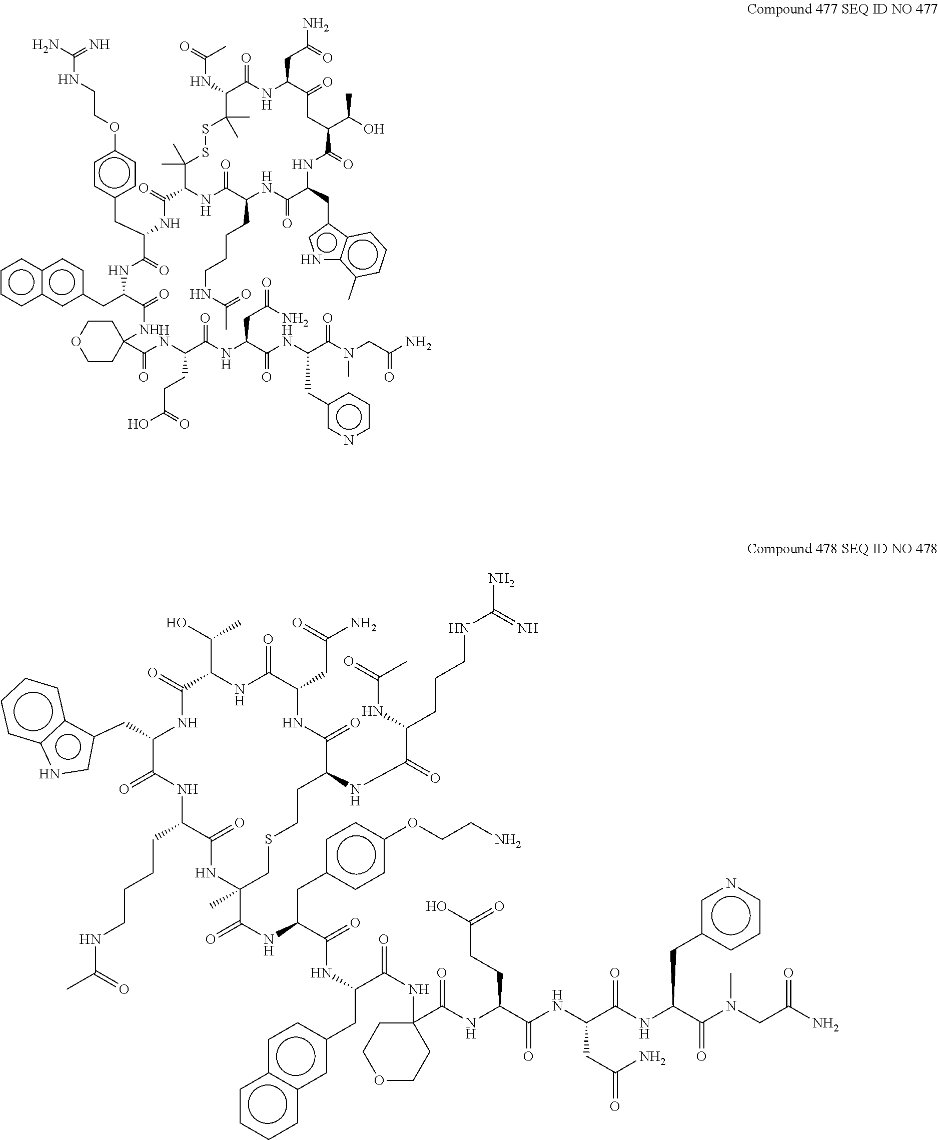

67. The peptide inhibitor of an interleukin-23 receptor of any of aspects 1-66, wherein D amino acids are present or substituted for a corresponding L amino acid only at
   (i) one or more of positions X3, X5, X6, X8, X13 and X16, and optionally one of positions X1-X2, X4, X7, X9 to X12, X14-X18 present in the inhibitor; or
   (ii) one or more of positions X3, X8 and X13, and optionally at one of positions X1-X2, X4-X7, X9 to X12, X14-X18 present in the inhibitor.
68. The peptide inhibitor of an interleukin-23 receptor of any of aspects 1-66, wherein D amino acids are present are present or substituted for a corresponding L amino acid only at
   (i) X3, and optionally at one of positions X1-X2, X4-X18 present in the inhibitor; or
   (ii) one of positions X3, and X8, and optionally one of positions X1-X2, X4-X7, X9-X18 present in the inhibitor.
69. The peptide inhibitor of an interleukin-23 receptor of any of aspects 1-66, wherein the inhibitor comprises amino acids of the D-isomeric form, or substituted with a D amino acid in place of the corresponding L amino acid, at only one or two of positions X1 to X18 appearing in the IL-23R inhibitors set forth herein.
70. The peptide inhibitor of an interleukin-23 receptor of any of aspects 1-66, wherein the inhibitor comprises amino acids of the D-isomeric form, or substituted with a D amino acid in place of the corresponding L amino acid, at only three or four of positions X1 to X18 appearing in the IL-23R inhibitors set forth herein.
71. The peptide inhibitor of an interleukin-23 receptor of any of aspects 1-66, wherein the inhibitor comprises amino acids of the D-isomeric form, or substituted with a D amino acid in place of the corresponding L amino acid, at only five or six of positions X1 to X18 appearing in the IL-23R inhibitors set forth herein.
72. The peptide inhibitor of an interleukin-23 receptor of any preceding aspect wherein the interleukin-23 receptor is a human interleukin receptor.
73. A pharmaceutical composition which comprises:
   (i) peptide inhibitor of an interleukin-23 receptor or pharmaceutically acceptable salt, solvate, or form thereof according to any one of aspects 1-58, and
   (ii) a pharmaceutically acceptable carrier, excipient, or diluent.
74. A pharmaceutical composition which comprises:
   (i) peptide inhibitor of an interleukin-23 receptor or pharmaceutically acceptable salt, solvate, or form thereof according to any one of aspects 59-66, and
   (ii) a pharmaceutically acceptable carrier, excipient, or diluent.
75. A pharmaceutical composition which comprises:
   (i) peptide inhibitor of an interleukin-23 receptor or pharmaceutically acceptable salt, solvate, or form thereof according to aspect 63 or 66: and
   (ii) a pharmaceutically acceptable carrier, excipient, or diluent.
76. A pharmaceutical composition which comprises:
   (i) peptide inhibitor of an interleukin-23 receptor or pharmaceutically acceptable salt, solvate, or form thereof according to aspect 63: and
   (ii) a pharmaceutically acceptable carrier, excipient, or diluent.
77. A pharmaceutical composition which comprises:
   (i) peptide inhibitor of an interleukin-23 receptor or pharmaceutically acceptable salt, solvate, or form thereof according to aspect 64: and
   (ii) a pharmaceutically acceptable carrier, excipient, or diluent.
78. A pharmaceutical composition which comprises:
   (i) peptide inhibitor of an interleukin-23 receptor or pharmaceutically acceptable salt, solvate, or form thereof according to aspect 65: and
   (ii) a pharmaceutically acceptable carrier, excipient, or diluent.
79. A pharmaceutical composition which comprises:
   (i) peptide inhibitor of an interleukin-23 receptor or pharmaceutically acceptable salt, solvate, or form thereof according to aspect 66: and
   (ii) a pharmaceutically acceptable carrier, excipient, or diluent.
80. A pharmaceutical composition which comprises:
   (i) peptide inhibitor of an interleukin-23 receptor or pharmaceutically acceptable salt, solvate, or form thereof according to aspect 67: and
   (ii) a pharmaceutically acceptable carrier, excipient, or diluent.
81. A pharmaceutical composition which comprises:
   (i) peptide inhibitor of an interleukin-23 receptor or pharmaceutically acceptable salt, solvate, or form thereof according to aspect 68 to 72: and
   (ii) a pharmaceutically acceptable carrier, excipient, or diluent.
82. The use of a peptide inhibitor of an interleukin-23 receptor according to any of aspects 1-73 for the preparation of a medicament.
83. The use of a peptide inhibitor of an interleukin-23 receptor according to any of aspects 1-73, or a pharmaceutical composition according to any of aspects 74-82, for the preparation of a medicament for the treatment of inflammatory, autoimmune inflammation diseases and/or related disorders.
84. The use of a peptide inhibitor of an interleukin-23 receptor according to any of aspects 1-73, or a pharmaceutical composition according to any of aspects 74-82, for the preparation of a medicament for the treatment of inflammatory, autoimmune inflammation diseases and/or related disorders including, but not limited to: multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ilcoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.
85. The use of aspect 84, wherein the diseases or disorders are selected from Inflammatory Bowel Disease (IBD), Ulcerative colitis (UC), Crohn's Disease (CD), psoriasis (PsO) or psoriatic arthritis (PsA).
86. A method for treating a disease or disorder associated with Interleukin 23 (IL-23)/Interleukin 23 Receptor (IL-23R), which comprises administering:
   (i) an effective amount of a peptide inhibitor of an interleukin-23 receptor, or a pharmaceutically acceptable salt, solvate, or form thereof according to any one of aspects 1-73;
   or
   (ii) a pharmaceutical composition according to any one of aspects 74 to 82, respectively to a patient in need thereof.
87. The method of aspect 82, wherein the disease or disorder is associated with autoimmune inflammation.
88. The method of aspect 82, wherein the disease or disorder is associated with multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.
89. The method of aspect 82, wherein the disease or disorder is associated with Ulcerative colitis (UC), Crohn's Disease (CD), psoriasis (PsO), or psoriatic arthritis (PsA).
90. The method of aspect 82, wherein the disease or disorder is Ulcerative colitis (UC).
91. The method of aspect 82, wherein the disease or disorder is Crohn's Disease (CD).
92. The method of aspect 82, wherein the disease or disorder is psoriasis (PsO).
93. The method of aspect 82, wherein the disease or disorder is psoriasis psoriatic arthritis (PsA).
94. A kit which comprises a peptide inhibitor of an interleukin-23 receptor of an of aspects 1-72, or a pharmaceutical composition according to any of aspects 73-82, and instructions for the use of the inhibitor of an interleukin-23 receptor or pharmaceutical composition.
95. The kit of aspect 94, wherein the instructions are directed to the treatment of an inflammatory disease or disorder.
96. The kit of aspect 95, wherein the disease is inflammatory bowel disease (IBD), Crohn's disease (CD), ulcerative colitis (UC), psoriasis (PsO), and psoriatic arthritis (PsA).

### EXAMPLES

The following examples illustrate the invention. These examples are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.

Some abbreviations useful in describing the invention are defined below in the following Table 2A to Table 2D.

TABLE 2A
Amino Acid Abbreviations

| Abbreviation | Definition | Smiles |
|---|---|---|
| dR, arg, or r | D-Arginine | |
| dK, (D)Lys, (D)-Lys, | D-lysine | |
| lys, or k | | |
| 5Apa | 5AminoPentanoicAcid | |
| 2-Nal or 2Nal | 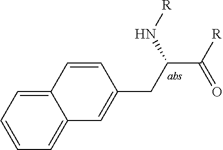 | O═C([C@H](Cc1cc2ccccc2cc1)N[R])[R] |
| | C₁₃H₁₁NOR₂ | |
| 3MeH | 3-methyl-L-histidine | Cn1cncc1C[C@H](N[R])C([R])═O |
| 3Pya, 3Pal, 3-(2- pyridyl)-alanine | 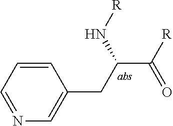 | O═C([C@H](Cc1cnccc1)N[R])[R] |
| THP, 4- aminotetrahydro- 2H-pyran- 4-carboxylic acid | 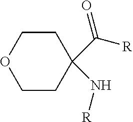 | O═C(C1(CCOCC1)N[R])[R] |
| | 4-amino-4-carboxy-tetrahydropyran | |
| 7PhW, 7PhTrp or W(7-Ph) | 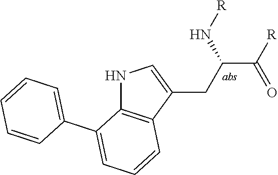 | O═C([C@H](Cc1c[nH]c2c1cccc2-c1cccc1)N[R])[R] |
| | 7-phenyl-L-tryptophan | |
| 7MeW, 7(MeW), 7MeTrp, 7-methyl-L- tryptophan | 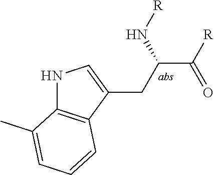 | Cc1cccc2c1[nH]cc2C[C@@H](C([R])═O)N[R] |
| | 7-methyl-L-tryptophan | |
| Abu | 2-aminobutyric acid | C[C@@H](C═O)N |
| AEF, Phe(4-(2- aminoethoxy)), or F(4- 2ae) | 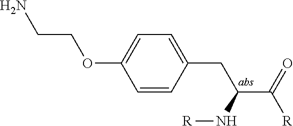 | NCCOc1ccc(C[C@@H](C([R])═O)N[R])cc1 |
| | 4-(2-aminoethoxy)-L-phenylalanine | |
| Ahp, 7Ahp, 7AHP, or | 7-aminoheptanoic acid | O═C([R])CCCCCCN[R] |
| 7AHP(2) | | |
| Ahx or 6Ahx, 6Ahx, | 6-aminohexanoic acid | O═C(CCCCCN[R])[R] |
| 6Ahx(2), | | |
| 6-aminohexanoic acid | | |
| aMeF, aMePhe, or aMe-Phe | 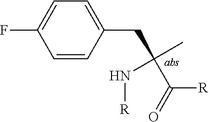 | C[C@](Cc(cc1)ccc1F)(C([R])═O)N[R] |
| | alpha-methyl L-phenylalinine | |
| aMeK, aMeLys, or | alpha-methyl L-lysine | |
| aMe-Lys | | |
| Arg or R | L-arginine | |
| dR, arg, r or (D)Arg | D-arginine | |
| Asn or N | L-asparagine | |
| Ava, 5Ava(2), or 5Ava | 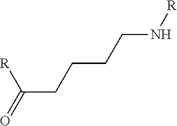 | O═C(CCCCN[R])[R] |
| | 5-Aminovaleric Acid | |
| bAla, b-ALA, beta- Alanine, bA | 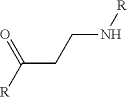 | O═C(CCN[R])[R] |
| | beta-alanine | |
| Bis-amino-PEG2 | 1,2-bis(2-aminoethoxy)ethane | |
| Cys or C | L-cysteine | |
| Dbu, Dab, (S)-2,4- diaminobutanoic acid, or DAB | 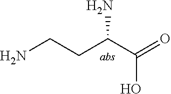 | NCC[C@@H](C(O)═O)N |
| | L-2,4-diaminobutyric acid | |
| Dap, Dap, DAP, Dpr or (S)-2,3- diaminopropanoic acid | 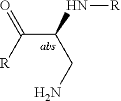 | NC[C@@H](C([R])═O)N[R] |
| | L-2,3-diaminopropionic acid | |
| dDab, D(Dab), dDpr, | D-2,4-diaminobutyric acid | NC[C@H](C([R])═O)N[R] |
| (R)-2,3- | | |
| diaminopropanoic acid | | |
| dDap, D(Dap), dDap, dap, dDbu, (R)-2,3- diaminopropanoic acid | 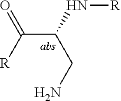 | NC[C@H](C([R])═O)N[R] |
| | D-2,3-diaminopropionic acid | |
| Fmoc-2Nal | 2-((((9H-fluoren-9- | |
| | yl)methoxy)carbonyl)amino)-3- | |
| | (naphthalen-2-yl)propanoic acid | |
| Fmoc-3Pya | (S)-2-((((9H-fluoren-9-yl)methoxy)carbonyl) | |
| | amino)-4-(pyridin-3-yl)butanoic acid | |
| Fmoc-7MeW | (S)-2-((((9H-fluoren-9- | |
| | yl)methoxy)carbonyl)amino)-3- | |
| | (7-methyl-1H-indol-3- | |
| | yl)propanoic acid | |
| Fmoc-AEF | (S)-2-((((9H-fluoren-9- | |
| | yl)methoxy)carbonyl)amino)-3- | |
| | (4-(2-((tert- | |
| | butoxycarbonyl)amino)ethoxy) | |
| | phenyl)propanoic acid | |
| Fmoc-aMePhe | (((9H-fluoren-9- | |
| | yl)methoxy)carbonyl)- | |
| | alphamethyl-L-phenylalanine | |
| Fmoc-arg or Fmoc-r | N-alpha-(9- | |
| | fluorenylmethyloxycarbonyl)-N′-2,2,4,6,7- | |
| | pentamethyldihydrobenzofuran- | |
| | 5-sulfonyl-D-arginine | |
| Fmoc-Asn or Fmoc-N | N²-(((9H-fluoren-9- | |
| | yl)methoxy)carbonyl)-N4-trityl- | |
| | L-asparagine | |
| Fmoc-Dap(DDe) | N2-(Fmoc)-N6-(1-(4,4- | |
| | dimethyl-3,5- | |
| | dioxocyclohexylidene)ethyl)-L- | |
| | Dap | |
| Fmoc-DDe-Lys(Fmoc)- | N6-(((9H-fluoren-9- | |
| OH | yl)methoxy)carbonyl)-N2-(1- | |
| | (4,4-dimethyl-3,5- | |
| | dioxocyclohexylidene)ethyl)-L- | |
| | lysine | |
| Fmoc-Glu or Fmoc-E | (S)-2-((((9H-fluoren-9- | |
| | yl)methoxy)carbonyl)amino)-5- | |
| | (tert-butoxy)-2-methyl-5- | |
| | oxopentanoic acid | |
| Fmoc-Lys(Ac) or | N2-(((9H-fluoren-9- | |
| Fmoc-K(Ac) | yl)methoxy)carbonyl)-N6- | |
| | acetyl-L-lysine | |
| Fmoc-Lys(DDe) or | N2-(Fmoc)-N6-(1-(4,4- | |
| Fmoc-K(DDe) | dimethyl-3,5- | |
| | dioxocyclohexylidene)ethyl)-L- | |
| | lysine | |
| Fmoc-Lys(NMeAc) or | N2-(((9H-fluoren-9- | |
| Fmoc-K(NMeAc) | yl)methoxy)carbonyl)-N6- | |
| | acetyl-N6-methyl-L-lysine | |
| Fmoc-NMeLys(DDe) or | (9H-fluoren-9-yl)methyl (1-amino- | |
| Fmoc-NMeK(DDe) | 6-((1-(4,4-dimethyl-3,5- | |
| | dioxocyclohexylidene)ethyl)amino)- | |
| | 1-oxohexan-2-yl)(methyl)carbamate | |
| Fmoc-Pen-Trt | (R)-2-((((9H-fluoren-9- | |
| | yl)methoxy)carbonyl)amino)-3- | |
| | methyl-3-(tritylthio)butanoic | |
| | acid | |
| Fmoc-Pro or | Fmoc-proline-OH | |
| Fmoc-P | | |
| Fmoc-pro or | Fmoc-D-proline-OH | |
| Fmoc-p | | |
| Fmoc-R5H | (R)-2-((((9H-fluoren-9- | |
| | yl)methoxy)carbonyl)amino)hept-6- | |
| | enoic acid | |
| Fmoc-Sar or Fmoc-Sarc | N-(((9H-fluoren-9- | |
| | yl)methoxy)carbonyl)-N- | |
| | methylglycine | |
| Fmoc-THP | 4-((((9H-fluoren-9- | |
| | yl)methoxy)carbonyl)amino) | |
| | tetrahydro-2H-pyran-4-carboxylic | |
| | acid | |
| Fmoc-Thr or Fmoc-T | N-(((9H-fluoren-9- | |
| | yl)methoxy)carbonyl)-O-(tert- | |
| | butyl)-L-threonine | |
| GABA, Gaba, Gaba(2), Gaba2, or 4Abu | 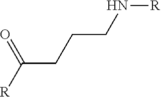 | O═C(CCCN[R])[R] |
| | 4-aminobutyric acid | |
| Glu or E | L-glutamic acid | |
| glu or e or D(Glu) | D-glutamic acid | |
| His or H | L-histidine | |
| Lys or K | L-lysine | |
| lys or k or (D)Lys | D-lysine | |
| hCys, hC | L-Homocysteine | C(CS)[C@@H](C(═O)O)N |
| KAc, Lys(Ac), K(Ac), | N-ϵ-acetyl-L-Lysine | CC(NCCCC[C@@H](C([R])═O)N[R])═O) |
| k(COMe), or K-Ac | N6-Acetyl-L-lysine | |
| MeK, N-MeLys, | N-methyl-Lysine | |
| NMeLys, NMeK, or MeLys | (2S)-2-amino-6- | |
| | (methylamino)hexanoic acid | |
| Pen | 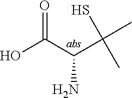 | CC(C)([C@@H](C(O)═O)N)S |
| | L-penicillamine, 3-Mercapto-L-valine | |
| | (R)-2-Amino-3-mercapto-3- | |
| | methylbutanoic acid | |
| F4CONH2, Phe(4-CONH₂) or | 4-carbamoyl-L-phenylalanine | N[C@H](C([R])═O)Cc1ccc(C(N[R])═O)cc1 |
| Phe(4-CONH2) or Phe(Cmd) | (S)-2-amino-3-(4- | |
| or Phe_4Ad | carbamoylphenyl)propanoic acid | |
| F4OMe, Phe(4-OMe), | 4-methoxy-L-phenylalanine | N[C@@H](CC1═CC═C(OC)C═C1)C(O)═O |
| or Phe_4OMe | | |
| Quin, 3Quin, 3-Quin, 3QuinolAla, or 3QuinA | 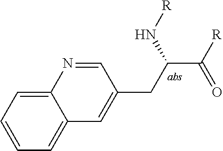 | O═C([C@H](Cc1cc2ccccc2nc1)N[R])[R] |
| | (S)-2-amino-3-(quinolin-3- | |
| | yl)propanoic acid | |
| R5H, | (R)-2-aminopentanoic acid 5-diyl | |
| R6H, (R,E)-2-amino-8- | (R)-2-aminohexanoic acid 6-diyl | C═CCCCC[C@H](C([R])═O)N[R] |
| hydroxyoct-7-enoic acid | | |
| R7H, (R,E)-2-amino-9- | (R)-2-aminoheptanoic acid 7-diyl | C═CCCCCC[C@H](C([R])═O)N[R] |
| hydroxynon-8-enoic acid | | |
| S5H | (S)-2-aminopentanoic acid 5-diyl | C═CCCC[C@H](N[R])C([R])═O |
| meG, Sarc, MeGly, Sar, Sarc, MeGly, Sarcosine, Methylamino-Acetic Acid, N-methylglycine | 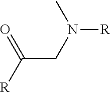 | CN(CC([R])═O[R] |
| | sarcosine or N-methylglycine | |
| Thr or T | L-threonine | |
| nFEtOH, Phe(4- | Fc1c(F)c([H])c(F)c(F)c1NC[C@@H](C([R])═O)N[R] | N[C@@H](C═O)c(cc1)ccc1OCC═O |
| OCH2COOH, or 2-amino-2-[4- | (R)-2-amino-2-(4-(carboxymethoxy)phenyl)acetic acid | |
| (carboxymethoxy)phenyl]acetic acid, | | |
| DappF6 | tetra-fluoro-phenylalanine | Fc1c(F)c([H])c(F)c(F)c1NC[C@@H](C([R])═O)N[R] |
| Dap(pF(6)) | | |

TABLE 2B
Abbreviations for Substituents, Reagents, and Solvents

| Abbreviation | Definition | Smiles |
|---|---|---|
| Ac or MeCO | acetyl | |
| ACN | acetonitrile | |
| Boc | tert-butoxy-carbonyl | |
| CONH₂ | carboxamide | |
| COOH | carboxylic Acid | |
| DCM | dichloromethane | |
| Dde | N-(1-(4,4-dimethyl-2,6- | |
| | dioxocyclohexylidene)ethyl | |
| DIC | N,N′-diisopropylcarbodiimide | |
| DMF | N,N-dimethylformamide | |
| Et₂O | di-ethylether | |
| FMOC or Fmoc | ((9H-fluoren-9-yl)methoxy)carbonyl | |
| HOAT or HOAt | 1-hydroxy-7-azabenzotriazole | |
| MeOH | methanol | |
| MTBE | methyl tert-butyl ether | |
| MW | microwave | |
| Oxyma | ethyl cyanohydroxyiminoacetate | |
| PEG2_DiAcid or PEG2DA | 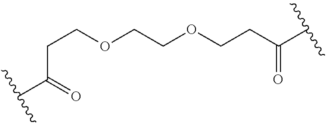 [SMILES: *C(=O)CCOCCOCCC(*)=O] | |
| | 3,3′-(ethane-1,2- | |
| | diylbis(oxy))dipropanecarbonyl | |
| pF | 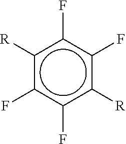 | Fc1c(F)c([R])c(F)c(F)c1[R] |
| | 2,3,5,6-tetrafluorophen-1,4-diyl linker | |
| pFS | 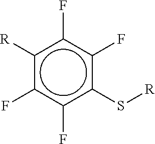 | Fc(c(S[R])c(c(F)c1[R])F)c1F |
| | 2,3,5,6-tetrafluoro-4-mercaptophenol | |
| RT | room temperature | |
| TFA | trifluoroacetic acid | |
| TIPS | triisopropylsilane | |

TABLE 2C
Monomers

| # | Symbol/Name | Structure | Smiles |
|---|---|---|---|
| 1 | bMeW(2S3R) bMeW(2S,3R) | 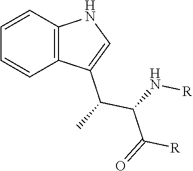 | C[C@H](C1═CNC2═C1C═CC═C2) [C@H](N[R])C([R])═O |
| | | C₁₂H₁₂N₂OR₂ | |
| 2 | bMeW(2S3S), bMeW(2S,3S) | 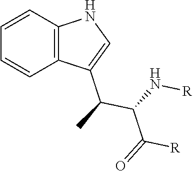 | C[C@@H](C1═CNC2═C1C═CC═C2) [C@H](N[R])C([R])═O |
| | | C₁₂H₁₂N₂OR₂ | |
| 3 | 6OH2Nal | 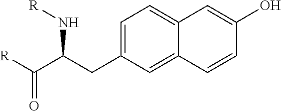 | [R]C([C@H]CC1═CC═C(C═C(O) C═C2)C2═C1)N[R])═O |
| | | (S)-2-amino-3-(6-hydroxynaphthalen-2-yl)propanoic acid | |
| 4 | NMe7MeW | 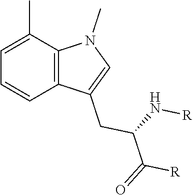 | [R]C([C@@H](N[R])CC1═CN(C) C2═C1C═CC═C2C)═O |
| | | C₁₃H₁₄N₂OR₂ | |
| 5 | 7(4Paz)W | 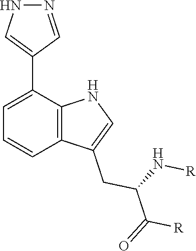 | [R]C([C@@H](N[R]) CC1═CNC2═C1C═CC═C2C3═CNN═ C3)═O |
| | | C₁₄H₁₂N₄OR₂ | |
| 6 | 7(7(124TAZP))W | 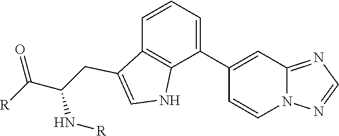 | [R]C([C@@H](N[R]) CC1═CNC2═C1C═CC═C2C3═CC4═ NC═NN4C═C3)═O |
| | | C₁₇H₁₃N₅OR₂ | |
| 7 | 7(3UrPh)W | 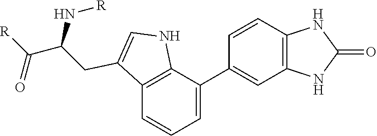 | [R]C([C@@H](N[R]) CC1═CNC2═C1C═CC═C2C3═ CC(NC(N4)═O)═C4C═C3)═O |
| | | C₁₈H₁₄N₄O₂R₂ | |
| 8 | 7(7Imzpy)W | C₁₈H₁₄N₄OR₂ | [R]C([C@@H](N[R]) |
| | | | CC1═CNC2═C1C═ |
| | | | CC═C2C3═CC4═NC═CN4C═C3)═O] |
| 9 | 7(4OMePh)W | 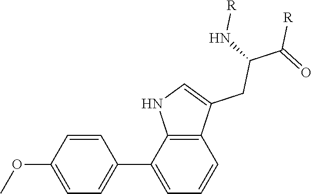 | [R]C([C@@H](N[R]) CC1═CNC2═C1C═ CC═C2C3═CC═C(OC)C═C3)═O |
| | | C₁₈H₁₆N₂O₂R₂ | |
| 10 | 7(3(6AzaInd1Me))W | 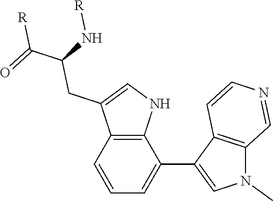 | [R]C([C@@H](N[R]) CC1═CNC2═C1C═ CC═C2C3═CN(C) C4═C3C═CN═C4)═O |
| | | C₁₉H₁₆N₄OR₂ | |
| 11 | 7(6(2MeNDAZ))W | 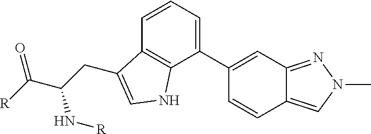 | [R]C([C@@H](N[R]) CC1═CNC2═C1C═CC═ C2C3═CC4═NN(C)C═C4C═C3)═O |
| | | C₁₉H₁₆N₄OR₂ | |
| 12 | NMebAla | 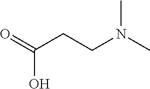 | CN(C)CCC═O |
| | | C₅H₁₁NO | |
| 13 | AcMorp, Ethyl- morpholino | 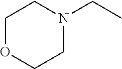 | CN1CCOCC1 |
| | | C₅H₁₁NO | |
| 14 | dOrN, D-Orn D-Ornithine | 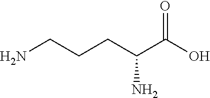 | NCCC[C@H](C(O)═O)N |
| | | C₅H₁₂N₂O | |
| 15 | 3Hyp, 3-Hydroxy- L-proline | 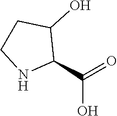 | OC1[C@@H](C═O)NCC1 |
| | | C₅H₉NO₂ | |
| 16 | aMaE aMeGlu, alpha- methyl glutamic acid | 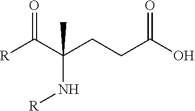 | C[C@](CCC(O)═O)(C([R])═O)N[R] C[C@](CCC(O)═O)(C═O)N |
| | | C₆H₁₁NO₂ | |
| 17 | hGlu, (S)-2- aminohexanedioic acid | 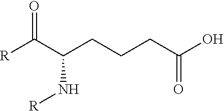 | N[C@@H](CCCC═O) C═OOC(CCC[C@@H](C([R])═O) N[R])═O |
| | | C₆H₁₁NO₂ | |
| 18 | CON(NMePip) | 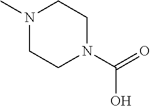 | CN(CC1)CCN1C═O |
| | | C₆H₁₂N₂O | |
| 19 | -CODiFPip, CO(DiFPip) | 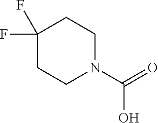 [SMILES: O=C(O)N1CCC(F)(F)CC1] | O═CN(CC1)CCC1(F)F |
| | | C₆H₉F₂NO | |
| 20 | CO(OAZBO) | 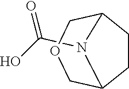 [SMILES: O=C(O)N1C2CCC1COC2] | CC(N1C2COCC1C2)═O |
| | | C₈H₁₃NO₂ | |
| 21 | Me1Pya, (S)-3-(2- amino-2- carboxyethyl)-1- methylpyridin- 1-ium | 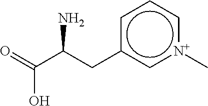 | C[n+]1cccc(C[C@@H](C═O)N)c1 |
| | | C₉H₁₃N₂O⁺ | |
| 22 | DappF6, tetra-fluoro- phenylalanine | 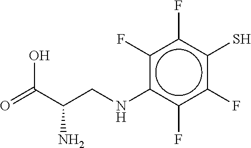 | N[C@@H](CNc(c(F)c(c(S)c1F)F) c1F)C═O |
| | | C₉H₈F₄N₂O | |
| 23 | bMePhe(2S,3R) bMePhe(SR), bMePhe(2S,3R) | 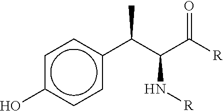 | C[C@@H]([C@H](C([R])═O)N[R]) c1ccccc1 |
| | | C₁₀H₁₁NOR₂ | |
| 24 | N4AmBenzylGly, N(4AmBenzyl)Gly | 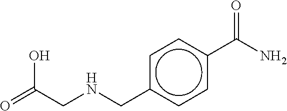 | NC(c1ccc(CNCC═O)cc1)═O |
| | | C₁₀H₁₂N₂O₂ | |
| 25 | -Dec, 1,10- Decanedioic Acid | 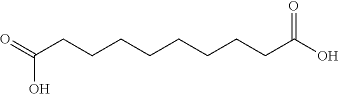 [SMILES: O=C(O)CCCCCCCCC(=O)O] | OC(CCCCCCCCCC═O)═O |
| | | C₁₀H₁₈O₃ | |
| 26 | 2OH3Pyrimid5Ala | 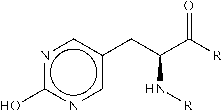 | CC(C)(C)Oc1ncc(C[C@@H] (C([R])═O)N[R])cn1 |
| | | C₁₁H₁₅N₃O₂R₂ | |
| 27 | KacMorph, K(AcMorph), KAcMorph, L- Lysine(ac- Morpholino | 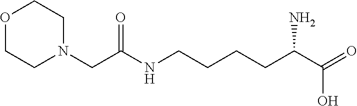 [SMILES: N[C@@H](CCCCNC(=O)CN1CCOCC1)C(=O)O] | N[C@@H] (CCCCNC(CN1CCOCC1) ═O)C═O |
| | | C₁₂H₂₃N₃O₃ | |
| 28 | 6OH2Nal | 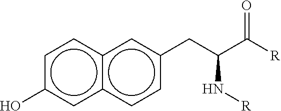 | N[C@@H](Cc1cc2ccccc2cc1) C═ON[C@@H] (Cc1cc2ccccc2cc1) C═OOc1ccc(cc(C[C@@H] (C([R])═O)N[R])cc2)c2c1 |
| | | C₁₃H₁₃NO | |
| 29 | DabNMecarn, Dab(NMecarn) | 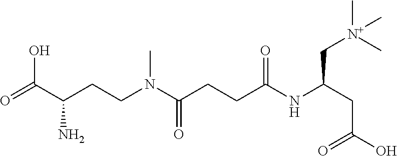 [SMILES: CN(CC[C@H](N)C(=O)O)C(=O)CCC(=O)N[C@H](CC(=O)O)C[N+](C)(C)C] | CN(CC[C@@H] (C═O)N)C(CCC(N[C@H] (CC═O)C[N+](C)(C)C)═O)═O CN(CC[C@@H] (C═O)N)C(CCC(N[C@H] (CC═O)C[N+](C)(C)C)═O)═O CN(CC[C@@H] (C═O)N)C(CCC(N[C@H] (CC(O)═O)C[N+](C)(C)C)═O)═O |
| | | C₁₆H₃₁N₄O₄⁺ | |
| 30 | DabNMeCarn, Dab(NMeCarn) | 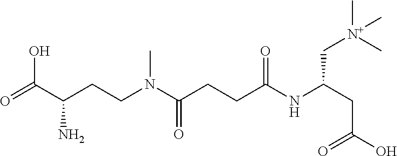 [SMILES: CN(CC[C@H](N)C(=O)O)C(=O)CCC(=O)N[C@@H](CC(=O)O)C[N+](C)(C)C] | CN(CC[C@@H](C═O) N)C(CCC(N[C@@H] (CC(O)═O)C[N+](C)(C)C)═O)═O |
| | | C₁₆H₃₁N₄O₅⁺ | |
| 31 | F(4TzlTMA4) | 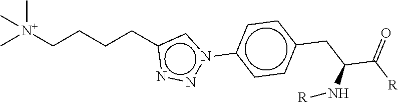 | C[N+](C)(C) CCCCc1cn(-c2ccc(C[C@@H] (C([R])═O)N[R])cc2)nn1 |
| | | C₁₈H₂₆N₅OR₂⁺ | |
| 32 | NMeK(d), NMeKdCar | 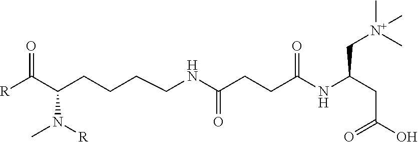 | CN([C@@H](CCCCNC(CCC(N[C@H] (CC(O)═O)C[N+] (C)(C)C)═O)═O)C([R])═O)[R] |
| | | C₁₈H₃₃N₄O₅R₂⁺ | |
| 33 | 7(5(Ina7Pyr))W | 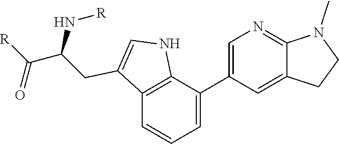 | [R]C([C@@H](N[R]) CC1═CNC2═C1C═CC═ C2C3═CN═C(N(C)CC4)C4═C3)═O |
| | | C₁₉H₁₈N₄OR₂ | |
| 34 | F(4TzlTMA5) | 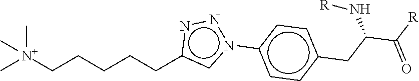 | C[N+](C)(C)CCCCCc1cn (-c2ccc(C[C@@H] (C([R])═O)N[R])cc2)nn1 |
| | | C₁₉H₂₈N₅OR₂⁺ | |
| 35 | CF3COF3CO | 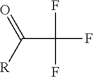 | O═C(C(F)(F)F)[R] |
| | | C₂F₃OR | |
| 36 | CF3Propylamide | 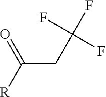 | O═C(CC(F)(F)F)[R] |
| | | C₃H₂F₃OR | |
| 37 | C(1*) (*pure but configuration unknown) | 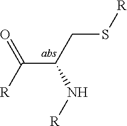 | O═C(C([C@H](CS[R])N[R])[R] |
| | | C₃H₄NOSR₃ | |
| 38 | bAla, b-ALA, beta- Alanine, bA | 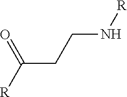 | O═C(CCN[R])[R] |
| | | C₃H₅NOR₂ | |
| 39 | CON(Me)2 | 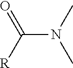 | CN(C)C([R])═O |
| | | C₃H₆NOR | |
| 40 | D(2) | 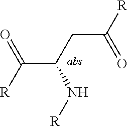 | O═C(C[C@@H](C([R])═O)N[R])[R] |
| | | C₄H₄NO₂R₃ | |
| 41 | cPrCO | 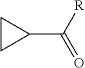 | O═C(C1CC1)[R] |
| | | C₄H₅OR | |
| 42 | hS, hS, , Hse, L- homoserine, homoSm, or homoSer | 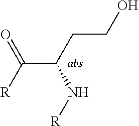 | OCC[C@@H](C([R])═O)N[R] |
| | | C₄H₇NO₂R₂ | |
| 43 | T, dThr, dT | 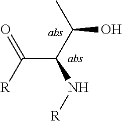 | C[C@H]([C@H](C([R])═O)N[R])O |
| | | C₄H₇NO₂R₂ | |
| 44 | 4sb, 4SB | 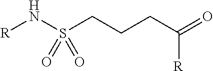 | O═C([R])CCCS(═O)(N[R])═O |
| | | C₄H₇NO₃SR₂ | |
| 45 | Aib, AIB, 2- Aminoisobutyric acid, Alpha- aminoisobutyric acid, (2-aminoalanine) | 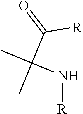 | CC(C)(C([R])═O)N[R] |
| | | C₄H₇NOR₂ | |
| 46 | | | |
| 47 | NMebAla | 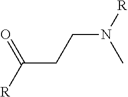 | CN(CCC([R])═O)[R] |
| | | C₄H₇NOR₂ | |
| 48 | aMeC | 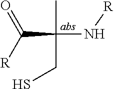 | C[C@](CS)(C([R])═O)N[R] C[C@](CS)(C═O)N |
| | | C₄H₇NOSR₂ | |
| 49 | hC, hCys, homoC, or homoCys | 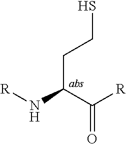 | O═C([C@H](CCS)N[R])[R] |
| | | C₄H₇NOSR₂ | |
| 50 | iPrCO | 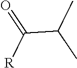 | CC(C)C([R])═O |
| | | C₄H₇OR | |
| 51 | dDab, dab, (R)-2,4- diaminobutanoic acid | 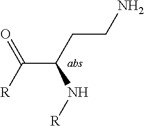 | NCC[C@H](C([R])═O)N[R] |
| | | C₄H₈N₂OR₂ | |
| 52 | homobAla | 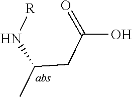 | C[C@@H](CC(O)═O)N[R] |
| | | C₄H₈NO₂R | |
| 53 | Bua, Butanoic acid | 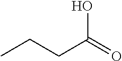 | CCCC(O)═O |
| | | C₄H₈O₂ | |
| 54 | Orn, ORN, Ornithine | 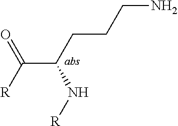 | NCCC[C@@H](C([R])═O)N[R] |
| | | C₅H₁₀N₂OR₂ | |
| 55 | | | |
| 56 | Orn, L-ornithine | 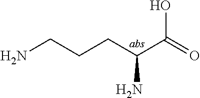 | NCCC[C@@H](C(O)═O)N |
| | | ₅H₁₂N₂O₂ | |
| 57 | 4diFPro | 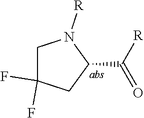 | O═C([C@H](CC(C1)(F)F)N1[R]) [R]O═C[C@H](C1)NCC1(F)F |
| | | C₅H₅F₂NOR₂ | |
| 58 | prG, prG, Fmoc-L- propargyl-Gly-OH, Pra | 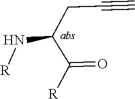 | C#CC[C@@H](C[R])═O)N[R] |
| | | C₅H₅NOR₂ | |
| 59 | 4TriazolAla | 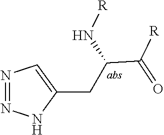 | O═C([C@H](Cc1cnn[nH]1)N[R])[R] |
| | | C₅H₆N₄OR₂ | |
| 60 | Tzl | 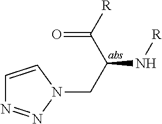 | O═C([C@H](Cn1nncc1)N[R])[R] |
| | | C₅H₆N₄OR₂ | |
| 61 | PyE, PyE (S)-5- oxopyrrolidine- 2-carboxylic acid | 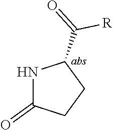 | O═C([C@H](CC1)NC1═O) [R]O═C[C@H](CC1)NC1═O |
| | | C₅H₆NO₂R | |
| 62 | E(2) | 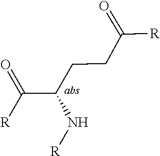 | O═C(CC[C@@H](C([R])═O)N[R])[R] |
| | | C₅H₆NO₂R₃ | |
| 63 | Tetrazole | 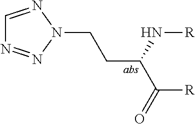 | O═C([C@H](CCn1nncn1)N[R])[R] N[C@@H](CCn1ncn1)C═O |
| | | C₅H₇N₅OR₂ | |
| 64 | 3OHPro | 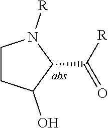 | OC(CC1)[C@@H](C([R])═O)N1[R] |
| | | C₅H₇NO₂R₂ | |
| 65 | 4(R)HydroxyPro | 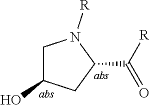 | O[C@H](C[C@H]1C([R])═O)CN1[R] |
| | | C₅H₇NO₂R₂ | |
| 66 | Hyp | 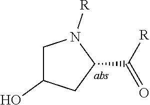 | OC(C[C@H]1C([R])═O)CN1[R] |
| | | C₅H₇NO₂R₂ | |
| 67 | AllylGly | 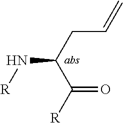 | C═CC[C@@H](C([R])═O)N[R] |
| | | C₅H₇NOR₂ | |
| 68 | Dap(Ac) | 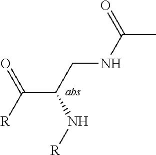 | CC(NC[C@@H](C([R])═O)N[R])═O |
| | | C₅H₈N₂O₂R₂ | |
| 69 | N(NMe), NNMe, NMeAsn | 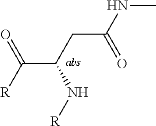 | CNC(C[C@@H](C([R])═O)N[R])═O |
| | | C₅H₈N₂O₂R₂ | |
| 70 | aMeN, aMeAsn | 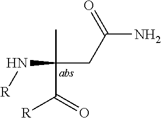 | C[C@](CC(N)═O)(C([R])═O)N[R] |
| | | C₅H₈N₂O₂R₂ | |
| 71 | 4(S)AminoPro | 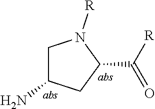 | N[C@@H](C[C@H] 1C([R])═O)CN1[R] |
| | | C₅H₈N₂OR₂ | |
| 72 | CO(Morph) | 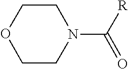 | O═C(N1CCOCC1)[R] |
| | | C₅H₈NO₂R | |
| 73 | -COMorph, CO(Morph) | 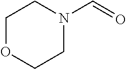 | O═CN1CCOCC1 |
| | | C₅H₉NO₂ | |
| 74 | | | |
| 75 | Nva | 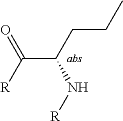 | CCC[C@@H](C([R])═O)N[R] |
| | | C₅H₉NOR₂ | |
| 76 | dM, dMet, D- Methionine | 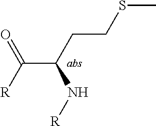 | CSCC[C@H](C([R])═O)N[R] |
| | | C₅H₉NOSR₂ | |
| 77 | dPen, pen | 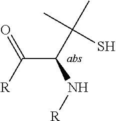 | CC(C)([C@H](C([R])═O)N[R])S |
| | | C₅H₉NOSR₂ | |
| 78 | BuCO | 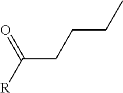 | CCCCC([R])═O |
| | | C₅H₉OR | |
| 79 | iBuCO | 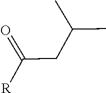 | CC(C)CC([R])═OCC[C@H] (C)C([R])═O |
| | | C₅H₉OR | |
| 80 | tBuCO | 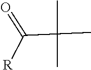 | CC(C)(C)C([R])═O |
| | | C₅H₉OR | |
| 81 | N(N(Me)2), NNMe2 | 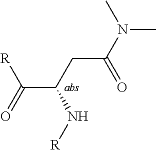 | CN(C)C(C[C@@H] (C([R])═O)N[R])═O |
| | | C₆H₁₀N₂O₂R₂ | |
| 82 | MorphCO, 2- morpholinoacetic acid | 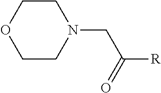 | O═C(CN1CCOCC1)[R] |
| | | C₆H₁₀NO₂R | |
| 83 | CON(NMePip) | 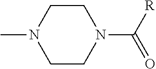 | CN(CC1)CCN1C([R])═O |
| | | C₆H₁₁N₂OR | |
| 84 | eK | 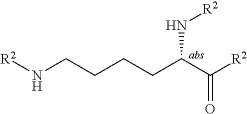 | O═C(O)[C@@H](N[R])CCCCN[R] |
| | | C₆H₁₁N₂OR₃ | |
| 85 | Cit, Citrulline | 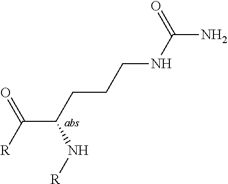 | NC(NCCC[C@@H] (C([R])═ON[R])═ON[C@@H] (CCCNC(N)═O)C(O)═O |
| | | C₆H₁₁N₃O₂R₂ | |
| 86 | D(NEtNH2) | 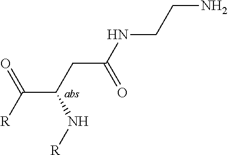 | NCCNC(C[C@@H] (C([R])═O)N[R])═O |
| | | C₆H₁₁N₃O₂R₂ | |
| 87 | Aad, 2-Aminoadipic acid | 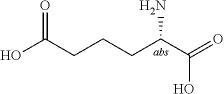 [SMILES: N[C@@H](CCCC(=O)O)C(=O)O] | N[C@@H](CCCC(O)═O)C(O)═O |
| | | C₆H₁₁NO₄ | |
| 88 | N(Isobutyl)Gly | 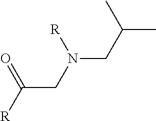 | CC(C)CN(CC([R])═O)[R] |
| | | C₆H₁₁NOR₂ | |
| 89 | PentCO | 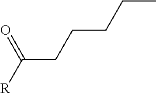 | CCCCCC([R])═O |
| | | C₆H₁₁OR | |
| 90 | NMeQ, NMeGln, N- Methyl-Glutamine | 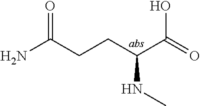 | CN[C@@H](CCC(N)═O)C(O)═O |
| | | C₆H₁₂N₂O₃ | |
| 91 | SP6 | 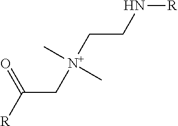 | C[N+](C)(CCN[R]) CC([R])═OC[N+](C)(CCN)CC═O |
| | | C₆H₁₃N₂OR₂⁺ | |
| 92 | 3IOxa4Ala | 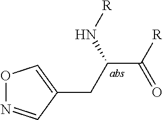 | O═C([C@H](Cc1conc1)N[R])[R] |
| | | C₆H₆N₂O₂R₂ | |
| 93 | 3Oxa4Ala | 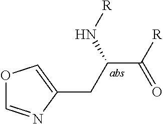 | O═C([C@H](Cc1cocn1)N[R])[R] |
| | | C₆H₆N₂O₂R₂ | |
| 94 | diFCpx | 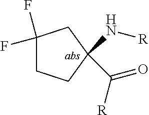 | O═C([C@](CC1)(CC1(F)F)N[R])[R] |
| | | C₆H₇F₂NOR₂ | |
| 95 | aMePra | 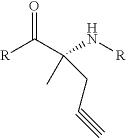 | C[C@](CC#C)(C([R])═O)N[R] |
| | | C₆H₇NOR₂ | |
| 96 | CO(DiFPip) | 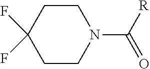 | O═C(N(CC1)CCC1(F)F)[R] |
| | | C₆H₈F₂NOR | |
| 97 | dab(COCH2(1*)) dab(COCH2)(1*) | 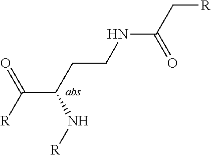 | O═C(C[R]) NCC[C@@H](C([R])═O)N[R] |
| | | C₆H₉N₂O₂R₃ | |
| 98 | Tetrazole(NMe) | 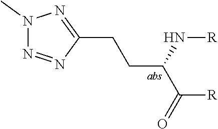 | Cn1nnc(CC[C@@H](C([R])═O)N[R])n1 |
| | | C₆H₉N₅OR₂ | |
| 99 | | | |
| 100 | dhE | 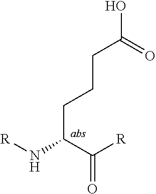 | OC(CCC[C@H](C([R])═O)N[R])═O |
| | | C₆H₉NO₃R₂ | |
| 101 | Acpx | 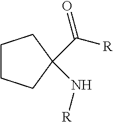 | O═C(C1(CCCC1)N[R])[R] NC1(CCCC1)C═O |
| | | C₆H₉NOR₂ | |
| 102 | aMeP, aMePro | 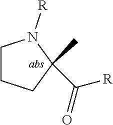 | C[C@](CCC1)(C([R])═O)N1[R] |
| | | C₆H₉NOR₂ | |
| 103 | D(N2AmIm) | 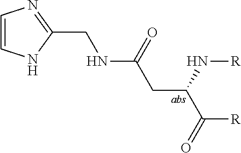 | O═C(C[C@@H] (C([R])═O)N[R])NCc1ncc[nH]1 |
| | | C₈H₁₀N₄O₂R₂ | |
| 104 | KTfa, K(Tfa), L-Lys(Tfa) | 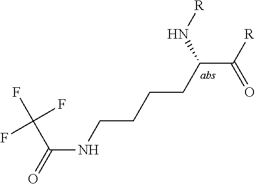 | O═C([C@H] (CCCCNC(C(F)(F)F)═O)N[R])[R] |
| | | C₈H₁₁F₃N₂O₂R₂ | |
| 105 | E(OAll) | 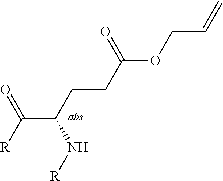 | C═CCOC(CC[C@@H] (C([R])═O)N[R])═O |
| | | C₈H₁₁NO₃R₂ | |
| 106 | D(NPyr) | 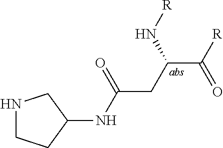 | O═C(C[C@@H] (C([R])═O)N[R])NC1CNCC1 |
| | | C₈H₁₃N₃O₂R₂ | |
| 107 | Chg | 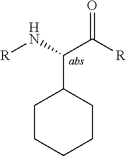 | O═C([C@H](C1CCCCC1)N[R])[R] |
| | | C₈H₁₃NOR₂ | |
| 108 | R5Me, aMeR5H | 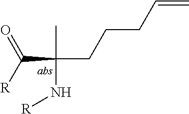 | C[C@@](CCCC═C)(C([R])═O)N[R] |
| | | C₈H₁₃NOR₂ | |
| 109 | R6H, (R,E)- 2-amino- 8-hydroxyocty- 7-enoic acid | 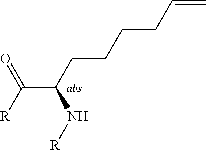 | C═CCCCC[C@H] (C([R])═O)N[R]C═CCCCC)[C@H] (C═O)N |
| | | C₈H₁₃NOR₂ | |
| 110 | S5Me aMeS5H | 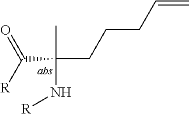 | C[C@](CCCC═C)(C([R])═O)N[R] |
| | | C₈H₁₃NOR₂ | |
| 111 | S6H | 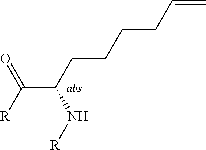 | C═CCCCC[C@@H](C([R])═O)N[R] |
| | | C₈H₁₃NOR₂ | |
| 112 | KAc, K(Ac), K(COMe), K-Ac, N6-acetyl-L-Lysine | 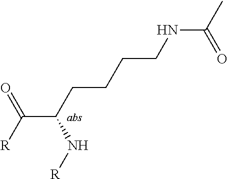 | CC(NCCCC[C@@H] (C([R])═O)N[R])═O |
| | | C₈H₁₄N₂O₂R₂ | |
| 113 | Pip(NMe2) | 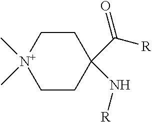 | C[N+](C)(CC1)CCC1(C([R])═O)N[R] |
| | | C₈H₁₅N₂OR₂⁺ | |
| 114 | K(Gly) | 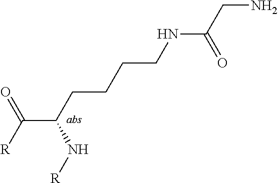 | NCC(NCCCC[C@@H] (C([R])═O)N[R])═O |
| | | C₈H₁₅N₃O₂R₂ | |
| 115 | 8Aoc, 8Aoc(2) | 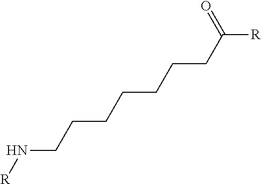 | O═C(CCCCCCCN[R])[R] |
| | | C₈H₁₅NOR₂ | |
| 116 | 2Benzyl | 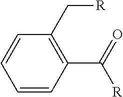 | O═C(c1c(C[R])cccc1)[R] |
| | | C₈H₆OR₂ | |
| 117 | 6OH3Pya | 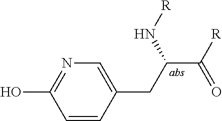 | Oc1ncc(C[C@@H] (C([R])═O)N[R])cc1 |
| | | C₈H₈N₂O₂R₂ | |
| 118 | 3Pya, 3Pal, 3-(2- pyridyl)-alanine | 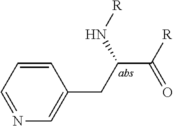 | O═C([C@H](Cc1cnccc1)N[R])[R] |
| | | C₈H₈N₂OR₂ | |
| 119 | 4Pya, 4Pra, 4Pal, (S)-2-amino-3- (pyridin-4- yl)propanoic acid 4PyridinAla | 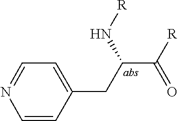 | O═C([C@H](Cc1ccncc1)N[R])[R] |
| | | C₈H₈N₂OR₂ | |
| 120 | dPal, dpal, d3Pya, 3pya, 3- pyridylalanine, (R)- 2-amino-3-(pyridin- 3-yl)propanoic acid | 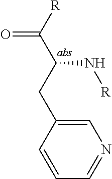 | O═C([C@@H](Cc1cnccc1)N[R])[R] |
| | | C₈H₈N₂OR₂ | |
| 121 | 6MePyridazAla | 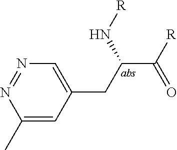 | Cc1cc(C[C@@H] (C([R])═O)N[R])cnn1 |
| | | C₈H₉N₃OR₂ | |
| 122 | 5MePyridinAla | 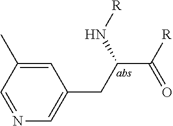 | Cc1cc(C[C@@H] (C([R])═O)N[R])cnc1 |
| | | C₉H₁₀N₂OR₂ | |
| 123 | J, Aph, 4- aminophenylalanine | 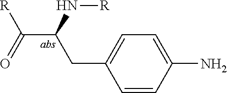 | Nc1ccc(C[C@@H](C[R])═O)N[R])cc1 |
| | | C₉H₁₀N₂OR₂ | |
| 124 | NMe3Pya | 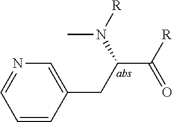 | CN([C@@H](Cc1cnccc1) C([R])═O)[R]CN[C@@H] (Cc1cnccc1)C═O |
| | | C₉H₁₀N₂OR₂ | |
| 125 | SMSBCO | 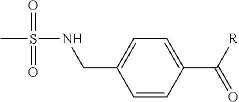 | CS(NCc(cc1)ccc1C([R])═O)(═O)═O |
| | | C₉H₁₀NO₃SR | |
| 126 | Me3Pya | 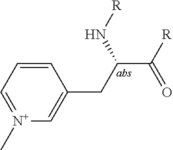 | C[n+]1cccc(C[C@@H] (C([R])═O)N[R])c1 |
| | | C₉H₁₁N₂OR₂⁺ | |
| 127 | D(Pip), (S)-2-amino- 4-oxo-4- (piperidin-1- yl)butanoic acid | 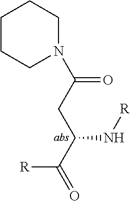 | O═C(C[C@@H] (C([R])═O)N[R])N1CCCCC1 |
| | | C₉H₁₄N₂O₂R₂ | |
| 128 | D(NPip) | 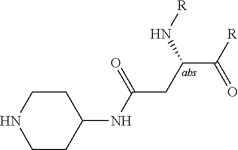 | O═C(C[C@@H] (C([R])═O)N[R])NC1CCNCC1 |
| | | C₉H₁₅N₃O₂R₂ | |
| 129 | N(Cyclohexyl)Gly | 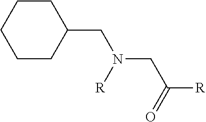 | O═C(CN(CC1CCCCC1)[R])[R] |
| | | C₉H₁₅NOR₂ | |
| 130 | R7H, (R,E)- 2-amino- 9-hydroxynon-8- enoic acid | 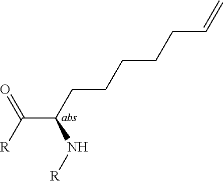 | C═CCCCCC[C@H] (C([R])═O)N[R]C═CCCCCC[C@H] (C═O)N |
| | | C₉H₁₅NOR₂ | |
| 131 | K(COEt) | 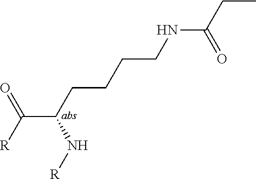 | CCC(NCCCC[C@@H] (C([R])═O)N[R])═O |
| | | C₉H₁₆N₂O₂R₂ | |
| 132 | K(NMeAc), KNMeAc | 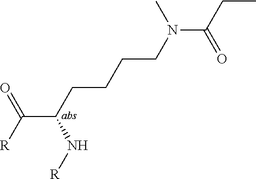 | CC(N(C)CCCC[C@@H] (C([R])═O)N[R])═O |
| | | C₉H₁₆N₂O₂R₂ | |
| 133 | Q(NHtBu) | 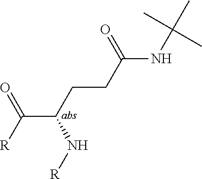 | CC(C)(C)NC(CC[C@@H] (C([R])═O)N[R])═O |
| | | C₉H₁₆N₂O₂R₂ | |
| 134 | K(Me)3 | 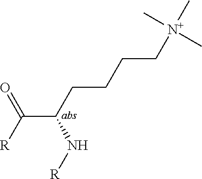 | C[N+](C)(C)CCCC[C@@H] (C([R])═O)N[R] |
| | | C₉H₁₉N₂OR₂⁺ | |
| 135 | dK(Me)3, K(Me)3 | 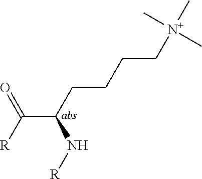 | C[N+](C)(C)CCCC[C@H] (C([R])═O)N[R] |
| | | C₉H₁₉N₂OR₂⁺ | |
| 136 | 5cpaCO | 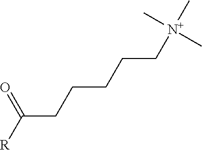 | C[N+](C)(C)CCCCCC([R])═O |
| | | C₉H₁₉NOR⁺ | |
| 137 | tetraFPhe | 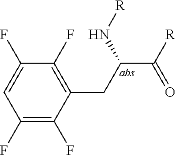 | O═C([C@H](Cc(c(F)c(cc1F)F) c1F)N[R])[R] |
| | | C₉H₅F₄NOR₂ | |
| 138 | 5CF33Pya | 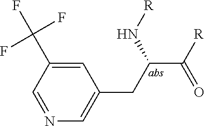 | O═C([C@H](Cc1cncc(C(F)(F)F)c1) N[R])[R]N[C@@H](Cc1cc(C(F)(F)F) cnc1)C═O |
| | | C₉H₇F₃N₂OR₂ | |
| 139 | 3,4diFPhe, 4diFPhe | 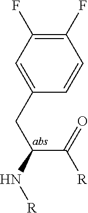 | O═C([C@H](Cc(cc1)cc(F)c1F)N[R])[R] |
| | | C₉H₇F₂NOR₂ | |
| 140 | F(4N3) | 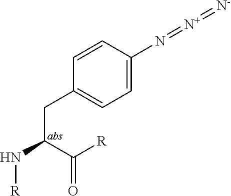 | [N−]═[N+]═Nc1ccc(C[C@@H] (C([R])═O)N[R])cc1 |
| | | C₉H₈N₄OR₂ | |
| 141 | 3FTyr | 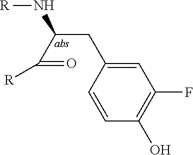 | Oc(ccc(C[C@@H](C([R])═O) N[R])c1)c1F |
| | | C₉H₈FNO₂R₂ | |
| 142 | 2BrPhe, 2BrF | 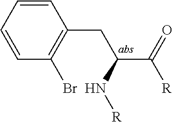 | O═C([C@H](Cc(cccc1)c1Br)N[R])[R] |
| | | C₉H₈BrNOR₂ | |
| 143 | 2FPHE, 2FPhe | 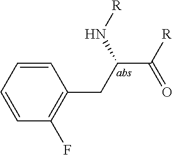 | O═C([C@H](Cc(cccc1)c1F)N[R])[R] |
| | | C₉H₈FNOR₂ | |
| 144 | 3FPHE, 3FPhe | 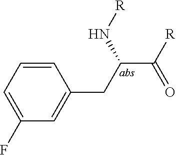 | O═C([C@H](Cc1cc(F)ccc1)N[R])[R] |
| | | C₉H₈FNOR₂ | |
| 145 | BHCO | 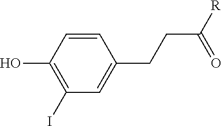 | Oc(ccc(CCC([R])═O)c1)c1I |
| | | C₉H₈IO₂R | |
| 146 | 5AmPyridinAla | 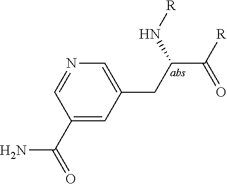 | NC(c1cc(C[C@@H] (C([R])═O)[R])cnc1)═O |
| | | C₉H₉N₃O₂R₂ | |
| 147 | mTYR, mY, mTyr | 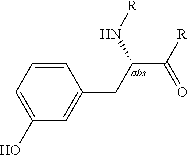 | Oc1cccc(C[C@@H](C([R])═O)N[R])c1 |
| | | C₉H₉NO₂R₂ | |
| 148 | 6OHQuin | 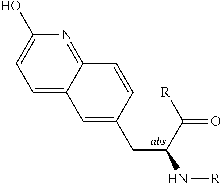 | Oc1ccc(cc(C[C@@H] (C([R])═O)N[R])cc2)c2n1 |
| | | C₁₂H₁₀N₂O₂R₂ | |
| 149 | 4AmF, 4AmPhe | 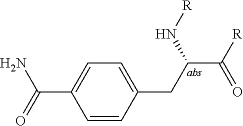 | NC(c1ccc(C[C@@H] (C([R])═O)N[R])cc1)═ON[C@@H] (Cc(cc1)ccc1C(N)═O)C═O |
| | | C₁₀H₁₀N₂O₂R₂ | |
| 150 | AEF(NMe(2)) | 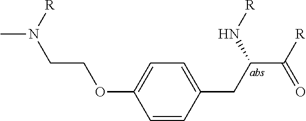 | CN(CCOc1ccc(C[C@@H] (C([R])═O)N[R])cc1)[R] |
| | | C₁₂H₁₅N₂O₂R₃ | |
| 151 | aMeY01 | 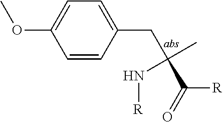 | C[C@](Cc(cc1)ccc1OC)(C([R])═O)N[R] |
| | | C₁₁H₁₃NO₂R₂ | |
| 152 | BiF | 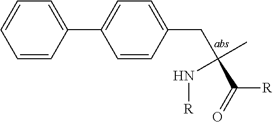 | C[C@](Cc(cc1)ccc1-c1ccccc1) (C([R])═O)N[R] |
| | | C₁₆H₁₅NOR₂ | |
| 153 | hdKMe3, hk(Me)3 | 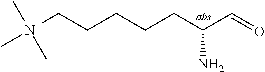 | C[N+](C)(C)CCCCC[C@H](C═O)N |
| | | C₁₀H₂₃N₂O⁺ | |
| 154 | Y(OTzl) | 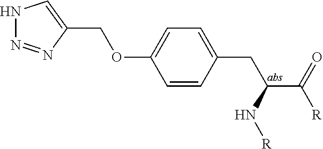 | O═C([C@H](Cc(cc1)ccc1OCc1c[nH] nn1)N[R])[R] |
| | | C₁₂H₁₂N₄O₂R₂ | |
| 155 | 3CONH2F | 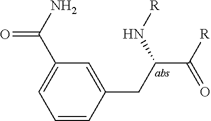 | NC(c1cccc(C[C@@H](C([R])═O) N[R])c1)═O |
| | | C₁₀H₁₀N₂O₂R₂ | |
| 156 | 4AmDF, 4AmDPhe | 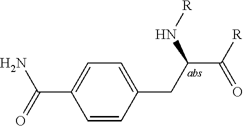 | NC(c1ccc(C[C@H](C([R])═O) N[R])cc1)═O |
| | | C₁₀H₁₀N₂O₂R₂ | |
| 157 | 4AmF, 4AmPhe | 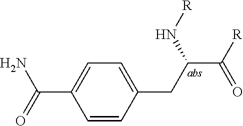 | NC(c1ccc(C[C@@H] (C([R])═O)N[R])cc1)═ON[C@@H] (Cc(cc1)ccc1C(N)═O)C═O |
| | | C₁₀H₁₀N₂O₂R₂ | |
| 158 | D(NPh) | 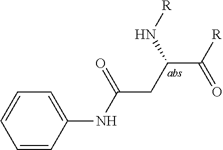 | O═C(C[C@@H] (C([R])═O)N[R])Nc1ccccc1 |
| | | C₁₀H₁₀N₂O₂R₂ | |
| 159 | N(3AmBenzyl)Gly | 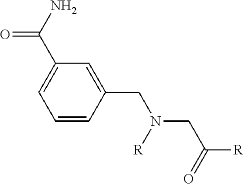 | NC(c1cccc(CN(CC([R])═O)[R])c1)═O |
| | | C₁₀H₁₀N₂O₂R₂ | |
| 160 | N(4AmBenzyl)Gly | 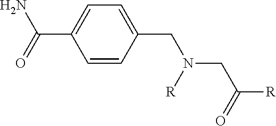 | NC(c1ccc(CN(CC([R])═O)[R])cc1)═O |
| | | C₁₀H₁₀N₂O₂R₂ | |
| 161 | 2AmTyr | 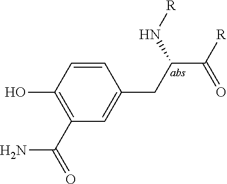 | NC(c(cc(C[C@@H] (C([R])═O)N[R])cc1)c1O)═O |
| | | C₁₀H₁₀N₂O₃R₂ | |
| 152 | aMeFPhe | 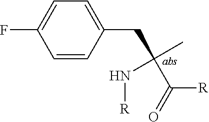 | C[C@](Cc(cc1)ccc1F)(C([R])═O)N[R] |
| | | C₁₀H₁₀FNOR₂ | |
| 163 | D(NmAn) | 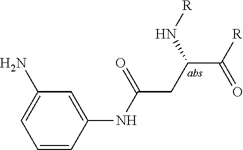 | Nc1cccc(NC(C[C@@H] (C([R])═O)N[R])═O)c1 |
| | | C₁₀H₁₁N₃O₂R₂ | |
| 164 | D(NoAn) | 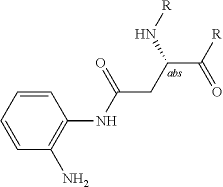 | Nc(cccc1)c1NC(C[C@@H] (C([R])═O)N[R])═O |
| | | C₁₀H₁₁N₃O₂R₂ | |
| 165 | D(NpAn) | 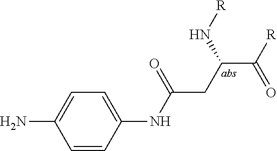 | Nc(cc1)ccc1NC(C[C@@H] (C([R])═O)N[R])═O |
| | | C₁₀H₁₁N₃O₂R₂ | |
| 166 | 4MeOF | 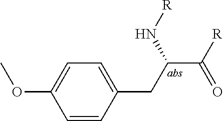 | COc1ccc(C[C@@H](C([R])═O) N[R])cc1COc1ccc(C[C@@H] (C═O)N)cc1 |
| | | C₁₀H₁₁NO₂R₂ | |
| 167 | NMeDTyr, NMeDY, NMedTyr, NMedY, N-Methyl-D- tyrosine, dNMeTyr dNMeY | 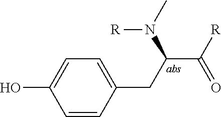 | CN([C@H](Cc(cc1)ccc1O) C([R])═O)[R] |
| | | C₁₀H₁₁NO₂R₂ | |
| 168 | aMe3OHPhe | 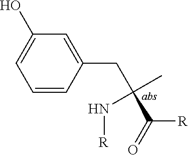 | C[C@](Cc1cc(O)ccc1)(C([R])═O)N[R] |
| | | C₁₀H₁₁NO₂R₂ | |
| 169 | aMeY, aMeTyr | 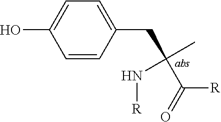 | C[C@](Cc(cc1)ccc1O)(C([R])═O)N[R] |
| | | C₁₀H₁₁NO₂R₂ | |
| 170 | bMeDTyr(2R3S) bMeDTyr(2R,3S) | 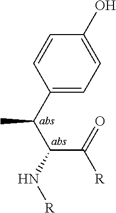 | C[C@H]([C@H] (C([R])═O)N[R])c(cc1)ccc1O |
| | | C₁₀H₁₁NO₂R₂ | |
| 171 | 4MeF | 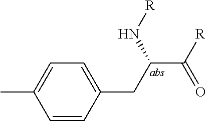 | Cc1ccc(C[C@@H](C([R])═O)N[R])cc1 |
| | | C₁₀H₁₁NOR₂ | |
| 172 | aMeF, aMeF alpha-methyl phenylalanine | 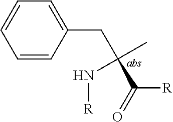 | C[C@](Cc1ccccc1)(C([R])═O)N[R] C[C@](Cc1ccccc1)(C═O)N |
| | | C₁₀H₁₁NOR₂ | |
| 173 | bMePhe | 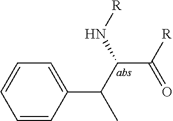 | CC([C@@H](C([R])═O)N[R])c1ccccc1 |
| | | C₁₀H₁₁NOR₂ | |
| 174 | bMePhe(2S3S) bMePhe(2S,3S) | 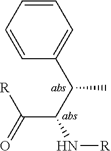 | C[C@H]([C@@H](C([R])═O) N[R])c1ccccc1 |
| | | C₁₀H₁₁NOR₂ | |
| 175 | hF, hPhe, homoF, homoPhe | 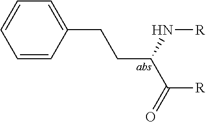 | O═C([C@H](CCc1ccccc1)N[R])[R] |
| | | C₁₀H₁₁NOR₂ | |
| 176 | F4CONH2, 4-carbamoyl-L- phenylalanine | 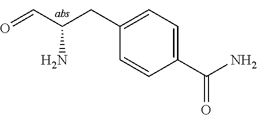 [SMILES: NC(=O)c1ccc(C[C@H](N)C=O)cc1] | N[C@@H](Cc(cc1)ccc1C(N)═O)C═O |
| | | C₁₀H₁₂N₂O₂ | |
| 177 | Maf | 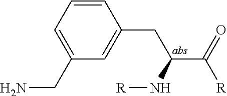 | NCc1cccc(C[C@@H] (C([R])═O)N[R])c1 |
| | | C₁₀H₁₂N₂OR₂ | |
| 178 | Paf | 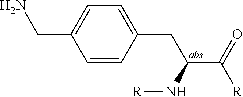 | NCc1ccc(C[C@@H](C([R])═O)N[R]) ccc1NCc1ccc(C[C@@H](C═O)N)cc1 |
| | | C₁₀H₁₂N₂OR₂ | |
| 179 | dMaf, maf | 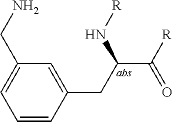 | NCc1cccc(C[C@H](C([R])═O)N[R])c1 |
| | | C₁₀H₁₂N₂OR₂ | |
| 180 | dPaf | 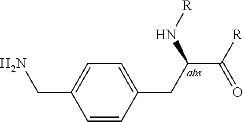 | NCc1ccc(C[C@H](C([R])═O)N[R])cc1 |
| | | C₁₀H₁₂N₂OR₂ | |
| 181 | oAMPhe | 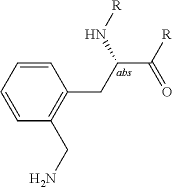 | NCc1c(C[C@@H] (C([R])═O)N[R])cccc1 |
| | | C₁₀H₁₂N₂OR₂ | OC([C@@H](N[H]) |
| | | | CC1═CC═C(C═C1)NC(N)═N)═O |
| | F(G) | | |
| 182 | F(4G) | 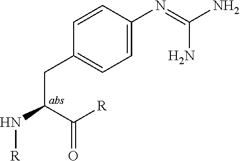 | NC(N)═Nc1ccc(C[C@@H] (C([R])═O)N[R])cc1 |
| | | C₁₀H₁₂N₄OR₂ | |
| 183 | NMeDTyr | 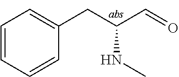 | CN[C@H](Cc1ccccc1)C═O |
| | | C₁₀H₁₃NO | |
| 184 | dNMeTyr dNMeY, D-N-methyl tyrosine N-Methyl- D-tyrosine | 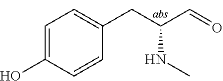 | CN[C@H](Cc(cc1)ccc1O)C═O |
| | | C₁₀H₁₃NO₂ | |
| 185 | biotin | 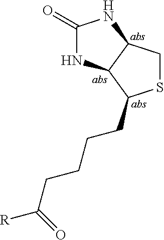 | O═C(CCCC[C@@H]([C@H] 1N2)SC[C@@H]1NC2═O)[R] |
| | | C₁₀H₁₅N₂O₂SR | |
| 186 | K(CO2allyl) | 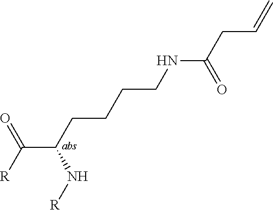 | C═CCC(NCCCC[C@@H] (C([R])═O) N[R])═OC═CCOC(NCCCC[C@@H] (C([R])═O)N[R])═O |
| | | C₁₀H₁₆N₂O₂R₂ | |
| 187 | K(COcPr) | 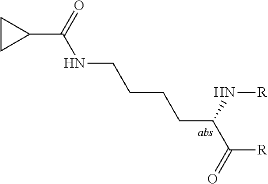 | O═C([C@H](CCCCNC(C1CC1)═O) N[R])[R] |
| | | C₁₀H₁₆N₂O₂R₂ | |
| 188 | DAGSuc | 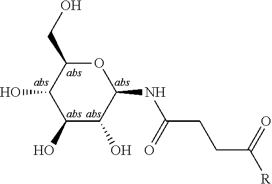 | OC[C@H]([C@H]([C@@H]([C@H] 1O)O)O)O[C@H] 1NC(CCC([R])═O)═O |
| | | C₁₀H₁₆NO₇R | |
| 189 | K(COPr) | 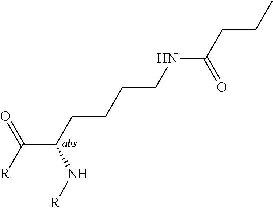 | CCCC(NCCCC[C@@H] (C([R])═O)N[R])═O |
| | | C₁₀H₁₈N₂O₂R₂ | |
| 190 | K(COiPr) | 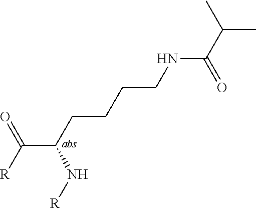 | CC(C)C(NCCCC[C@@H] (C([R])═O)N[R])═O |
| | | C₁₀H₁₈N₂O₂R₂ | |
| 191 | Tzl(Ch) | 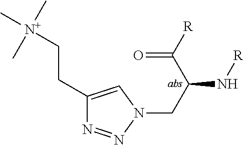 | C[N+](C)(C)CCc1cn(C[C@@H] (C([R])═O)N[R])nn1 |
| | | C₁₀H₁₈N₅OR₂⁺ | |
| 192 | hK(Me)3, hKMe3 | 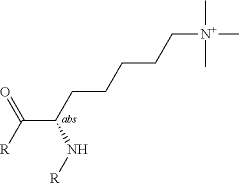 | C[N+](C)(C)CCCCC[C@@H] (C([R])═O)N[R] |
| | | C₁₀H₂₁N₂OR₂⁺ | |
| 193 | hdK(Me)3, hk(Me)3, hdKMe3 | 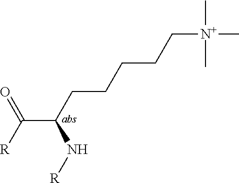 | C[N+](C)(C)CCCCC[C@H] (C([R])═O)N[R] |
| | | C₁₀H₂₁N₂OR₂⁺ | |
| 194 | Dap(pF(6)) | 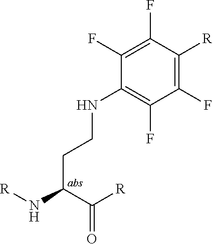 | O═C([C@H](CCNc(c(F) c(c([R])c1F)F)c1F)N[R])[R] |
| | | C₁₀H₇F₄N₂OR₃ | |
| 195 | 4OCF3DPhe | 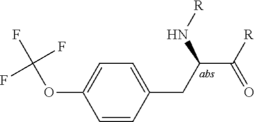 | O═C([C@@H](Cc(cc1) ccc1OC(F)(F)F)N[R])[R] |
| | | C₁₀H₈F₃NO₂R₂ | |
| 196 | CF3F | 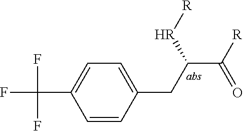 | O═C([C@H](Cc1ccc(C(F)(F)F) cc1)N[R])[R] |
| | | C₁₀H₈F₃NOR₂ | |
| 197 | 7AzaW | 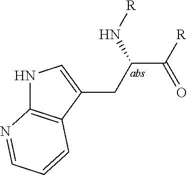 | O═C([C@H](Cc1c[nH]c2c1cccn2) N[R])[R] |
| | | C₁₀H₉N₃OR₂ | |
| 198 | Y(CHF2) | 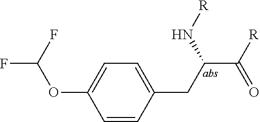 | O═C([C@H](Cc(cc1)ccc1OC(F)F) N[R])[R] |
| | | C₁₀H₉F₂NO₂R₂ | |
| 199 | CXF | 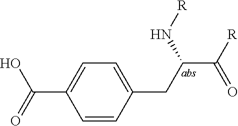 | OC(c1ccc(C[C@@H](C([R])═O) N[R])cc1)═O |
| | | C₁₀H₉NO₃R₂ | |
| 200 | CHF2Phe | 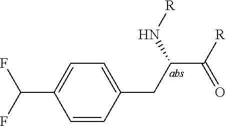 | O═C([C@H](Cc1ccc(C(F)F)cc1) N[R])[R] |
| | | C₁₀H₉F₂NOR₂ | |
| 201 | TetraFAEF | 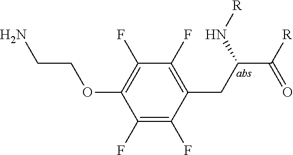 | NCCOc(c(F)c(c(C[C@@H] (C([R])═O)N[R])c1F)F)c1F |
| | | C₁₁H₁₀F₄N₂O₂R₂ | |
| 202 | 5OHW | 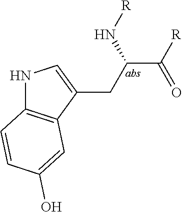 | Oc(cc1)cc2c1[nH]cc2C[C@@H] (C([R]═O)N[R] |
| | | C₁₁H₁₀N₂O₂R₂ | |
| 203 | 4AcDPhe | 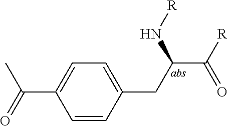 | CC(c1ccc(C[C@H](C([R])═O) N[R])cc1)═O |
| | | C₁₁H₁₁NO₂R₂ | |
| 204 | D(NBz) | 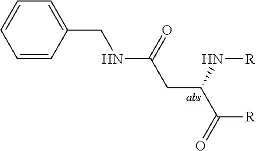 | O═C(C[C@@H](C([R])═O) N[R])NCc1ccccc1 |
| | | C₁₁H₁₂N₂O₂R₂ | |
| 205 | aMe2AmTyr | 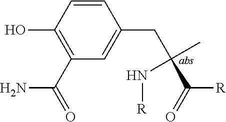 | C[C@](Cc(cc1)cc(C(N)═O) c1O)(C([R])═O)N[R] |
| | | C₁₁H₁₂N₂O₃R₂ | |
| 206 | psiW | 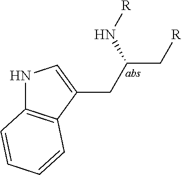 | [R]C[C@H](Cc1c[nH]c2c1cccc2)N[R] |
| | | C₁₁H₁₂N₂R₂ | |
| 207 | aMeY01 | 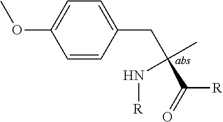 | C[C@](Cc(cc1)ccc1OC) (C([R])═O)N[R] |
| | | C₁₁H₁₃NO₂R₂ | |
| 208 | 3OMeY01 | 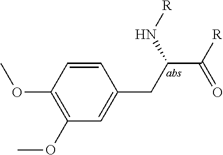 | COc(ccc(C[C@@H](C([R])═O) N[R])c1)c1OC |
| | | C₁₁H₁₃NO₃R₂ | |
| 209 | | | |
| 210 | dAEF | 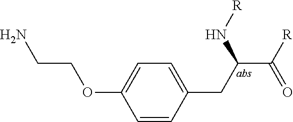 | NCCOc1ccc(C[C@H] (C([R])═O)N[R])cc1 |
| | | C₁₁H₁₄N₂O₂R₂ | |
| 211 | K(COBu) | 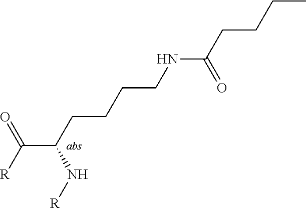 | CCCCC(NCCCC[C@@H] (C([R])═O)n[R])═O |
| | | C₁₁H₂₀N₂O₂R₂ | |
| 212 | K(COiBu) | 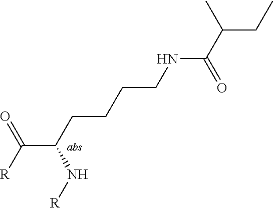 | CCC(C)C(NCCCC[C@@H] (C([R])═O)N[R])═OCC(C) CC(NCCCC[C@@H](C([R])═O) N[R])═O |
| | | C₁₁H₂₀N₂O₂R₂ | |
| 213 | K(COtBu) | 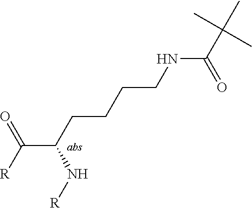 | CC(C)(C)C(NCCCC[C@@H] (C([R])═O)N[R])═O |
| | | C₁₁H₂₀N₂O₂R₂ | |
| 214 | succiniccarn | 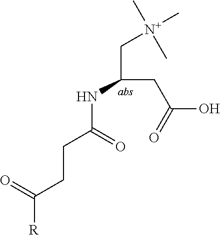 | C[N+](C)(C)C[C@@H] (CC(O)═O)NC(CCC([R])═O)═O |
| | | C₁₁H₂₀N₂O₄R⁺ | |
| 215 | Aun | 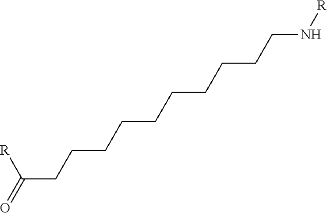 | O═C(CCCCCCCCCCN[R])[R] |
| | | C₁₁H₂₁NOR₂ | |
| 216 | 5BrW, 5BrTrp | 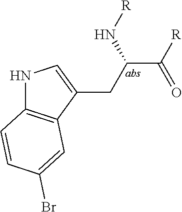 | O═C([C@H](Cc1c[nH]c(cc2) c1cc2Br)N[R])[R] |
| | | C₁₁H₉BrN₂OR₂ | |
| 217 | 7BrTrp, 7BrW | 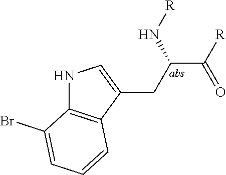 | O═C([C@H](Cc1c[nH] c2c1cccc2Br)N[R])[R] |
| | | C₁₁H₉BrN₂OR₂ | |
| 218 | 7ClW, 7ClTrp | 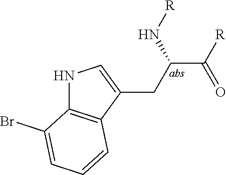 | O═C([C@H](Cc1c[nH] c2c1cccc2Cl)N[R])[R] |
| | | C₁₁H₉ClN₂OR₂ | |
| 219 | 5FW, 5FTrp | 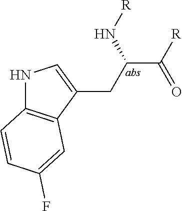 | O═C([C@H](Cc1c[nH] c(cc2)c1cc2F)N[R])[R] |
| | | C₁₁H₉FN₂OR₂ | |
| 220 | 7FW, 7FTrp | 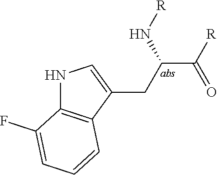 | O═C([C@H](Cc1c[nH] c2c1cccc2F)N[R])[R] |
| | | C₁₁H₉FN₂OR₂ | |
| 221 | BT, L-3- Benzothienyl- alanine | 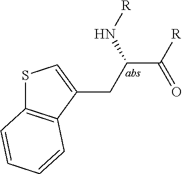 | O═C([C@H](Cc1csc2c1cccc2)N[R])[R] |
| | | C₁₁H₉NOSR₂ | |
| 222 | 2Quin 6OHQui | 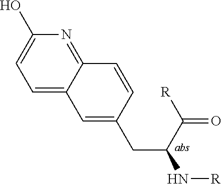 | Oc1ccc(cc(C[C@@H] (C([R])═O)N[R])cc2)c2n1 |
| | | C₁₂H₁₀N₂O₂R₂ | |
| 223 | 7CF2H | 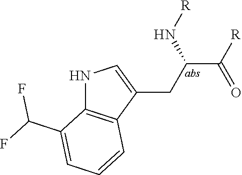 | O═C([C@H](Cc1c[nH] c2c1cccc2C(F)F)N[R])[R] |
| | | C₁₂H₁₀F₂N₂OR₂ | |
| 224 | 3QuinolAla | 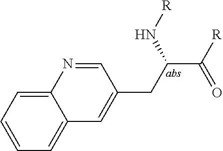 | O═C([C@H](Cc1cc2ccccc2nc1) N[R])[R] |
| | | C₁₂H₁₀N₂OR₂ | |
| 225 | 2MeTrp, 2MeW | 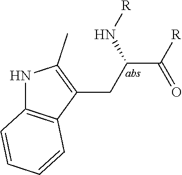 | Cc1c(C[C@@H](C([R])═O) N[R])c(cccc2)c2[nH]1 |
| | | C₁₂H₁₂N₂OR₂ | |
| 226 | 5MeW, 5MeTrp | 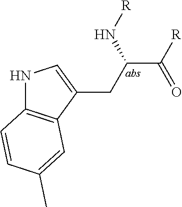 | Cc(cc1)cc2c1[nH]cc2C[C@@H] (C([R])═O)N[R] |
| | | C₁₂H₁₂N₂OR₂ | |
| 227 | 7MeW, 7(MeW), 7MeTrp | 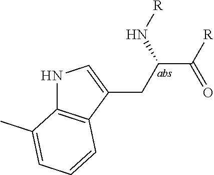 | Cc1cccc2c1[nH]c2C[C@@H] (C([R])═O)n[R]Cc1cccc2c1[nH] c2C[C@@H](C═O)N |
| | | C₁₂H₁₂N₂OR₂ | |
| 228 | aMeW | 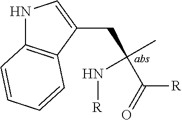 | C[C@](Cc1c[nH]c2c1cccc2) (C([R])═O)N[R] |
| | | C₁₂H₁₂N₂OR₂ | |
| 229 | dW7Me, 7Mew, 7MedW | 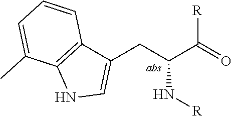 | Cc1cccc2c1[nH]c2C[C@H] (C([R])═O)N[R] |
| | | C₁₂H₁₂N₂OR₂ | |
| 230 | Y(OTzl) | 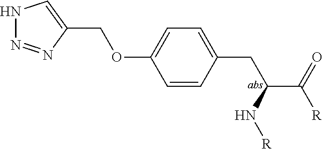 | O═C[C@H](Cc(cc1) ccc1OCc1c[nH]nn1)N[R])[R] |
| | | C₁₂H₁₂N₄O₂R₂ | |
| 231 | 4AllylY | 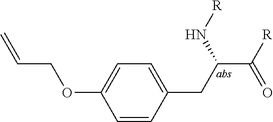 | C═CCOc1ccc(C[C@@H] (C([R])═O)N[R])cc1 |
| 232 | 4AllylF | 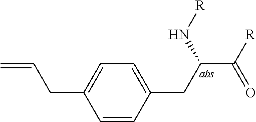 | C═CCc1ccc(C[C@@H] (C([R])═O)N[R])cc1 |
| | | C₁₂H₁₃NOR₂ | |
| 233 | meW, NMeW, NMeTrp, N-Methyl- Tryptophan | 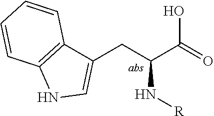 | CN[C@@H](Cc1c[nH] c2c1cccc2)C(O)═O |
| | | C₁₂H₁₄N₂O₂ | |
| | AEF(G) | | [R]C([C@H] |
| | | | (CC1═CC═C(OCCNC(N)═N)C═C1) |
| | | | N[R])═O |
| 234 | AAMPhe | 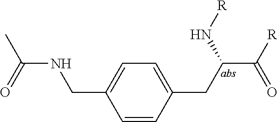 | CC(NCc1ccc(C[C@@H] (C([R])═O)N[R])cc1)═OCC (NCc1ccc(C[C@@](C═O)N)cc1)═O |
| | | C₁₂H₁₄N₂O₂R₂ | |
| 235 | hC(pXyl) | 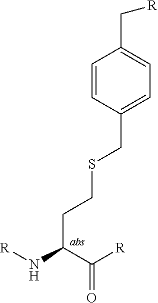 | O═C([C@H] (CCSCc1ccc(C[R])cc1)N[R])[R] |
| | | C₁₂H₁₄NOSR₃ | |
| 236 | AEF(NMe(2)) | 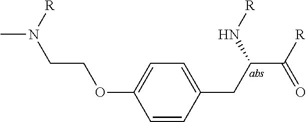 | CN(CCOc1ccc(C[C@@H] (C([R])═O)N[R])cc1)[R] |
| | | C₁₂H₁₅N₂O₂R₃ | |
| 237 | DY02 | 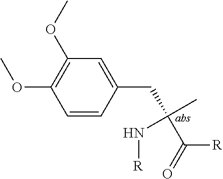 | C[C@@H](Cc(cc1)cc(OC) c1OC)(C([R])═O)N[R] |
| | | C₁₂H₁₅NO₃R₂ | |
| 238 | Y02 | 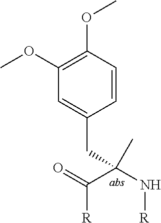 | C[C@](Cc(cc1)cc(OC)c1OC) (C([R])═O)N[R] |
| | | C₁₂H₁₅NO₃R₂ | |
| 239 | AEF(NMe) | 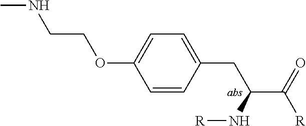 | CNCCOc1ccc(C[C@@H] (C([R])═O)N[R])cc1 |
| | | C₁₂H₁₆N₂O₂R₂ | |
| 240 | NMeAEF | 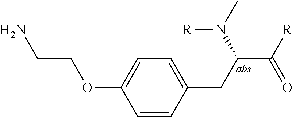 | CN([C@@H](Cc(cc1) ccc1OCCN)C([R])═O)[R] CN[C@@H](Cc(cc1) ccc1OCCN)C═O |
| | | C₁₂H₁₆N₂O₂R₂ | |
| 241 | aMeAEF | 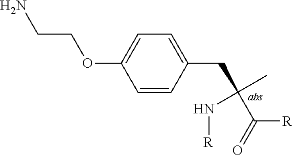 | C[C@](Cc(cc1)ccc1OCCN) (C([R])═O)N[R] CC(C)(C)OC(NCCOc1ccc(C[C@@] (C)(C([R])═O)N[R])cc1)═O |
| | | C₁₂H₁₆N₂O₂R₂ | |
| 242 | bMeAEF | 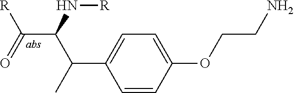 | CC([C@@H](C([R])═O)N[R])c(cc1) ccc1OCCN |
| | | C₁₂H₁₆N₂O₂R₂ | |
| 243 | bMeAEF(2S,3R*), bMeAEF(2S3R*) (*pure but configuration unknown) | 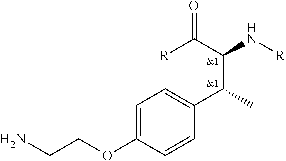 | C[C@@H](C@@H] (C([R])═O)N[R])c(cc1)ccc1OCCN |
| | | C₁₂H₁₆N₂O₂R₂ | |
| 244 | bMeAEF(2S3S*), bMeAEF(2S,3S*) (*pure but configuration unknown) | 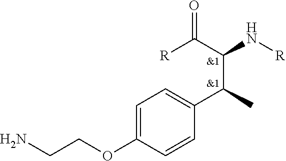 | C[C@H]([C@@H] (C([R])═O)N[R])c(cc1)ccc1OCCN |
| | | C₁₂H₁₆N₂O₂R₂ | |
| 245 | K(Morph) | 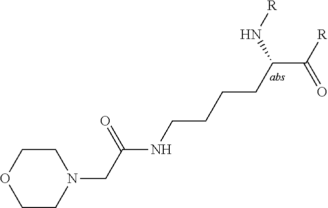 | O═C(CN1CCOCC1) NCCCC[C@@H](C([R])═O)n[R] |
| | | C₁₂H₂₁N₃O₃R₂ | |
| 246 | K(COPent) | 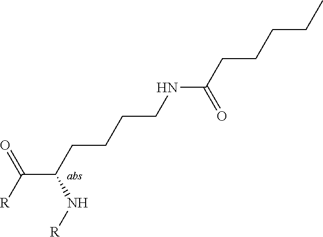 | CCCCCC(NCCCC[C@@H] (C([R])═O)N[R])═O |
| | | C₁₂H₂₂N₂O₂R₂ | |
| 247 | aMeK(Boc) | 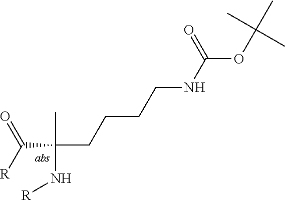 | CC(C)(C)OC(NCCCC[C@@] (C)(C([R])═O)N[R])═O |
| | | C₁₂H₂₂N₂O₃R₂ | |
| 248 | E(C) | 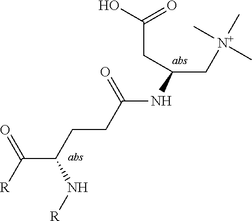 | C[N+](C)(C)C[C@H](CC(O)═O) NC(CC[C@@H](C([R])═O)N[R])═O |
| | | C₁₂H₂₂N₃O₄R₂⁺ | |
| 249 | E(c) (R)-2-((R)-4- amino-4-carboxy- butanamido)- 3-carboxy-N,N,N- trimethylpropan-1- aminium, E(c) | 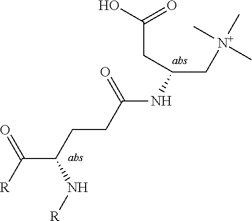 | C[N+](C)(C)C[C@@H](CC(O)═O) NC(CC[C@@H](C([R])═O)N[R])═O |
| | | C₁₂H₂₂N₃O₄R₂⁺ | |
| 250 | e(C), dE(C) | 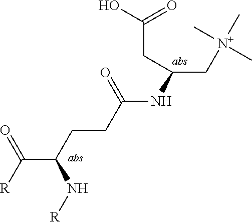 | C[N+](C)(C)C[C@H](CC(O)═) NC(CC[C@H](C([R])═O)N[R])═O |
| | | C₁₂H₂₂N₃O₄R₂⁺ | |
| 251 | e(c), dE(c) | 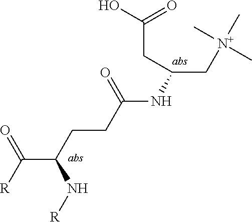 | C[N+](C)(C)C[C@@H](CC(O)═O) NC(CC[C@H](C([R])═O)N[R])═O |
| | | C₁₂H₂₂N₃O₄R₂⁺ | |
| 252 | dK(SP6), k(SP6) | 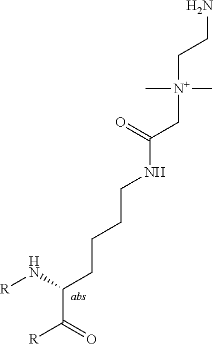 | C[N+](C)(CCN)CC(NCCCC[C@H] (C([R])═O)N[R])═O |
| | | C₁₂H₂₅N₄O₂R₂⁺ | |
| 253 | 7CF3W, (S)-2- amino-3-(7- (trifluoromethyl)- 1H-indol-3-yl) propanoic acid | 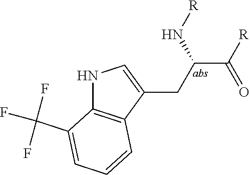 | O═C([C@H](Cc1c[nH] c2c1cccc2C(F)(F)F)N[R])[R] N[C@@H](Cc1c[nH] c2c(C(F)(F)F)cccc12)C═O N[C@@H](Cc1c[nH] c2c(C(F)(F)F)cccc12)C═O |
| | | C₁₂H₉F₃N₂OR₂ | |
| 254 | 5Br2Nal | 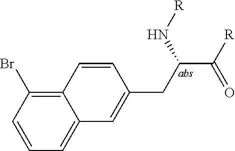 | O═C([C@H](Cc1cc2cccc(Br) c2cc1)N[R])[R] |
| | | C₁₃H₁₀BrNOR₂ | |
| 255 | 6Br2Nal | 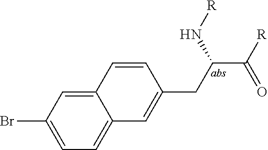 | O═C([C@H](Cc(cc1c2)cc1ccc2Br) N[R])[R] |
| | | C₁₃H₁₀BrNOR₂ | |
| 256 | 7Br2Nal | 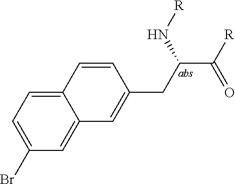 | O═C([C@H](Cc1cc2cc(Br)ccc2cc1) N[R])[R] |
| | | C₁₃H₁₀BrNOR₂ | |
| 257 | 6F2Nal | 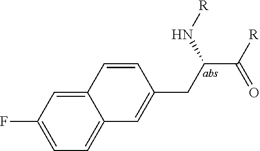 | O═C([C@H](Cc(ccc1c2)cc1ccc2F )N[R])[R] N[C@@H(Cc1ccc(cc(cc2)F) c2c1)C═O |
| | | C₁₃H₁₀FNOR₂ | |
| 258 | 7OH2Nal | 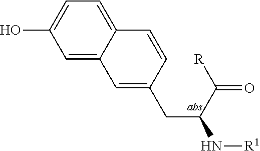 | Oc1ccc(ccc(C[C@@H] (C([R])═O)N[R1])c2)c2c1 |
| | | C₁₃H₁₁NO₂R₂ | |
| 259 | 1Nal, Nal, | 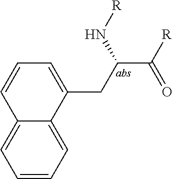 | O═C([C@H] (Cc1cccc2ccccc12)N[R])[R] |
| | | C₁₃H₁₁NOR₂ | |
| 260 | 2Nal | 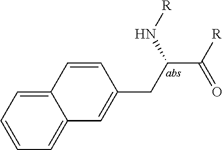 | O═C([C@H] (Cc1cc2ccccc2cc1)N[R])[R] |
| | | C₁₃H₁₁NOR₂ | |
| 261 | dNal, d2Nal | 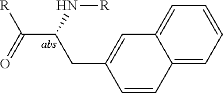 | O═C([C@@H] (Cc1cc2ccccc2cc1)N[R])[R] |
| | | C₁₃H₁₁NOR₂ | |
| 262 | 6MeQui | 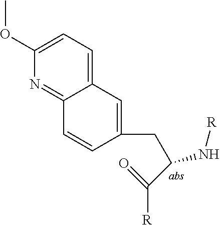 | COc1ccc(cc(C[C@@H] (C([R])═O)N[R])cc2)c2n1 |
| | | C₁₃H₁₂N₂O₂R₂ | |
| 263 | D(N5In) | 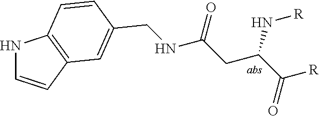 | O═C(C[C@@H](C([R])═O) N[R])NCc(cc1)cc2c1[nH]cc2 |
| | | C₁₃H₁₃N₃O₂R₂ | |
| 264 | psi2Nal | 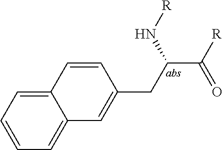 | [R]C[C@H](Cc1cc2ccccc2cc1)N[R] |
| | | C₁₃H₁₃NR₂ | |
| 265 | 7EtW | 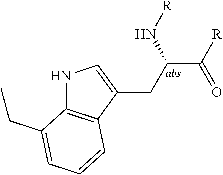 | CCc1cccc2c1[nH] cc2C[C@@H](C([R])═O)N[R] |
| | | C₁₃H₁₄N₂OR₂ | |
| 266 | F(4TzlMME) | 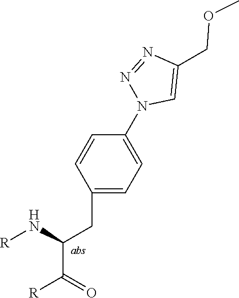 | COCc1cn(-c2ccc(C[C@@H] (C([R])═O)N[R])cc2)nn1 |
| | | C₁₃H₁₄N₄O₂R₂ | |
| 267 | AcAEF | 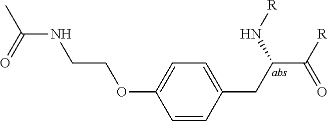 | CC(NCCOc1ccc(C[C@@H] (C([R])═O)N[R])cc1)═O |
| | | C₁₃H₁₆N₂O₃R₂ | |
| 268 | tButY, Y(tBu) | 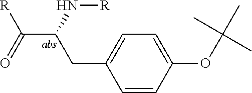 | CC(C)(C)Oc1ccc(C[C@@H] (C([R])═O)N[R])cc1 |
| | | C₁₃H₁₇NO₂R₂ | |
| 269 | AEF(Me)2 | 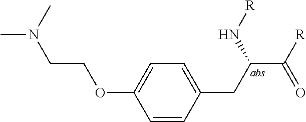 | CN(C)CCOc1ccc(C[C@@H] (C([R])═O)N[R])cc1 |
| | | C₁₃H₁₈N₂O₂R₂ | |
| 270 | Z, Amp | 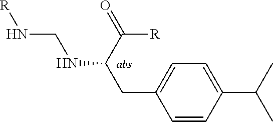 | CC(C)c1ccc(C[C@@H] (C([R])═O)NCN[R])cc1 |
| | | C₁₃H₁₈N₂OR₂ | |
| 271 | 5amido2Nal | 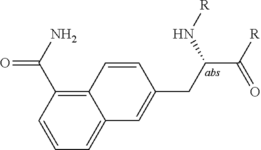 | NC(c1c(ccc(C[C@@H] (C([R])═O)N[R])c2)c2ccc1)═O |
| | | C₁₄H₁₂N₂O₂R₂ | |
| 272 | 6amido2Nal | 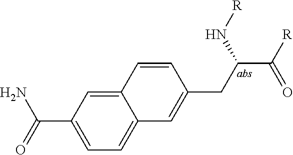 | NC(c1ccc(cc(C[C@@H] (C([R])═O)N[R])cc2)c2c1)═O |
| | | C₁₄H₁₂N₂O₂R₂ | |
| 273 | 5OMe2Nal | 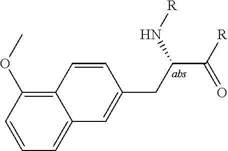 | COc1c(ccc(C[C@@H] (C([R])═O)N[R])c2)c2ccc1 |
| | | C₁₄H₁₃NO₂R₂ | |
| 274 | 6OMe2Nal | 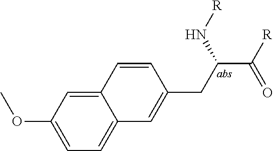 | COc1ccc(cc(C[C@@H] (C([R])═O)N[R])cc2)c2c1 |
| | | C₁₄H₁₃NO₂R₂ | |
| 275 | 5Me2Nal | 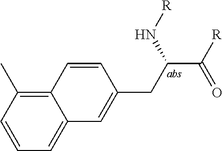 | Cc1c(ccc(C[C@@H] (C([R])═O)N[R])c2)c2ccc1 |
| | | C₁₄H₁₃NOR₂ | |
| 276 | NMe2NAL | 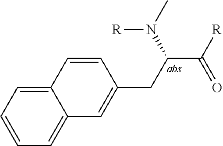 | CN([C@@H](Cc1cc2ccccc2cc1) C([R])═O)[R] CN[C@@H](Cc1cc2ccccc2cc1)C═O |
| | | C₁₄H₁₃NOR₂ | |
| 277 | aMe2Nal | 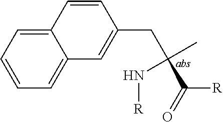 | C[C@](Cc1cc2ccccc2cc1) (C([R])═O)N[R] |
| | | C₁₄H₁₃NOR₂ | |
| 278 | bMe2Nal(2S,3R), bMe2Nal(2S3R) | 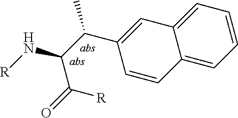 | C[C@@H](C@@H](C([R])═O) N[R])c1cc2ccccc2cc1 |
| | | C₁₄H₁₃NOR₂ | |
| 279 | bMe2Nal(2S3S), bMe2Nal(2S3R) | 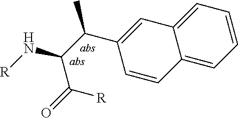 | C[C@H]([C@@H](C([R])═O) N[R])c1cc2ccccc2cc1 |
| | | C₁₄H₁₃NOR₂ | |
| 280 | AEF(EtCO) | 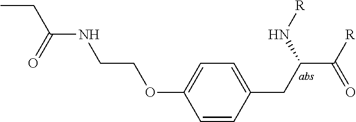 | CCC(NCCOc1ccc(C[C@@H] (C([R])═O)N[R])cc1)═O |
| | | C₁₄H₁₈N₂O₃R₂ | |
| 281 | NMeY(tBu) | 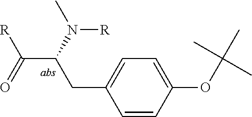 | CC(C)(C)Oc1ccc(C[C@H] (C([R])═O)N(C)[R])cc1 |
| | | C₁₄H₁₉NO₂R₂ | |
| 282 | AEF(NMe3) | 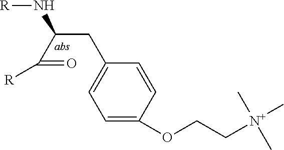 | C[N+](C)(C)CCOc1ccc(C[C@@H] (C([R])═O)N[R])cc1 |
| | | C₁₄H₂₁N₂O₂R₂⁺ | |
| 283 | 6O(COCF3)2Nal | 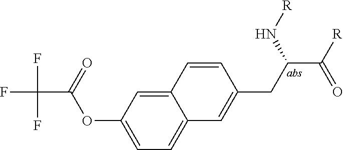 | O═C([C@H] (Cc(ccc1c2) cc1ccc2OC(C(F)(F)F)═O)N[R])[R] |
| | | C₁₅H₁₀F₃NO₃R₂ | |
| 284 | BIF | 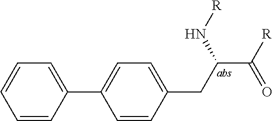 | O═C([C@H](Cc(cc1)ccc1- c1ccccc1)N[R])[R] |
| | | C₁₅H₁₃NOR₂ | |
| 285 | DiPhAla | 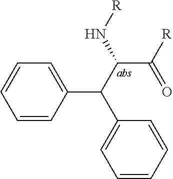 | O═C([C@H](C(c1ccccc1) c1ccccc1)N[R])[R] |
| | | C₁₅H₁₃NOR₂ | |
| 286 | 5Et2Nal | 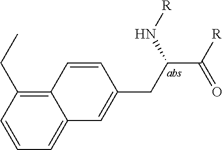 | CCc1(ccc(C[C@H] (C([R])═O)N[R])c2)c2ccc1 |
| | | C₁₅H₁₅NOR₂ | |
| 287 | CMF | 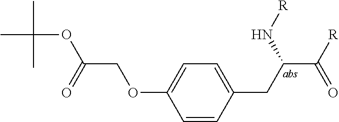 | CC(C)(C)OC(COc1ccc(C[C@@H] (C([R])═O)N[R])cc1)═O |
| | | C₁₅H₁₉NO₄R₂ | |
| 288 | F(4TzlTMA1) | 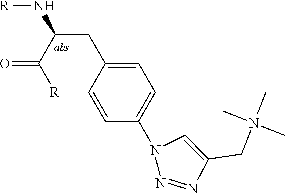 | C[N+](C)(C)Cc1cn(-c2ccc(C[C@@H] (C([R])═O)N[R])cc2)nn1 |
| | | C₁₅H₂₀N₅OR₂⁺ | |
| 289 | PiperazinequatF | 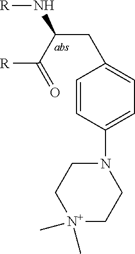 | C[N+](C)(CC1)CCN1c1ccc(C[C@@H] (C([R])═O)N[R])cc1 |
| | | C₁₅H₂₂N₃OR₂⁺ | |
| 290 | TMA3F | 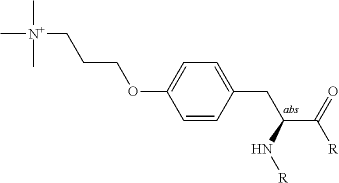 | C[N+](C)(C)CCCOc1ccc(C[C@@H] (C([R])═O)N[R])cc1 |
| | | C₁₅H₂₃N₂O₂R₂⁺ | |
| 291 | TMA4F | 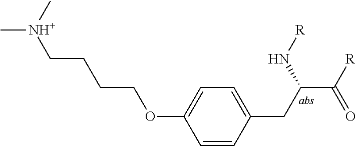 | C[NH+](C)CCCCOc1ccc(C[C@@H] (C([R])═O)N[R])cc1 |
| | | C₁₅H₂₃N₂O₂R₂⁺ | |
| 292 | K5cpa, K(5cpa), K(5cpaCO) | 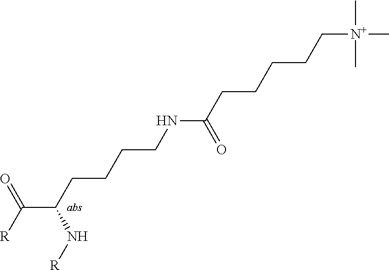 | C[N+](C)(C) CCCCCC(NCCCC[C@@H] (C([R])═O)N[R])═O |
| | | C₁₅H₃₀N₃O₂R₂⁺ | |
| 293 | dK(5cpa), k(5cpa), k(5cpaCO) | 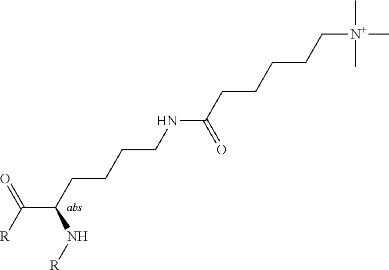 | C[N+](C)(C)CCCCCC(NCCCC[C@H] (C([R])═O)N[R])═O |
| | | C₁₅H₃₀N₃O₂R₂⁺ | |
| 294 | 2Nal6(3pyrazole) | 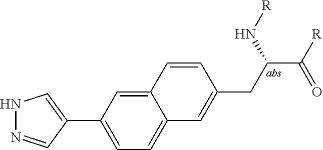 | O═C([C@H] (Cc(ccc1c2)cc1ccc2-c1c[nH] nc1)N[R])[R] |
| | | C₁₆H₁₃N₃OR₂ | |
| 295 | 7PyrTrp | 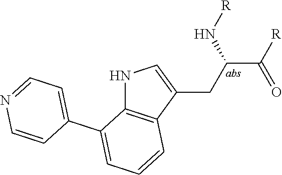 | O═C([C@H](Cc1c[nH] c2c1cccc2-c1ccncc1)N[R])[R] |
| | | C₁₆H₁₃N₃OR₂ | |
| 296 | 4BzF | 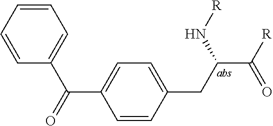 | O═C([C@H](Cc(cc1) ccc1C(c1ccccc1)═O)N[R])[R] |
| | | | C₁₆H₁₃NO₂R₂ |
| 297 | aMeBiF | 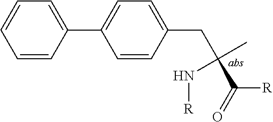 | C[C@](Cc(cc1)ccc1-c1ccccc1) (C([R])═O)N[R] |
| | | C₁₆H₁₅NOR₂ | |
| 298 | NPyEF | 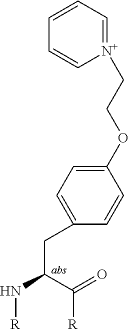 | O═C([C@H](Cc(cc1)ccc1OCC[n+] 1ccccc1)N[R])[R] |
| | | C₁₆H₁₇N₂O₂R₂⁺ | |
| 299 | 5iPr2Nal | 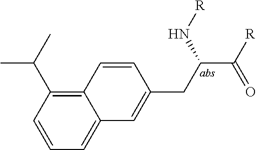 | CC(C)c1c(ccc(C[C@@H] (C([R])═O)N[R])c2)c2ccc1 |
| | | C₁₆H₁₇NOR₂ | |
| 300 | TetraFAEF(Boc) | 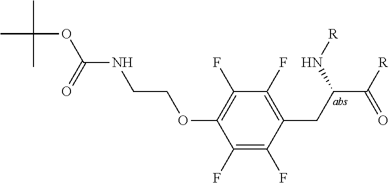 | CC(C)(C)OC(NCCOc(c(F) c(c(C[C@@H] (C([R])═O)N[R])c1F)F)c1F)═O |
| | | C₁₆H₁₈F₄N₂O₄R₂ | |
| 301 | 4TMABYF | 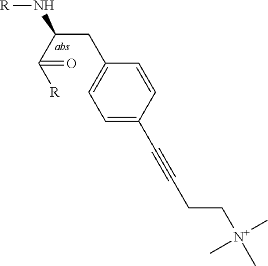 | C[N+](C)(C)CCC#Cc1ccc(c[C@@H] (C([R])═O)N[R])cc1 |
| | | C₁₆H₂₁N₂OR₂⁺ | |
| 302 | AEF(Boc) | 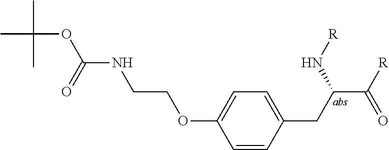 | CC(C)(C)OC(NCCOc1ccc(C[C@@H] (C([R])═O)N[R])cc1)═O |
| | | C₁₆H₂₂N₂O₄R₂ | |
| 303 | F(4TzlTMA2) | 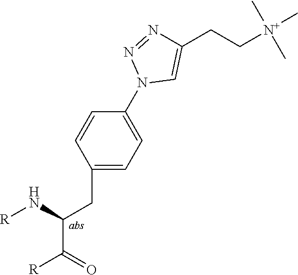 | C[N+](C)(C)CCc1cn(-c2ccc(C[C@@H] (C([R])═O)N[R])cc2)nn1 |
| | | C₁₆H₂₂N₅OR₂⁺ | |
| 304 | DMPMF | 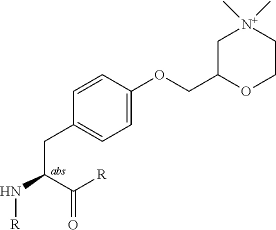 | C[N+]1(C)CC(COc2ccc(C[C@@H] (C([R])═O)N[R])cc2)OCC1 |
| | | C₁₆H₂₃N₂O₃R₂⁺ | |
| 305 | KDde, K(Dde) | 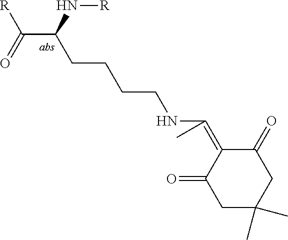 | CC(C)(CC(C1═C(C)NCCCC[C@@H] (C([R])═O)N[R])═O)CC1═O |
| | | C₁₆H₂₄N₂O₃R₂ | |
| 306 | dKDde, k(Dde), dK(Dde) | 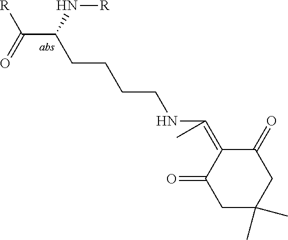 | CC(C)(CC(C1═C(C)NCCCC[C@H] (C([R])═O)N[R])═O)CC1═O |
| | | C₁₆H₂₄N₂O₃R₂ | |
| 307 | Y(OEOXIMECh) | 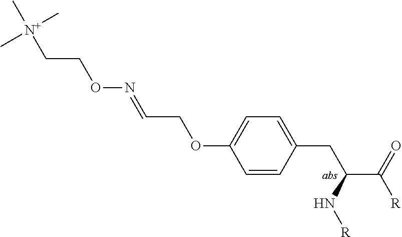 | C[N+](C)(C)CCO/ N═C/COc1ccc(C[C@@H] (C([R])═O)N[R])cc1 |
| | | C₁₆H₂₄N₃O₃R₂⁺ | |
| 308 | Y(OZOXIMECh) | 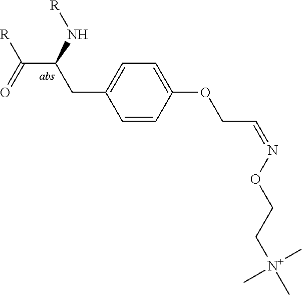 | C[N+](C)(C)CCO/ N═C\COc1ccc(C[C@@H] (C([R])═O)N[R])cc1 |
| | | C₁₆H₂₄N₃O₃R₂⁺ | |
| 309 | AEF(NHCh) | 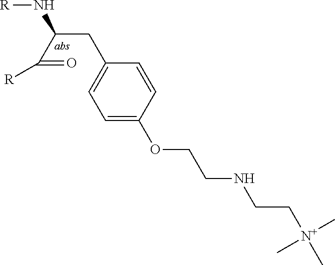 | C[N](C)(C)CCNCCOc1ccc(C[C@@H] (C([R])═O)N[R])cc1 |
| | | C₁₆H₂₆N₃O₂R₂⁺ | |
| 310 | K(Biotina), K(Biotin) | 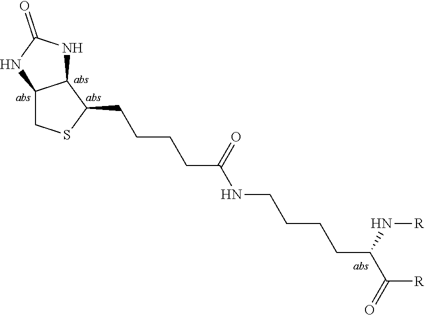 | O═C(CCCC[C@@H[([C@@H]1N2) SC[C@H]1NC2═O)NCCCC[C@@H] (C([R])═O)N[R] |
| | | C₁₆H₂₆N₄O₃SR₂ | |
| 311 | K(DAGSuc) | 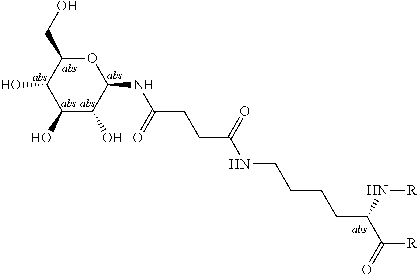 | OC[C@H]([C@H]([C@@H]([C@H] 1O)O)O)O[C@H] 1NC(CCC(NCCCC[C@@H] (C([R]═O)N[R])═O═O |
| | | C₁₆H₂₇N₃O₈R₂ | |
| 312 | k(DAGSuc), dK(DAGSuc) | 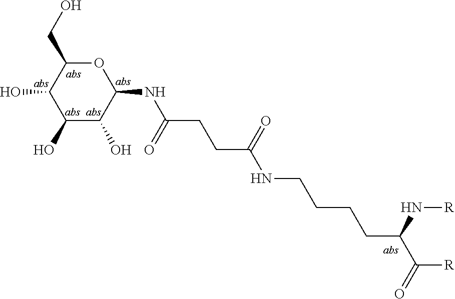 | OC[C@H]([C@H]([C@@H]([C@H] 1O)O)O)O[C@H] 1NC(CCC(NCCCC[C@H] (C([R])═O)N[R])═O)═O |
| | | C₁₆H₂₇N₃O₈R₂ | |
| 313 | DOTA | 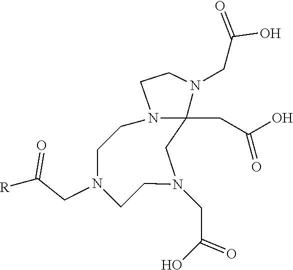 | OC(CN1CCN(CC(O)═O) CCN(CC([[R])═O)CCN(CC(O)═O) CC1)═O |
| | | C₁₆H₂₇N₄O₇R | |
| 314 | Dab(NMeCarn) | 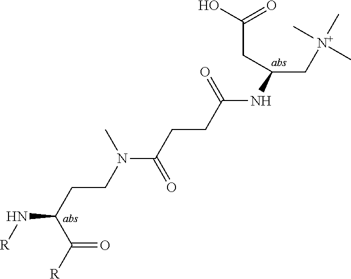 | CN(CC[C@@H](C([R])═O)N[R]) C(CCC(N[C@@H] (CC(O)═O)C[N+](C)(C)C)═O)═O |
| | | C₁₆H₂₉N₄O₅R₂⁺ | |
| 315 | Dab(NMecarn | 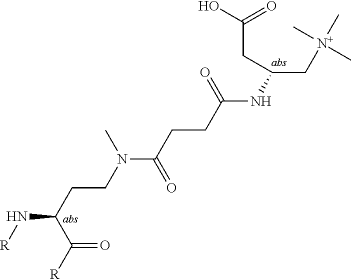 | CN(CC[C@@H](C([R])═O)N[R]) C(CCC(N[C@H] (CC(O)═O)C[N +](C)(C)C)═O)═O |
| | | C₁₆H₂₉N₄O₅R₂⁺ | |
| 316 | orn(d) | 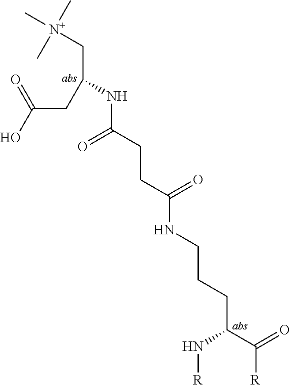 | C[N+](C)(C)C[C@@H](CC(O)═O) NC(CCC(NCCC[C@H](C([R])═O) N[R])═O)═O |
| | | C₁₆H₂₉N₄O₅R₂⁺ | |
| 317 | 2Nal6((5CF3) 3pyrazole) | 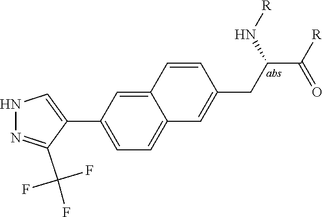 | O═C([C@H](Cc(cc1c2cc1ccc2- c1c[nH]nc1C(F)(F)F)N[R])[R] |
| | | C₁₇H₁₂F₃N₃OR₂ | |
| 318 | 7(2ClPh)W | 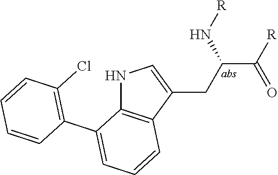 | O═C([C@H](Cc1c[nH]c2c1ccc2- c(cccc1)c1Cl)N[R])[R] |
| | | C₁₇H₁₃ClN₂OR₂ | |
| 319 | TMAPF | | C[N+](C)(CCCCCOc1ccc(C[C@H] |
| | | | (N[R])C([R])═O)cc1)C |
| 320 | 7(2OMe5Pyr)W | 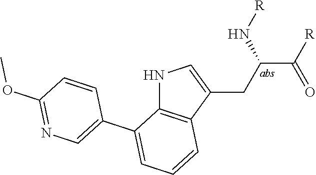 | COc(cc1)ncc1-c1cccc2c1[nH] cc2C[C@@H](C([R])═O)N[R] |
| | | C₁₇H₁₅N₃O₂R₂ | |
| 321 | W-7Ph, 7-phenyl-L- tryptophan | 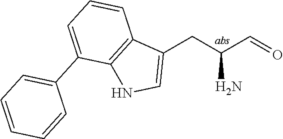 [SMILES: N[C@H](C=O)Cc1c[nH]c2c(-c3ccccc3)cccc12] | N[C@@H](Cc1c[nH]c2c1ccc2- c1ccccc1)C═O |
| | | C₁₇H₁₆N₂O | |
| 322 | 5OH2Nal | 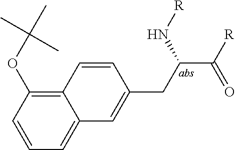 | CC(C)(C)Oc1c(ccc(C[C@@H] (C([R])═O)N[R])c2)c2ccc1 |
| | | C₁₇H₁₉NO₂R₂ | |
| 323 | 5tBu2Nal | C₁₇H₁₉NOR₂ | CC(C)(C)c1c(ccc(C[C@@H] |
| | | | C([R])═O)N[R])c2)c2ccc1 |
| 324 | hFTMAPF | 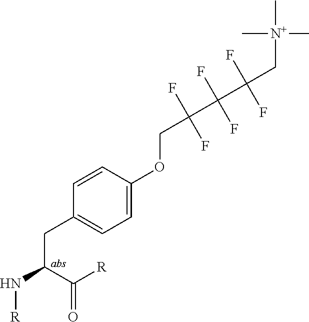 | C[N+](C)(C) CC(C(C(COc1ccc(C[C@@H] (C([R])═O)N[R])cc1)(F)F)(F)F)(F)F |
| | | C₁₇H₂₁F₆N₂O₂R₂⁺ | |
| 325 | F(4TzlTMA3) | 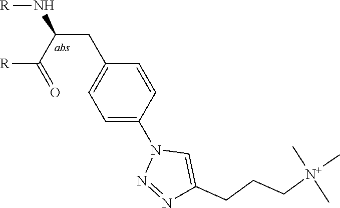 | C[N+](C)(C)CCCc1cn(-c2ccc(C[C@@H] (C([R])═O)N[R])cc2)nn1 |
| | | C₁₇H₂₄N₅OR₂⁺ | |
| 326 | DMMMF | 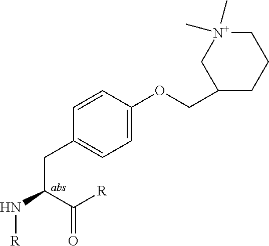 | C[N+]1(C)CC(COc2ccc(C[C@@H] (C([R])═O)N[R])cc2)CCC1 |
| | | C₁₇H₂₅N₂O₂R₂⁺ | |
| 327 | MMoEF | 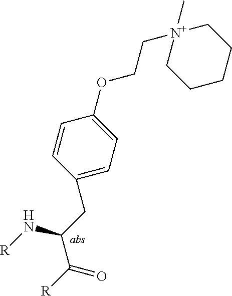 | C[N+]1(CCOc2ccc(C[C@@H] (C([R])═O)N[R])cc2)CCCCC1 |
| | | C₁₇H₂₅N₂O₂R₂⁺ | |
| 328 | MMoPF | 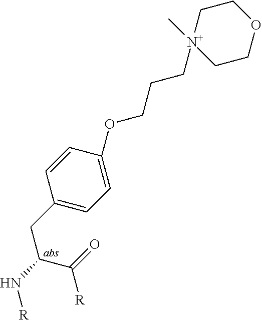 | C[N+1(CCCOc2ccc(C[C@H] (C([R])═O)N[R])cc2)CCOCC1 |
| | | C₁₇H₂₅N₂O₃R₂⁺ | |
| 329 | AEF(MEP) | 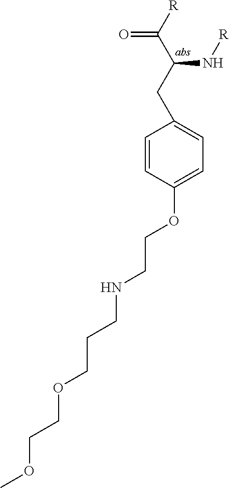 | COCCOCCCNCCOc1ccc(C[C@@H] (C([R])═O)N[R])cc1 |
| | | C₁₇H₂₆N₂O₄R₂ | |
| 330 | 4DMPzEF | 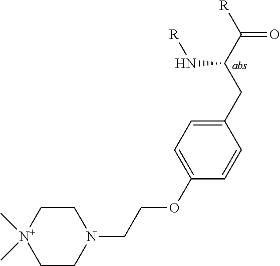 | C[N+]1(C)CCN(CCOc2ccc(C[C@@H] (C([R])═O)N[R])cc2)CC1C [N+]1(C)CCN(CCOc2ccc(C[C@@H] (C═O)N)cc2)CC1 |
| | | C₁₇H₂₆N₃O₂R₂⁺ | |
| 331 | TMAPF | 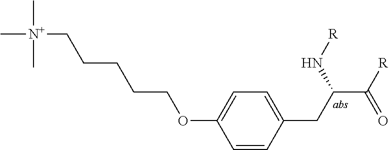 | C[N+](C)(C)CCCCCOc1ccc(C[C@@H] (C([R]═O)N[R])cc1 C[N+](C)(C)CCCCCOc1ccc(C[C@H] (C═O)N)cc1 |
| | | C₁₇H₂₇N₂O₂R₂⁺ | |
| 332 | K(D), KCar | 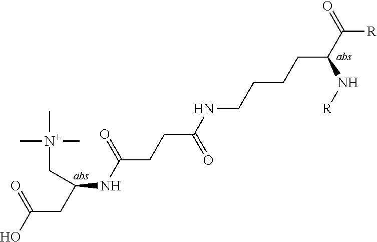 | C[N+](C)(C)C[C@H](CC(O)═O) NC(CCC(NCCCC[C@@H] (C([R])═ON[R])═O)═O |
| | | C₁₇H₃₁N₄O₅R₂⁺ | |
| 333 | K(d), KdCar | 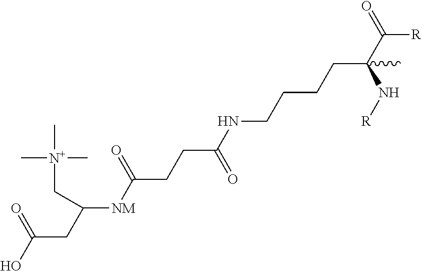 | C[N+](C)(C)C[C@@H](CC(O)═O) |
| | | | NC(CCC(NCCCC[C@@H](C([R])═O) |
| | | | N[R])═O)═O |
| | | C₁₇H₃₁N₄O₅R₂⁺ | |
| 334 | k(D), dKCar | 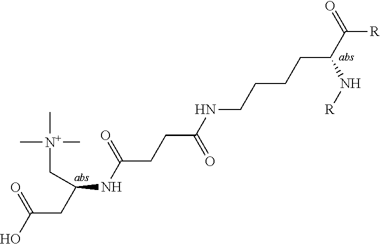 | C[N+](C)(C)C[C@H](CC(O)═O) NC(CCC(NCCCC[C@H](C([R])═O) N[R])═O)═O |
| | | C₁₇H₃₁N₄O₅R₂⁺ | |
| 335 | k(d), dKdCar | 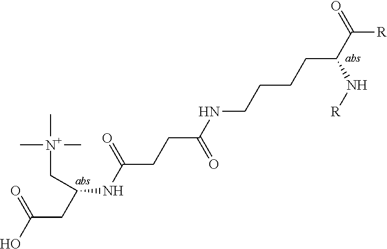 | C[N+](C)(C)C[C@@H](CC(O)═O) NC(CCC(NCCCC[C@H](C([R])═O) N[R])═O)═O |
| | | C₁₇H₃₁N₄O₅R₂⁺ | |
| 336 | 7(3CF3TAZP)W | 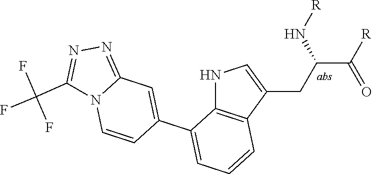 | O═C([C@H](Cc1[nH]c2c1cccc2- c1cc1nnc(C(F)(F)F)n2cc1)N[R])[R] |
| | | C₁₈H₁₂F₃N₅OR₂ | |
| 337 | 7(4OCF3Ph)W | 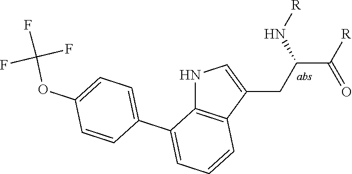 | O═C([C@H](Cc1c[nH]c2c1cccc2- c(cc1)ccc1OC(F)(F)F)N[R])[R] |
| | | C₁₈H₁₃F₃N₂O₂R₂ | |
| 338 | 7(4CF3Ph)W | 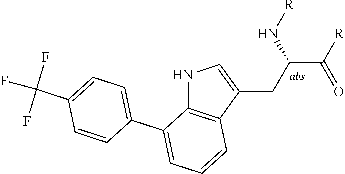 | O═C([C@H](Cc1c[nH]c1c1cccc2- c1ccc(C(F)(F)F)cc1)N[R])[R] |
| | | C₁₈H₁₃F₃N₂OR₂ | |
| 339 | 7(7ImidPyr)W | 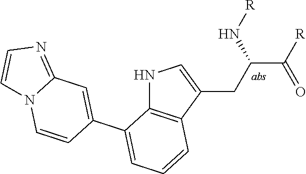 | O═C([C@H](Cc1c[nH]c2c1cccc2- c1cc2nccn2cc1)N[R])[R] |
| | | C₁₈H₁₄N₄OR₂ | |
| 340 | Y(C9OH) | 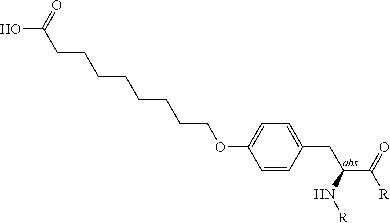 | OC(CCCCCCCCOc1ccc(C[C@@H] (C([R])═O)N[R])cc1)═O |
| | | C₁₈H₂₅NO₄R₂ | |
| 341 | Y(OTzlCh) | 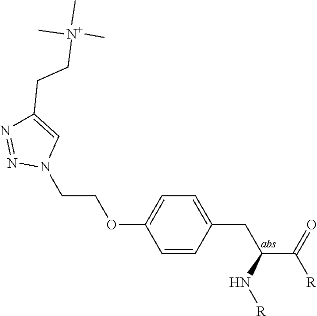 | C[N+](C)(C) CCc1cn(CCOc2ccc(C[C@@H] (C([R])═O)N[R])cc2)nn1 |
| | | C₁₈H₂₆N₅O₂R₂⁺ | |
| 342 | 4DMPEF | 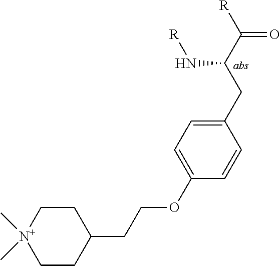 | C[N+]1(C)CCC(CCOc2ccc(C[C@@H] (C([R])═O)N[R]cc2)CC1 C[N+]1(C)CCC(CCOc2ccc(C[C@@H] (C═O)N)cc2)CC1 |
| | | C₁₈H₂₇N₂O₂R₂⁺ | |
| 343 | AEF(AcCh) | 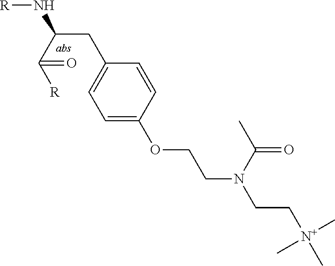 | CC(N(CC[N+] (C)(C)C)CCOc1ccc(C[C@@H] (C([R])═O)N[R])cc1)═O |
| | | C₁₈H₂₈N₃O₃R₂⁺ | |
| 344 | TMA6F | 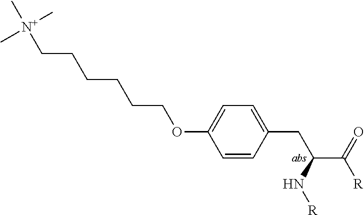 | C[N+](C)(C) CCCCCCOc1ccc(C[C@@H] (C([R])═O)N[R])cc1 |
| | | C₁₈H₂₉N₂O₂R₂⁺ | |
| 345 | AEF(MePrpa) | 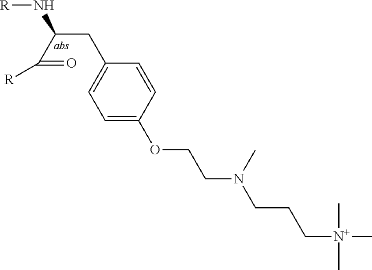 | CN(CCC[N+](C)(C)C) CCOc1ccc(C[C@@H] (C([R])═O)N[R])cc1 |
| | | C₁₈H₃₀N₃O₂R₂⁺ | |
| 346 | 2Nal6(Ph2OH) | 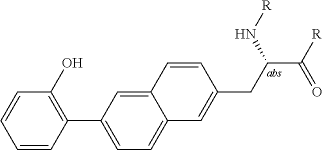 | Oc(cccc1)c1- c1ccc(cc(C[C@@H] (C([R])═O)N[R])cc2)c2c1 |
| | | C₁₉H₁₅NO₂R₂ | |
| 347 | 7(3NAcPhW | 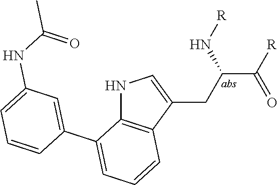 | CC(Nc1cccc(-c2cccc3c2[nH] cc3C[C@@H](C([R])═O)N[R])c1)═O |
| | | C₁₉H₁₇N₃O₂R₂ | |
| 348 | 7(4NAcPh) | 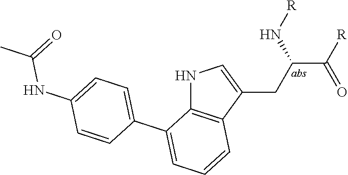 | CC(Nc(cc1)ccc1-c1cccc2c1[nH] cc2C[C@@H](C([R])═O)N[R])═O |
| | | C₁₉H₁₇N₃O₂R₂ | |
| 349 | 4PipPhe | C₁₉H₂₆N₂O₃R₂ | CC(C)(C)OC(N(CC1) |
| | | | CCC1c1ccc(C[C@@H] |
| | | | (C([R])═O)N[R])cc1)═O |
| 350 | A | 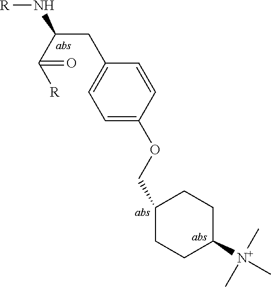 | C[N+](C)(C)[C@@9H]1CC[C@H] (COc2ccc(C[C@@H] (C([R])═O)N[R])cc2)CC1 |
| | | C₁₉H₂₉N₂O₂R₂⁺ | |

TABLE 2D
Peg Moeties and Peg-modified Monomers

| 1 | Structure | Names and Synonyms | Smiles Structure |
|---|---|---|---|
| 2 | C7H15NO3 | CON(MePEG2) | CN(CCOCCOC)C═O |
| 3 | C7H14O4 | mPEG3CO | COCCOCCOCC═O |
| 4 | C14H28O7 | mPEG6CO | COCCOCCOCCO |
| | | | CCOCCOCCC═O |
| 5 | C21H36N3O5+ | AEFNMePEG3a, | C[N+](C)(C)CCOC |
| | | AEF(NHcPEG3a) | COCCC(NCCOc1c |
| | | | cc(C[C@@H](C═ |
| | | | O)N)cc1)═O |
| 6 | C24H42N2O8 | AEFNmPEG6, | COCCOCCOCCO |
| | | AEF(NmPEG6) | CCOCCOCCNCC |
| | | | Oc1ccc(C[C@@H] |
| | | | (C═O)N)cc1 |
| 7 | 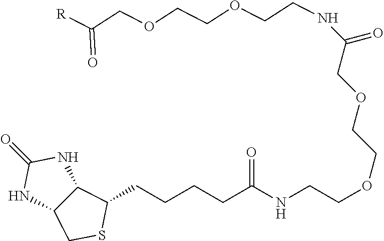 | BiotinPEG2PEG2, Biotin(PEG2PEG2) | O═C(CCCC[C@@ H]([C@H]1N2)SC [C@@H]1NC2═O) NCCOCCOCC(NC COCCOCC([R])═ O)═O |
| | C22H37N4O8SR | | |
| 8 | 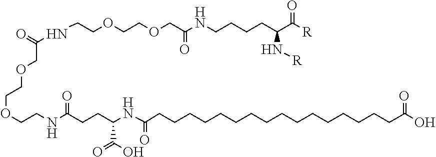 | K(PEG2PEG2gEC18 OH) | OC(CCCCCCCCC CCCCCCCC(N[C @@H](CCC(NCC OCCOCC(NCCOC COCC(NCCCC[C @@H](C([R])═O) N[R])═O)═O)═O)C (O)═O)═O)═O |
| | C41H73N5O13R2 | | |
| 9 | 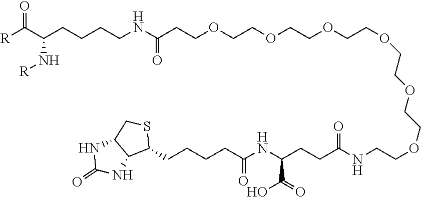 | K(PEG6gEBiotin) | OC([C@H](CCC (NCCOCCOCCOC COCCOCCOCCC (NCCCC[C@@H] (C([R])═O)N[R]) O)═O)NC(CCCC [C@H]([C@@H]1 N2)SC[C@H]1NC 2═O)═O)═O |
| | C36H62N6O13SR2 | | |
| 10 | 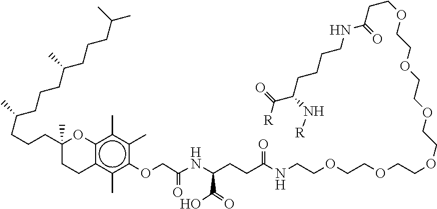 | K(PEG6gEVitE) | CC(C)CCC[C@@H] (C)CCC[C@@H](C) CCC[C@](C)(CC1) Oc(c(C)c2C)c1c(C) c2OCC(N[C@@H] (CCC(NCCOCCOC COCCOCCOCCOC CC(NCCCC[C@@H] (C([R])═O)N[R])═ O)═O)C(O)═O)═O |
| | C57H98N4O14R2 | | |
| 11 | 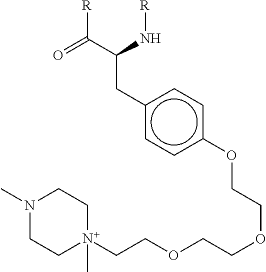 | MPzPEG3F | CN1CC[N+](C)(CC OCCOCCOc2ccc(C [C@@H](C([R])═ O)N[R])cc2)CC1 |
| | C21H34N3O4R2+1 | | |
| 12 | 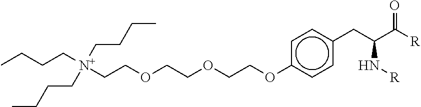 | TBAPEG3F | CCCC[N+](CCCC) (CCCC)CCOCCO CCOc1ccc(C[C@ @H](C([R])═O)N [R])cc1 |
| | C27H47N2O4R2+ | | |
| 13 | 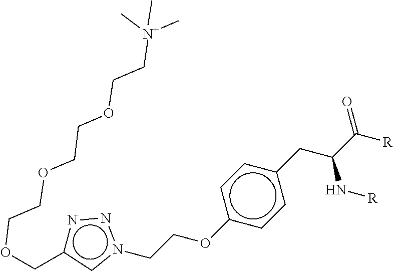 | Y(OTzlPEG3a) | C[N+](C)(C)CCOC COCCOCc1cn(CC Oc2ccc(C[C@@H] (C([R])═O)N[R])cc 2)nn1 |
| | C23H36N5O5R2+ | | |
| 14 | 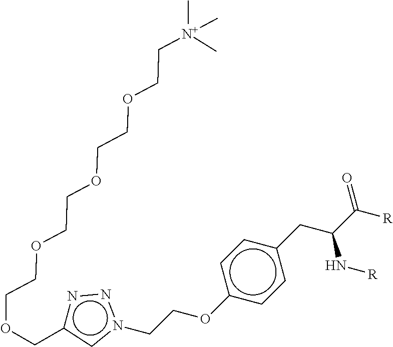 | Y(OTzlPEG4a) | C[N+](C)(C)CCOC COCCOCCOCc1c n(CCOc2ccc(C[C @@H](C([R])═O) N[R])cc2)nn1 |
| | C25H40N5O6R2+ | | |
| 15 | 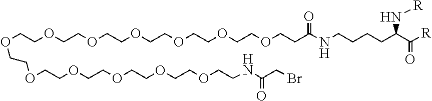 | k(PEG6Biotin), dK(PEG6Biotin) | O═C(CCOCCOCC OCCOCCOCCOC COCCOCCOCCO CCOCCOCCNC(C Br)═O)NCCCC[C @H](C([R])═O)N[R] |
| | C35H66BrN3O15R2 | | |
| 16 | 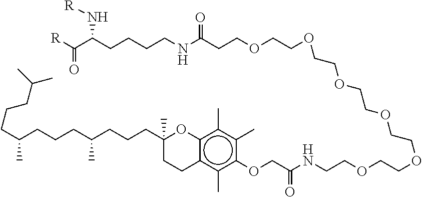 | k(dPEG12Ac), dK(dPEG12Ac) | CC(C)CCC[C@@ H](C)CCC[C@@H] (C)CCC[C@](C) (CC1)Oc(c(C)c2C)c 1c(C)c2OCC(NCC OCCOCCOCCOC COCCOCCC(NCC CC[C@H](C([R])═ O)N[R])═O)═O |
| | C52H91N3O11R2 | | |
| 17 | 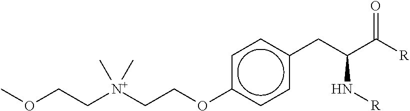 | mPEG2TMA2F | C[N+](C)(CCOC)C COc1ccc(C[C@@ H](C([R]═)O)N[R]) cc1 |
| | C16H25N2O3R2+ | | |
| 18 | 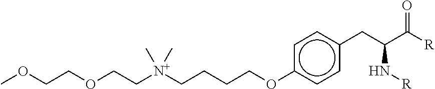 | mPEG3TMA4F | C[N+](C)(CCCCO c1ccc(C[C@@H](C ([R])═O)N[R])cc1) CCOCCOC |
| | C20H33N2O4R2+ | | |
| 19 | 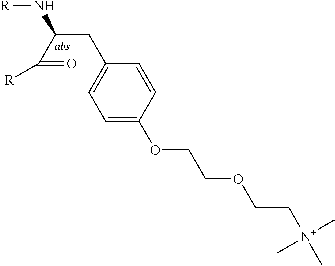 | C[N+](C)(C)CCOCCO c1ccc(C[C@@H](C ([R])═O)N[R])cc1 | |
| | C16H25N2O3R2+ | | |
| 20 | 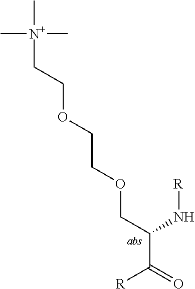 | C[N+](C)(C)CCOCCO C[C@@H](C([R])═O) N[R] | |
| | C10H21N2O3R2+ | | |
| 21 | 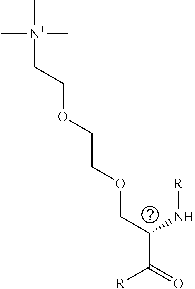 | C[N+](C)(C)CCOCCOC C(C([R])═O)N[R] | |
| | C10H21N2O3R2+ | | |
| 22 | 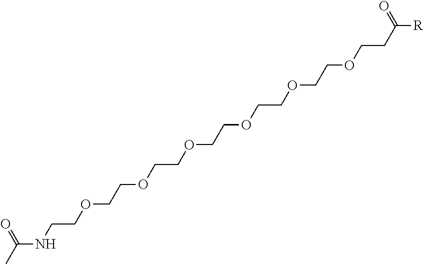 | CC(NCCOCCOCCOC COCCOCCOCCC ([R])═O)═O | |
| | C17H32NO8R | | |
| 23 | 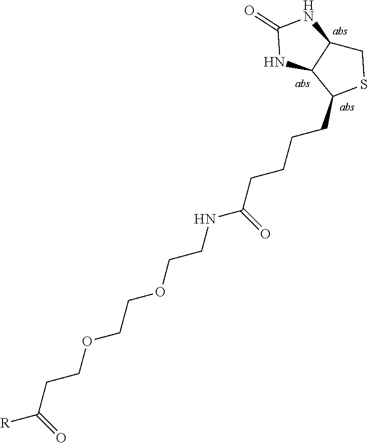 | O═C(CCCC[C@@H] ([C@H]1N2)SC[C@ @H]1NC2═O)NCCO CCOCCC([R])═O | |
| | C17H28N3O5SR | | |
| 24 | 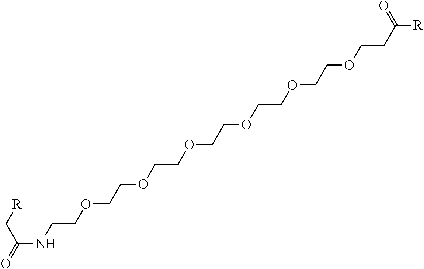 | O═C(CBr)NCCOCCOC COCCOCCOCCOCCC ([R])═O | |
| | C17H31BrNO8R | | |
| 25 | 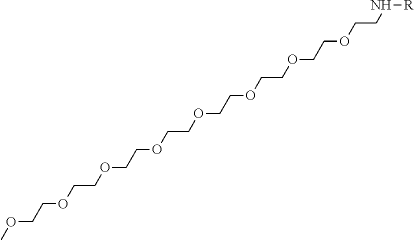 | COCCOCCOCCOCCO CCOCCOCCOCCN[R] | |
| | C17H36NO8R | | |
| 26 | 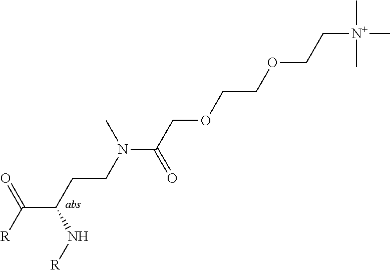 | CN(CC[C@@H](C ([R])═O)N[R])C(COC COCC[N+](C)(C)C)═O | |
| | C14H28N3O4R2+ | | |
| 27 | 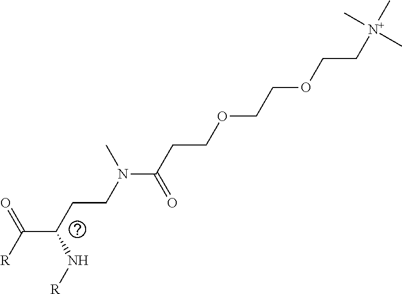 | CN(CC[C@@H](C ([R])═O)N[R])C(CCOC COCC[N+](C)(C)C)═O | |
| | C15H30N3O4R2+ | | |
| 28 | 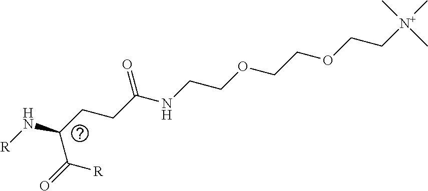 | C[N+](C)(C)CCOCCOC CNC(CC[C@@H](C ([R])═O)N[R])═O | |
| | C14H28N3O4R2+ | | |
| 29 | 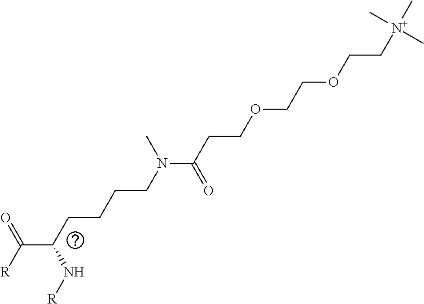 | CN(CCCC[C@@H](C ([R])═O)N[R])C(CCOC COCC[N+](C)(C)C)═O | |
| | C17H34N3O4R2+ | | |
| 30 | 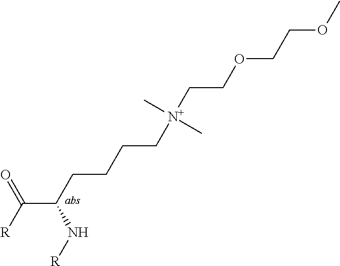 | C[N+](C)(CCCC[C@@ H](C([R])═O)N[R])CC OCCOC | |
| | C13H27N2O3R2+ | | |
| 31 | 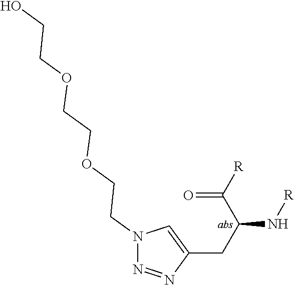 | OCCOCCOCCn1nnc(C [C@@H](C([R])═O)N [R])c1 | |
| | C11H18N4O4R2 | | |
| 32 | 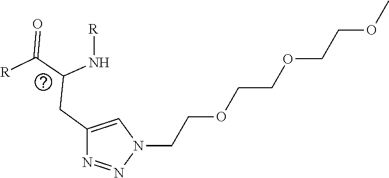 | COCCOCCOCCn1nnc(C [C@@H](C([R])═O)N [R])c1 | |
| | C12H20N4O4R2 | | |
| 33 | 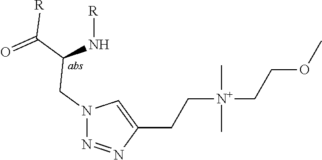 | C[N+](C)(CCclcn(C[C@ @H](C([R])═O)N[R]) nn1)CCOC | |
| | C12H22N5O2R2+ | | |
| 34 | 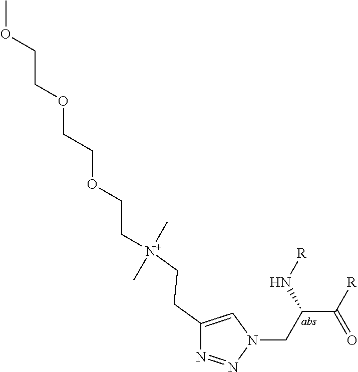 | C[N+](C)(CCclcn(C[C@ @H](C([R])═O)N[R]) nn1)CCOCCOCCOC | |
| | C16H30N5O4R2+ | | |
| 35 | 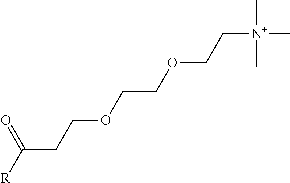 | C[N+](C)(C)CCOCCO CCC([R])═O | |
| | C10H21NO3R+ | | |
| 36 | 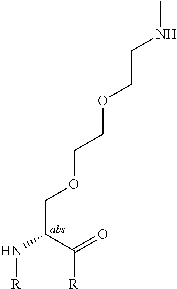 | CNCCOCCOC[C@H] (C([R])═O)N[R] | |
| | C8H16N2O3R2 | | |
| 37 | 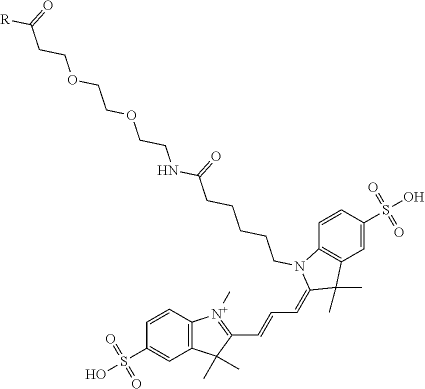 | (SulfoCy3dPEG2) | CC1(C)c(cc(cc2)S (O)(═O)═O)c2[N+] (C)═C1/C═C/C═C (/C1(C)C)\N(CCCC CC(NCCOCCOCC C([R])═O)═O)c(cc 2)c1cc2S(O)(═O)═O |
| | C37H49N3O10S2R+ | | |
| 38 | 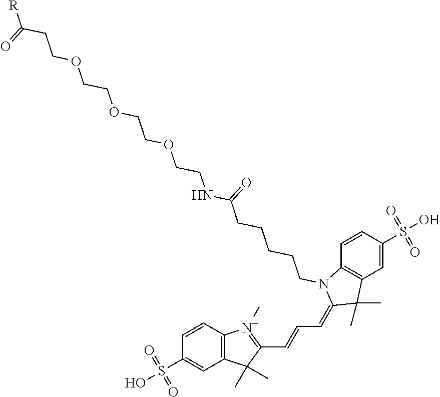 | (SulfoCy3dPEG3) | CC1(C)c(cc(cc2)S (O)(═O)═O)c2[N+] (C)═C1/C═C/C═C (/C1(C)C)\N(CCCC CC(NCCOCCOCC OCCC([R])═O)═O) c(cc2)c1cc2S(O) (═O)═O |
| | C39H53N3O11S2R+ | | |
| 39 | 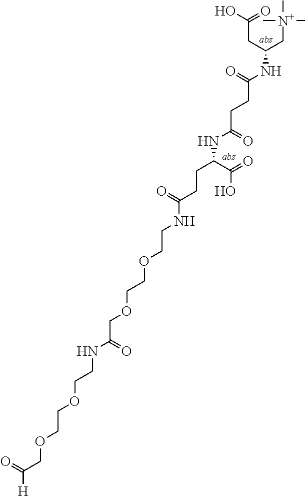 | (d)gEPEG2PEG2 | C[N+](C)(C)C[C@ @H](CC(O)═O)N C(CCC(N[C@@H] (CCC(NCCOCCO CC(NCCOCCOCC ([R])═O)═O)═O)C (O)═O)═O)═O |
| | C28H49N5O13R+ | | |
| 40 | 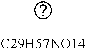 | AcdPEG12CO | CC(NCCOCCOCC OCCOCCOCCOC COCCOCCOCCO |
| | | | CCOCCOCCC═ |
| | | | O)═O |
| 41 | 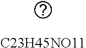 | AcdPEG9CO | CC(NCCOCCOCC OCCOCCOCCOC COCCOCCOCCC═ |
| | | | O)═O |
| 42 | 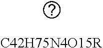 | AEEP(PEG2PEG2gEC 18OH) | OC(CCCCCCCCC CCCCCCCC(N[C @@H](CCC(NCC |
| | | | OCCOCC(NCCOC |
| | | | COCC(NCCOCCO |
| | | | CCC([R])═O)═O)═ |
| | | | O)═O)C(O)═O)═) |
| | | | O═O |
| 43 | 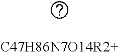 | AEEPPEG2PEG2gEC18 OH, k(PEG2Sp6PEG2g EC18OH), dK(PEG2Sp6 PEG2gEC18OH) | C[N+](C)(CCNC(C OCCOCCNC(CC[C @@H](C(O)═O) NC(CCCCCCCCC |
| | | | CCCCCCCC(O)═ |
| | | | O)═O)═O)═O)CC |
| | | | (NCCOCCOCC(NC |
| | | | CCC[C@H](C([R])═ |
| | | | O)N[R])═O)═O |
| 44 | 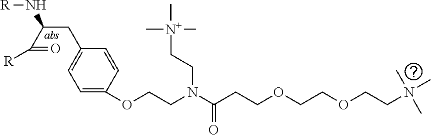 | AEF((Ch)cPEG3a) | C[N+](C)(C)CCN(C COc1ccc(C[C@@H] (C([R])═O)N[R])cc1) C(CCOCCOCC[N+] (C)(C)C)═O |
| | C26H46N4O5R2+2 | | |
| 45 | 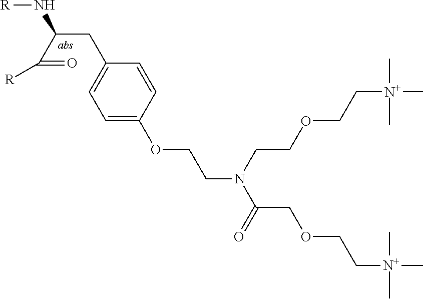 | AEF(BisPEG2a)(RS) AEF(BisPEG2a)(S*) (The RS and the S* indicates the stereo- chemistry) | C[N+](C)(C)CCOCC N(CCOCC[N+](C)(C) C)CCOc1ccc(CC(C ([R])═O)N[R])cc1 |
| | C25H46N4O4R2+2 | | |
| 46 | 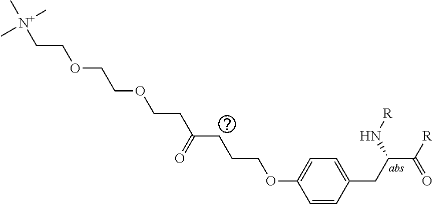 | AEF(NMePEG3a), AEF(NMecPEG3aCO) | C[N+](C)(C)CCOCC COCCC(NCCOc1ccc (C[C@@H](C([R])═ O)N[R])cc1)═O |
| | C21H34N3O5R2+ | | |
| 47 | 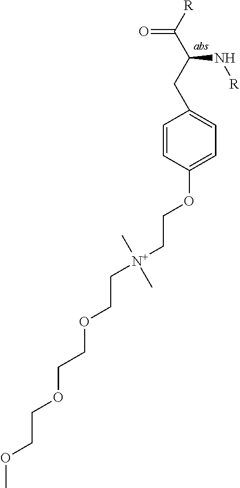 | AEF(NMe2mPEG3) | C[N+](C)(CCOCCOC COC)CCOc1ccc(C [C@@H](C([R])═O) N[R])cc1 |
| | C20H33N2O5R2+ | | |
| 48 | 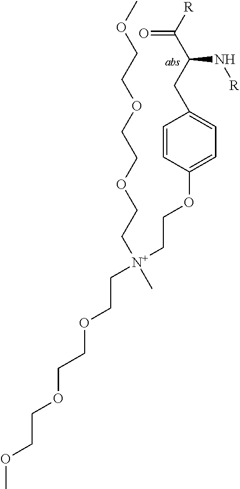 | AEF(NMeBismPEG3) | C[N+](CCOCCOCCO C)(CCOCCOCCOC) CCOc1ccc(C[C@@H] (C([R])═O)N[R])cc1 |
| | C26H45N2O8R2+ | | |
| 49 | 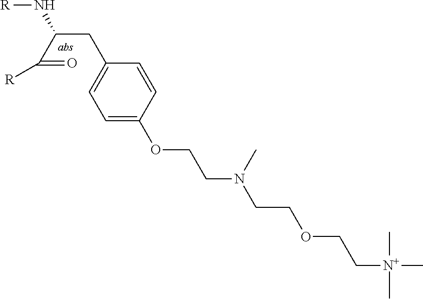 | AEF(NMePEG2a) | CN(CCOCC[N+] (C)(C)C)CCOc1ccc (C[C@H](C([R])═O) N[R])cc1 |
| | C19H32N3O3R2+ | | |
| 50 | 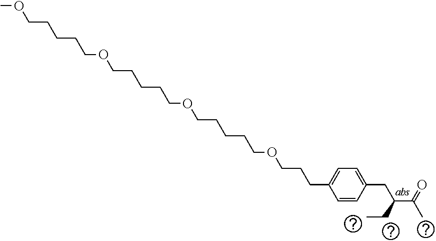 | AEF(NmPEG6) | COCCOCCOCCO CCOCCOCCNCC Oc1ccc(C[C@@H] (C([R])═O)N[R])cc1 |
| | C24H40N2O8R2 | | |
| 51 | 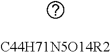 | AEF(PEG2PEG2gEC16 OH) | OC(CCCCCCCCC CCCCCC(N[C@@ H](CCC(NCCOCC OCC(NCCOCCOC |
| | | | C(NCCOc1ccc(C[C |
| | | | @@H](C([R])═O) |
| | | | N[R])cc1)═O)═O)═ |
| | | | O)C(O)═O)═O)═O |
| 52 | 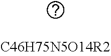 | AEF(PEG2PEG2gEC18 OH | OC(CCCCCCCCC CCCCCCCC(N[C @@H](CCC(NCC OCCOCC(NCCOCC |
| | | | OCC(NCCOc1ccc(C |
| | | | [C@@H](C([R])═O) |
| | | | N[R])cc1)═O)═O)═ |
| | | | O)C(O)═O)═O)═O |
| 53 | 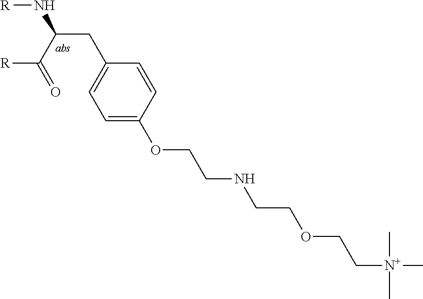 | AEF(Peg2a), AEF(PEG 2a) | C[N+](C)(C)CCOC CNCCOc1ccc(C[C @@H](C([R])═O) N[R])cc1 |
| | C18H30N3O3R2+ | | |
| 54 | 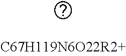 | AEF(SP6PEG12gEC18 OH) | C[N+](C)(CCNC(C COCCOCCOCCOCC OCCOCCOCCOCCO CCOCCOCCOCCNC |
| | | | (CC[C@@H](C(O)═ |
| | | | O)NC(CCCCCCCC |
| | | | CCCCCCCCC(O)═ |
| | | | O)═O)═O)═O)CC(N |
| | | | CCOc1ccc(C[C@@ |
| | | | H](C([R])═O)N[R]) |
| | | | cc1)═O |
| 55 | 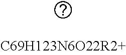 | AEF(SP6PEG12gEC20 OH) | C[N+](C)(CCNC(C COCCOCCOCCO CCOCCOCCOCC OCCOCCOCCOC |
| | | | COCCNC(CC[C@ |
| | | | @H](C(O)═O)NC(C |
| | | | CCCCCCCCCCC |
| | | | CCCCCCC(O)═O)═ |
| | | | O)═O)═O)CC(NC |
| | | | COc1ccc(C[C@@ |
| | | | H](C([R])═O)N[R]) |
| | | | cc1)═O |
| 56 | 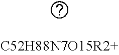 | AEF(SP6PEG2PEG2g EC18OH) | C[N+](C)(CCNC(C OCCOCCNC(COC COCCNC(CC[C@ @H](C(O)═O)NC(C |
| | | | CCCCCCCCCCCC |
| | | | CCCC(O)═O)═O)═ |
| | | | O)═O)═O)CC(NC |
| | | | COc1ccc(C[C@@ |
| | | | H](C([R])═O)N |
| | | | [R])cc1)═O |
| 57 | 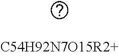 | AEF(SP6PEG2PEG2g EC20OH) | C[N+](C)(CCNC(C OCCOCCNC(COC COCCNC(CC[C@ @H](C(O)═O)NC(C |
| | | | CCCCCCCCCCCC |
| | | | CCCCCC(O)═O)═ |
| | | | O)═O)═O)═O)CC |
| | | | (NCCOc1ccc(C[C |
| | | | @@H](C([R])═O) |
| | | | N[R])cc1)═O |
| 58 | 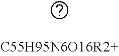 | AEF(SP6PEG6gEC18 OH) | C[N+](C)(CCNC(C COCCOCCOCCO CCOCCOCCNC(C C[C@@H](C(O)═ |
| | | | O)NC(CCCCCCC |
| | | | CCCCCCCCCC(O)═ |
| | | | O)═O)═O)═O)C |
| | | | C(NCCOc1ccc(C[C |
| | | | @@H](C([R])═O) |
| | | | N[R])cc1)═O |
| 59 | 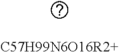 | AEF(SP6PEG6gEC20 OH) | C[N+](C)(CCNC(C COCCOCCOCCO CCOCCOCCNC(C C[C@@H](C(O)═ |
| | | | O)NC(CCCCCCC |
| | | | CCCCCCCCCCCC |
| | | | (O)═O)═O)═O)═ |
| | | | O)CC(NCCOc1ccc |
| | | | (C[C@@H](C([R])═ |
| | | | O)N[R])cc1)═O |
| 60 | 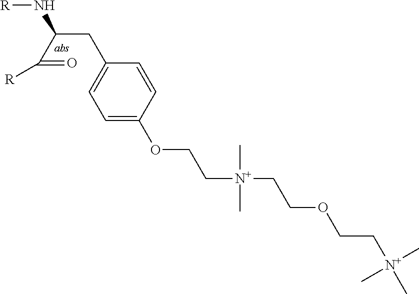 | AEF(aPEG2a) | C[N+](C)(C)CCOC C[N+](C)(C)CCOc 1ccc(C[C@@H](C ([R])═O)N[R])cc1 |
| | C20H35N3O3R2+2 | | |
| 61 | 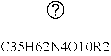 | k(PEG2gEC18OH), dK(PEG2gEC18OH) | OC(CCCCCCCCC CCCCCCCC(N[C @@H](CCC(NCC OCCOCC(NCCCC |
| | | | [C@H](C([R])═O) |
| | | | N[R])═O)═O)C |
| | | | (O)═O)═O)═O |
| 62 | 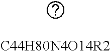 | k(PEG6gEC18OH), dK(PEG6gEC18OH) | OC(CCCCCCCCC CCCCCCCC(N[C @@H](CCC(NCC OCCOCCOCCOC |
| | | | COCCOCCC(NCC |
| | | | CC[C@H](C([R])═ |
| | | | O)N[R])═O)═O)C |
| | | | (O)═O)═O)═O |
| 63 | 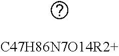 | k(Sp6PEG2PEG2gEC18 OH), dK(Sp6PEG2PEG 2gEC18OH) | C[N+](C)(CCNC(C OCCOCCNC(COC COCCNC(CC[C@ @H](C(O)═O)NC(C |
| | | | CCCCCCCCCCCC |
| | | | CCCC(O)═O)═O)═ |
| | | | O)═O)═O)CC(NCCC |
| | | | C[C@H](C([R])═O) |
| | | | N[R])═O |
| 64 | 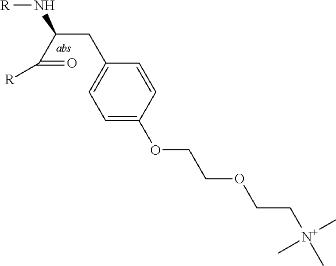 | APEG2F | C[N+](C)(C)CCOC COc1ccc(C[C@@ H](C([R])═O)N[R]) cc1 |
| | C16H25N2O3R2+ | | |
| 65 | 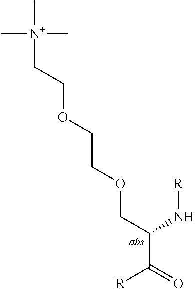 | APEG2ser | C[N+](C)(C)CCOC COC[C@@H](C ([R])═O)N[R] |
| | C10H21N2O3R2+ | | |
| 66 | 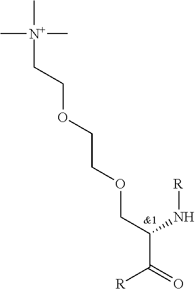 | APEG2Ser(R*) APEG2Ser(S*) | C[N+](C)(C)CCOC COCC(C([R])═O) N[R] |
| | C10H21N2O3R2+ | | |
| 67 | 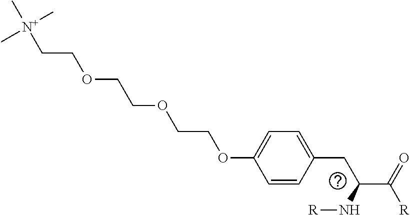 | APEG3F | C[N+](C)(C)CCOC COCCOc1ccc(C[C @@H](C([R])═O) N[R])cc1 C[N+](C)(C)CCOC COCCOc1ccc(C[C @@H](C═O)N)cc1 |
| | C18H29N2O4R2+ | | |
| 68 | 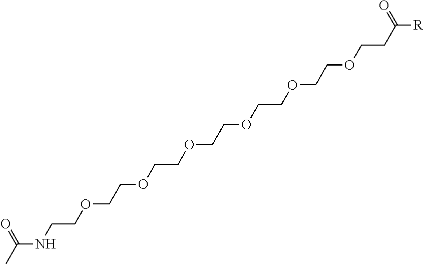 | AcdPEG6CO | CC(NCCOCCOCC OCCOCCOCCOC CC([R])═O)═O |
| | C17H32NO8R | | |
| 69 | 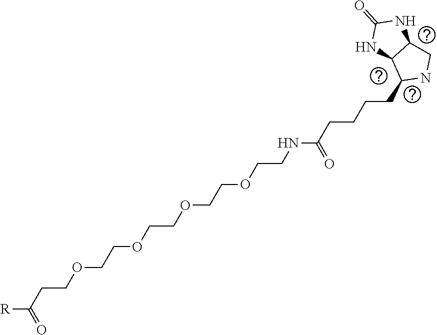 | BiotinPEG4CO, Biotin(PEG4CO), Biotin(PEG4) | O═C(CCCC[C@@ H]([C@H]1N2)SC[C @@H]1NC2═O) NCCOCCOCCOC COCCC([R])═O |
| | C21H36N3O7SR | | |
| 70 | 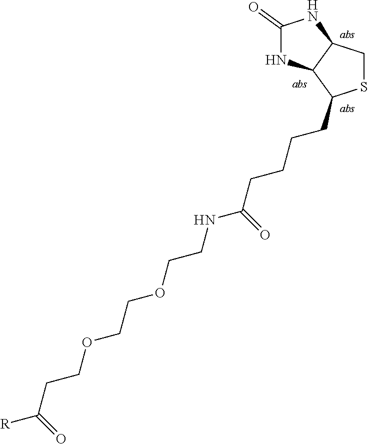 | Biotinyl(dPEG2), Biotin(dPEG2) | O═C(CCCC[C@@ H]([C@H]1N2)SC[C @@H]1NC2═O)NCC OCCOCCC([R])═O |
| | C17H28N3O5SR | | |
| 71 | 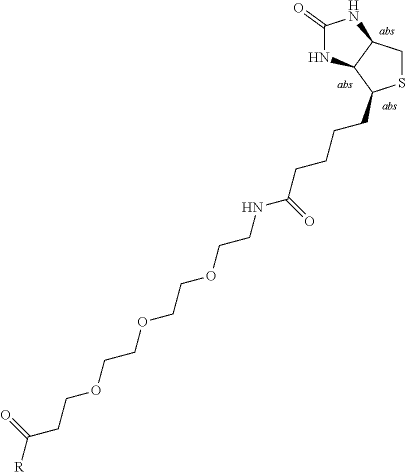 | Biotinyl(dPEG3), Biotin(dPEG3) | O═C(CCCC[C@@ H]([C@H]1N2)SC[C @@H]1NC2═O) NCCOCCOCCOC CC([R])═O |
| | C19H32N3O6SR | | |
| 72 | 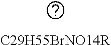 | BrAcdPEG12CO | O═C(CBr)NCCOC COCCOCCOCCO CCOCCOCCOCC |
| | | | OCCOCCOCCOC |
| | | | CC([R])═O |
| 73 | 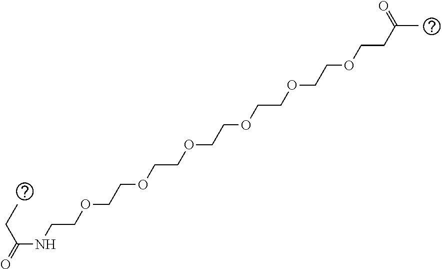 | BrAcdPEG6CO | O═C(CBr)NCCOC COCCOCCOCCO CCOCCC([R])═O |
| | C17H31BrNO8R | | |
| 74 | 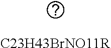 | BrAcdPEG9CO | O═C(CBr)NCCOC COCCOCCOCCO CCOCCOCCOCC |
| | | | OCCC([R])═O |
| 75 | 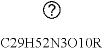 | C12gEPEG2PEG2, C12gEPEG2PEG2CO | CCCCCCCCCCCC (N[C@@H](CCC(NC COCCOCC(NCCOCC OCC([R])═O)═O)═ |
| | | | O)C(O)═O)═O |
| 76 | 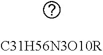 | C14gEPEG2PEG2, C14gEPEG2PEG2CO | CCCCCCCCCCCC CC(N[C@@H](CCC (NCCOCCOCC(NC COCCOCC([R])═ |
| | | | O)═O)═O)C(O)═ |
| | | | O)═O |
| 77 | 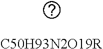 | C18OHgEPEG12, HOC18gEPEG12 | OC(CCCCCCCCC CCCCCCCC(N[C @@H](CCC(NCC |
| | | | OCCOCCOCCOC |
| | | | COCCOCCOCCO |
| | | | CCOCCOCCOCC |
| | | | OCCC([R])═O)═O) |
| | | | C(O)═O)═O)═O |
| 78 | 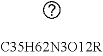 | C18OHgEPEG2PEG2, HOC18gEPEG2PEG2 PEG2PEG2gEC18OH | OC(CCCCCCCCCC CCCCCCC(N[C@ @H](CCC(NCCOC COCC(NCCOCCO |
| | | | CC([R])═O)═O)═O) |
| | | | C(O)═O)═O)═O |
| | | | OC(CCCCCCCCC |
| | | | CCCCCCCC(N[C |
| | | | @@H](CCC(NCC |
| | | | OCCOCC(NCCOC |
| | | | COCC([R])═O)═O)═ |
| | | | O)C(O)═O)═O)═O |
| | | | OC(CCCCCCCCC |
| | | | CCCCCCCC(N[C |
| | | | @@H](CCC(NCC |
| | | | OCCOCC(NCCOC |
| | | | COCC═O)═O)═O) |
| | | | C(O)═O)═O)═O |
| 79 | 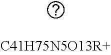 | C18OHgEPEG2PEG2 SP6, HOC18gEPEG2PE G2SP6 | C[N+](C)(CCNC(C OCCOCCNC(COC COCCNC(CC[C@ @H](C(O)═O)NC(C |
| | | | CCCCCCCCCCCC |
| | | | CCCC(O)═O)═O)═ |
| | | | O)═O)═O)CC([R])═ |
| | | | O |
| 80 | 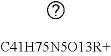 | C18OHgEPEG2SP6PE G2, HOC18gEPEG2SP 6PEG2 | C[N+](C)(CCNC(C OCCOCCNC(CC[C @@H](C(O)═O)NC (CCCCCCCCCCC |
| | | | CCCCCC(O)═O)═ |
| | | | O)═O)═O)CC(NCC |
| | | | OCCOCC([R])═O)═ |
| | | | O |
| 81 | 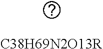 | C18OHgEPEG6, HOC18gEPEG6 | OC(CCCCCCCCCC CCCCCCC(N[C@@ H](CCC(NCCOCCO |
| | | | CCOCCOCCOCCO |
| | | | CCC([R])═)═O)C |
| | | | O(O)═O)═O)═O |
| 82 | 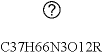 | C20OHgEPEG2PEG2, HOC20gEPEG2PEG2 | OC(CCCCCCCCC CCCCCCCCCC(N[C @@H](CCC(NCCO CCOCC(NCCOCC |
| | | | OCC([R])═O)═O)═ |
| | | | O)C(O)═O)═O)═O |
| 83 | 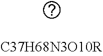 | C20gEPEG2PEG2 | CCCCCCCCCCCC CCCCCCCC(N[C@ @H](CCC(NCCOC COCC(NCCOCCO |
| | | | CC([R])═O)═O)═O) |
| | | | C(O)═O)═O |
| 84 | 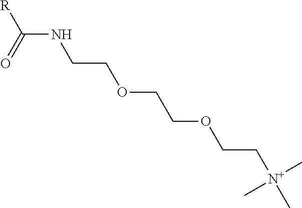 | CO(NHPEG3a) CON(PEG3a) CONHPEG3a | C[N+](C)(C)CCOC COCCNC([R])═O |
| | C10H22N2O3R+ | | |
| 85 | 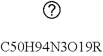 | CO(PEG12gEC18OH) | OC(CCCCCCCCC CCCCCCCC(N[C @@H](CCC(NCC OCCOCCOCCOC |
| | | | COCCOCCOCCO |
| | | | CCOCCOCCOCC |
| | | | OCCNC([R])═O)═ |
| | | | O)C(O)═O)═O)═O |
| 86 | 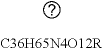 | CO(PEG2PEG2gEC18 OH) | OC(CCCCCCCCCC CCCCCCC(N[C@ @H](CCC(NCCOC |
| | | | COCC(NCCOCCO |
| | | | CCNC([R])═O)═ |
| | | | O)═O)C(O)═O)═ |
| | | | O)═O |
| 87 | 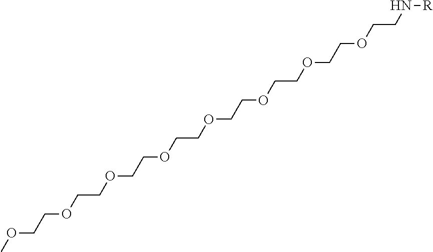 | CO(mPEG8) | COCCOCCOCCO CCOCCOCCOCC OCCN[R] |
| | C17H36NO8R | | |
| 88 | 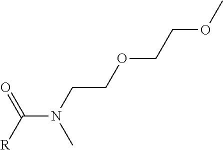 | CON(MePEG2) | CN(CCOCCOC)C ([R])═O |
| | C7H14NO3R | | |
| 89 | 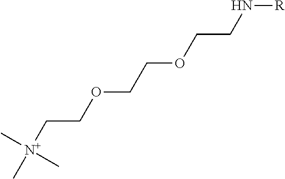 | CONH(PEG3a) | C[N+](C)(C)CCOC COCCN[R] |
| | C9H22N2O2R+ | | |
| 90 | 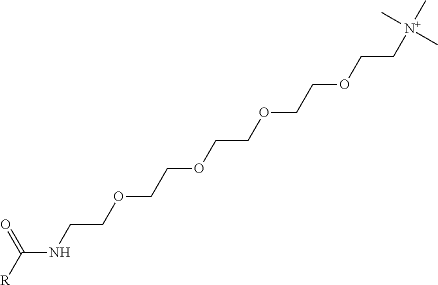 | CONH(PEG5a) | C[N+](C)(C)CCOC COCCOCCOCCN C([R])═O |
| | C14H30N2O5R+ | | |
| 91 | 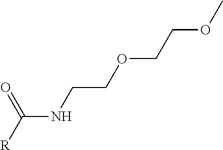 | CONH(mPEG2) | COCCOCCNC ([R])═O |
| | C6H12NO3R | | |
| 92 | 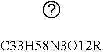 | PEG2PEG2gEC16OH | OC(CCCCCCCCC CCCCCC(N[C@@ H](CCC(NCCOCC OCC(NCCOCCOC |
| | | | C([R])═O)═O)═O) |
| | | | C(O)═O)═O)═O |
| | | | OC(CCCCCCCCC |
| | | | CCCCCC(N[C@@ |
| | | | H](CCC(NCCOCC |
| | | | OCC(NCCOCCOC |
| | | | C═O)═O)═O)C |
| | | | (O)═O)═O)═O |
| 93 | 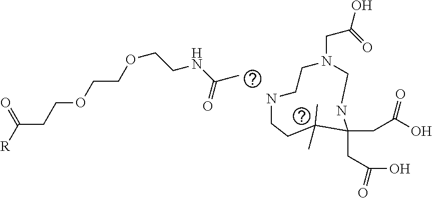 | DOTA(dPEG2) | OC(CN1CCN(CC (O)═O)CCN(CC (O)═O)CCN(CC(NC COCCOCCC([R])═ O)═O)CC1)═O |
| | C23H40N5O10R | | |
| 94 | 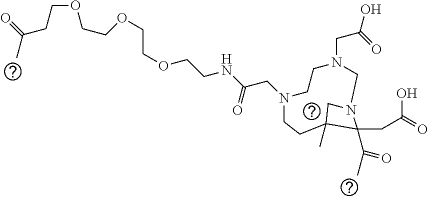 | DOTA(dPEG3) | OC(CN1CCN(CC (O)═O)CCN(CC(O)═ O)CCN(CC(NCCOC COCCOCCC([R])═ O)═O)CC1)═O |
| | C25H44N5O11R | | |
| 95 | 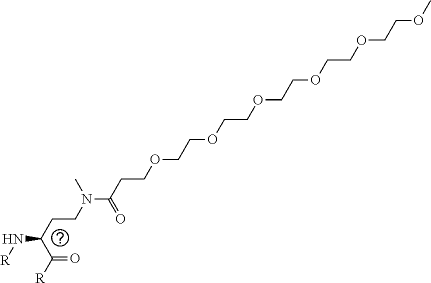 | Dab(NMeCOmPEG6) | CN(CC[C@@H](C ([R])═O)N[R])C(C COCCOCCOCCOC COCCOC)═O |
| | C19H36N2O8R2 | | |
| 96 | 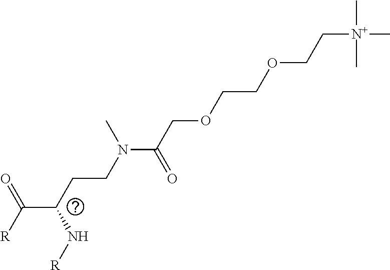 | Dab(NMecPEG2aCO), Dab(NMecPEG2a) | CN(CC[C@@H](C ([R])═O)N[R])C(CO CCOCC[N+](C)(C) C)═O |
| | C14H28N3O4R2+ | | |
| 97 | 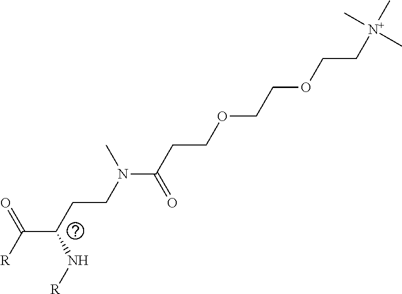 | Dab(NMecPEG3aCO), Dab(NMecPEG3a) | CN(CC[C@@H](C ([R])═O)N[R])C(C COCCOCC[N+](C) (C)C)═O |
| | C15H30N3O4R2+ | | |
| 98 | 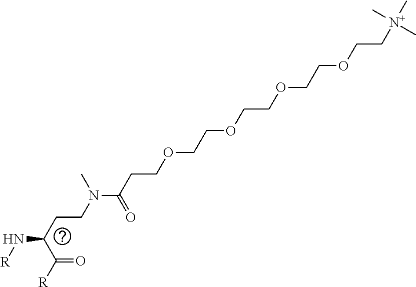 | Dab(NMecPEG5aCO), Dab(NMecPEG5a) | CN(CC[C@@H](C ([R])═O)N[R])C(C COCCOCCOCCO CC[N+](C)(C)C)═O |
| | C19H38N3O6R2+ | | |
| 99 | 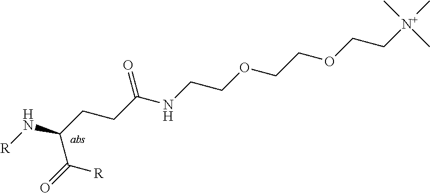 | E(COcPEG3a)) | C[N+](C)(C)CCOC COCCNC(CC[C@ @H](C([R])═O)N [R])═O |
| | C14H28N3O4R2+ | | |
| 100 | 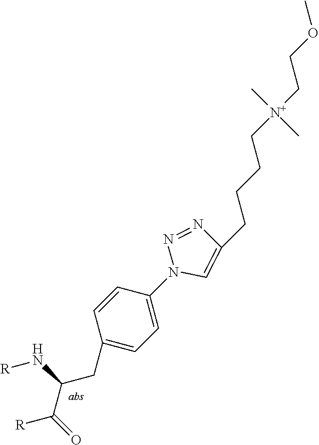 | F(4TzlDMA4mPEG) | C[N+](C)(CCCCc1 cn(-c2ccc(C[C@@H] (C([R])═O)N[R])cc 2)nn1)CCOC |
| | C20H30N5O2R2+ | | |
| 101 | 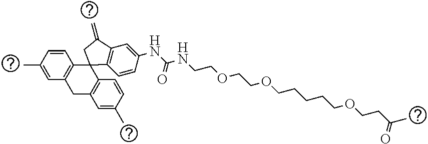 | FITCPEG4CO | Oc1cc(Oc2c(C3(c (cc4)c5cc4NC(NCC OCCOCCOCCOC CC([R])═O)═S)OC 5═O)ccc(O)c2)c3cc1 |
| | C32H33N2O10SR | | |
| 102 | 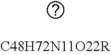 | FlagTag(dPEG2) | NCCCC[C@@H](C (NCCOCCOCCC ([R])═O)═O)NC([C @H](CC(O)═O)N |
| | | | C([C@H](CC(O)═ |
| | | | O)NC([C@H](CC |
| | | | (O)═O)NC([C@H] |
| | | | (CC(O)═O)NC([C@ |
| | | | H](CCCCN)NC([C |
| | | | @H](Cc(cc1)ccc1 |
| | | | O)NC([C@H] (CC |
| | | | (O)═O)N)═O)═O)═ |
| | | | O)═O)═O)═O)═O |
| 103 | 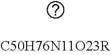 | FlagTag(dPEG3) | NCCCC[C@@H](C (NCCOCCOCCOC CC([R])═O)═O)NC ([C@H](CC(O)═O) |
| | | | NC([C@H] (CC(O)═ |
| | | | O)NC([C@H](CC |
| | | | (O)═O)NC([C@H] |
| | | | (CC(O)═O)NC([C |
| | | | @H](CCCCN)NC |
| | | | ([C@H](Cc(cc1)ccc1 |
| | | | O)NC([C@H](CC |
| | | | (O)═O)N)═O)═O)═ |
| | | | O)═O)═O)═O)═O |
| 104 | 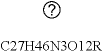 | HOC10gEPEG2PEG2, HOC10gEPEG2PEG2 CO | OC(CCCCCCCCC (N[C@@H](CCC(N CCOCCOCC(NCCO CCOCC([R])═O)═ |
| | | | O)═O)C(O)═O)═ |
| | | | O)═O |
| | | | OC(CCCCCCCCC |
| | | | (N[C@@H](CCC(N |
| | | | CCOCCOCC(NCCO |
| | | | CCOCC═O)═O)═ |
| | | | O)C(O)═O)═O)═O |
| 105 | 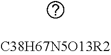 | HOC16gEPEG2PEG2 orn, HOC16OHgEPEG 2PEG2orn(2) | OC(CCCCCCCCC CCCCCC(N[C@@ H](CCC(NCCOCC OCC(NCCOCCOC |
| | | | C(N[C@H](CCCN |
| | | | [R])C([R])═O)═ |
| | | | O)═O)═O)C(O)═ |
| | | | O)═O)═O |
| | | | NCCC[C@H](C═O) |
| | | | NC(COCCOCCNC |
| | | | (COCCOCCNC(CC |
| | | | [C@@H](C(O)═O) |
| | | | NC(CCCCCCCCC |
| | | | CCCCCC(O)═O) |
| | | | O)═O)═O)═O |
| 106 | 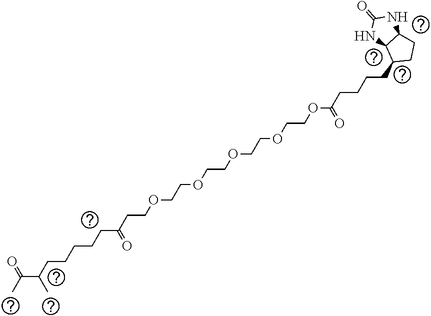 | K(BiotinPEG4) | O═C(CCCC[C@@ H]([C@H]1N2)SC[C @@H]1NC2═O) NCCOCCOCCOC COCCC(NCCCC[C @@H](C([R])═O) N[R])═O |
| | C27H47N5O8SR2 | | |
| 107 | 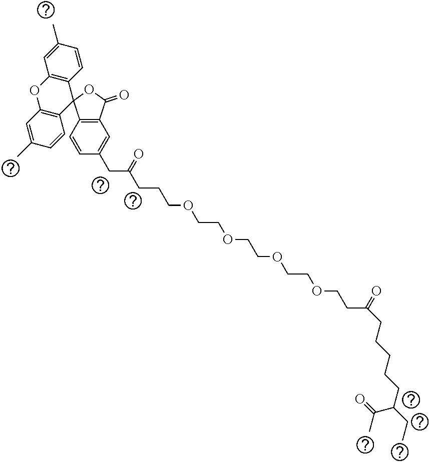 | K(FITCPEG4) | Oclcc(Oc2c(C3(c (cc4)c5cc4NC(NCC OCCOCCOCCOC CC(NCCCC[C@@ H](C([R])═O)N[R])═ O)═S)OC5═O)ccc (O)c2)c3cc1 |
| | C38H44N4O11SR2 | | |
| 108 | 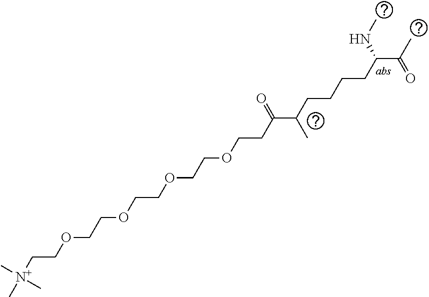 | K(NMeCOPEG4N+ Me3) | CN(CCCC[C@@H] (C([R])═O)N[R])C (CCOCCOCCOCCO CC[N+](C)(C)C)═O |
| | C21H42N3O6R2+ | | |
| 109 | 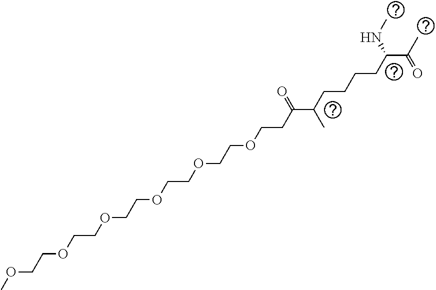 | K(NMeCOmPEG6) | CN(CCCC[C@@H] (C([R])═O)N[R])C (CCOCCOCCOCC OCCOCCOC)═O |
| | C21H40N2O8R2 | | |
| 110 | 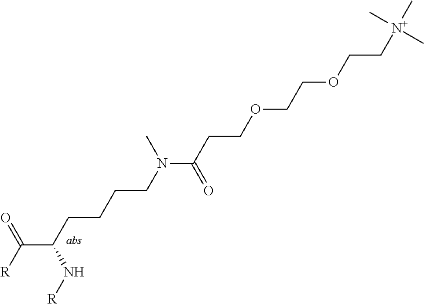 | K(NMePEG3a), K(NMecPEG3a), K(NMecPEG3aCO) | CN(CCCC[C@@H (C([R])═O)N[R])C (CCOCCOCC[N+] (C)(C)C)═O |
| | C17H34N3O4R2+ | | |
| 111 | 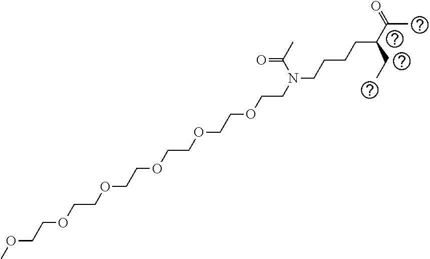 | K(NmPEG6Ac) | CC(N(CCCC[C@ @H](C([R])═O)N [R])CCOCCOCCO CCOCCOCCOC)═ O |
| | C21H40N2O8R2 | | |
| 112 | 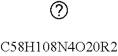 | K(PEG12NMegENMe C18OH) | CN(CCOCCOCCOC COCCOCCOCCOC COCCOCCOCCOC COCCC(NCCCC[C |
| | | | @@H](C([R])═O) |
| | | | N[R])═O)C(CC[C |
| | | | @@H](C(O)═O)N |
| | | | (C)C(CCCCCCCC |
| | | | CCCCCCCCC |
| | | | (O)═O)═O)═O |
| 113 | 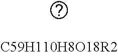 | K(PEG12NMegENMe C18 Tetrazole) | CN(CCOCCOCCO CCOCCOCCOCC OCCOCCOCCOC COCCOCCC(NCC |
| | | | CC[C@@H](C([R])═ |
| | | | O)N[R])═O)C(C |
| | | | C[C@@H](C(O)═ |
| | | | O)N(C)C(CCCCC |
| | | | CCCCCCCCCCCC |
| | | | c1nnn[nH]1)═O)═O |
| 114 | 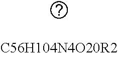 | K(PEG12gEC18OH) | OC(CCCCCCCCC CCCCCCCC(N[C @@H](CCC(NCC OCCOCCOCCOC |
| | | | COCCOCCOCCO |
| | | | CCOCCOCCOCC |
| | | | OCCC(NCCCC[C |
| | | | @@H](C([R])═O) |
| | | | N[R])═O)═O)C |
| | | | (O)═O)═O)═O |
| 115 | 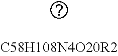 | K(PEG12gEC20OH) | OC(CCCCCCCCC CCCCCCCCCC(N [C@@H](CCC(NC COCCOCCOCCO |
| | | | CCOCCOCCOCC |
| | | | OCCOCCOCCOC |
| | | | COCCC(NCCCC[C |
| | | | @@H](C([R])═O) |
| | | | N[R])═O)═O)C |
| | | | (O)═O)═O)═O |
| 116 | 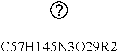 | K(PEG24C18OH) | OC(CCCCCCCCC CCCCCCCC(NCC OCCOCCOCCOC COCCOCCOCCO |
| | | | CCOCCOCCOCC |
| | | | OCCOCCOCCOC |
| | | | COCCOCCOCCO |
| | | | CCOCCOCCOCC |
| | | | OCCOCCC(NCC |
| | | | CC[C@@H](C |
| | | | ([R])═O)N[R])═ |
| | | | O)═O)═O |
| 117 | 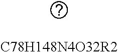 | K(PEG24gEC16OH) | OC(CCCCCCCCC CCCCCC(N[C@@ H](CCC(NCCOCC OCCOCCOCCOC |
| | | | COCCOCCOCCO |
| | | | CCOCCOCCOCC |
| | | | OCCOCCOCCOC |
| | | | COCCOCCOCCO |
| | | | CCOCCOCCOCC |
| | | | OCCC(NCCCC[C |
| | | | @@H](C([R])═O) |
| | | | N[R])═O)═O)C |
| | | | (O)═O)═O)═O |
| 118 | 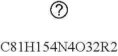 | K(PEG24gEC18OH) | C[C@](CCCCNC (CCOCCOCCOCC OCCOCCOCCOC COCCOCCOCCO |
| | | | CCOCCOCCOCC |
| | | | OCCOCCOCCOC |
| | | | COCCOCCOCCO |
| | | | CCOCCOCCNC(C |
| | | | C[C@@H](C(O)═ |
| | | | O)NC(CCCCCCC |
| | | | CCCCCCCCCC |
| | | | (O)═O)═O)═O)═ |
| | | | O)(C([R])═O)N[R] |
| 119 | 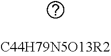 | K(PEG2NMePEG2NM egENMeC18OH) | CN(CCOCCOCCN ((C)CCOCCOCC(N CCCC[C@@H](C ([R])═O)N[R])═O)═ |
| | | | O)C(CC[C@@H](C |
| | | | (O)═O)N(C)C(CCC |
| | | | CCCCCCCCCCCC |
| | | | CC(O)═O═O)═O |
| 120 | 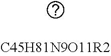 | K(PEG2NMePEG2NM egENMeC18Tetrazole) | CN(CCOCCOCC(N (C)CCOCCOCC(N CCCC[C@@H](C ([R])═O)N[R])═O)═ |
| | | | O)C(CC[C@@H](C |
| | | | (O)═O)N(C)C(CCC |
| | | | CCCCCCCCCCCC |
| | | | CCc1nnn[nH]1)═ |
| | | | O)═O |
| 121 | 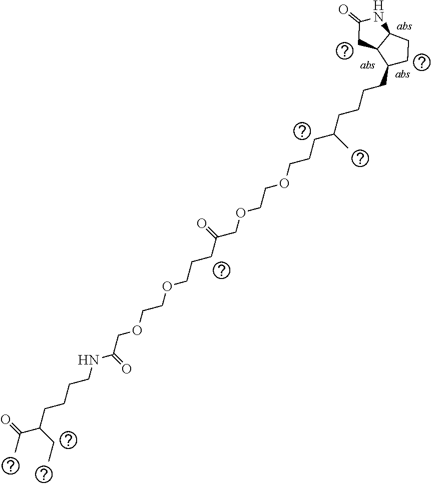 | K(PEG2PEG2Biotin) | O═C(CCCC[C@@ H]([C@H]1N2)SC[C @@H]1NC2═O) NCCOCCOCC(NC COCCOCC(NCCC C[C@@H](C([R])═ O)N[R])═O)═O |
| | C28H48N6O9SR2 | | |
| 122 | 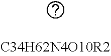 | K(PEG2PEG2C16OH) | OC(CCCCCCCCC CCCCCC(NCCOC COCC(NCCOCCO CC(NCCCC[C@@ |
| | | | H](C([R])═O)N[R])═ |
| | | | O)═O)═O)═O |
| 123 | 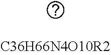 | K(PEG2PEG2C18OH) | OC(CCCCCCCCC CCCCCCCC(NCCO CCOCC(NCCOCCO CC(NCCCC[C@@ |
| | | | H](C([R])═O)N[R])═ |
| | | | O)═O)═O)═O |
| 124 | 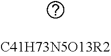 | K(PEG2PEG2DgEC18 OH) | OC(CCCCCCCCC CCCCCCCC(N[C @H](CCC(NCCO CCOCC(NCCOCC |
| | | | OCC(NCCCC[C@ |
| | | | @H](C([R])═O)N |
| | | | [R])═O)═O)═O)C |
| | | | (O)═O)═O)═O |
| 125 | 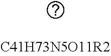 | K(PEG2PEG2PC18OH) | OC(CCCCCCCCC CCCCCCCC(N(C CC1)[C@@H]1C(N CCOCCOCC(NCC |
| | | | OCCOCC(NCCCC |
| | | | [C@@H](C([R])═ |
| | | | O)N[R])═O)═O)═ |
| | | | O)═O)═O |
| 126 | 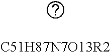 | K(PEG2PEG2PPPC18 OH) | OC(CCCCCCCCC CCCCCCCC(N(C CC1)[C@@H]1C(N (CCC1)[C@@H] |
| | | | 1C(N(CCC1)[C@ |
| | | | @H]1C(NCCOCC |
| | | | OCC(NCCOCCOC |
| | | | C(NCCCC[C@@H] |
| | | | (C([R])═O)N[R])═ |
| | | | O)═O)═O)═O)═ |
| | | | O)═O)═O |
| 127 | 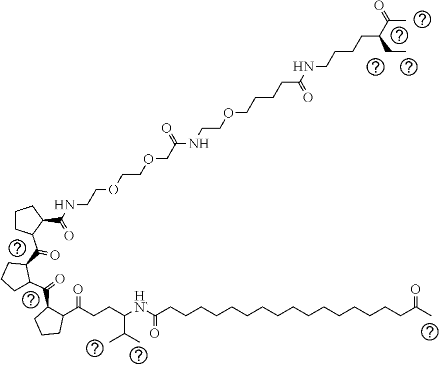 | K(PEG2PEG2PPPgEC 18OH) | OC(CCCCCCCCCCC CCCCCC(N[C@@ H](CCC(N(CCC1) [C@@H]1C(N(CC C1)[C@@H]1C(N(C CC1)[C@@H]1C(NC COCCOCC(NCCOC COCC(NCCCC[C @@H](C([R])═O)N [R])═O)═O)═O)═ O)═O)═O)C(O)═ O)═O)═O |
| | C56H94N8O16R2 | | |
| 128 | 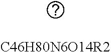 | K(PEG2PEG2PgEC18 OH) | OC(CCCCCCCCC CCCCCCCC(N[C @@H](CCC(N(CC C1)[C@@H]1C(N |
| | | | CCOCCOCC(NCC |
| | | | OCCOCC(NCCCC |
| | | | [C@@H](C([R])═O) |
| | | | N[R])═O)═O)═O)═ |
| | | | O)C(O)═O)═O)═O |
| 129 | 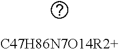 | K(PEG2PEG2Sp6gEC18 OH) | C[N+](C)(CCNC(C C[C@@H](C(O)═ O)NC(CCCCCCCC CCCCCCCCC(O)═ |
| | | | O)═O)═O)CC(N |
| | | | CCOCCOCC(NCC |
| | | | OCCOCC(NCCCC |
| | | | [C@@H](C([R])═O) |
| | | | N[R])═O)═O)═O |
| 130 | 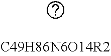 | K(PEG2PEG2TrxgEC18 OH) | OC(CCCCCCCCC CCCCCCCC(N[C@ @H](CCC(NC[C@ H](CC1)CC[C@@ |
| | | | H]1C(NCCOCCOC |
| | | | C(NCCOCCOCC(N |
| | | | CCCC[C@@H](C |
| | | | ([R])═O)N[R])═O)═ |
| | | | O)═O)═O)C(O)═ |
| | | | O)═O)═O |
| 131 | 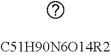 | K(PEG2PEG2TrxgEC20 OH) | OC(CCCCCCCCC CCCCCCCCCC(N [C@@H](CCC(NC [C@H](CC1)CC[C |
| | | | @@H]1C(NCCOC |
| | | | COCC(NCCOCCO |
| | | | CC(NCCCC[C@@ |
| | | | H](C([R])═O)N[R])═ |
| | | | O)═O)═O)═O)C |
| | | | (O)═O)═O)═O |
| 132 | 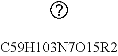 | K(PEG2PEG2TrxgETrx C20OH) | OC(CCCCCCCCC CCCCCCCCCC(N C[C@H](CC1)CC[C @@H]1C(N[C@ |
| | | | @H](CCC(NC[C@ |
| | | | H](CC1)CC[C@H] |
| | | | 1C(NCCOCCOCC(N |
| | | | CCOCCOCC(NCCC |
| | | | C[C@@H] (C([R])═ |
| | | | O)N[R])═O)═O)═ |
| | | | O)═O)C(O)═O)═ |
| | | | O)═O)═O |
| 133 | 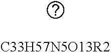 | K(PEG2PEG2gEC10 OH) | OC(CCCCCCCCC (N[C@@H](CCC(N CCOCCOCC(NCCO CCOCC(NCCCC[C |
| | | | @@H](C([R])═O)N |
| | | | [R])═O)═O)═O)C |
| | | | (O)═O)═O)═O |
| 134 | 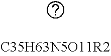 | K(PEG2PEG2gEC12) | CCCCCCCCCCCC (N[C@@H](CCC(N CCOCCOCC(NCCO CCOCC(NCCCC[C |
| | | | @@H](C([R])═O) |
| | | | N[R])═O)═O)═O)C |
| | | | (O)═O)═O |
| 135 | 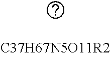 | K(PEG2PEG2gEC14) NMeK(PEG2PEG2gEC 14) | CCCCCCCCCCCC CC(N[C@@H](CCC (NCCOCCOCC(N CCOCCOCC(NCCC |
| | | | C[C@@H](C([R])═ |
| | | | O)N[R])═O)═O)═O) |
| | | | C(O)═O)═O |
| 136 | 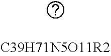 | K(PEG2PEG2gEC16) | CCCCCCCCCCCC CCCC(N[C@@H](C CC(NCCOCCOCC(N CCOCCOCC(NCCC |
| | | | C[C@@H](C([R])═ |
| | | | O)N[R])═O)═O)═ |
| | | | O)C(O)═O)═O |
| 137 | 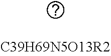 | K(PEG2PEG2gEC16 OH) | OC(CCCCCCCCCC CCCCC(N[C@@H] (CCC(NCCOCCOC C(NCCOCCOCC(N |
| | | | CCCC[C@@H](C |
| | | | ([R])═O)N[R])═O)═ |
| | | | O)═O)C(O)═O)═ |
| | | | O)═O |
| 138 | 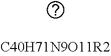 | K(PEG2PEG2gEC16 tetrazole) | OC([C@H](CCC(N CCOCCOCC(NCCO CCOCC(NCCCC[C @@H](C([R])═O)N |
| | | | [R])═O)═O)═O)NC |
| | | | (CCCCCCCCCCCC |
| | | | CCCc1nnn[nH]1)═ |
| | | | O)═O |
| 139 | 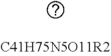 | K(PEG2PEG2gEC18) | CCCCCCCCCCCCC CCCCC(N[C@@H] (CCC(NCCOCCOC C(NCCOCCOCC(N |
| | | | CCCC[C@@H](C |
| | | | ([R])═O)N[R])═O)═ |
| | | | O)═O)C(O)═O)═O |
| 140 | 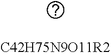 | K(PEG2PEG2gEC18 tetrazole) | OC([C@H](CCC(NC COCCOCC(NCCOC COCC(NCCCC[C@ @H](C([R])═O)N |
| | | | [R])═O)═O)═O)NC |
| | | | (CCCCCCCCCCCC |
| | | | CCCCCc1nnn[nH] |
| | | | 1)═O)═O |
| 141 | 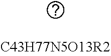 | K(PEG2PEG2gEC20 OH) | OC(CCCCCCCCC CCCCCCCCCC(N [C@@H](CCC(NC COCCOCC(NCCO |
| | | | CCOCC(NCCCC[C |
| | | | @@H](C([R])═O) |
| | | | N[R])═O)═O)═O) |
| | | | C(O)═O)═O)═O |
| 142 | 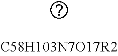 | KPEG2PEG2gEDap(C 16OH)2, K(PEG2PEG2g EDAP(C16OH)2) | OC(CCCCCCCCCCC CCCC(NC[C@@H] (C(N[C@@H](CCC (NCCOCCOCC(NC |
| | | | COCCOCC(NCCCC |
| | | | [C@@H](C([R])═O) |
| | | | N[R])═O)═O)═O) |
| | | | C(O)═O)═O)NC(CC |
| | | | CCCCCCCCCCCC |
| | | | C(O)═O)═O)═O)═ |
| | | | O |
| 143 | 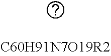 | K(PEG2PEG2gEDAP (mXOH)2) KPEG2PEG2gEDAP (mXOH)2 | OC([C@H](CCC(NC COCCOCC(NCCOC COCC(NCCCC[C@ @H](C([R])═O)N |
| | | | [R])═O)═O)═O)N |
| | | | C([C@H](CNC(C |
| | | | CCCCCCCCOc1cc |
| | | | (C(O)═O)ccc1)═O) |
| | | | NC(CCCCCCCCC |
| | | | Oc1cc(C(O)═O) |
| | | | ccc1)═O)═O)═O |
| 144 | 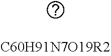 | K(PEG2PEG2gEDAP (pXOH)2) KPEG2PEG2gEDAP (pXOH)2 | OC([C@H](CCC(NC COCCOCC(NCCOC COCC(NCCCC[C@ @H](C([R])═O)N |
| | | | [R])═O)═O)═O)NC |
| | | | ([C@H](CNC(CCC |
| | | | CCCCCCOc(cc1)ccc |
| | | | 1C(O)═O)═O)NC(C |
| | | | CCCCCCCCOc(cc1) |
| | | | ccc1C(O)═O)═O)═ |
| | | | O)═O |
| 145 | 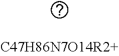 | K(PEG2PEG2gESp6C 18OH) | C[N+](C)(CCNC(CC CCCCCCCCCCCCC CC(O)═O)═O)CC(N [C@@H](CCC(NCC |
| | | | OCCOCC(NCCOCC |
| | | | OCC(NCCCC[C@@ |
| | | | H](C([R])═O)N[R])═ |
| | | | O)═O)═O)C(O)═ |
| | | | O)═O |
| 146 | 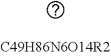 | K(PEG2PEG2gETrxC18 OH) | OC(CCCCCCCCCC CCCCCCC(NC[C@ H](CC1)CC[C@@H] 1C(N[C@@H](CCC |
| | | | (NCCOCCOCC(NC |
| | | | COCCOCC(NCCC |
| | | | C[C@@H](C([R])═ |
| | | | O)N[R])═O)═O)═ |
| | | | O)C(O)═O)═O)═ |
| | | | O)═O |
| 147 | 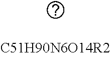 | K(PEG2PEG2gETrxC20 OH) | OC(CCCCCCCCCC CCCCCCCCC(NC [C@H](CC1)CC[C@ @H]1C(N[C@@H] |
| | | | (CCC(NCCOCCOC |
| | | | C(NCCOCCOCC(N |
| | | | CCCC[C@@H](C |
| | | | ([R])═O)N[R])═O)═ |
| | | | O)═O)C(O)═O)═ |
| | | | O)═O)═O |
| 148 | 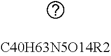 | K(PEG2PEG2gEmXOH) | OC([C@H](CCC(N CCOCCOCC(NCC OCCOCC(NCCCC [C@@H](C([R])═ |
| | | | O)N[R])═O)═O)═ |
| | | | O)NC(CCCCCCC |
| | | | CCOc1cc(C(O)═O) |
| | | | ccc1)═O)═O |
| 149 | 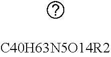 | K(PEG2PEG2gEpXOH) | OC([C@H](CCC(NC COCCOCC(NCCO CCOCC(NCCCC[C @@H](C([R])═O)N |
| | | | [R])═O)═O)═O)NC |
| | | | (CCCCCCCCCOc |
| | | | (cc1)ccc1C(O)═O)═ |
| | | | O)═O |
| 150 | 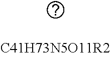 | K(PEG2PEG2pC18OH) | OC(CCCCCCCCCC CCCCCCC(N(CCC 1)[C@H]1C(NCCO CCOCC(NCCOCCO |
| | | | CC(NCCCC[C@@ |
| | | | H](C([R])═O)N |
| | | | [R])═O)═O)═O)═ |
| | | | O)═O |
| 151 | 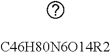 | K(PEG2PEG2pgEC18 OH) | OC(CCCCCCCCC CCCCCCCC(N[C @@H](CCC(N(CC C1)[C@H]1C(NC |
| | | | COCCOCC(NCCO |
| | | | CCOCC(NCCCC[C |
| | | | @@H](C([R])═O)N |
| | | | [R])═O)═O)═O)═ |
| | | | O)C(O)═O)═O)═O |
| 152 | 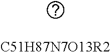 | K(PEG2PEG2pppC18 OH) | OC(CCCCCCCCCC CCCCCCC(N(CCC 1)[C@H]1C(N(CC C1)[C@H]1C(N(C |
| | | | CC1)[C@H]1C(NC |
| | | | COCCOCC(NCCO |
| | | | CCOCC(NCCCC[C |
| | | | @@H](C([R])═O)N |
| | | | [R])═O)═O)═O)═ |
| | | | O)═O)═O)═O |
| 153 | 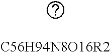 | K(PEG2PEG2pppgEC 18OH) | OC(CCCCCCCCCCC CCCCCC(N[C@@H] (CCC(N(CCC1)[C@ H]1C(N(CCC1)[C@ |
| | | | H]1C(N(CCC1)[C |
| | | | @H]1C(NCCOCCO |
| | | | CC(NCCOCCOCC |
| | | | (NCCCC[C@@H](C |
| | | | ([R])═O)N[R])═O)═ |
| | | | O)═O)═O)═O)═O) |
| | | | C(O)═O)═O)═O |
| 154 | 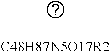 | K(PEG2PEG6gEC16 OH) | OC(CCCCCCCCCC CCCCC(N[C@@H] (CCC(NCCOCCOC COCCOCCOCCOC |
| | | | CC(NCCOCCOCC |
| | | | (NCCCC[C@@H] |
| | | | (C([R])═O)N[R])═ |
| | | | O)═O)═O)C(O)═ |
| | | | O)═O)═O |
| 155 | 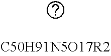 | K(PEG2PEG6gEC18 OH) | OC(CCCCCCCCCC CCCCCCC(N[C@@ H](CCC(NCCOCCO CCOCCOCCOCCO |
| | | | CCC(NCCOCCOC |
| | | | C(NCCCC[C@@H] |
| | | | (C([R])═O)N[R])═ |
| | | | O)═O)═O)C(O)═ |
| | | | O)═O)═O |
| 156 | 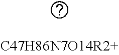 | K(PEG2Sp6PEG2gEC 18OH) | C[N+](C)(CCNC(C OCCOCCNC(CC[C @@H](C(O)═O)NC (CCCCCCCCCCCC |
| | | | CCCCC(O)═O)═ |
| | | | O)═O)═O)CC(NCC |
| | | | OCCOCC(NCCCC |
| | | | [C@@H](C([R])═ |
| | | | O)N[R])═O)═O |
| 157 | 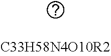 | K(PEG2gEC16OH) | OC(CCCCCCCCCC CCCCC(N[C@@H] (CCC(NCCOCCOC C(NCCCC[C@@H] |
| | | | (C([R])═O)N[R])═ |
| | | | O)═O)C(O)═O)═ |
| | | | O)═O |
| 158 | 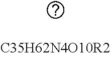 | K(PEG2gEC18OH) | OC(CCCCCCCCCC CCCCCCC(N[C@@ H](CCC(NCCOCC OCC(NCCCC[C@ |
| | | | @H](C([R])═O)N |
| | | | [R])═O)═O)C(O)═ |
| | | | O)═O)═O |
| 159 | 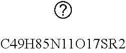 | K(PEG2gEgEPEG24S BC16Tetrazole) | OC([C@H](CCC(NC COCCOCC(NCCCC [C@@H](C([R])═O) N[R])═O)═O)NC(CC |
| | | | [C@@H](C(O)═O)N |
| | | | C(COCCOCCNC(CC |
| | | | CS(NC(CCCCCCCC |
| | | | CCCCCCCc1nnn[nH] |
| | | | 1)═O)(═O)═O)═ |
| | | | O)═O)═O)═O |
| 160 | 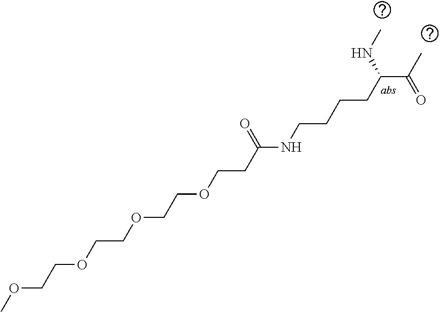 | K(PEG3OMe) K(mPEG4) | COCCOCCOCCOC CC(NCCCC[C@@H] (C([R])═O)N[R])═O |
| | C16H30N2O6R2 | | |
| 161 | 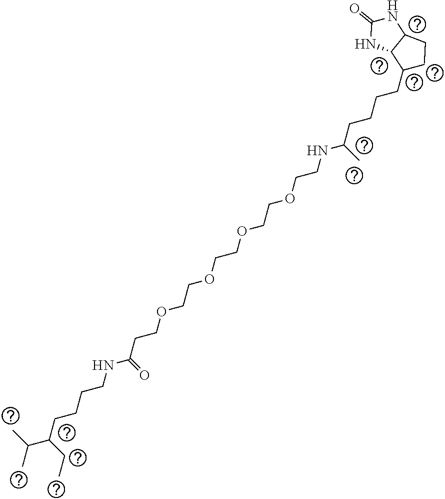 | K(PEG4Biotina), K(PEG4Biotin) | O═C(CCCC[C@H] ([C@@H]1N2)SC[C @H]1NC2═O)NCC OCCOCCOCCOCC C(NCCCC[C@@H] (C([R])═O)N[R])═O |
| | C27H47N5O8SR2 | | |
| 162 | 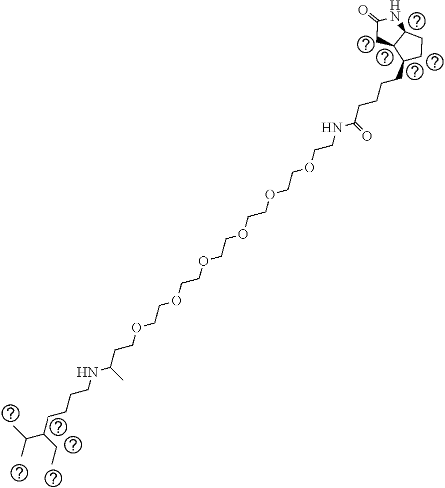 | K(PEG6Biotin) | O═C(CCCC[C@@H] ([C@H]1N2)SC[C@ @H]1NC2═O)NCCO CCOCCOCCOCCOC COCCC(NCCCC[C@ @H](C([R])═O)N [R])═O |
| | C31H55N5O10SR2 | | |
| 163 | 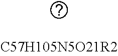 | K(PEG6PEG6gEC16 OH) | OC(CCCCCCCCCC CCCCC(N[C@@H] (CCC(NCCOCCOC COCCOCCOCCOC |
| | | | CC(NCCOCCOCCO |
| | | | CCOCCOCCOCCC |
| | | | (NCCCC[C@@H](C |
| | | | ([R])═O)N[R])═O)═ |
| | | | O)═O)C(O)═O)═ |
| | | | O)═O |
| 164 | 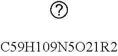 | K(PEG6PEG6gEC18 OH) | OC(CCCCCCCCCC CCCCCCC(N[C@@ H](CCC(NCCOCCO CCOCCOCCOCCO |
| | | | CCC(NCCOCCOCC |
| | | | OCCOCCOCCOCC |
| | | | C(NCCCC[C@@H] |
| | | | (C([R])═O)N[R])═ |
| | | | O)═O)═O)C(O)═ |
| | | | O)═O)═O |
| 165 | 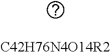 | K(PEG6gEC16OH) | OC(CCCCCCCCCC CCCCC(N[C@@H] (CCC(NCCOCCOC COCCOCCOCCOC |
| | | | CC(NCCCC[C@@ |
| | | | H](C([R])═O)N |
| | | | [R])═O)═O)C(O)═ |
| | | | O)═O)═O |
| 166 | 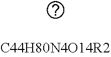 | K(PEG6gEC18OH) | OC(CCCCCCCCCC CCCCCCC(N[C@@ H](CCC(NCCOCCO CCOCCOCCOCCOC |
| | | | CC(NCCCC[C@@H] |
| | | | (C([R])═O)N[R])═ |
| | | | O)═O)C(O)═O)═ |
| | | | O)═O |
| 167 | 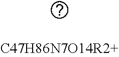 | K(Sp6PEG2PEG2gEC 18OH) | C[N+](C)(CCNC(CO CCOCCNC(COCCO CCNC(CC[C@@H] (C(O)═O)NC(CCC |
| | | | CCCCCCCCCCCCC |
| | | | C(O)═O)═O)═O)═ |
| | | | O)═O)CC(NCCCC |
| | | | [C@@H](C([R])═O) |
| | | | N[R])═O |
| 168 | 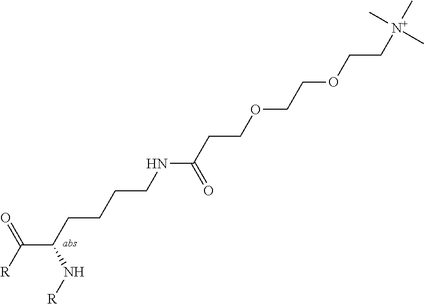 | K(cPEG3a), K(cPEG3aCO) | C[N+](C)(C)CCOCC OCCC(NCCCC[C@ @H](C([R])═O)N [R])═O |
| | C16H32N3O4R2+ | | |
| 169 | 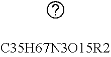 | K(dPEG12Ac) | CC(NCCOCCOCCO CCOCCOCCOCCOC COCCOCCOCCOCC OCCC(NCCCC[C@ |
| | | | @H](C([R])═O)N |
| | | | [R])═O)═O |
| 170 | 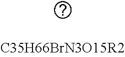 | K(dPEG12AcBr) | O═C(CCOCCOCCO CCOCCOCCOCCO CCOCCOCCOCCO CCOCCNC(CBr)═ |
| | | | O)NCCCC[C@@H] |
| | | | (C([R])═O)N[R] |
| 171 | 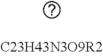 | K(dPEG6Ac) | CC(NCCOCCOCCO CCOCCOCCOCCC (NCCCC[C@@H](C ([R])═O)N[R])═ |
| | | | O)═O |
| 172 | 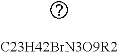 | K(dPEG6AcBr) | O═C(CCOCCOCCO CCOCCOCCOCCN C(CBr)═O)NCCCC [C@@H](C([R])═O) |
| | | | N[R] |
| 173 | 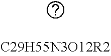 | K(dPEG9Ac) | CC(NCCOCCOCCO CCOCCOCCOCCO CCOCCOCCC(NCC CC[C@@H](C([R])═ |
| | | | O)N[R])═O)═O |
| 174 | 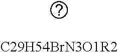 | K(dPEG9AcBr) | O═C(CCOCCOCCO CCOCCOCCOCCOC COCCOCCNC (CBr)═O)NCCCC[C |
| | | | @@H](C([R])═O)N |
| | | | [R] |
| 175 | 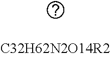 | K(mPEG12) | COCCOCCOCCOC COCCOCCOCCOC COCCOCCOCCOC CC(NCCCC[C@@ |
| | | | H](C([R])═O)N |
| | | | [R])═O |
| 176 | 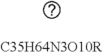 | PEG2PEG2gEC18 | CCCCCCCCCCCCC CCCCC(N[C@@H] (CCC(NCCOCCOC C(NCCOCCOCC |
| | | | ([R])═O)═O)═O)C |
| | | | (O)═O)═O |
| | | | CCCCCCCCCCCCC |
| | | | CCCCC(N[C@@H] |
| | | | (CCC(NCCOCCOC |
| | | | C(NCCOCCOCC═ |
| | | | O)═O)═O)C(O)═ |
| | | | O)═O |
| 177 | 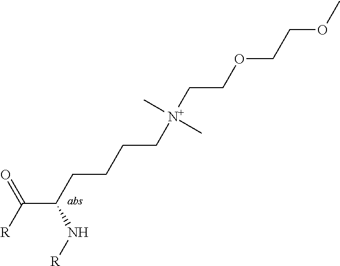 | Lys(N+Me2mPEG3) | C[N+](C)(CCCC[C @@H](C([R])═O)N [R])CCOCCOC |
| | C13H27N2O3R2+ | | |
| 178 | 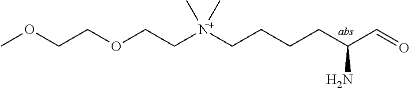 | LysQuatMe2mPEG3, Lys(N+(Me)2mPEG3) | C[N+](C)(CCCC[C@ @H](C═O)N)CCOC COC |
| | C13H29N2O3+ | | |
| 180 | 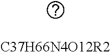 | N(PEG2PEG2gEC18 OH)Gly | OC(CCCCCCCCCC CCCCCCC(N[C@@ H](CCC(NCCOCCO CC(NCCOCCOCCN |
| | | | (CC([R])═O)[R])═ |
| | | | O)═O)C(O)═O)═ |
| | | | O)═O |
| 181 | 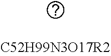 | NMeK(PEG12C18OH) | CN([C@@H](CCCC NC(CCOCCOCCOC COCCOCCOCCOC COCCOCCOCCOC |
| | | | COCCNC(CCCCCC |
| | | | CCCCCCCCCCC |
| | | | (O)═O)═O)═O)C |
| | | | ([R])═O)[R] |
| 182 | 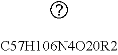 | NMeK(PEG12gEC18 OH) | CN([C@@H](CCCC NC(CCOCCOCCOC COCCOCCOCCOCC OCCOCCOCCOCCO |
| | | | CCNC(CC[C@@H] |
| | | | (C(O)═O)NC(CCC |
| | | | CCCCCCCCCCCC |
| | | | CC(O)═O)═O)═ |
| | | | O)═O)C([R])═O)[R] |
| 183 | 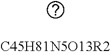 | NMeK(PEG2NMePEG 2NMegENMeC18OH) | CN(CCOCCOCC(N (C)CCOCCOCC(NC CCC[C@@H](C ([R])═O)N(C)[R])═ |
| | | | O)═O)C(CC[C@@ |
| | | | H](C(O)═O)N(C)C |
| | | | (CCCCCCCCCCCC |
| | | | CCCCC(O)═O)═ |
| | | | O)═O) |
| 184 | 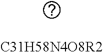 | NMeK(PEG2PEG2C12) | CCCCCCCCCCCC(N CCOCCOCC(NCCO CCOCC(NCCCC[C@ @H](C([R])═O)N(C) |
| | | | [R])═O)═O)═O |
| 185 | 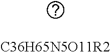 | NMeK(PEG2PEG2gE C12) | CCCCCCCCCCCC(N [C@@H](CCC(NCC OCCOCC(NCCOCC OCC(NCCCC[C@@ |
| | | | H](C([R])═O)N(C) |
| | | | [R])═O)═O)═O)C |
| | | | (O)═O)═O |
| 186 | 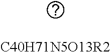 | NMeK(PEG2PEG2gEC 16OH) | CN([C@@H](CCCC NC(COCCOCCNC (COCCOCCNC(CC [C@@H](C(O)═O) |
| | | | NC(CCCCCCCCC |
| | | | CCCCCC(O)═O)═ |
| | | | O)═O)═O)═O)C |
| | | | ([R])═O)[R] |
| 187 | 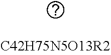 | NMeK(PEG2PEG2gEC 18OH) | CN([C@@H](CCCC NC(COCCOCCNC(C OCCOCCNC(CC[C @@H](C(O)═O)NC |
| | | | (CCCCCCCCCCCC |
| | | | CCCCC(O)═O)═ |
| | | | O)═O)═O)═O)C |
| | | | ([R])═O)[R] |
| 188 | 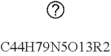 | NMeK(PEG2PEG2gEC 20OH) | CN([C@@H](CCCC NC(COCCOCCNC (COCCOCCNC(CC [C@@H](C(O)═ |
| | | | O)NC(CCCCCCC |
| | | | CCCCCCCCCCCC |
| | | | (O)═O)═O)═O)═ |
| | | | O)═O)C([R])═O)[R] |
| 189 | 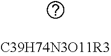 | NMeK(PEG6C18OH) | CN([R])[R](CCCCN C(CCOCCOCCOCC OCCOCCOCCNC(C CCCCCCCCCCCC |
| | | | CCCC(O)═O)═ |
| | | | O)═O)C([R])═O |
| 190 | 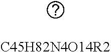 | NMeK(PEG6gEC18OH) | CN([C@@H](CCCC NC(CCOCCOCCOC COCCOCCOCCNC (CC[C@@H](C(O)═ |
| | | | O)NC(CCCCCCCC |
| | | | CCCCCCCCC(O)═ |
| | | | O)═O)═O)═O)C |
| | | | ([R])═O)[R] |
| 191 | 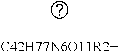 | NMeK(SP6PEG2gEC18 OH) | CN([C@@H](CCCC NC(C[N+](C)(C)CC NC(COCCOCCNC(C C[C@@H](C(O)═O) |
| | | | NC(CCCCCCCCC |
| | | | CCCCCCCC(O)═ |
| | | | O)═O)═O)═O)═O)C |
| | | | ([R])═O)[R] |
| 192 | 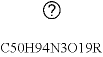 | PEG12gEC18OH | OC(CCCCCCCCCC CCCCCCC(N[C@@ H](CCC(NCCOCCO CCOCCOCCOCCO |
| | | | CCOCCOCCOCCO |
| | | | CCOCCOCCC(N |
| | | | [R])═O)═O)C(O)═ |
| | | | O)═O)═O |
| 193 | 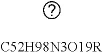 | PEG12gEC20OH | OC(CCCCCCCCCC CCCCCCCCC(N[C @@H](CCC(NCCO CCOCCOCCOCCO |
| | | | CCOCCOCCOCCO |
| | | | CCOCCOCCOCCC |
| | | | (N[R])═O)═O)C(O)═ |
| | | | O)═O)═O |
| 194 | 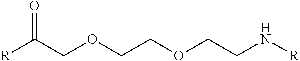 | PEG2, PEG2(2) | O═C(COCCOCCN [R])[R] |
| | C6H11NO3R2 | | |
| 195 | 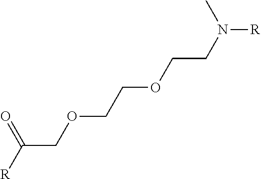 | PEG2(NMe(2)) PEG2NMe | CN(CCOCCOCC ([R])═O)[R] |
| | C7H13NO3R2 | | |
| 196 | 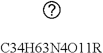 | PEG2PEG2eKC16OH | OC(CCCCCCCCCC CCCCC(N[C@@H] (CCCCNC(COCCOC CNC(COCCOCCN |
| | | | [R])═O)═O)C(O)═ |
| | | | O)═O)═O |
| | | | NCCOCCOCC(NC |
| | | | COCCOCC(NCCC |
| | | | C[C@@H](C(O)═ |
| | | | O)NC(CCCCCCC |
| | | | CCCCCCCC(O)═ |
| | | | O)═O)═O)═O |
| 197 | 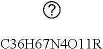 | PEG2PEG2eKC18OH | OC(CCCCCCCCCC CCCCCCC(N[C@@ H](CCCCNC(COCC |
| | | | OCCNC(COCCOC |
| | | | CN[R])═O)═O))C |
| | | | (O)═O)═O)═O |
| | | | NCCOCCOCC(NC |
| | | | COCCOCC(NCCC |
| | | | C[C@@H](C(O)═O) |
| | | | NC(CCCCCCCCCC |
| | | | CCCCCCC(O)═O)═ |
| | | | O)═O)═O |
| 198 | 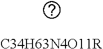 | PEG2PEG2gDabC18OH | OC(CCCCCCCCCC CCCCCCC(N[C@@ H](CCNC(COCCOC |
| | | | CNC(COCCOCCN |
| | | | [R])═O)═O)C(O)═ |
| | | | O)═O)═O |
| | | | NCCOCCOCC(NC |
| | | | COCCOCC(NCC[C |
| | | | @@H](C(O)═O)NC |
| | | | (CCCCCCCCCCCC |
| | | | CCCCC(O)═O)═ |
| | | | O)═O)═O |
| 199 | 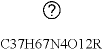 | PEG2PEG2gEC20OH | OC(CCCCCCCCCC CCCCCCCCC(N[C @@H](CCC(NCCO |
| | | | CCOCC(NCCOCCO |
| | | | CC(N[R])═O)═O)═ |
| | | | O)C(O)═O)═O)═O |
| 200 | 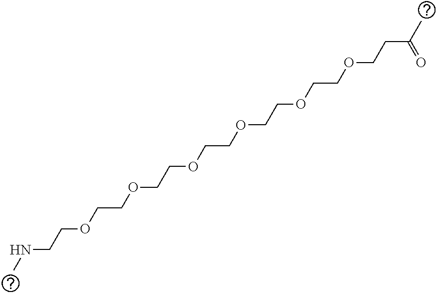 | PEG6 | O═C(CCOCCOCCO CCOCCOCCOCCN [R])[R] |
| | C15H29NO7R2 | | |
| 201 | 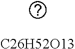 | Peg12-Ome Peg12OMe, Polyethylene12-O-Methyl | COCCOCCOCCOC COCCOCCOCCOC COCCOCCOCCOC |
| | | Peg12-O-methyl | CC═O |
| 202 | 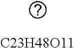 | Peg120Me, Peg12- Omethyl | CCOCCOCCOCCOC COCCOCCOCCOCC OCCOCCOC |
| 203 | 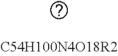 | Pip(PEG12gEC16), Spiral_Pip_PEG12_Iso Glu_Palm | CCCCCCCCCCCCC CCC(N[C@@H](CC C(NCCOCCOCCOC COCCOCCOCCOC |
| | | | COCCOCCOCCOC |
| | | | COCCC(N(CC1)CC |
| | | | C1(C([R])═O)N[R])═ |
| | | | O)═O)C(O)═O)═O |
| 204 | 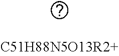 | TMAPF(PEG2PEG2gE C18OH) | C[N+](C)(CCCCCO c1ccc(C[C@@H](C ([R])═O)N[R])cc1)C COCCOCCNC(CO |
| | | | CCOCCNC(CC[C@ |
| | | | @H](C(O)═O)NC(C |
| | | | CCCCCCCCCCCC |
| | | | CCCC(O)═O)═O)═ |
| | | | O)═O |
| 205 | 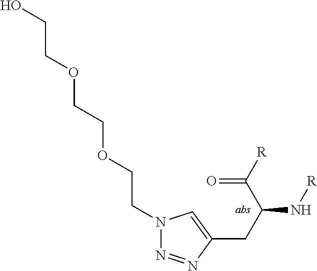 | Tzl(PEG3OH) | OCCOCCOCCn1nnc (C[C@@H](C([R])═ O)N[R])c1 |
| | C11H18N4O4R2 | | |
| 206 | 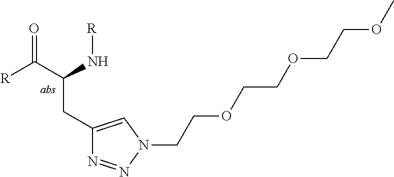 | Tzl(mPEG3) | COCCOCCOCCn1nn c(C[C@@H](C([R])═ O)N[R])c1 |
| | C12H20N4O4R2 | | |
| 207 | 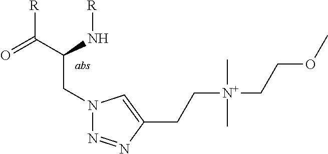 | TzlChmPEG | C[N+](C)(CCclcn(C [C@@H](C([R])═O) N[R])nn1)CCOC |
| | C12H22N5O2R2+ | | |
| 208 | 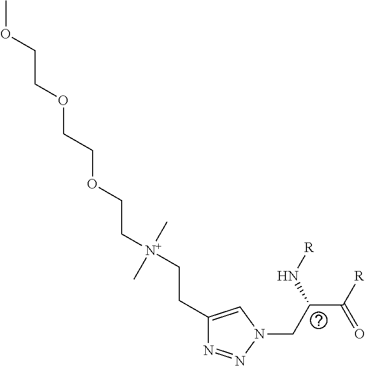 | TzlChmPEG3 | C[N+](C)(CCclcn(C [C@@H](C([R])═O) N[R])nn1)CCOCCO CCOC |
| | C16H30N5O4R2+ | | |
| 209 | 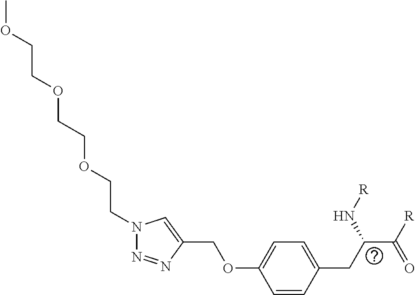 | Y(OTzl(mPEG3)) | COCCOCCOCCn1nn c(COc2ccc(C[C@@ H](C([R])═O)N[R])c c2)c1 |
| | C19H26N4O5R2 | | |
| 210 | 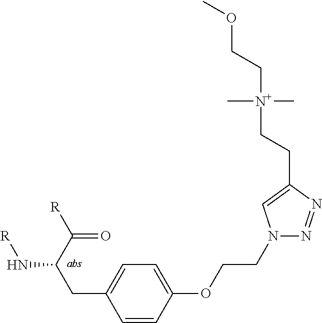 | Y(OTzlChmPEG) | C[N+](C)(CCc1cn(C COc2ccc(C[C@@H] (C([R])═O)N[R])cc2) nn1)CCOC |
| | C20H30N5O3R2+ | | |
| 211 | 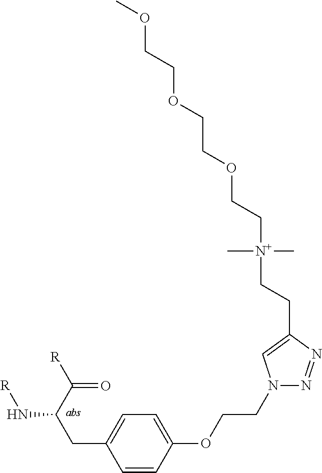 | Y(OTzlChmPEG3) | C[N+](C)(CCc1cn(C COc2ccc(C[C@@H] (C([R])═O)N[R])cc2) nn1)CCOCCOCCOC |
| | C24H38N5O5R2+ | | |
| 212 | 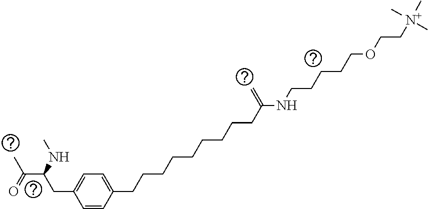 | YC8CO(NHPEG3a) | C[N+](C)(C)CCOCC OCCNC(CCCCCCC COc1ccc(C[C@@H] (C([R])═O)N[R]) cc1)═O |
| | C27H46N3O5R2+ | | |
| 213 | 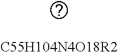 | aMeK(PEG12gEC16) | CCCCCCCCCCCCC CCC(N[C@@H](CC C(NCCOCCOCCOC COCCOCCOCCOC |
| | | | COCCOCCOCCOC |
| | | | COCCC(NCCCC[C |
| | | | @@](C)(C([R])═O) |
| | | | N[R])═O)═O)C |
| | | | (O)═O)═O |
| 214 | 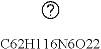 | aMeK(PEG12gEC18 OH) | C[C@@H](C═O)NC ([C@](C)(CCCCNC (CCOCCOCCOCCO CCOCCOCCOCCO |
| | | | CCOCCOCCOCCO |
| | | | CCNC(CC[C@@H] |
| | | | (C(O)═O)NC(CCCC |
| | | | CCCCCCCCCCCCC |
| | | | (O)═O)═O)═O)═ |
| | | | O)NC(CN)═O)═O |
| 215 | 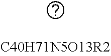 | aMeK(PEG2PEG2gEC 16OH | C[C@](CCCCNC(C OCCOCCNC(COCC OCCNC(CC[C@@H] (C(O)═O)NC(CCCCC |
| | | | CCCCCCCCCC(O)═ |
| | | | O)═O)═O)═O)═O) |
| | | | (C([R])═O)N[R] |
| 216 | 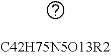 | aMeK(PEG2PEG2gEC1 8OH | C[C@](CCCCNC(CO CCOCCNC(COCCO CCNC(CC[C@@H] (C(O)═O)NC(CCCC |
| | | | CCCCCCCCCCCCC |
| | | | (O)═O)═O)═O)═ |
| | | | O)═O)(C([R])═O) |
| | | | N[R] |
| 217 | 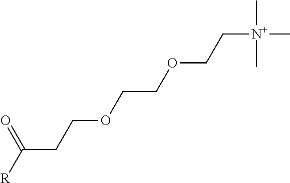 | cPEG3aCO, cPEG3a | C[N+](C)(C)CCOCC OCCC([R])═O |
| | C10H21NO3R+ | | |
| 218 | 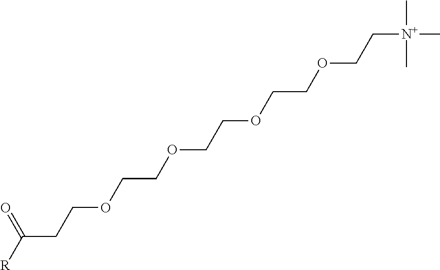 | cPEG5aCO, cPEG5a | C[N+](C)(C)CCOCC OCCOCCOCCC ([R])═O |
| | C14H29NO5R+ | | |
| 219 | 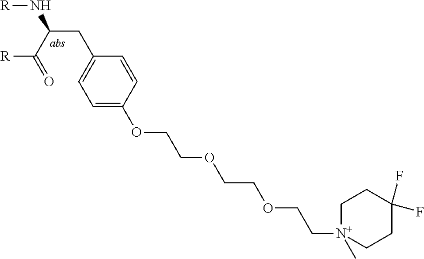 | dFPPEG3F | C[N+](CCOCCOCC Oc1ccc(C[C@@H](C ([R])═O)N[R])cc1)(C C1)CCC1(F)F |
| | C21H31F2N2O4R2+ | | |
| 220 | 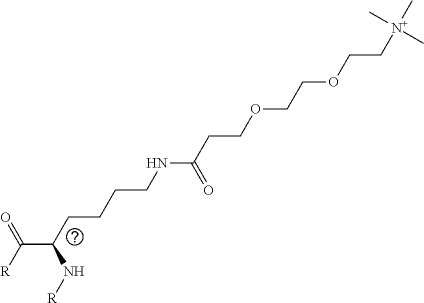 | dK(cPEG3a), k(cPEG3a), dK(cPEG3aCO), k(cPEG3aCO) | C[N+](C)(C)CCOCC OCCC(NCCCC[C@ H](C([R])═O)N [R])═O |
| | C16H32N3O4R2+ | | |
| 221 | 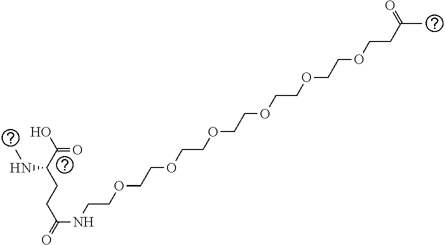 | gEPEG6 | OC([C@H](CCC(NC COCCOCCOCCOCC OCCOCCC([R])═O)═ O)N[R])═O |
| | C20H36N2O10R2 | | |
| 222 | 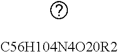 | k(PEG12gEC18OH), dK(PEG12gEC18OH) | OC(CCCCCCCCCC CCCCCCC(N[C@@ H](CCC(NCCOCCO CCOCCOCCOCCO |
| | | | CCOCCOCCOCCO |
| | | | CCOCCOCCC(NCC |
| | | | CC[C@H](C([R])═ |
| | | | O)N[R])═O)═O)C |
| | | | (O)═O)═O)═O |
| 223 | 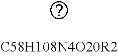 | k(PEG12gEC20OH) dK(PEG12gEC20OH) | OC(CCCCCCCCCC CCCCCCCCC(N[C@ @H](CCC(NCCOCC OCCOCCOCCOCCO |
| | | | CCOCCOCCOCCOC |
| | | | COCCOCCC(NCCC |
| | | | C[C@H](C([R])═O) |
| | | | N[R])═O)═O)C(O)═ |
| | | | O)═O)═O |
| 224 | 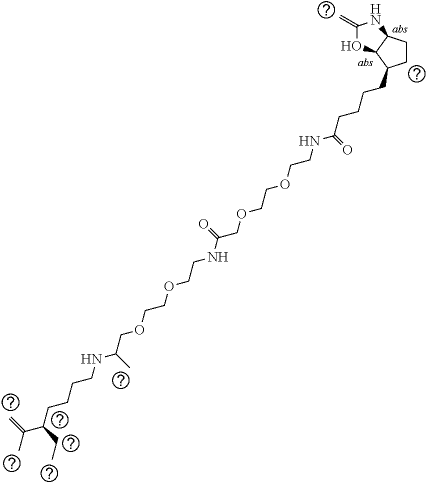 | dK(PEG2PEG2Biotin), k(PEG2PEG2Biotin) | O═C(CCCC[C@@H] ([C@H]1N2)SC[C@ @H]1NC2═O)NCCO CCOCC(NCCOCCO CC(NCCCC[C@H](C ([R])═O)N[R])═O)═ O |
| | C28H48N6O9SR2 | | |
| 225 | 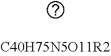 | k(PEG2PEG2C18GolB), dK(PEG2PEG2C18Go lB) | CN(CCOCCOCC(NC COCCOCC(NCCCC [C@H](C([R])═O)N [R])═O)═O)C(CCCC |
| | | | CCCCCCCCCCCCC |
| | | | (NC(CO)CO)═O)═O |
| 226 | 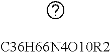 | k(PEG2PEG2C18OH), dK(PEG2PEG2C18OH) | OC(CCCCCCCCCC CCCCCCC(NCCOC COCC(NCCOCCOC C(NCCCC[C@H](C |
| | | | ([R])═O)N[R])═O)═ |
| | | | O)═O)═O |
| 227 | 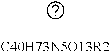 | k(PEG2PEG2GolAC18 OH), dK(PEG2PEG2G olAC18OH) | OCC(CO)(C(NCCOC COCC(NCCOCCOC C(NCCCC[C@H](C ([R])═O)N[R])═ |
| | | | O)═O)═O)NC(CCC |
| | | | CCCCCCCCCCCCC |
| | | | C(O)═O)═O |
| 228 | 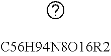 | k(PEG2PEG2PPPgEC18 OH) dK(PEG2PEG2PPP gEC18OH) | OC(CCCCCCCCCC CCCCCCC(N[C@@ H](CCC(N(CCC1)[C @@H]1C(N(CCC1) |
| | | | [C@@H]1C(N(CCC |
| | | | 1)[C@@H]1C(NCCO |
| | | | CCOCC(NCCOCCO |
| | | | CC(NCCCC[C@H] |
| | | | (C([R])═O)N[R])═ |
| | | | O)═O)═O)═O)═O)═ |
| | | | O)C(O)═O)═O)═O |
| 229 | 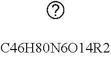 | k(PEG2PEG2PgEC18 OH), dK(PEG2PEG2 PgEC18OH) | OC(CCCCCCCCCC CCCCCCC(N[C@@ H](CCC(N(CCC1)[C @@H]1C(NCCOCC |
| | | | OCC(NCCOCCOC |
| | | | C(NCCCC[C@H](C |
| | | | ([R])═O)N[R])═O)═ |
| | | | O)═O)═O)C(O)═ |
| | | | O)═O)═O |
| 230 | 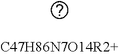 | k(PEG2PEG2Sp6gEC 18OH), dK(PEG2PEG2 Sp6gEC18OH) | C[N+](C)(CCNC(CC [C@@H](C(O)═O)N C(CCCCCCCCCCC CCCCCC(O)═O)═ |
| | | | O)═O)CC(NCCOCC |
| | | | OCC(NCCOCCOCC |
| | | | (NCCCC[C@H](C |
| | | | ([R])═O)N[R])═O)═ |
| | | | O)═O |
| 231 | 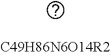 | k(PEG2PEG2TrxgEC18 OH), dK(PEG2PEG2 TrxgEC18OH) | OC(CCCCCCCCCC CCCCCCC(N[C@@ H](CCC(NC[C@H] (CC1)CC[C@@H] |
| | | | 1C(NCCOCCOCC(N |
| | | | CCOCCOCC(NCCC |
| | | | C[C@H](C([R])═O) |
| | | | N[R])═O)═O)═O)═ |
| | | | O)C(O)═O)═O)═O |
| 232 | 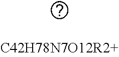 | k(PEG2PEG2gE(C)C12, dK(PEG2PEG2gE(C) C12 | CCCCCCCCCCCC(N [C@@H](CCC(NCC OCCOCC(NCCOCC OCC(NCCCC[C@H] |
| | | | (C([R])═O)N[R])═ |
| | | | O)═O)═O)C(N[C@ |
| | | | @H](CC(O)═O)C |
| | | | [N+](C)(C)C)═O)═O |
| 233 | 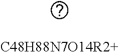 | k(PEG2PEG2gE(C)C18 OH, dK(PEG2PEG2g E(C)C180H | C[N+](C)(C)C[C@H] (CC(O)═O)NC([C@ H](CCC(NCCOCCO CC(NCCOCCOCC(N |
| | | | CCCC[C@H](C |
| | | | ([R])═O)N[R])═ |
| | | | O)═O)═O)NC(CCC |
| | | | CCCCCCCCCCCC |
| | | | CC(O)═O)═O)═O |
| 234 | 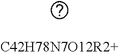 | k(PEG2PEG2gE(c)C12, dK(PEG2PEG2gE(c)C12 | CCCCCCCCCCCC(N [C@@H](CCC(NCC OCCOCC(NCCOCC OCC(NCCCC[C@H] |
| | | | (C([R])═O)N[R])═ |
| | | | O)═O)═O)C(N[C@ |
| | | | H](CC(O)═O)C[N+] |
| | | | (C)(C)C)═O)═O |
| 235 | 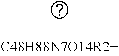 | k(PEG2PEG2gE(c)C18 OH, dK(PEG2PEG2gE (c)C18OH | C[N+](C)(C)C[C@@ H](CC(O)═O)NC([C @H](CCC(NCCOCC OCC(NCCOCCOCC |
| | | | (NCCCC[C@H](C |
| | | | ([R])═O)N[R])═O)═ |
| | | | O)═O)NC(CCCCCC |
| | | | CCCCCCCCCCC |
| | | | (O)═O)═O)═O |
| 236 | 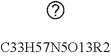 | k(PEG2PEG2gEC10OH), dK(PEG2PEG2gEC10 OH) | OC(CCCCCCCCC(N [C@@H](CCC(NCC OCCOCC(NCCOCC OCC(NCCCC[C@H] |
| | | | (C([R])═O)N[R])═ |
| | | | O)═O)═O)C(O)═ |
| | | | O)═O)═O |
| 237 | 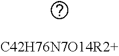 | k(PEG2PEG2gEC12O H(C), dK(PEG2PEG2g EC12OH(C) | C[N+](C)(C)C[C@H] (CC(O)═O)NC(CCC CCCCCCCC(N[C@ @H](CCC(NCCOCC |
| | | | OCC(NCCOCCOCC |
| | | | (NCCCC[C@H](C |
| | | | ([R])═O)N[R])═O)═ |
| | | | O)═O)C(O)═O)═ |
| | | | O)═O |
| 238 | 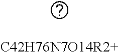 | k(PEG2PEG2gEC12O H(c), dK(PEG2PEG2g EC12OH(c) | C[N+](C)(C)C[C@@ H](CC(O)═O)NC(CC CCCCCCCCC(N[C@ @H](CCC(NCCOCC |
| | | | OCC(NCCOCCOCC |
| | | | (NCCCC[C@H](C |
| | | | ([R])═O)N[R])═O)═ |
| | | | O)═O)C(O)═O)═ |
| | | | O)═O |
| 239 | 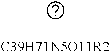 | k(PEG2PEG2gEC16), dK(PEG2PEG2gEC16) | CCCCCCCCCCCCC CCC(N[C@@H](CC C(NCCOCCOCC(NC COCCOCC(NCCCC |
| | | | [C@H](C([R])═O)N |
| | | | [R])═O)═O)═O)C |
| | | | (O)═O)═O |
| 240 | 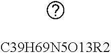 | k(PEG2PEG2gEC16OH), dK(PEG2PEG2gEC16 OH) | OC(CCCCCCCCCC CCCCC(N[C@@H] (CCC(NCCOCCOCC (NCCOCCOCC(NCC |
| | | | CC[C@H](C([R])═ |
| | | | O)N[R])═O)═O)═ |
| | | | O)C(O)═O)═O)═O |
| 241 | 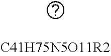 | k(PEG2PEG2gEC18), dK(PEG2PEG2gEC18) | CCCCCCCCCCCCC CCCCC(N[C@@H] (CCC(NCCOCCOCC (NCCOCCOCC(NCC |
| | | | CC[C@H](C([R])═ |
| | | | O)N[R])═O)═O)═O) |
| | | | C(O)═O)═O |
| 242 | 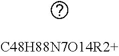 | k(PEG2PEG2gEC18OH (C), dK(PEG2PEG2gEC 18OH(C) | C[N+](C)(C)C[C@H] (CC(O)═O)NC(CCC CCCCCCCCCCCCC C(N[C@@H](CCC(N |
| | | | CCOCCOCC(NCCO |
| | | | CCOCC(NCCCC[C@ |
| | | | H](C([R])═O)N[R])═ |
| | | | O)═O)═O)C(O)═ |
| | | | O)═O)═O |
| 243 | 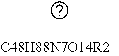 | k(PEG2PEG2gEC18OH (c), dK(PEG2PEG2gEC 18OH(c) | C[N+](C)(C)C[C@@ H](CC(O)═O)NC(CC CCCCCCCCCCCCC CC(N[C@@H](CCC |
| | | | (NCCOCCOCC(NCC |
| | | | OCCOCC(NCCCC[C |
| | | | @H](C([R])═O)N |
| | | | [R])═O)═O)═O) |
| | | | C(O)═O)═O)═O |
| 244 | 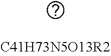 | k(PEG2PEG2gEC18OH), dK(PEG2PEG2gEC18 OH) | OC(CCCCCCCCCC CCCCCCC(N[C@@ H](CCC(NCCOCCO CC(NCCOCCOCC |
| | | | (NCCCC[C@H](C |
| | | | ([R])═O)N[R])═O)═ |
| | | | O)═O)C(O)═O)═ |
| | | | O)═O |
| 245 | 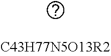 | k(PEG2PEG2gEC20OH), dK(PEG2PEG2gEC20 OH) | OC(CCCCCCCCCC CCCCCCCCC(N[C@ @H](CCC(NCCOCC OCC(NCCOCCOCC |
| | | | (NCCCC[C@H](C |
| | | | ([R])═O)N[R])═O)═ |
| | | | O)═O)C(O)═O)═ |
| | | | O)═O |
| 246 | 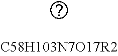 | k(PEG2PEG2gEDAP(C 16OH)2), dK(PEG2PEG 2gEDAP(C16OH)2) | OC(CCCCCCCCCC CCCCC(NC[C@@H] (C(N[C@@H](CCC (NCCOCCOCC(NC |
| | | | COCCOCC(NCCCC |
| | | | [C@H](C([R])═O)N |
| | | | [R])═O)═O)═O)C |
| | | | (O)═O)═O)NC(CCC |
| | | | CCCCCCCCCCC |
| | | | C(O)═O)═O)═O)═O |
| 247 | 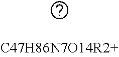 | kPEG2PEG2gEDAP(C 16OH)2; kPEG2PEG2 gEDap(C16OH)2, k(PE G2PEG2gEDAP(C16O | C[N+](C)(CCNC(CC CCCCCCCCCCCCC CC(O)═O)═O)CC(N [C@@H](CCC(NCC |
| | | H)2), dKPEG2PEG2g | OCCOCC(NCCOCC |
| | | EDAP(C16OH)2; dKP | OCC(NCCCC[C@H] |
| | | EG2PEG2gEDap(C16 | (C([R])═O)N[R])═ |
| | | OH)2, dK(PEG2PEG | O)═O)═O)C(O)═ |
| | | 2gEDAP(C16OH)2) | O)═O |
| 248 | 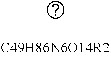 | kPEG2PEG2gEDAP(C16 OH)2, dKPEG2PEG2g EDAP(C16OH)2 | OC(CCCCCCCCCC CCCCCCC(NC[C@ H](CC1)CC[C@@H] 1C(N[C@@H](CCC |
| | | | (NCCOCCOCC(NC |
| | | | COCCOCC(NCCCC |
| | | | [C@H](C([R])═O)N |
| | | | [R])═O)═O)═O)C |
| | | | (O)═O)═O)═O)═O |
| 249 | 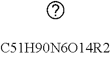 | k(PEG2PEG2gESp6C18 OH), dK(PEG2PEG2gE Sp6C18OH) | OC(CCCCCCCCCC CCCCCCCCC(NC[C @H](CC1)CC[C@@ H]1C(N[C@@H](CC |
| | | | C(NCCOCCOCC(N |
| | | | CCOCCOCC(NCCC |
| | | | C[C@H](C([R])═O) |
| | | | N[R])═O)═O)═O)C |
| | | | (O)═O)═O)═O)═O |
| 250 | 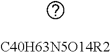 | k(PEG2PEG2gETrxC18 OH), dK(PEG2PEG2gE TrxC18OH) | OC([C@H](CCC(NC COCCOCC(NCCOC COCC(NCCCC[C@ H](C([R])═O)N[R])═ |
| | | | O)═O)═O)NC(CCC |
| | | | CCCCCCOc1cc(C |
| | | | (O)═O)ccc1)═O)═O |
| 251 | 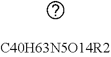 | k(PEG2PEG2gETrxC20 OH), dK(PEG2PEG2gE TrxC200H) | OC([C@H](CCC(NC COCCOCC(NCCOC COCC(NCCCC[C@ H](C([R])═O)N[R])═ |
| | | | O)═O)═O)NC(CCC |
| | | | CCCCCCOc(cc1)cc |
| | | | c1C(O)═O)═O)═O |
| 252 | 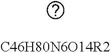 | k(PEG2PEG2gEmXOH), dK(PEG2PEG2gEmX OH) | OC(CCCCCCCCCC CCCCCCC(N[C@@ H](CCC(N(CCC1)[C @H]1C(NCCOCCO |
| | | | CC(NCCOCCOCC(N |
| | | | CCCC[C@H](C([R])═ |
| | | | O)N[R])═O)═O)═ |
| | | | O)═O)C(O)═O)═ |
| | | | O)═O |
| 253 | 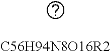 | k(PEG2PEG2gEpXOH), dK(PEG2PEG2gEpX OH) | OC(CCCCCCCCCC CCCCCCC(N[C@@ H](CCC(N(CCC1)[C @H]1C(N(CCC1)[C |
| | | | @H]1C(N(CCC1)[C |
| | | | @H]1C(NCCOCCO |
| | | | CC(NCCOCCOCC |
| | | | (NCCCC[C@H](C |
| | | | ([R])═O)N[R])═O)═ |
| | | | O)═O)═O)═O)═O) |
| | | | C(O)═O)═O)═O |
| 254 | 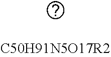 | k(PEG2PEG2pgEC18O H), dK(PEG2PEG2pg EC18OH) | OC(CCCCCCCCCC CCCCCCC(N[C@@ H](CCC(NCCOCCO CCOCCOCCOCCO |
| | | | CCC(NCCOCCOCC |
| | | | (NCCCC[C@H](C |
| | | | ([R])═O)N[R])═O)═ |
| | | | O)═O)C(O)═O)═ |
| | | | O)═O |
| 255 | 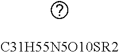 | k(PEG2PEG2pppgEC18 OH), dK(PEG2PEG2pp pgEC18OH) | O═C(CCCC[C@@H] ([C@H]1N2)SC[C@ @H]1NC2═O)NCCO CCOCCOCCOCCO |
| | | | CCOCCC(NCCCC[C |
| | | | @H](C([R])═O)N |
| | | | [R])═O |
| 256 | 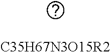 | k(PEG2PEG6gEC18OH), dK(PEG2PEG6gEC18 H) | CC(NCCOCCOCCO CCOCCOCCOCCOC COCCOCCOCCOCC OCCC(NCCCC[C@ |
| | | | H](C([R])═O)N[R])═ |
| | | | O)═O |
| 257 | 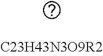 | k(dPEG12AcBr), dK(dP EG12AcBr) | CC(NCCOCCOCCO CCOCCOCCOCCC (NCCCC[C@H](C ([R])═O)N[R])═ |
| | | | O)═O |
| 258 | 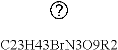 | k(dPEG12AcVitE), dK(dPEG12AcVitE) | O═C(CCOCCOCCO CCOCCOCCOCCN C(CBr)═O)NCCCC [C@H](C([R])═O)N |
| | | | [R] |
| 259 | 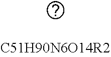 | k(dPEG6Ac), dK(dPE G6Ac) | CC(NCCOCCOCCO CCOCCOCCOCCOC COCCOCCC(NCCC C[C@H](C([R])═O) |
| | | | N[R])═O)═O |
| 260 | 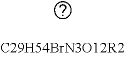 | k(dPEG6AcBr), dK(dPE G6AcBr) | O═C(CCOCCOCCO CCOCCOCCOCCO CCOCCOCCNC (CBr)═O)NCCCC[C |
| | | | @H](C([R])═O)N[R] |
| 261 | 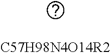 | k(dPEG9Ac), dK(dPEG 9Ac) | CC(C)CCC[C@@H] (C)CCC[C@@H](C) CCC[C@](C)(CC1) Oc(c(C)c2C)c1c(C)c |
| | | | 2OCC(N[C@@H](C |
| | | | CC(NCCOCCOCCO |
| | | | CCOCCOCCOCCC |
| | | | (NCCCC[C@H](C |
| | | | ([R])═O)N[R])═O)═ |
| | | | O)C(O)═O)═O |
| 262 | 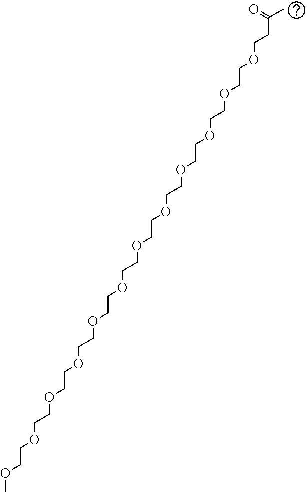 | mPEG12CO | COCCOCCOCCOC COCCOCCOCCOC COCCOCCOCCOC CC([R])═O |
| | C26H51O13R | | |
| 263 | 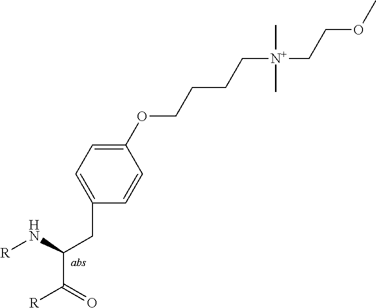 | mPEG2TMA4F | C[N+](C)(CCCCOc1 ccc(C[C@@H](C ([R])═O)N[R])cc1) CCOC |
| | C18H29N2O3R2+ | | |
| 264 | 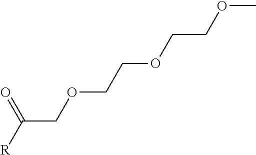 | mPEG3CO | COCCOCCOCC ([R])═O |
| | C7H13O4R | | |
| 265 | 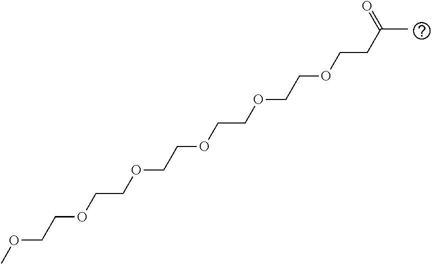 | mPEG6CO | COCCOCCOCCOC COCCOCCC([R])═O |
| | C14H2707R | | |

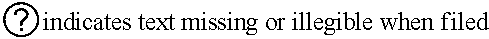

### General Peptide Synthetic Procedure 1

IL-23R inhibitor compounds described herein were synthesized from amino acids monomers using Merrifield solid phase synthesis techniques on Protein Technology's Symphony multiple channel synthesizer. The peptides were assembled using HBTU (O-Benzotriazole-N,N,N′,N′-tetramethyl-uronium-hexafluoro-phosphate), Diisopropylethylamine (DIEA) coupling conditions. For some amino acid couplings PyAOP(7-Azabenzotriazol-1-yloxy)tripyrrolidinophosponium hexafluorophosphate) and DIEA conditions were used. Rink Amide MBHA resin (100-200 mesh, 0.57 mmol/g) was used for peptide with C-terminal amides and pre-loaded Wang Resin with N-α-Fmoc protected amino acid was used for peptide with C-terminal acids. The coupling reagents (HBTU and DIEA premixed) were prepared at 100 mmol concentration. Similarly, amino acids solutions were prepared at 100 mmol concentration. Peptide inhibitors of the present invention were identified based on medical chemistry optimization and/or phage display and screened to identify those having superior binding and/or inhibitory properties.

### Preparation of Certain Modified Amino Acids

### Synthesis of 7-(3-Nacetyl-phenyl)-tryptophan (7(3NAcPh)W) (S)-2-((((9H-fluoren-9-yl)methoxy)carbonyl)amino)-3-(7-(3-acetamidophenyl)-1H-indol-3-yl)propanoic acid

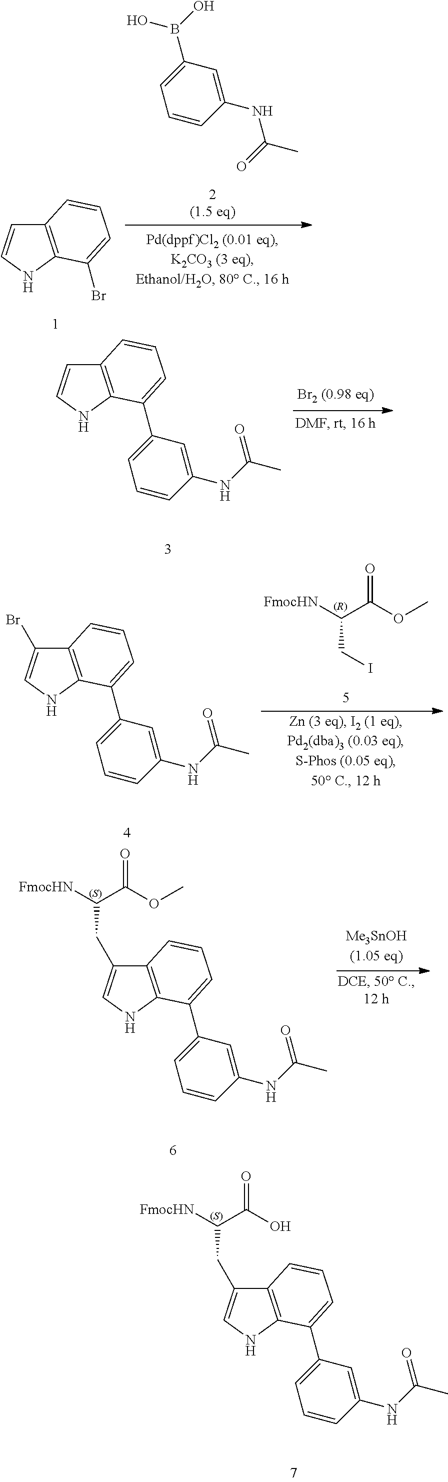

To a solution of 1 (30.0 g, 153 mmol), compound 2 (41.1 g, 230 mmol) and K3PO4 (97.4 g, 459 mmol) in H₂O/ethanol (500 mL) and, Pd(dppf)Cl2 (1.12 g, 1.53 mmol) was added under an N₂ atmosphere. The mixture was stirred at 80° C. for 16 h. The mixture was filtered. The mixture was concentrated, then extracted with ethyl acetate (500 mL×2), dried with anhydrous Na₂SO₄. The organic layer was concentrated and purified by FCC (eluent: petroleum ether/ethyl acetate=1:0 to 55:45) to give 3 (25.0 g, yield: 62.5%) as yellow oil MS (ESI): mass calculated for C16H14N2O, 250.295, m/z found 251.0 [M+].

To a 1 L round-bottomed flask containing a solution of 3 (12.0 g, 47.9 mmol) in DMF (300 mL) bromine (Br₂, 2.422 mL, 47.0 mmol) was slowly added. The mixture was stirred at 25° C. for 16 hours. The solution was added to aqueous sodium sulfite (500 mL), the mixture was stirred at 25° C. for 2 hours. The mixture was filtered, the filter cake was mixed with H₂O (400 mL) and stirred at 25° C. for 1 h. The mixture was filtered, the solid was collected to give 4 as a crude product, which was purified by preparative high-performance liquid chromatography (Column: Phenomenex C18 250×50 mm×10 um, Condition: water (FA)-CAN (20%-60%)). The mixture was concentrated, extracted with CH₂Cl₂ (1 L×2), washed with brine, dried with anhydrous Na₂SO₄. The organic layers was filtered and concentrated to give 4 (9.70 g, yield: 60.8%) as a pale white. MS (ESI): mass calculated For C₁₆H₁₃BrN₂O, 329.191, m/z found 328.8 [M].

A 250 mL three neck round-bottomed flask was charged with activated Zn powder (5.84 g, 89.3 mmol), DMF (120 mL) and I₂ (382 mg, 1.50 mmol) was added under an N₂ atmosphere at room temperature. After stirring for 20 min, a solution of 5 (13.6 g, 30.1 mmol) in DMF (30 mL) was added to the mixture. The reaction mixture was stirred for 30 min at room temperature, after which 4 (9.70 g, 29.5 mmol), tris(dibenzylideneacetone)-palladium (826 mg, 0.902 mmol), 2-dicyclohexylphosphino-2′,6′-dimethoxybiphenyl (617 mg, 1.50 mmol) were added under an N₂ atmosphere. The reaction mixture was stirred at 50° C. for 12 hours, after which solvent was removed under reduced pressure to give crude product 6. The crude product was extracted with ethyl acetate (1500 mL). The extract was washed with H₂O (500 mL×2), followed by brine (500 mL), after which it was dried over anhydrous Na₂SO₄, filtered, and concentrated to dryness in vacuo to give crude intermediate 6, which was purified by silica gel chromatography (0-100% ethyl acetate/petroleum ether (EtOAc/PE)) to afford 6 (11.0 g, yield: 63.8%) as a brown-yellow oil. MS (ESI): mass calculated for C₃₅H₃₁N₃O₅, 573.638, m/z found 574.1 [M+1].

Intermediate 6 (11.0 g, 19.2 mmol), a stir bar, Me₃SnOH (3.64 g, 20.1 mmol) and DCE (150 mL) were added to a 250 mL round-bottomed flask and stirred at 50° C. for 12 hours. To the reaction mixture 2 N HCl was added to adjust the to pH to 6. A second reaction starting from intermediate 6 was conducted and the products were combined for further workup. The combined reaction mixture was concentrated under reduced pressure to give the crude, which was purified by preparative HPLC using a Xtimate C18 150×40 mm×5 um (eluent: 38% to 68% (v/v) CH₃CN and H₂O with 0.05% HCl) to afford product 7. The product was suspended in water (100 mL), the mixture frozen using dry ice/ethanol, and then lyophilized to dryness to afford 7 (7(3NAcPh)W. 11.8 g, yield: 66.8%) as a white solid. MS (ESI): mass calculated For C₃₄H₂₉N₃O₅, 559.611, m/z found 560.0 [M+1]. ¹H NMR DMSO-d₆ (400 MHz) δ 10.73 (s, 1H), 10.10 (s, 1H), 7.52-8.02 (m, 7H), 6.96-7.52 (m, 9H), 4.03-4.44 (m, 3H), 3.25 (d, J=13.2 Hz, 2H), 3.01-3.15 (m, 1H), 2.08 (s, 3H).

Synthesis of 5-methyl-pyridyl-alanine (5MePyridinAla)

### (S)-2-((((9H-fluoren-9-yl)methoxy)carbonyl)amino)-3-(5-methylpyridin-3-yl)propanoic acid

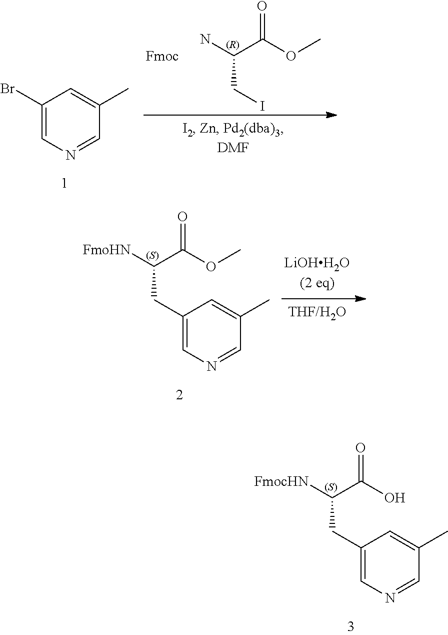

Activated Zn powder (8.18 g, 125 mmol), DMF (150 mL) and I₂ (0.534 g, 2.11 mmol) were stirred under an N₂ atmosphere at room temperature for 20 min., after which (R)-methyl 2-((((9H-fluoren-9-yl)methoxy)carbonyl)amino)-3-iodopropanoate (19.0 g, 42.1 mmol) in DMF (25 mL) was added. The reaction mixture was stirred for 30 min at room temperature, after which a mixture of 1 (7.97 g, 46.3 mmol), tris(dibenzylideneacetone)-palladium (1.16 g, 1.26 mmol) and 2-dicyclohexylphosphino-2′,6′-dimethoxybiphenyl (0.864 g, 2.11 mmol) in DMF (25 mL) was added under an N₂ atmosphere. The resulting reaction mixture was stirred at 50° C. for 12 h. The solvent was removed under reduced pressure to give the crude, which was purified by FCC (eluent: petroleum ether:ethyl acetate=1:0 to 0:1 and ethyl acetate:methanol=1:0 to 2:1) to afford the product 2 (10.00 g, 57.0% yield) as a pale yellow liquid. MS (ESI): mass calculated for C₂₅H₂₄N₂O₄, 416.469, m/z found 417.1 [M+H]⁺.

To a mixture of 2 (9.50 g, 22.8 mmol) in THF (100 mL) was added LiOH·H₂O (1.91 g, 45.6 mmol) in H₂O (10 mL). The mixture was stirred for 1 h at 0° C. TLC showed most SM were consumed. To the reaction mixture was added HCl (1 N) dropwise at ice bath to pH=5. The reaction mixture was concentrated under reduced pressure, then poured into water (200 mL) the mixture was extracted with THF (200 mL×3). The organic layers were combined, washed with brine (100 mL), dried over anhydrous Na₂SO₄. After filtering the organic layers were concentrated under reduced pressure to afford crude product 3, which was purified by FCC (eluent:ethyl acetate:methanol=1:0 to 2:1) to obtain 3 (5MePyridinAla, 6.716 g, yield: 72.3%) as a white powder. MS (ESI): mass calculated For C₂₄H₂₂N₂O₄, 402.442, m/z found 403.1 [M+H]⁺. ¹H NMR DMSO-d₆ (Bruker_400 MHz): δ 8.18 (s, 2H), 7.88 (d, J=7.6 Hz, 2H), 7.63 (d, J=7.2 Hz, 2H), 7.45-7.26 (m, 5H), 6.81 (s, 1H), 4.33-4.21 (m, 1H), 4.20-4.09 (m, 2H), 3.95 (s, 1H), 3.06-3.05 (m, 1H), 2.92-2.89 (m, 1H), 2.18 (s, 3H).

### Synthesis of AEF(G)

### (S)-2-((((9H-fluoren-9-yl)methoxy)carbonyl)amino)-3-(4-(2-(3-((2,2,4,6,7-pentamethyl-2,3-dihydrobenzofuran-5-yl)sulfonyl)guanidino)ethoxy)phenyl)propanoic acid

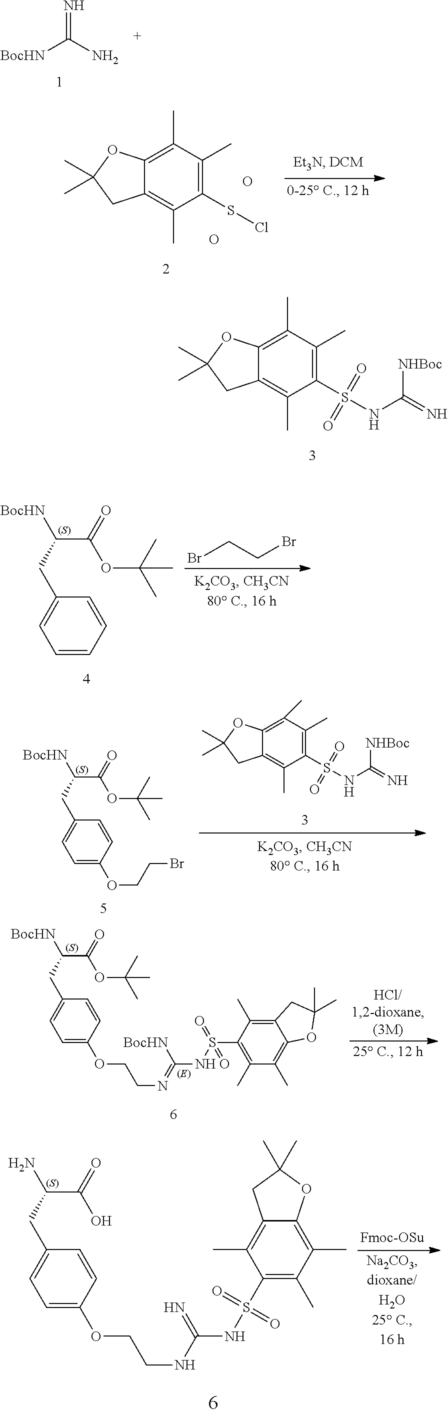

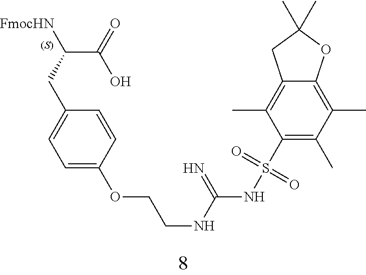

Starting material 1 (9.9 g, 62.2 mmol), a stir bar, Et₃N (14 mL, 101 mmol), and dichloromethane (DCM, 250 mL) were added to a 500 mL round-bottomed flask. The resulting mixture was treated with 2 (10 g, 34.6 mmol) in portions under ice-water bath. Then the reaction mixture was stirred at 25° C. for 12 hours. The reaction mixture was diluted with H₂O (800 mL), extracted with DCM (400 mL×2). The organic phase extracts were combined, washed with brine (800 mL), and concentrated to give the crude intermediate 3 as a yellow solid. The crude intermediate was triturated with ethyl acetate (50 mL) and the suspension isolated via filtration. The filter cake was washed with ethyl acetate (20 mL×3) before drying under reduced pressure to give the 3 (7.12 g, 49%) as a white solid. MS (ESI): mass calculated for C₁₉H₂₉N₃O₅S₆, 411.5, m/z found 412.1 [M+H]⁺.

Starting material 4 (50.0 g, 148 mmol), a stir bar, DMF (300 mL), and K₂CO₃ (102 g, 739 mmol) were added to a nitrogen-purged 1000 mL round-bottomed flask. The flask was subsequently evacuated and refilled with nitrogen (×3), after which 1,2-dibromoethane (154 mL, 1.78 mol) was added, and the resulting mixture was stirred at 80° C. for 16 h under a N₂ atmosphere. The reaction mixture was filtered and concentrated to dryness under reduced pressure to give the crude product, which was subjected to silica gel chromatography (eluent:EtOAc:pet ether=0-60%) to give the 5 (64 g, 96%) as a light yellow oil. MS (ESI): mass calculated for C₂₀H₃₀BrNO₅, 444.36, m/z found 466.1 [M+Na]⁺.

Intermediate 5 (6.1 g, 13.7 mmol), 3 (6.2 g, 15.1 mmol), K₂CO₃ (7.6 g, 55.0 mmol), a stir bar, and CH₃CN(100 mL) were charged into a 250 mL round-bottomed flask. The reaction mixture was stirred at 80° C. for 16 h under a N₂ atmosphere. The reaction mixture was cooled to room temperature, diluted with H₂O (200 mL), extracted with ethyl acetate (100 mL×2). The organic phases were combined and washed with brine (300 mL) and concentrated to give the crude intermediate 6. The crude intermediate was purified by flash column chromatography (FCC, eluent:ethyl acetate/petroleum ether=0:1 to 2:1) to give the 6 (6.62 g, 44.2%) as a white solid. MS (ESI): mass calculated for C₃₉H₅₈N₄O₁₀S, 774.9, m/z found 775.5 [M+H]⁺.

Intermediate 6 (6.6 g, 8.52 mmol), HCl/1, 4-dioxane (90 mL, 4M), a stir bar, and 1, 4-dixoane (30 mL) were charged into a 250 mL round bottomed flask. The resulting mixture was stirred at 25° C. for 12 hr. The solvent was removed under reduced pressure to give intermediate 7 (7.8 g, crude product) as a colourless oil, which was directly used to next step. MS (ESI): mass calculated for C₂₅H₃₄N₄O₆S, 518.6, m/z found 519.2 [M+H]⁺.

Intermediate 7 (7.80 g, 15.0 mmol), a stir bar, Na₂CO₃ (3.19 g, 30.1 mmol), Fmoc-OSu (5.58 g, 16.5 mmol), 1,4-dioxane (50 mL), and H₂O (50 mL) were added into a 250 mL round-bottomed flask at 25° C. The reaction mixture was stirred at 25° C. for 16 hours, after which it was adjusted to pH=5-6 with HCl (2M) and the resulting reaction mixture was extracted with EtOAc (150 mL×3). The organic phases from the extraction were combined and washed with brine (200 mL) and concentrated to give the crude intermediate 7. The crude intermediate was purified by preparative HPLC with a Column: Phenomenex C18 150×40 mm×5 um, (eluent: 42% to 72% (v/v) CH₃CN and H₂O with 0.1% HCl) to afford pure product. The product was suspended in water (100 mL), the mixture frozen using dry ice/ethanol, and then lyophilized to dryness to afford desired product 8 (AEF(G), 4 g, 36%) as a white solid. MS (ESI): mass calculated for C₄₀H₄₄N₄O₈S, 740.9, m/z found 741.3 [M+H]⁺. ¹H NMR (400 MHZ, DMSO-d₆): 7.87 (d, J=7.2 Hz, 2H), 7.71-7.62 (m, 2H), 7.39 (td, J=4.0, 7.2 Hz, 2H), 7.29 (td, J=7.6, 12.0 Hz, 2H), 7.14 (br d, J=8.0 Hz, 2H), 6.99-6.85 (m, 1H), 6.77 (br d, J=8.4 Hz, 2H), 6.59-6.50 (m, 1H), 4.21-4.06 (m, 4H), 3.88 (br s, 2H), 3.42-3.36 (m, 4H), 2.99 (br dd, J=4.4, 14.0 Hz, 1H), 2.92 (s, 2H), 2.78 (br dd, J=10.8, 13.6 Hz, 1H), 2.47 (br s, 3H), 2.41 (s, 3H), 1.97 (s, 3H), 1.38 (s, 6H).

### Assembly

The peptides may be assembled using standard Symphony protocols. The peptide sequences were assembled as follows: Resin (250 mg. 0.14 mmol) in each reaction vial was washed twice with 4 ml of DMF followed by treatment with 2.5 ml of 20% 4-methyl piperidine (Fmoc de-protection) for 10 min. The resin was then filtered and washed two times with DMF (4 ml) and re-treated with N-methyl piperidine for additional 30 minute. The resin was again washed three times with DMF (4 ml) followed by addition 2.5 ml of amino acid and 2.5 ml of HBTU-DIEA mixture. After 45 min of frequent agitations, the resin was filtered and washed three timed with DMF (4 ml each). For a typical peptide of the present invention, double couplings were performed. After completing the coupling reaction, the resin was washed three times with DMF (4 ml each) before proceeding to the next amino acid coupling.

### Ring Closing Metathesis to Form Olefins

An an example of ring closing metathesis a the resin (100 μmol) was washed with 2 ml of DCM (3×1 min) and then with 2 ml of DCE (3×1 min) before being treated with a solution of 2 ml of a 6 mM solution of Grubbs' first-generation catalyst in DCE (4.94 mg ml-1; 20 mol % with regard to the resin substitution). The solution was refluxed overnight (12 h) under nitrogen before being drained. The resin was washed three times with DMF (4 ml each); DCM (4 ml) before being dried and cleaved.

### Cleavage

Following completion of the peptide assembly, the peptide was cleaved from the resin by treatment with cleavage reagent, such as reagent K (82.5% trigluoroacetic acid, 5% water, 5% thioanisole, 5% phenol, 2.5% 1,2-ethanedithiol). The cleavage reagent was able to successfully cleave the peptide from the resin, as well as all remaining side chain protecting groups.

The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile: water (7:3 with 1% TFA) and filtered. The quality of linear peptide was then verified using electrospray ionization mass spectrometry (ESI-MS) (Micromass/Waters ZQ) before being purified.

### Disulfide Bond Formation Via Oxidation

The peptide containing the free thiol (for example diPen) was assembled on a Rink Amide-MBHA resin following general Fmoc-SPPS procedure. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile: water (7:3 with 1% TFA) and filtered giving the wanted unoxidized peptide crude peptide.

The crude, cleaved peptide with positions X4 and X9, for example, possessing either Cys, Pen, hCys, (D)Pen, (D)Cys or (D)hCys, was dissolved in 20 ml of water: acetonitrile. Saturated Iodine in acetic acid was then added drop wise with stirring until yellow color persisted. The solution was stirred for 15 minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC machine (Luna C18 support, 10 u, 100 A, Mobile phase A: water containing 0.1% TFA, mobile phase B: Acetonitrile (ACN) containing 0.1% TFA, gradient began with 5% B, and changed to 50% B over 60 minutes at a flow rate of 15 ml/min). Fractions containing pure product were then freeze-dried on a lyophilyzer.

### Thioether Bond Formation

The peptide containing the free thiol (e.g., Cys) and hSer(OTBDMS) was assembled on a Rink Amide-MBHA resin following general Fmoc-SPPS procedure. Chlorination was carried out by treating the resin with PPh₃ (10 equiv.) and Cl₃CCN (10 equiv.) in DCM for 2 h. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile: water (7:3 with 1% TFA) and filtered giving the wanted uncyclized crude peptide

The crude peptide possessing a free thiol (e.g., Cys, Pen, aMeCys, hCys, (D)Pen, (D)Cys or (D)hCys and the alkyl halide (hSer(Cl)) at either the X4 and X9 position or X9 and X4 position was dissolved in 0.1 M TRIS buffer pH 8.5. Cyclization was allowed to take place overnight at RT. The solvent mixture was then purified by first being diluted two-fold with water and then loaded onto a reverse phase HPLC machine (Luna C18 support, 10 u, 100 A, Mobile phase A: water containing 0.1% TFA, mobile phase B: Acetonitrile (ACN) containing 0.1% TFA, gradient began with 5% B, and changed to 50% B over 60 minutes at a flow rate of 15 ml/min). Fractions containing pure product were then freeze-dried on a lyophilizer

### Purification

Analytical reverse-phase, high performance liquid chromatography (HPLC) was performed on a Gemini C18 column (4.6 mm×250 mm) (Phenomenex). Semi-Preparative reverse phase HPLC was performed on a Gemini 10 μm C18 column (22 mm×250 mm) (Phenomenex) or Jupiter 10 μm, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative).

### General Procedure 1A:

IL-23R inhibitor compounds described herein were synthesized from amino acids monomers using standard Fmoc solid phase synthesis techniques on a CEM Liberty Blue™ microwave peptide synthesizer. The peptides were assembled using Oxyma/DIC (ethyl cyanohydroxyiminoacetate/diisopropyl-carbodiimide) with microwave heating. Rink Amide-MBHA resin (100-200 mesh, 0.66 mmol/g) was used for peptides with C-terminal amides and pre-loaded Wang Resin with N-α-Fmoc protected amino acid was used for peptide with C-terminal acids. Oxyma was prepared as a 1M solution in DMF with 0.1M DIEA. DIC was prepared as 0.5M solution in DMF. The Amino acids were prepared at 200 mM. Peptide inhibitors of the present invention were identified based on medicinal chemistry optimization and/or phage display and screened to identify those having superior binding and/or inhibitory properties.

### Assembly

The peptides may also be made using standard CEM Liberty Blue™ protocols. The peptide sequences were assembled as follows: Resin (400 mg, 0.25 mmol) was suspended in 10 ml of 50/50 DMF/DCM. The resin was then transferred to the reaction vessel in the microwave cavity. The peptide was assembled using repeated Fmoc deprotection and Oxyma/DIC coupling cycles. For deprotection, 20% 4-methylpiperidine in DMF was added to the reaction vessel and heated to 90° C. for 65 seconds. The deprotection solution was drained and the resin washed three times with DMF. For most amino acids, 5 equivalents of amino acid, Oxyma and DIC were then added to the reaction vessel and microwave irradiation rapidly heated the mixing reaction to 90° C. for 4 min. For Arginine and Histidine residues, milder conditions using respective temperatures of 75 and 50° C. for 10 min were used to prevent racemization. Rare and expensive amino acids were often coupled manually overnight at room temperature using only 1.5-2 eq of reagents. Difficult couplings were often double coupled 2×4 min at 90° C. After coupling the resin was washed with DMF and the whole cycle was repeated until the desired peptide assembly was completed.

### Cleavage

Following completion of the peptide assembly, the peptide was then cleaved from the resin by treatment with a standard cleavage cocktail of 91:5:2:2 TFA/H₂O/TIPS/DODT for 2 hrs. If more than one Arg (pbf) residue was present the cleavage was allowed to go for an additional hour.

The cleaved peptides were precipitated in cold diethyl ether. The filtrate was decanted off and a second aliquot of cold ether was added, and the procedure was repeated. The quality of linear peptide was then verified using electrospray ionization mass spectrometry (ESI-MS) (Waters® Micromass® ZQ™) before being purified.

### Disulfide Bond Formation Via Oxidation

The peptide containing the free thiol (for example diPen) was assembled on a Rink Amide-MBHA resin following general Fmoc solid phase synthesis, cleavage and isolation as described above.

The crude cleaved peptide comprising two thiol containing amino acids selected independently from Cys, Pen, hCys, (D)Pen, (D)Cys and (D)hCys was dissolved ˜2 mg/ml in 50/50 acetonitrile/water. Saturated iodine in acetic acid was then added dropwise with stirring until yellow color persisted. The solution was stirred for a few minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC Column (Luna® C18 support, 10 u, 100 A, Mobile phase A: water containing 0.1% TFA, mobile phase B: acetonitrile (ACN) containing 0.1% TFA, gradient began with 15% B, and changed to 50% B over 60 minutes at a flow rate of 15 ml/min). Fractions containing pure product were then freeze-dried on a lyophilizer.

### Purification

Analytical reverse-phase, high performance liquid chromatography (HPLC) was performed on a Gemini® C18 column (4.6 mm×250 mm) (Phenomenex). Semi-Preparative reverse phase HPLC was performed on a Gemini® 10 μm C18 column (22 mm×250 mm) (Phenomenex) or Jupiter® 10 μm, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 20 mL/min (preparative).

### Example 1. Preparation of Peptide of SEQ ID NO.: 1

Ac-[Pen]*-N-T-[W (7-Me)]-[Lys(Ac)]-[Pen]*-Phe [4-(2-aminoethoxy)]-[2-Nal]-[THP]-E-N-[3-Pal]-Sarc-NH₂ (*Pen-Pen form disulfide bond) (SEQ ID NO.: 1)

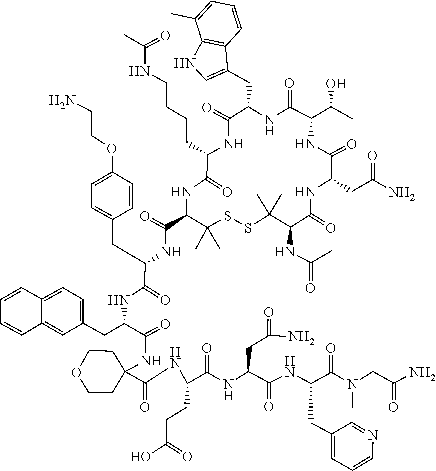 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

The synthesis of SEQ ID NO.: 1 is prepared using FMOC solid phase peptide synthesis techniques.

The peptide is constructed on Rink Amide MBHA resin using standard FMOC protection synthesis conditions reported in the literature. The constructed peptide is isolated from the resin and protecting groups by cleavage with strong acid followed by precipitation. Oxidation to form the disulfide bond is performed followed by purification by reverse phase HPLC (RP-HPLC) and counter ion exchange. Lyophilization of pure fractions gives the final product.

Swell Resin: 10 g of Rink Amide MBHA solid phase resin (0.66 mmol/g loading) is transferred to a 250 ml peptide vessel with filter frit, ground glass joint and vacuum side arm. The resin is washed 3× with DMF.
Step 1: Coupling of FMOC-Sarc-OH: Deprotection of the resin bound FMOC group is realized by adding 2 resin-bed volumes of 20% 4-methyl-piperidine in DMF to the swollen resin and shaking for 3-5 min prior to draining and adding a second, 2-resin-bed volume of the 4-methyl piperidine solution and shaking for an additional 20-30 min. After deprotection the resin is washed 3x DMF with shaking. FMOC-Sarc-OH (3 eq. 6.2 g) is dissolved in 100 ml DMF along with Oxyma (4.5 eq. 4.22 g). Preactivation of the acid is accomplished by addition of DIC (3.9 eq. 4 ml) with shaking for 15 min prior to addition to the deprotected resin. An additional aliquot of DIC (2.6 eq. 2.65 ml) is then added after ˜15 min of coupling. The progress of the coupling reaction is monitored by the colorimetric Kaiser test. Once the reaction is judged complete the resin is washed 3×DMF with shaking prior to starting the next deprotection/coupling cycle.
Step 2: Coupling of FMOC-3Pal-OH: FMOC deprotection is again accomplished by adding two sequential, 2-resin-bed volumes of 20% 4-methyl-piperidine in DMF, one times 3-5 minutes and one times 20-30 minutes, draining in between treatments. The resin is then washed 3 times prior to coupling with protected 3-pyridyl alanine (3Pal). FMOC-3Pal-OH (3 eq, 7.8 g) is dissolved in DMF along with Oxyma (4.5 eq. 4.22 g). Preactivation with DIC (3.9 eq, 4 ml) for 15 minutes is done prior to addition to the Sarc-Amide resin. After 15 minutes, an additional aliquot of DIC (2.6 eq. 2.65 ml) is added to the reaction. Once the reaction is complete as determined by the Kaiser test, the resin is again washed 3× with DMF prior to starting the next deprotection/coupling cycle.
Step 3: Coupling of FMOC-Asn(Trt)-OH: The FMOC is removed from the N-terminus of the resin bound 3Pal and washed as previously described. FMOC-Asn(Trt)-OH (2 eq, 8 g) is dissolved in 100 ml of DMF along with Oxyma (3 eq. 2.81 g). DIC (2.6 eq. 2.65 ml) is added for preactivation of the acid for ˜15 minutes prior to addition to the 3Pal-Sarc-Amide resin. After ˜15 minutes, an additional aliquot of DIC (1.4 eq. 1.43 ml) is added to the reaction. Once the reaction is complete as determined by the Kaiser test, the resin is washed 3× with DMF prior to starting the next deprotection/coupling cycle.
Step 4: Coupling of FMOC-Glu(OtBu)-OH: The FMOC is removed from the N-terminus of the resin bound Asparagine and the resin washed with DMF as previously described. FMOC-Glu(OtBu)-OH (2 eq. 5.91 g) is dissolved in 100 ml of DMF along with Oxyma (3 eq, 2.81 g). DIC (2.6 eq. 2.65 ml) is added for preactivation of the acid ˜15 minutes prior to addition to the Asn(Trt)-3Pal-Sarc-Amide resin. After ˜15 minutes, an additional aliquot of DIC (1.4 eq. 1.43 ml) is added to the reaction. Once the reaction is complete as determined by the Kaiser test the resin is washed 3× with DMF prior to starting the next deprotection/coupling cycle.
Step 5: Coupling of FMOC-THP-OH: The FMOC is removed from the N-terminus of the resin bound peptide and the resin is washed as previously described. FMOC-THP-OH (3 eq. 7.36 g) is dissolved in 100 ml of DMF along with Oxyma (4.5 eq. 4.22 g). DIC (3.9 eq. 4 ml) is added for preactivation of the acid ˜15 minutes prior to addition to the Glu(OtBu)-Asn(Trt)-3Pal-Sarc-Amide resin. After ˜15 minutes, an additional aliquot of DIC (2.6 eq, 2.65 ml) is added to the reaction. Once the reaction is complete as determined by the Kaiser test the resin is washed 3× with DMF prior to starting the next deprotection/coupling cycle.
Step 6: Coupling of FMOC-L-Ala (2-Naphthyl)-OH (Nal): The FMOC is removed from the N-terminus of the resin bound peptide and the resin washed as previously described. FMOC-L-Ala (2-Naphthyl)-OH (3 eq. 8.66 g) is dissolved in 100 ml of DMF along with Oxyma (4.5 eq, 4.22 g). DIC (3.9 eq, 4 ml) is added for preactivation of the acid ˜15 minutes prior to addition to the THP-Glu(OtBu)-Asn(Trt)-3Pal-Sarc-Amide resin. After ˜15 minutes, an additional aliquot of DIC (2.6 eq. 2.65 ml) is added. Once the reaction is complete as determined by the Kaiser test the resin was again washed 3× with DMF prior to starting the next deprotection/coupling cycle.
Step 7: Coupling of FMOC-4-[2-(Boc-amino-ethoxy)]-L-Phenylalanine (FMOC-AEF): The FMOC is removed from the N-terminus of the resin bound peptide and the resin washed as previously described. FMOC-4-[2-(Boc-amino-ethoxy)]-L-Phenylalanine (3 eq, 10.8 g) is dissolved in 100 ml of DMF along with Oxyma (4.5 eq. 4.22 g). DIC (3.9 eq, 4 ml) is added for preactivation of the acid ˜15 minutes prior to addition to the Nal-THP-Glu(OtBu)-Asn(Trt)-3Pal-Sarc-Amide resin. After ˜15 minutes, an additional aliquot of DIC (2.6 eq. 2.65 ml) is added to the reaction. Once the reaction is complete as determined by the Kaiser test the resin is washed 3× with DMF prior to starting the next deprotection/coupling cycle.
Step 8: Coupling of FMOC-Pen (Trt)-OH: The FMOC is removed from the N-terminus of the resin bound peptide and the resin washed as previously described. FMOC-Pen (Trt)-OH (3 eq. 12.14 g) is dissolved in 100 ml of DMF along with Oxyma (4.5 eq, 4.22 g). DIC (3.9 eq. 4 ml) is added for preactivation of the acid ˜15 minutes prior to addition to the AEF-Nal-THP-Glu(OtBu)-Asn(Trt)-3Pal-Sarc-Amide resin. After ˜15 minutes, an additional aliquot of DIC (2.6 eq. 2.65 ml) is added to the reaction. Once the reaction is complete as determined by the Kaiser test, the resin is again washed 3× with DMF prior to starting the next deprotection/coupling cycle.
Step 9: Coupling of FMOC-Lys(Ac)-OH: The FMOC is removed from the N-terminus of the resin bound peptide and the resin washed as previously described. FMOC-Lys(Ac)-OH (2 eq. 5.4 g) is dissolved in 100 ml of DMF along with Oxyma (3 eq. 2.81 g). DIC (2.6 eq. 2.65 ml) is added for preactivation of the acid ˜15 minutes prior to addition to the Pen (Trt)-AEF-Nal-THP-Glu(OtBu)-Asn(Trt)-3Pal-Sarc-Amide resin. After ˜15 minutes, an additional aliquot of DIC (1.4 eq. 1.43 ml) is added to the reaction. Once the reaction was complete as determined by the Kaiser test, the resin is again washed 3× with DMF prior to starting the next deprotection/coupling cycle.
Step 10: Coupling of FMOC-7-Me-Trp-OH: The FMOC is removed from the N-terminus of the resin bound peptide and the resin washed as previously described. FMOC-7-Me-Trp-OH (2 eq. 5.81 g) is dissolved in 100 ml of DMF along with Oxyma (3 eq. 2.81 g). DIC (2.6 eq. 2.65 ml) is added for preactivation of the acid ˜15 minutes prior to addition to the Lys(Ac)-Pen (Trt)-AEF-Nal-THP-Glu(OtBu)-Asn(Trt)-3Pal-Sarc-Amide resin. After ˜15 minutes, an additional aliquot of DIC (1.4 eq. 1.43 ml) is added to the reaction. Once the reaction is complete as determined by the Kaiser test, the resin is again washed 3× with DMF prior to starting the next deprotection/coupling cycle.
Step 11: Coupling of FMOC-Thr(tBu)-OH: The FMOC is removed from the N-terminus of the resin bound peptide and the resin washed as previously described. FMOC-Thr(tBu)-OH (4 eq. 10.5 g) is dissolved in 100 ml of DMF along with Oxyma (6 eq. 5.62 g). DIC (5.2 eq. 5.3 ml) is added for preactivation of the acid ˜15 minutes prior to addition to the 7MeTrp-Lys(Ac)-Pen (Trt)-AEF-Nal-THP-Glu(OtBu)-Asn(Trt)-3Pal-Sarc-Amide resin. After ˜15 minutes, an additional aliquot of DIC (2.6 eq. 2.65 ml) is added to the reaction. Once the reaction is complete as determined by the Kaiser test, the resin is again washed 3× with DMF prior to starting the next deprotection/coupling cycle.
Step 12: Coupling of FMOC-Asn(Trt)-OH: The FMOC is removed from the N-terminus of the resin bound peptide and the resin washed as previously described. FMOC-Asn(Trt)-OH (4 eq. 15.8 g) is dissolved in 100 ml of DMF along with Oxyma (6 eq. 5.62 g). DIC (5.2 eq. 5.3 ml) is added for preactivation of the acid ˜15 minutes prior to addition to the Thr (tBu)-7MeTrp-Lys(Ac)-Pen (Trt)-AEF-Nal-THP-Glu(OtBu)-Asn(Trt)-3Pal-Sarc-Amide resin. After ˜15 minutes, an additional aliquot of DIC (2.6 eq. 2.65 ml) is added to the reaction. Once the reaction is complete as determined by the Kaiser test, the resin is again washed 3x with DMF prior to starting the next deprotection/coupling cycle.
Step 13: Coupling of FMOC-Pen (Trt)-OH: The FMOC is removed from the N-terminus of the resin bound peptide and the resin washed as previously described. FMOC-Pen (Trt)-OH (2 eq. 8.1 g) is dissolved in 100 ml of DMF along with Oxyma (3 eq. 2.81 g). DIC (2.6 eq. 2.65 ml) is added for preactivation of the acid ˜15 minutes prior to addition to the Asn(Trt)-Thr(tBu)-7MeTrp-Lys(Ac)-Pen (Trt)-AEF-Nal-THP-Glu(OtBu)-Asn(Trt)-3Pal-Sarc-Amide resin. After ˜15 minutes, an additional aliquot of DIC (2.6 eq. 2.65 ml) is added to the reaction. Once the reaction is complete as determined by the Kaiser test, the resin is again washed 3× with DMF prior to the final deprotection and acetic acid capping of the constructed peptide.
Step 14: Acetyl Capping: The FMOC is removed from the N-terminus of the resin bound peptide and the resin washed as previously described. 150 ml of Capping Reagent A (THF/Acetic anhydride/Pyridine, 80:10:10) is added to the constructed Pen (Trt)-Asn(Trt)-Thr (tBu)-7MeTrp-Lys(Ac)-Pen (Trt)-AEF-Nal-THP-Glu(OtBu)-Asn(Trt)-3Pal-Sarc-Amide resin and shaken for 30 min. The resin is washed 3× with DMF followed by 5× with DCM. The resin is divided into 5-50 ml centrifuge tubes and placed under vacuum for 1.5 hrs prior to cleavage with TFA.
Step 15: TFA Cleavage and Ether precipitation: 200 ml of the TFA cleavage cocktail (90/5/2.5/2.5 TFA/water/TIPS/DODT) is prepared. 40 ml of the cleavage cocktail is added to each of the 5 tubes containing the protected resin bound peptide and shaken for two hours. The spent resin is filtered away and the filtrate divided evenly into 18-50 ml centrifuge tubes for precipitation. Cold diethyl ether is added to each forming a white precipitate that is then centrifuged. The ether is decanted to waste and 2 more ether washes of the precipitate are performed. The resulting white precipitate cake is dried overnight in the hood to give the crude reduced peptide.
Step 16: Disulfide Oxidation: The crude peptide is oxidized and purified in four 1 L batches. ˜2.5 g of crude peptide is dissolved in 1 L 20% ACN/water. With stirring, a saturated solution of iodine in acetic acid/methanol is added dropwise to the 1 L peptide solution until the yellow/brown color of the I₂ remains and does not fade away. The light yellow solution is allowed to sit for 5 min prior to quenching the excess I₂ with a pinch of ascorbic acid.
Step 17: RP-HPLC purification: The RP-HPLC purification is performed s immediately following each I₂ oxidation. A preparative purification column (Phenomenex, Luna, C18(2), 100□, 250×50 mm) is equilibrated at 70 ml/min with 20% MPB in MPA (MPA=0.1% TFA/water, MPB=0.1% TFA in ACN). The 1 L of quenched oxidized peptide is loaded onto the equilibrated column at 70 ml/min. After the solvent front elutes, a gradient of 25-45% MPB at 70 ml/min is run over 60 min. The desired material is isolated in fractions and each are analyzed by analytical RP-HPLC. Pure fractions are combined from all four purifications and lyophilized to give purified TFA salt ready for counterion exchange.
Step 18: Counterion Exchange to Acetate: The same preparative RP-HPLC column is equilibrated with 5% MPB in MPA at 70 ml/min (MPA=0.3% AcOH in Water, MPB=0.3% AcOH in ACN, MPC=0.5M NH₄OAc in Water.) The purified peptide TFA salt is dissolved in 50/50 ACN/water and diluted to 15% ACN. The solution is loaded onto the equilibrated column at 70 ml/min and the solvent front is eluted. The captured peptide is washed with 5% MPB in MPA for 5 min. The captured peptide is then washed with 5% MPB in MPC for 40 min at 70 ml/min to exchange the counterions to Acetate. The captured peptide is washed with 5% MPB in MPA at 70 ml/min for 10 min to clear all NH₄OAc from the system. Finally, the peptide is eluted with a gradient of 5-70% MPB in MPA over 60 minutes and collected in fractions.
Step 19: Final Lyophilization and Analysis: The collected fractions are analyzed by analytical RP-HPLC, and all fractions >95% purity are combined. Lyophilization of the combined fractions gives SEQ ID NO.: 1 as a white powder with a purity >95% as determined by RP-HPLC. Peptide identity is confirmed with LC/MS of the purified Peptide of SEQ ID NO.: 1, giving 2 charged states of the peptide, M+2/2 of 950 amu and the molecular ion of 1899 amu.

### Example 2. Synthesis of MeCO-r-Pen-N-T-7MeW-K(Ac)-Pen-AEF-2Nal-THP-E-N-5MePyridinAla-Sar-CONH2 (Compound 345, SEQ ID NO:345)

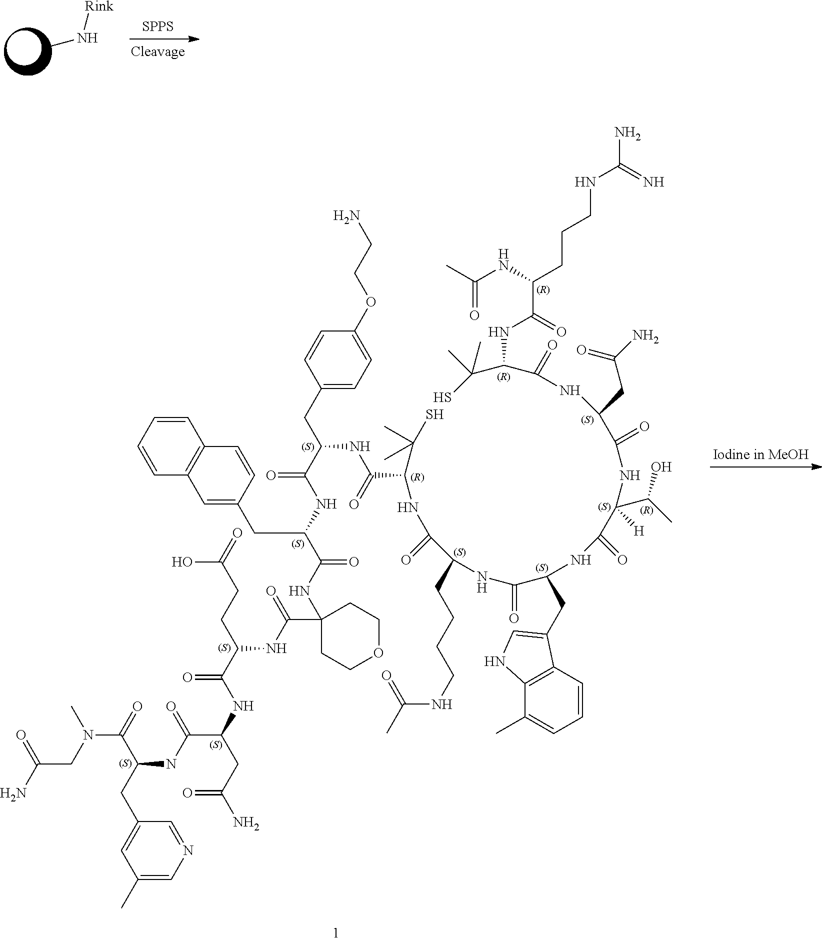

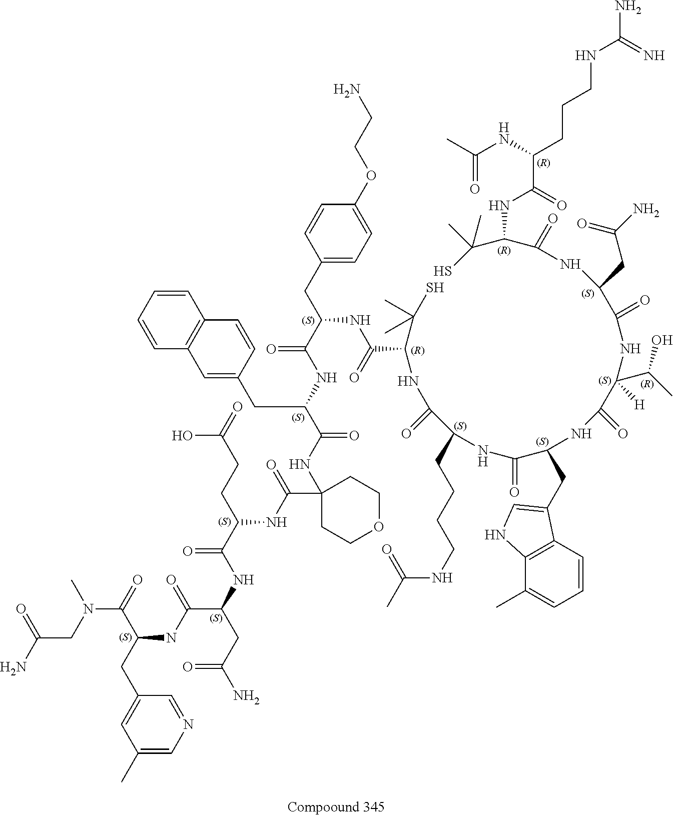

### Solid-Phase Peptide Synthesis:

The peptide was chemically synthesized using optimized 9-fluorenylmethoxy carbonyl (Fmoc) solid phase peptide synthesis protocols. For C-terminal amides, Rink-amide MBHA resin was used. The side chain protecting groups were as follows: D-Arg: Pbf; Thr, Glu: O-tButyl; Asn, Pen: Trityl; AEF: Boc. For coupling, a two to three-fold excess of a solution containing Fmoc amino acid, HATU and DIEA (1:0.95:2) in DMF was added to swelled resin for 1 to 4 hours. Double coupling is employed when coupling 2Nal. Fmoc protecting group removal was achieved by treatment with a DMF, piperidine (4:1) solution for 30 min. The cycles are repeated until the full-length peptide is obtained.

### Peptide Cleavage:

Peptied was cleaved from the rsin by addition of cleavage buffer (5.0% DTT/2.5% H2O/2.5% TIS/90% TFA) 75 mL to the flask containing the side chain protected peptide at room temperature and stir for 3 hrs. The resin was filtered and washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged. The lyophilized residue gave crude compound 1 (1.8 g).

### Peptide Cyclization and Purification:

Crude peptide Compound 1 (1.8 g, 0.86 mmol) was dissolved in 20% MeCN/H2O (1000 mL). To the stirred solution of the peptide was added the iodine solution in MeOH (0.1M, 5.0 mL) drop-wise until solution remains yellow. After ˜2 h LCMS showed the reaction was complete. Excess iodine was quenched by the addition of 1M Na2S2O3 in water (15 uL) (turned colorless instantly). Added 10-20 mL of MeCN to decrease turbidity. Purified the solution by Prep-HPLC (A: 0.075% TFA in H2O, B: ACN) to give compound 345 (371 mg, 96.4% purity, 17.0% yield for this step; over all yield: 14.8%) obtained as white solid. Analysis was performed using a C18 column with a flow rate of 1 mL/min. LCMS calculated MW: 2068.38, observed MW: 1034.5 [(M+2H)/2].

### Example 3. Synthesis of MeCO-Pen-N-T-7MeW-K(Ac)-Pen-AEF(G)-2Nal-THP-E-N-3Pya-Sar-CONH2 (Compound 477 SEQ ID NO:477)

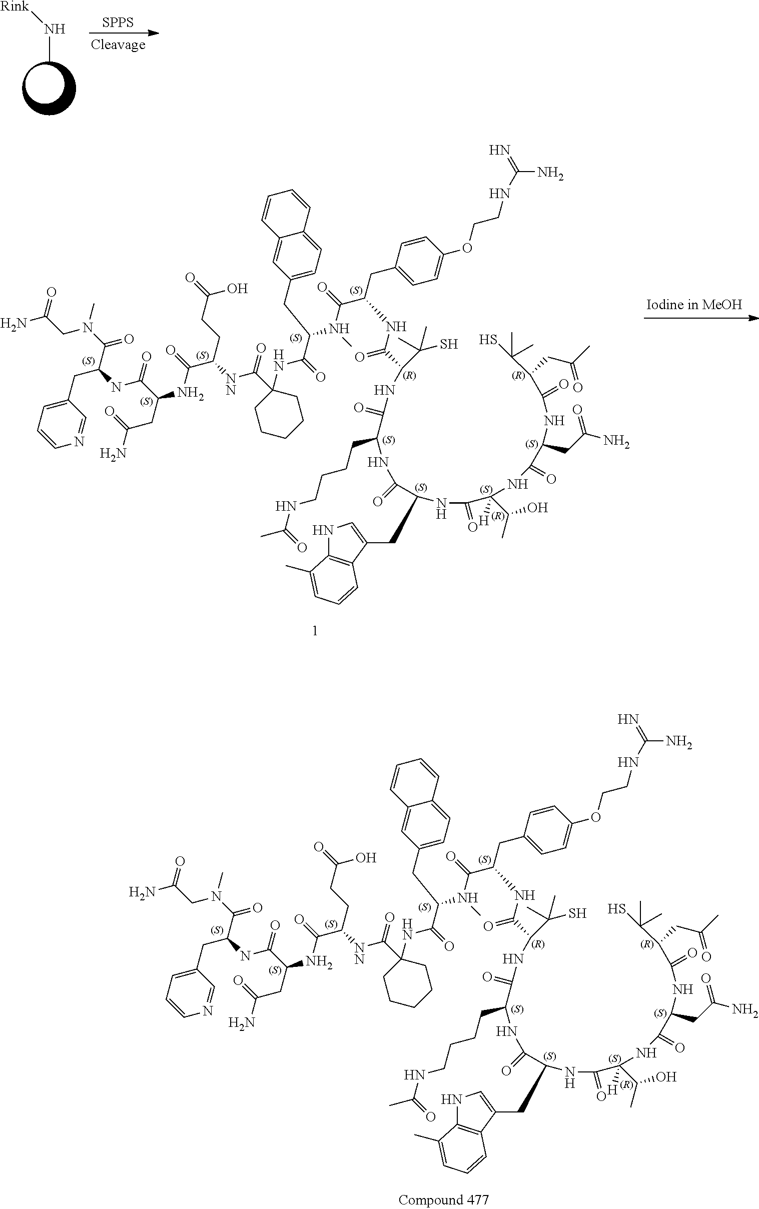

### Solid-Phase Peptide Synthesis:

Peptide were chemically synthesized using optimized 9-fluorenylmethoxy carbonyl (Fmoc) solid phase peptide synthesis protocols. For C-terminal amides, Rink-amide MBHA resin was used. The side chain protecting groups were as follows: AEF(G): Pbf; Thr, Glu: O-tButyl; Asn, Pen: Trityl. For coupling, a two to three-fold excess of a solution containing Fmoc amino acid, HATU and DIEA (1:0.95:2) in DMF was added to swelled resin for 1 to 4 hours. Double coupling is employed when coupling 2Nal. Fmoc protecting group removal was achieved by treatment with a DMF, piperidine (4:1) solution for 30 min. The cycles are repeated until the full-length peptide is obtained.

### Peptide Cleavage:

Peptide was cleaved from the resin by the addition of cleavage buffer (5.0% DTT/2.5% H2O/2.5% TIS/90% TFA) 75 mL to the flask containing the side chain protected peptide at room temperature and stir for 3 hrs. The resin was filtered washed with 5 mL TFA, and the combined filtrate was precipitated with cold methyl tertbutyl ether (MTBE). The mixture was centrifuged (3000 rpm, 3 min) and decanted. The pellet was washed with MTBE and centrifuged. Lyophilized residue gave crude compound 1 (1.6 g).

### Peptide Cyclization and Purification:

Crude peptide compound 1 (1.6 g, 0.824 mmol) was dissolved in 20% MeCN/H2O (1000 mL). To a stirred solution of the peptide was added the iodine solution in MeOH (0.1M, 2.0 mL) drop-wise until solution remains yellow. After ˜2 h LCMS showed the reaction was complete. Excess iodine was quenched by the addition of 1M Na2S2O3 in water (15 uL) (turned colorless instantly). Added 10-20 mL of MeCN to decrease turbidity. Purified the solution by Prep-HPLC (A: 0.075% TFA in H2O, B: ACN) to give compound 477 (575 mg, 96.4% purity, 31.0% yield for this step; over all yield: 25.5%) obtained as white solid. Analysis was performed using a C18 column with a flow rate of 1 mL/min (Analysis LCMS Method). LCMS calculated MW: 1940.21, observed MW: 970.7 [(M+2H)/2].

### Example 4. Synthesis of MeCO-r-Abu (1)-N-T-W-K(Ac)-aMeC (1)-AEF-2Nal-THP-E-N-3Pya-Sar-CONH2 (Compound 478, SEQ ID NO:478)

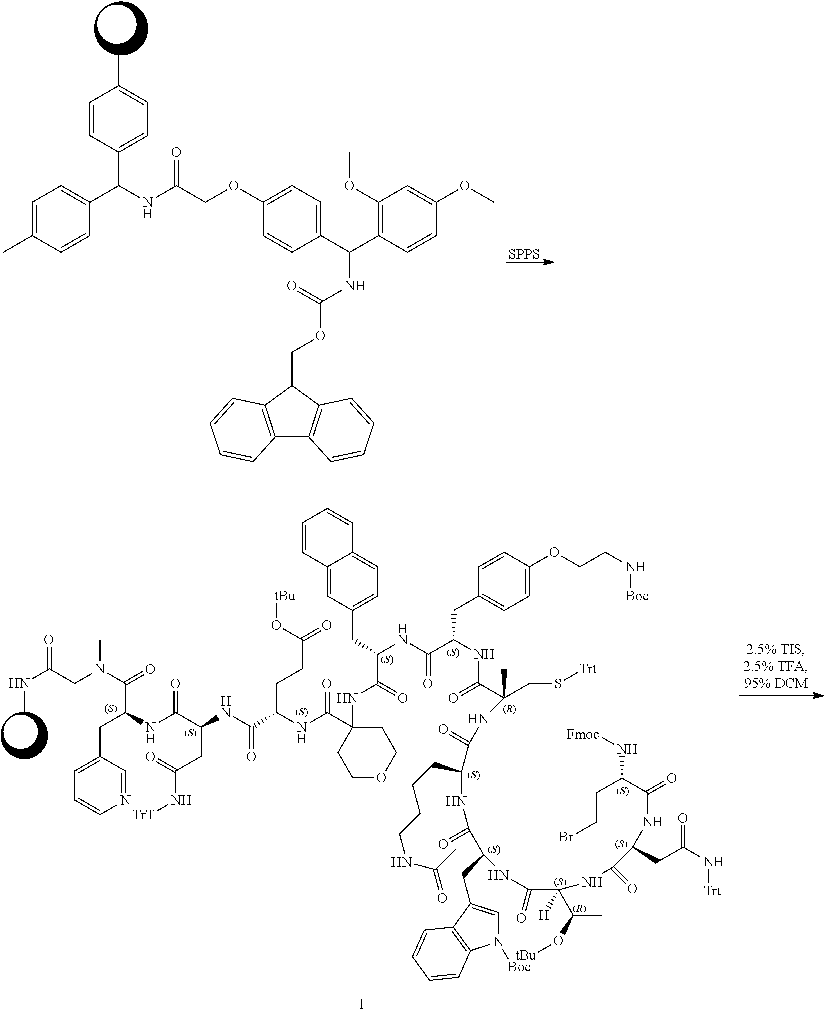

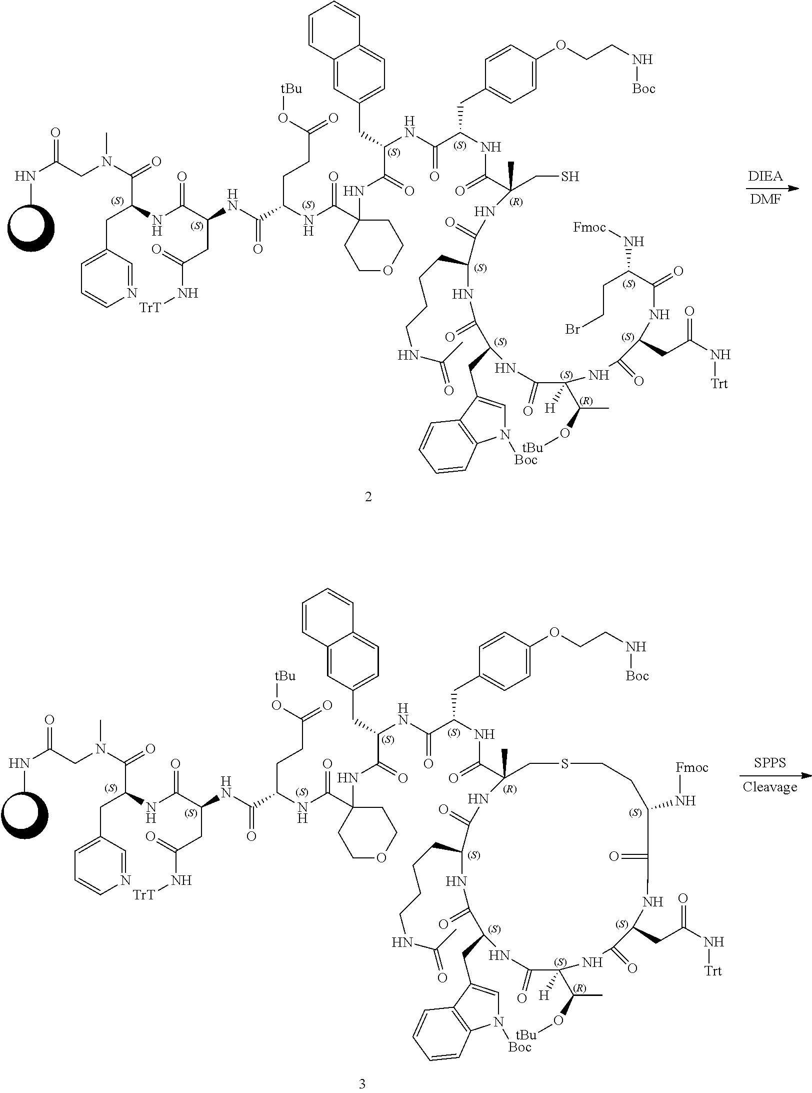

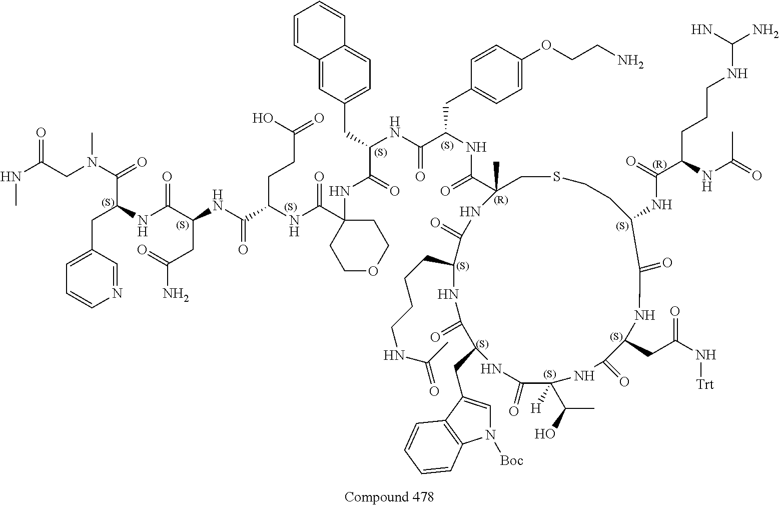

### Solid-Phase Peptide Synthesis:

Peptide were chemically synthesized using optimized 9-fluorenylmethoxy carbonyl (Fmoc) solid phase peptide synthesis protocols. For C-terminal amides, Rink-amide MBHA resin was used. The side chain protecting groups were as follows: D-Arg: Pbf; Thr, Glu: O-tButyl; Asn, aMeCys: Trityl; AEF, Trp: Boc. For coupling, a two to three-fold excess of a solution containing Fmoc amino acid, HATU and DIEA (1:0.95:2) in DMF or Fmoc amino acid, DIC and HOAT(1:1:1) was added to swelled resin for 1 to 32 hours. Double coupling is employed when coupling 2Nal, Lys(Ac) and Fmoc-4-Br-L-HomoAla-OH. Fmoc protecting group removal was achieved by treatment with a DMF, piperidine (4:1) solution for 30 min. Trityl (“Trt”) protecting group on aMeCys removal was achieved by treatment with trifluoroacetic acid, tri-isopropylsilane and DCM (2.5:2.5:95) solution for 3 min*10 times. For thioether cyclization, a solution containing DIEA (5 eq) in DMF was added to swelled resin for 1h*2 times. The cycles are repeated until the full-length peptide is obtained.

### Synthetic Method for Thioether Cyclization:

Coupling of Fmoc-4-Br-L-HomoAla-OH. After de-protection, the resin was washed with 30 mL of DMF (5×0.1 min) and followed by addition of 2.5 mL of Fmoc-4-Br-L-HomoAla-OH in DMF (400 mM) and 2.5 mL of coupling reagent HOAT in DMF (400 mM) and DIC (0.16 mL, 1.0 mmol). The coupling reaction was mixed for 16 hrs. Then washed with 30 mL of DMF (5×0.1 min) and repeat coupling one more time for 16˜32 hrs. After completing the coupling reaction, the resin was washed with 30 mL of DMF (3×0.1 min) prior to starting the next step.

Trityl group removal on aMeCys was accomplished by washing with 30 mL of DMF (5×0.1 min) and DCM (5×0.1 min) followed by addition of 3% TFA and 2.5% TIS in DCM (30 mL) for 3 min*10 times (the reaction solution changed from orange to colorless), washed with DCM, 5% DIEA in DMF and DMF for 3 times

Thioether cyclization on the resin was accomplished by washing the resin with 30 mL of DMF (5×0.1 min) followed by addition of DIEA (5 eq) in DMF (30 mL), the coupling reaction was mixed for 1h. Cleavage test and LCMS showed the reaction was finished. After completing the coupling reaction, the resin was washed with 30 mL of DMF (3×0.1 min) prior to starting the next step.

### Peptide Cleavage:

Peptide was claved from the resin by addition of cleavage buffer (5.0% DTT/2.5% H₂O/2.5% TIS/90% TFA) 75 mL to the flask containing the side chain protected peptide at room temperature and stir for 3 hrs. The resin was filtered washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged. Lyophilized the residue to give the compound 1 (750 mg, 75.7% yield, crude)

### Peptide Purification:

Crude peptide was purified by Prep-HPLC (A: 0.075% TFA in H₂O, B: ACN) to give compound 478 (82 mg, 95.2% purity, 6.72% yield) obtained as white solid. Analysis was performed using a C18 column with a flow rate of 1 mL/min. LCMS calculated MW: 1980.21, observed MW: 990.6 [(M+2H)/2].

### Example 5. Biological Assays

### IL23R Reporter Assay

Compounds were serially diluted in 100% (v/v) DMSO) and plated using an Echo acoustic dispenser (Labcyte) into 1536-well non-treated black assay plates (Corning #9146). 3 μL of HEK293 cells containing IL-23R, IL-12Rβ1 and a firefly luciferase reporter gene driven by a STAT-inducible promoter (Promega) were added to the plates (4000 cells/well), followed by 3 μL of 10 ng/ml IL-23 (equivalent to EC₉₀ concentration). After 5 h at 37° C., 5% CO₂, 95% relative humidity, cells were placed at 20° C. and treated with BioGlo reagent (Promega) according to the Manufacturer's instructions. Luminescence was measured on a Pherastar FSX (BMG LabTech). Data were normalized to IL-23 treatment (0% inhibition) and 30 μM of control inhibitor (100% inhibition), and IC₅₀ values were determined using a 4-parameter Hill equation. Data for exemplary compounds are shown in Table 3b.

TABLE 3
IL-23 Binding Data for the Compound Numbers 158 to 478.

| Number | IC₅₀ (μM) |
|---|---|
| 158 | 0.18 |
| 159 | 3.29 |
| 160 | 1.45 |
| 161 | 1.1 |
| 162 | 0.3 |
| 163 | 1.03 |
| 164 | 0.38 |
| 165 | 0.37 |
| 166 | 0.48 |
| 167 | 1.02 |
| 168 | 0.63 |
| 169 | 0.28 |
| 170 | 2.2 |
| 171 | 0.47 |
| 172 | 3.41 |
| 173 | >16.61 |
| 174 | 0.21 |
| 175 | 0.26 |
| 176 | 0.43 |
| 177 | 0.35 |
| 178 | 0.39 |
| 179 | 0.63 |
| 180 | 0.74 |
| 181 | 0.62 |
| 182 | 1.02 |
| 183 | 1.31 |
| 184 | 0.93 |
| 185 | 1.24 |
| 186 | 1.91 |
| 187 | 1.07 |
| 188 | 1.98 |
| 189 | 0.48 |
| 190 | 0.12 |
| 191 | >16.61 |
| 192 | 0.35 |
| 193 | 0.27 |
| 194 | 0.22 |
| 195 | 0.16 |
| 196 | 0.24 |
| 197 | 0.24 |
| 198 | >16.61 |
| 199 | 1.9 |
| 200 | 0.72 |
| 201 | 1.09 |
| 202 | 0.54 |
| 203 | 0.42 |
| 204 | 0.66 |
| 205 | >16.61 |
| 206 | ~12.82 |
| 207 | >16.61 |
| 208 | >16.61 |
| 209 | 0.32 |
| 210 | 0.49 |
| 211 | 0.047 |
| 212 | 0.49 |
| 213 | 0.085 |
| 214 | 0.13 |
| 215 | 0.19 |
| 216 | 0.35 |
| 217 | 0.17 |
| 218 | 0.12 |
| 219 | 0.066 |
| 220 | 0.6 |
| 221 | 0.54 |
| 222 | 0.065 |
| 223 | 0.099 |
| 224 | 0.082 |
| 225 | 0.066 |
| 226 | 0.19 |
| 227 | 6.13 |
| 228 | 1.31 |
| 229 | 3.54 |
| 230 | 2.58 |
| 231 | 11.7 |
| 232 | 4.48 |
| 233 | 4.87 |
| 234 | 3.65 |
| 235 | 0.034 |
| 236 | 0.17 |
| 237 | 0.019 |
| 238 | 0.013 |
| 239 | 2.37 |
| 240 | 0.23 |
| 241 | 0.24 |
| 242 | 0.19 |
| 243 | 0.11 |
| 244 | 0.16 |
| 245 | 0.18 |
| 246 | 0.11 |
| 247 | 0.26 |
| 248 | 0.13 |
| 249 | 0.31 |
| 250 | 0.22 |
| 251 | 0.15 |
| 252 | 0.18 |
| 253 | 0.055 |
| 254 | 0.037 |
| 255 | 0.05 |
| 256 | 2.06 |
| 257 | 1.25 |
| 258 | 1 |
| 259 | 0.87 |
| 260 | 0.48 |
| 261 | >16.61 |
| 262 | 5.72 |
| 263 | 0.0067 |
| 264 | 0.0095 |
| 265 | 0.34 |
| 266 | 0.22 |
| 267 | 6.28 |
| 268 | 1.8 |
| 269 | 0.26 |
| 270 | 0.38 |
| 271 | 0.17 |
| 272 | 0.03 |
| 273 | 0.032 |
| 274 | 0.034 |
| 275 | 0.0024 |
| 276 | 0.0031 |
| 277 | 0.0025 |
| 278 | 0.0032 |
| 279 | 0.33 |
| 280 | 0.61 |
| 281 | 0.31 |
| 282 | 0.24 |
| 283 | 0.054 |
| 284 | 0.39 |
| 285 | 0.43 |
| 286 | 0.24 |
| 287 | 0.43 |
| 288 | 0.056 |
| 289 | 0.24 |
| 290 | 0.2 |
| 291 | 2.24 |
| 292 | 2.08 |
| 293 | 0.17 |
| 294 | 0.13 |
| 295 | 0.54 |
| 296 | 0.0055 |
| 297 | 0.29 |
| 298 | 0.0069 |
| 299 | 3.34 |
| 300 | 1.12 |
| 301 | 0.0023 |
| 302 | 0.0028 |
| 303 | 0.21 |
| 304 | 0.091 |
| 305 | 0.045 |
| 306 | 0.068 |
| 307 | 0.004 |
| 308 | 0.52 |
| 309 | 0.6 |
| 310 | 0.13 |
| 311 | 0.65 |
| 312 | 0.64 |
| 313 | 0.15 |
| 314 | 0.0035 |
| 315 | 0.0038 |
| 316 | |
| 317 | |
| 318 | |
| 319 | 0.83 |
| 320 | 0.079 |
| 321 | 0.16 |
| 322 | 0.45 |
| 323 | 0.28 |
| 324 | 0.51 |
| 325 | 0.33 |
| 326 | 0.0037 |
| 327 | 0.44 |
| 328 | 0.097 |
| 329 | 0.0024 |
| 330 | 0.0037 |
| 331 | 0.0049 |
| 332 | 0.0053 |
| 333 | 0.0061 |
| 334 | 4.1 |
| 335 | 0.03 |
| 336 | 0.013 |
| 337 | 0.0029 |
| 338 | >16.61 |
| 339 | 0.0038 |
| 340 | 0.0042 |
| 341 | 0.0053 |
| 342 | 0.0087 |
| 343 | 0.004 |
| 344 | 0.0061 |
| 345 | 0.005 |
| 346 | 0.0062 |
| 347 | 0.029 |
| 348 | >16.61 |
| 349 | 0.012 |
| 350 | 0.15 |
| 351 | 1.06 |
| 352 | 0.0084 |
| 353 | 0.0055 |
| 354 | 0.015 |
| 355 | 0.014 |
| 356 | 0.0032 |
| 357 | 0.0085 |
| 358 | 0.14 |
| 359 | >16.61 |
| 360 | 2.19 |
| 361 | 0.0051 |
| 362 | 0.074 |
| 363 | 0.13 |
| 364 | 0.004 |
| 365 | 0.13 |
| 366 | 0.0041 |
| 367 | 0.096 |
| 368 | 0.018 |
| 369 | 0.0044 |
| 370 | 0.0053 |
| 371 | 0.007 |
| 372 | 0.0065 |
| 373 | 0.0079 |
| 374 | 0.073 |
| 375 | 0.0054 |
| 376 | 0.0068 |
| 377 | 0.0052 |
| 378 | 0.0074 |
| 379 | 0.0044 |
| 380 | 0.024 |
| 381 | 0.0058 |
| 382 | 0.0055 |
| 383 | 0.0071 |
| 384 | 0.0036 |
| 385 | 0.0043 |
| 386 | 0.0043 |
| 387 | 0.055 |
| 388 | 0.11 |
| 389 | 0.006 |
| 390 | 0.0059 |
| 391 | 0.014 |
| 392 | 0.036 |
| 393 | 0.0059 |
| 394 | 0.0066 |
| 395 | 0.0094 |
| 396 | 0.0053 |
| 397 | 0.092 |
| 398 | 0.003 |
| 399 | 0.0046 |
| 400 | 0.0078 |
| 401 | 0.0042 |
| 402 | 0.0035 |
| 403 | 0.007 |
| 404 | 0.0057 |
| 405 | 0.0035 |
| 406 | 0.0034 |
| 407 | 0.0035 |
| 408 | 0.0024 |
| 409 | 0.0033 |
| 410 | 0.0018 |
| 411 | 0.0044 |
| 412 | 0.0028 |
| 413 | 0.0068 |
| 414 | 0.01 |
| 415 | 0.015 |
| 416 | 0.013 |
| 417 | 0.0035 |
| 418 | 0.0036 |
| 419 | 0.0032 |
| 420 | 0.034 |
| 421 | 0.0033 |
| 422 | 0.0077 |
| 423 | 0.021 |
| 424 | 0.018 |
| 425 | 0.015 |
| 426 | 0.046 |
| 427 | 0.0032 |
| 428 | 0.0094 |
| 429 | 0.013 |
| 430 | 0.0051 |
| 431 | 0.0034 |
| 432 | 0.0034 |
| 433 | 0.0093 |
| 434 | 0.0036 |
| 435 | 0.0026 |
| 436 | 0.0037 |
| 437 | 0.0075 |
| 438 | 0.0084 |
| 439 | 0.0048 |
| 440 | 0.0032 |
| 441 | 0.0047 |
| 442 | 0.0058 |
| 443 | 3.22 |
| 444 | 0.04 |
| 445 | 0.0036 |
| 446 | 0.0023 |
| 447 | 0.0033 |
| 448 | 0.0077 |
| 449 | 0.014 |
| 450 | 0.0022 |
| 451 | 0.026 |
| 452 | 0.0069 |
| 453 | 0.013 |
| 454 | 0.002 |
| 455 | 0.001 |
| 456 | 0.0018 |
| 457 | 0.00073 |
| 458 | 0.0021 |
| 459 | 0.057 |
| 460 | 0.024 |
| 461 | 0.0076 |
| 462 | 0.0094 |
| 463 | 0.0012 |
| 464 | 0.024 |
| 465 | |
| 466 | 0.0014 |
| 467 | 0.0046 |
| 468 | 0.0014 |
| 469 | 0.002 |
| 470 | 3.43 |
| 471 | >16.61 |
| 472 | 0.0064 |
| 473 | 0.017 |
| 474 | >16.61 |
| 475 | 0.0069 |
| 476 | 0.0086 |
| 477 | 0.091 |
| 478 | 0.0013 |

DB Cells IL23R pSTAT3 Cell Assay

IL-23 is believed to play a central role in supporting and maintaining Th17 differentiation in vivo. This process is thought to be mediated primarily through the Signal Transducer and Activator of Transcription 3 (STAT3), with phosphorylation of STAT3 (to yield pSTAT3) leading to upregulation of RORC and pro-inflammatory IL-17. This cell assay examines the levels of pSTAT3 in IL-23R-expressing DB cells when stimulated with IL-23 in the presence of test compounds. Serial dilutions of test peptides and IL-23 (Humanzyme #HZ-1261) at a final concentration of 0.5 nM, were added to each well in a 96 well tissue culture plate (Corning #CLS3894). DB cells (ATCC #CRL-2289), cultured in RPMI-1640 medium (Thermo Scientific #11875093) supplemented with 10% FBS, were added at 5×10E5 cells/well and incubated for 30 minutes at 37° C. in a 5% CO₂ humidified incubator. Changes in phospho-STAT3 levels in the cell lysates were detected using the Cisbio HTRF pSTAT3 (Tyr705) Cellular Assay Kit (Cisbio #62AT3PEH), according to manufacturer's Two Plate Assay protocol. IC₅₀ values were determined from these data. IC₅₀ data for exemplary compounds are shown in Table 4.

TABLE 4
IL-23 Data

| Compound | IC₅₀ (nM) |
|---|---|
| 188 | 0.0507 |
| 190 | 0.064 |
| 191 | 0.042 |
| 195 | 0.0709 |

PBMC pSTAT3 Assay

Cryopreserved peripheral blood mononuclear cells (PBMCs) from healthy donors were thawed and washed twice in ImmunoCult-XF T cell expansion medium (XF-TCEM) supplemented with CTL anti-aggregate wash. The cells were counted, resuspended at 2×105 cells per mL XF-TCEM supplemented with penicillin/streptomycin and 100 ng/ml IL-1 (BioLegend, 579404), and cultured in tissue culture flasks coated with anti-CD3 (eBioscience, 16-0037-85 or BD Pharmingen, 555329) at 37° C. in 5% CO2. On day 4 of culture, PBMCs were collected, washed twice in RPMI-1640 supplemented with 0.1% BSA (RPMI-BSA), and incubated in RPMI-BSA in upright tissue culture flasks for 4 hours at 37° C. in 5% CO2. Following this ‘starvation,’ a total of 6×104 cells in 30 μL RPMI-BSA was transferred into each well of a 384-well plate pre-spotted with peptide or DMSO. The cells were incubated for 30 minutes prior to the addition of IL-23 at a final concentration of 5 ng/ml. The cells were stimulated with cytokine for 30 minutes at 370C in 5% CO2, transferred onto ice for 10 minutes, and lysed. Cell lysates were stored at −80° C. until phosphorylated STAT3 was measured using the phospho-STAT panel kit (Meso Scale Discovery, K15202D). Results are provided below.

| Compound/ | | PBMC pSTAT3 |
|---|---|---|
| Example | SEQ ID NO: | IC50 (nM) |
| 345 | 345 | 0.0076 |
| 477 | 477 | 0.0055 |
| 478 | 478 | 0.015 |

Although the foregoing invention has been described in some detail by way of illustration and Example for purposes of clarity of understanding, one of skill in the art will appreciate that certain changes and modifications may be practiced upon review of the specification and within the scope of the appended claims. The full scope of the invention should be determined by reference to the claims, along with their full scope of equivalents, and the specification, along with such variations. In addition, each reference provided herein is incorporated by reference in its entirety to the same extent as if each reference was individually incorporated by reference. Where a conflict exists between the instant application and a reference provided herein, the instant application shall dominate.

LENGTHY TABLES
The patent application contains a lengthy table section. A copy of the table is available in electronic form from the USPTO web site (https://seqdata.uspto.gov/?pageRequest=docDetail&DocID=US20240409598A1). An electronic copy of the table will also be available from the USPTO upon request and payment of the fee set forth in 37 CFR 1.19(b)(3).

What is claimed is:

1. A peptide inhibitor of an interleukin-23 receptor of Formula (I), comprising an amino acid sequence:
R1-X3-X4-X5-X6-X7-X8-X9-X10-X11-X12-X13-X14-X15-X16-R2
wherein:
R1 is hydrogen, CH₃C(O)—, EtC(O)—, MeSO2, AzCO, BHCO, FPrpTriazoleMeCO, SMSBCO, Biotin, BiotinPEG2PEG2CO, DAGSuc;
X3 is dR, dK, PEG6, gEPEG6, R, K, or absent;
X4 is Pen, aMeC, hC, or C;
X5 is A, N, Q, N-MeAsn, L, Asn(4C13_2N15), I, K (PEG2PEG2Biotin);
X6 is T, MeThr, V, K, Dbu, Dpr, A;
X7 is W7Me, W, W(4F7Me), 7MeW, 7PhW, 7EtW, 7FW, 7ClW, 5BrW, 7(3NAcPh)W′
X8 is KAc, Q, NMeGln, A, Cit, dK(Ac), dQ, dNMeGln, dA, or dCit;
X9 is Pen, aMeC, hC, or C;
X10 is F4OMe, AEF, F, F4Me, F4Ad, Nal, AEF(Boc), 4PipPhe, AEF(Ac), Y, 4OMeF, 4AmF, D(Pip), Tzl(mPEG3), 3FTyr, Y(OTzl), Y(OTzl(mPEG3)), Tzl, Tzl(PEG3OH);
X11 is Nal, Quin_3, Coumarin(7OMe), 2Nal, 3Quin;
X12 is aMeK, THP, Spiral_Pip_Ac, Spiral_Pip, MeK, aMeLeu, aMeL, aMeK(Boc)
X13 is KAc, K, dK(Ac), or dK;
X14 is A, N, L, N-MeAsn, MeLeu, Asn(4C13_2N15), I;
X15 is 3Pya, bAla, Thiozolidine, H, dL, N, A, F, aMePhe, Aib, dK, h, 3MeH, 1MeH, tetraFPhe, bMePhe(SR), 5PyrimidAla, v, dR, homoF, Y, y, F(CF3), Y(CHF2), THP, or absent;
X16 is MeGly, dMeGly, dL, MeLeu, dMeLeu, N-MeNle, dN-MeNle, y, paf, maf, d3Pya, bAla, dbAla, P, dP, N(3AmBenzyl)Gly, N(4AmBenzyl)Gly, 4(R)HydroxyPro, 4(S)AminoPro, 5(R)diMePro, or absent
R2 is —OH, —NH₂, —HN(C₁-C₄ alkyl), —N(C₁-C₄ alkyl)₂, MeNH, CONHMe; and
wherein the inhibitor of an interleukin-23 receptor is cyclized by a disulfide bond between penicillamine, cysteine, homocysteine, or alpha methylcysteine residues at positions X4 and X9.

2. A peptide inhibitor of an interleukin-23 receptor of Formula (II), comprising an amino acid sequence:
R1-X3-Abu-X5-T-X7-X8-X9-AEF-X11-X12-X13-X14-X15-X16-X17-R2 (II)
wherein:
R1 is hydrogen, or CH₃C(O)—;
X3 is dR, R, or absent;
X4 is Abu;
X5 is Q, N, or T;
X6 is T;
X7 is W or 7MeW;
X8 is Q, K, KAc, dQ, dK, or dK(Ac);
X9 is Pen, C, hC, or aMeC;
X10 is AEF;
X11 is 2Nal, or Nal;
X12 is THP, Acvc, or Achx;
X13 is E, KAc, aMeE, Q, AIB, Achx, aMedE, dE, dK(Ac), or dQ;
X14 is N or S;
X15 is H, bAla, N, 3Pya, F, aMeF, aMeW, 1Nal, 4AmPhe, 2Nal, aMeFPhe, aMePhe, 3,4diFPhe, DY02, 5FW, or absent;
X16 is MeGly, AIB, or absent;
X17 is aMeK or absent;
R2 is —OH, —NH₂, —HN(C₁-C₄ alkyl), —N(C₁-C₄ alkyl)₂; and
wherein the inhibitor of an interleukin-23 receptor is cyclized by a thioether bond between the Abu residue at X4 and a cysteine, homocysteine, or alpha methylcysteine residue at X9.

3. A peptide inhibitor of an interleukin-23 receptor of Formula (III), comprising an amino acid sequence:
R1-X3-X4-X5-X6-X7-X8-X9-X10-X11-X12-X13-X14-X15-X16-R2 (III)
wherein:
R1 is hydrogen, CH₃C(O)—, FPrpTriazoleMeCO, NH₂, EtCO, AzCO, or BHCO;
X3 is dR, R, K, or dK;
X4 is Pen, Abu, AIB, aMeC, C, hC, Ala, 4RAminoPro, or 4SAminoPro;
X5 is N, D, or E;
X6 is T, Hyp, or 3OHPro;
X7 is 7MeW, W, 3Pya, A, 7PyrW, or 7(3NAcPh)W;
X8 is KAc, or dKAc;
X9 is Pen, C, S5H, AIB, D, E, hC, aMeC;
X10 is AEF, AEF(EtCO), AEF(BH), AEF(Ac), bMeAEF(2S3R*), bMeAEF(2S3S*), Y, or A;
X11 is 2Nal, A, Nal, or W;
X12 is THP;
X13 is E, KAc, S5H, dE, dKAc, or R5H;
X14 is N, S, 3Pya;
X15 is 3Pya, H, bAla, v, dR, hF, PAF, F, THP, 1, 4Pya, oAMPhe, 3MeH, D3Pya, N, 5MePyridinAla, 5AmPyridinAla, 3QuinolAla, 6OH3Pya, A
X16 is MeGly;
R2 is —NH₂—HN(C₁-C₄ alkyl), —N(C₁-C₄ alkyl) 2 or —OH; and
wherein the inhibitor of an interleukin-23 receptor is cyclized by a disulfide bond between penicillamine, cysteine, homocysteine, or alpha methylcysteine residues at positions X4 and X9; or
wherein the inhibitor of an interleukin-23 receptor is cyclized by a thioether bond between the Abu residue at X4 and a cysteine, homocysteine, or alpha methylcysteine residue at X9;
or
wherein when X4 is 4RAminoPro or 4SAminoPro and X9 is E or D, the inhibitor of an interleukin-23 receptor is cyclized by an amide bond between X4 and X9;
or
wherein when X5 is D or E, and X10 comprises an AEF residue, the inhibitor of an interleukin-23 receptor is cyclized by an amide bond between X5 and X10;
or
wherein when X9 and X13 comprise S5H residues the inhibitor of an interleukin-23 receptor is cyclized by an aliphatic linkage between X9 and X13.

4. A peptide inhibitor of an interleukin-23 receptor of Formula IV, comprising an amino acid sequence:
R1-X3-X4-X5-X6-X7-X8-X9-X10-X11-X12-X13-X14-X15-X16-R2 (IV)
wherein:
R1 is hydrogen, CH₃C(O)—, Ac_Morph, or MorphCO;
X3 is K(AcMorp), Kmorp, dK(AcMorp), or absent;
X4 is Pen, C, hC, or aMeC;
X5 is L, N, or nLeu;
X6 is T or L;
X7 is W or 7MeW;
X8 is KAc, K(AcMorph), K(IsoButyl_Ac), K(Butyl_Ac), K(Benzyl_Ac), KMorph, K, dKAc, dK(AcMorph), dK(IsoButyl_Ac), dK(Butyl_Ac), dK(Benzyl_Ac), dKMorph, or dK;
X9 is Pen, C, hC, or aMeC;
X10 is F4OMe, F, AEF, F4Ad, L, F4CN, or 4OMeF;
X11 is 2Nal or Nal;
X12 is L, THP, Spiral_Pip, aMeK, or aMeL;
X13 is L, dL, or nL (i.e., norleucine);
X14 is N or L;
X15 is 3Pya or absent;
X16 is MeGly or absent;
R2 is NH (2-(pyridin3-1)ethyl), —NH₂, —HN(C₁-C₄ alkyl), —N(C₁-C₄ alkyl)₂, or —OH; and
wherein the inhibitor of an interleukin-23 receptor is cyclized by a disulfide bond between penicillamine, cysteine, homocysteine, or alpha methylcysteine residues at positions X4 and X9.

5. A peptide inhibitor of an interleukin-23 receptor of Formula V, comprising an amino acid sequence:
R1-X3-X4-X5-X6-X7-X8-X9-X10-X11-THP-X13-X14-X15-R2 (V)
wherein:
R1 is hydrogen, or CH₃C(O), Propionic_acid, EtCO, PentCO, AzCO, MeSO2, NH₂, BHCO, FPrpTriazoleMeCO, (SulfoCy3), (SulfoCy3dPEG2), (SulfoCy3dPEG3), or SMSBCO;
X3 is dR, R, or absent;
X4 is Abu, Pen, C, hC, aMeC, aG, or Dpr;
X5 is Q or N;
X6 is T;
X7 is W, W7Me, 7MeW, bMeW (2S3R), bMeW (2S3S), 7FW, 7ClW, 5BrW, or 5MeW;
X8 is Q, K, KAc, Q, dK, or dKAc;
X9 is C, Pen, hC, aMeC, aG, E, or D;
X10 is AEF, F4OMe, F4Ad, Phe(4(2(Ac)aminoethoxy)), ac, LY02, AEF(Boc), 4PipPhe, AEF(BH), or AEF(SMSB);
X11 is 2Nal or Nal;
X12 is THP;
X13 is E, KAc, K, Q, aMeE, AIB, dE, dKAc, dK, dQ, aMedE, or Achx;
X14 is N;
X15 is H, bAla, N, F, aMePhe, aMeF, aMeW, 1Nal, 4AmPhe, 2Nal, aMeFPhe, 3,4diFPhe, DY02, 5FW, D(NBzl), D(NPh), D(NoAn), D(NPip), D(NPyr), D(NpAn), D(NmAn), D(N4Pyz), D(N5In), D(NPrAm), dH, D(NEtNH2), 3MeH, 1MeH, tetraFPhe, bMePhe(SR), 5PyrimidAla, 3OHPhe, 4PyridinAla, 3Pya, 4TriazolAla, bMePhe(2S3S), 2AmTyr, bMeH(2S3S*), or 5MeH;
R2 is —NH₂, —OH, —HN(C₁-C₄ alkyl), —N(C₁-C₄ alkyl)₂, or CONHMe and
wherein the inhibitor of an interleukin-23 receptor is cyclized by a disulfide bond between penicillamine, cysteine, homocysteine, or alpha methylcysteine residues at positions X4 and X9; or
wherein the inhibitor of an interleukin-23 receptor is cyclized by a thioether bond between the Abu residue at X4 and a cysteine, homocysteine, or alpha methylcysteine residue at X9;
or
wherein when X4 is Dpr and X9 is E or D, the inhibitor of an interleukin-23 receptor is cyclized by an amide bond between X4 and X9; or
wherein when X4 and X9 are aG, the inhibitor of an interleukin-23 receptor is cyclized by an aliphatic bond (generated from a Ring Closing Metathesis “RCM” reaction) between X4 and X9.

6. A peptide inhibitor of an interleukin-23 receptor of Formula VI, comprising an amino acid sequence:
R1-X4-X5-X6-X7-X8-X9-X10-X11-X12-X13-X14-X15-X16-R2 (VI)
wherein:
R1 is hydrogen, or CH₃C(O);
X4 is Pen, Abu, C, hC, dPen, dC, or aMeC;
X5 is L, N, Q, T, dN or absent;
X6 is T, L, dT, or absent;
X7 is W7Me, W(4F7Me), 7PhW, 7MeW, 7EtW, W, 7BrW, 7(2ClPh)W, 7(4CF3Ph)W, 7(3CF3TAZP)W, 7(4NAcPh)W, 7(3NAcPh)W, 7(40CF3Ph)W, 7(40MePh)W, 7(4Paz)W, 7(7Imzpy)W, 7(6(1)7dMeNDAZ))W, 7(3UrPh)W, 7(5(Ina7Pyr))W, 7(4(CpCNPh))W, 7(6(2MeNDAZ))W, BT, D7MeW;
X8 is KAc, Q, K(Gly), dKAc, dQ, or dK(Gly);
X9 is Pen, C, hC, aMeC, or dPen;
X10 is AEF, F4Ad, F4OMe, F4Me, Nal, F, Spiral_Pip, L, 4AmF, AEF(G), dY, or Y;
X11 is Nal, 3Quin, 2Nal, 2Quin, d2Nal, or W;
X12 is THP, aMeLeu, Acvc, aMeK, or Acpx, A;
X13 is E or dE;
X14 is N, L, or dN;
X15 is 3Pya, THP, N, H, dK, dL, dPaf, PAF, 3MeH, 3pya, or F;
X16 is MeGly, dK, K, or absent; and
R2 is —NH₂, —OH, —HN(C₁-C₄ alkyl), —N(C₁-C₄ alkyl)₂, or CONHMe; and
wherein the inhibitor of an interleukin-23 receptor is cyclized by a disulfide bond between a Pen, C, hC, dPen, dC, or aMeC at X4 and a Pen, C, hC, aMeC, or dPen residue at X9; or
wherein the inhibitor of an interleukin-23 receptor is cyclized by a thioether bond between the Abu residue at X4 and a Pen, C, hC, or aMeC residue at X9.

7. A compound, or a pharmaceutically acceptable salt thereof, having a structure of a compound as set forth in Table 1A, Table 1B, Table 1C, Table 1D, Table 1E, Table 1F, Table 1G, Table 1H, Table 1I, An inhibitor of an interleukin-23 receptor which is selected from compound 345, 469, 477 or 478 as shown below:

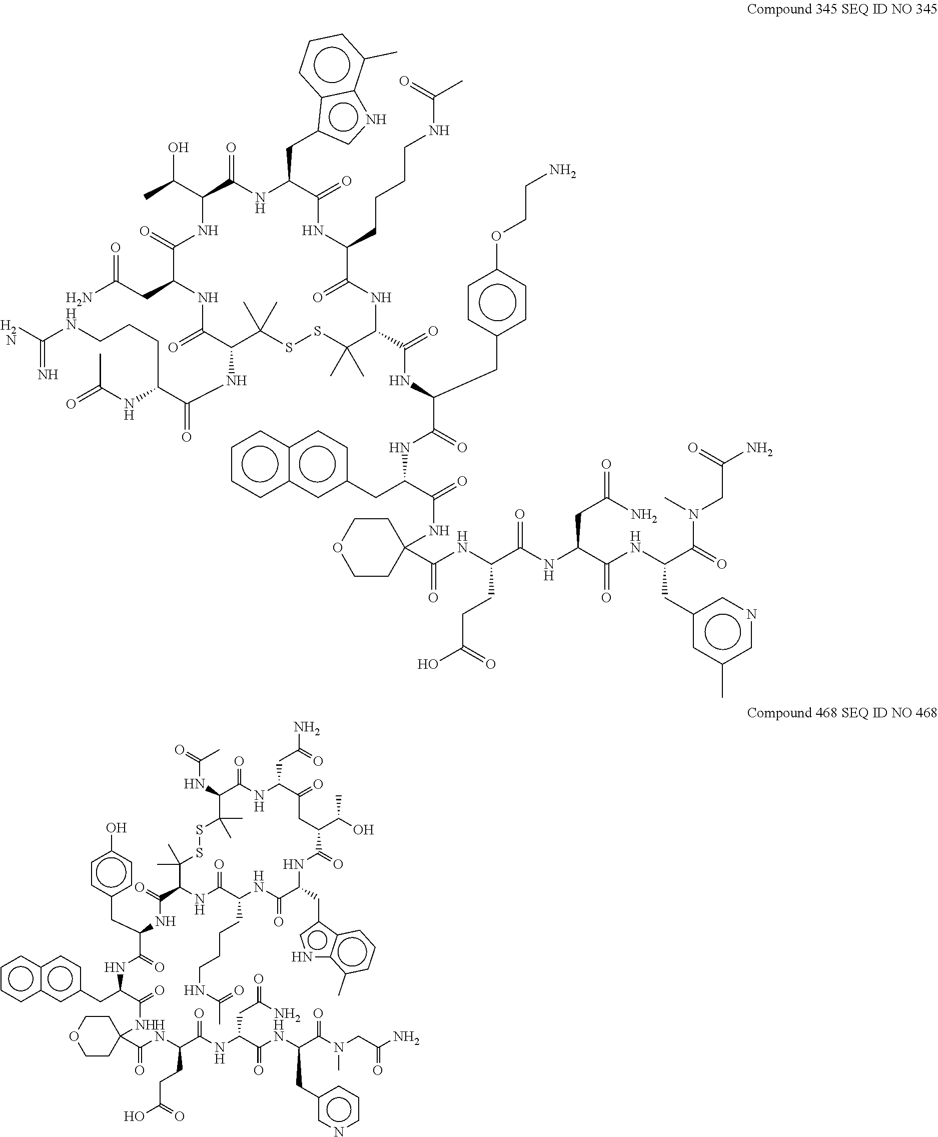

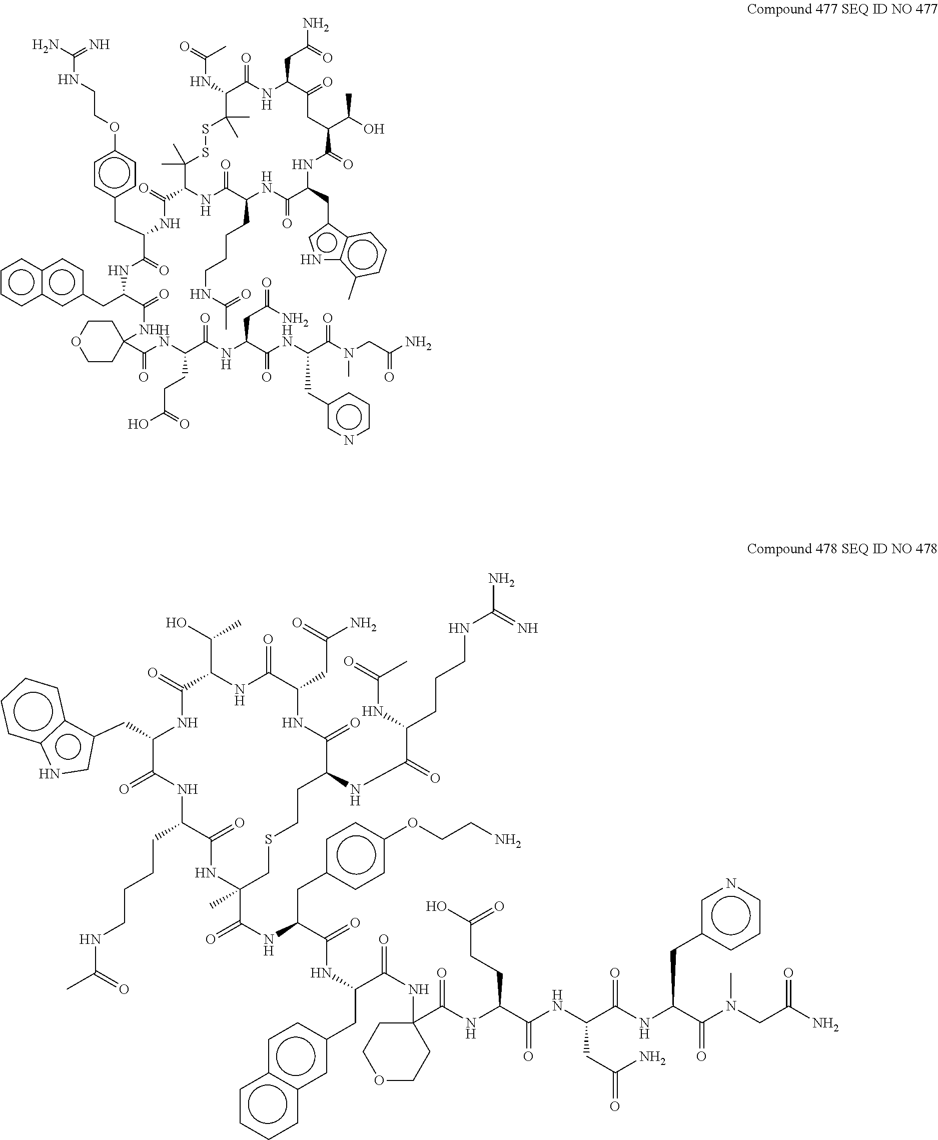

8. The peptide inhibitor of an interleukin-23 receptor of any of claims 1-8, wherein D amino acids are present or substituted for a corresponding L amino acid only at
(i) one or more of positions X3, X5, X6, X8 and X13, and optionally one of positions X1-X2, X4, X7, X9 to X12, X14-X18 present in the inhibitor; or
(ii) one or more of positions X3, X8 and X13, and optionally at one of positions X1-X2, X4-X7, X9 to X12, X14-X18 present in the inhibitor.

9. The peptide inhibitor of an interleukin-23 receptor of any of claims 1-8, wherein D amino acids are present or are substituted for a corresponding L amino acid only at
(i) X3, and optionally at one of positions X1-X2, X4-X18 present in the inhibitor; or
(ii) one of positions X3, and X8, and optionally one of positions X1-X2, X4-X7, X9-X18 present in the inhibitor.

10. The peptide inhibitor of an interleukin-23 receptor of any of claims 1-8, wherein the inhibitor comprises amino acids of the D-isomeric form, or is substituted with a D amino acid in place of the corresponding L amino acid, at one to six of positions X1 to X18 appearing in the inhibitor.

11. The peptide inhibitor of an interleukin-23 receptor of claim 11, wherein the inhibitor comprises amino acids of the D-isomeric form, or is substituted with a D amino acid in place of the corresponding L amino acid at one or two of positions X1 to X18 appearing in the inhibitor.

12. The peptide inhibitor of an interleukin-23 receptor of claim 11, wherein the inhibitor comprises amino acids of the D-isomeric form, or is substituted with a D amino acid in place of the corresponding L amino acid at three or four of positions X1 to X18 appearing in the inhibitor.

13. The peptide inhibitor of an interleukin-23 receptor of claim 11, wherein the inhibitor comprises amino acids of the D-isomeric form, or is substituted with a D amino acid in place of the corresponding L amino acid at five or six of positions X1 to X18 appearing in the inhibitor.

14. A pharmaceutical composition comprising:
(i) a peptide inhibitor of an interleukin-23 receptor or pharmaceutically acceptable salt, solvate, or form thereof according to any of claims 1 to 14, and
(ii) a pharmaceutically acceptable carrier, excipient, or diluent.

15. A pharmaceutical composition comprising:
(i) a peptide inhibitor of an interleukin-23 receptor or pharmaceutically acceptable salt, solvate, or form thereof according to claim 7, and
(ii) a pharmaceutically acceptable carrier, excipient, or diluent.

16. A pharmaceutical composition comprising:
(i) a peptide inhibitor of an interleukin-23 receptor or pharmaceutically acceptable salt, solvate, or form thereof according to claim 8, and
(ii) a pharmaceutically acceptable carrier, excipient, or diluent.

17. The use of a peptide inhibitor of an interleukin-23 receptor according to any of claims 1 to 14, or a pharmaceutical composition according to any of claims 15 to 17, for the preparation of a medicament for the treatment of an inflammatory, autoimmune inflammation diseases and/or related disorders.

18. The use according to claim 18, for the preparation of a medicament for the treatment of associated with inflammatory, autoimmune inflammation diseases and/or related disorders is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

19. The use according to claim 18 for the preparation of a medicament for the treatment of a disease or disorder which is selected from Inflammatory Bowel Disease (IBD), Ulcerative colitis (UC), Crohn's Disease (CD), psoriasis (PsO) or psoriatic arthritis (PsA).

20. A method for treating a disease or disorder associated with Interleukin 23 (IL-23)/Interleukin 23 Receptor (IL-23R), which comprises administering:
(i) an effective amount of a peptide inhibitor of an interleukin-23 receptor, or a pharmaceutically acceptable salt, solvate, or form thereof according to any of claims 1 to 14; or
(ii) a pharmaceutical composition according to any of claims 15 to 17, respectively to a patient in need thereof.

21. The method according to claim 21, wherein the disease or disorder is associated with inflammatory, autoimmune inflammation diseases and/or related disorders.

22. The method according to claim 22, wherein the disease or disorder associated with inflammatory, autoimmune inflammation diseases and/or related disorders is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

23. The method of claim 22, wherein the disease or disorder is associated with and autoimmune disease is selected from Ulcerative colitis (UC), Crohn's Disease (CD), psoriasis (PsO), or psoriatic arthritis (PsA).
